SEQUENCE LISTING (729 sequences)

SEQ ID NO: 1 - DNA, 4317 bp, Nicotiana tabacum
  source 1..4317: mol_type = genomic DNA
agtctcagacttaatccagtatatcccatcttatctcacattatcccatcaaatgtgagattattttatctcatctctcatgtggtataaattagtcatgaaattataatctcgggataatttagtccgcgtaccaaacgaccccaagtgctttattgttttcttattgacagagtaagtgtatgggaacttaccatagaaatccttcgctcaaaaggaaatacttgcaagaactggccaaacccaaagatgaataaactcaattacttgattcttaaactcttaaaaatgaattcaatggagaaggaaaatatttccagtgtaaacacaagtgaatgaagagaagccaaaataatctctatcattcaagccttaggtggagattaaaaaaattatttactttcttatcaaagtaataggtgatcaacagctttcgtaaaacgtcattaggagaatattataatctcttttatgctgaagaacccacataaggaagatcataaaatacatgactttcagatgacttcttggagctttatttttaaagagtggctagctggtcagcaaagaggtgctcgtcagatatcataaaattttactattatttgttttaagagggagatggggcacacatgcttgtgacaaaagtaagaggaagaaaggagacagaagaggaaatagatttggggggggggggggggtttcacaatcaaagaaaatttttaaaatggagagagaaatgagcacacacatatactaacaaaattttactaataattgcaccgagacaaacttatattttagttccaaaatgtcagtctaaccctgcacgttgtaatgaatttttaactattatattatatcgagttgcgccctccactcctcggtgtccaaattgtatttaaatgcatagatgtttattgggagtgtacagcaagctttcggaaaatacaaaccataatactttctcttcttcaatttgtttagtttaattttgaaaatggaagtcatatctaccaacacaaatggctctaccatcttcaagaatggtgccattcccatgaacggccaccaaaatggcacttctgaacacctcaacggctaccagaatggcacttccaaacaccaaaacgggcaccagaatggcactttcgaacatcggaacggccaccagaatgggacatccgaacaacagaacgggacaatcagccatgacaatggcaacgagctactgggaagctccgactctattaagcctggctggttttcagagtttagcgcattatggccaggttagtactaagaaagcaactcaaatgcatcggcctcttgttgctactaaatatagagagctatcatacttttagggactaactaaaaaggaaagattatcacagggacgaagtgagcagttaacttcgcatattatcagacgcattaatttgaaataatcgaattttgcaggtgaagcattctcacttaaggttgagaagttactattccaggggaagtctgattaccaagatgtcatgctctttgaggtaattaatattctaatacacatgctttaatttaaagtgatacttttaatttacttttagtttattgcatgtgcacgtacagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtaagtcaaacttcttttacccacataaagaaaatgatttagattgcaattctttttatttttctaaaagaataaatatattctctttttttttttaaaacaaaattctctttcttacaggtatccagaaaatttttcccttatctggcagctaattttaacgatcctcgtgtaaccctagttctcggagatggtgcgtatatgatagtctcgttttatattttatttcacttgatttttacctttttttgtggttaattaatcatctaccattggttctctttaccttcaggagctgcatttgtaaaggctgcacaagcgggatattatgatgctattatagtggactcttctgatcccattggtacgctattactatttaataccaagactattcttattaaataagctactaagaaactaattgaataattaataaacgtaactgtaattgatttctaaaataatatatataatttcaggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccacgtattctttttctctctctctcttcctgtctttttcgatgcaatgtaaatttataaaattggaagtccgttttacttttctatagacgtagatcctaaaattgtcaagaaatggagaattgacttacaagaaaaatcaacttcttttcatttactattctttttggtgacaaactttacttattatttcgttctaaaatgaaaatttatttttatattttaaaataatttagctttaaacttttaattttacttgttatatttttaataaaaaagatttatagtcaaataaatgttgtgaccatataaaaacctccgcatttttaagatcataagtttcagagtcaaacgagttaatttatttttagtatgccggtgcggagtcaaattatgtcataaaaattgaaacggagtgagaacatttttatttcgagtaaactttcaaggtattgtgtttaatttcaagtgatactgatcaatgatgtcttaaatattttgatttcagcggtgtgatcggttatatgctctgctctactgaagggccagaagttgacttcaagaatccagtaaatccaattgacaaagagacaactcaagtcaagtccaaattaggacctctcaagttctacaactctgatgtaacttcatatctcacaatttctttttccgttttactgtatgttcttcgtcaaattttataactaactcttttcatattgtcttttttttcagattcacaaagcagcattcattttaccatctttcgccagaagtatgatcgagtcttaatcaagtgaataatgaacactggtagtacaatcattggaccaagatcgagtcttaatcaagtgaataaataagtgaaatgcgacgtattgtaggagaattctgcagtaattatcataatttccaattcacaatcattgtaaaattctttctctgtggtgtttcgtactttaatataaattttcctgctgaagttttgaatcgacgtttcaactcaatcctcgcaaatcagttcattaccctctttcagtgtactatagtaaacaaatctcatagttaccgtggtgttgttttttgactatgagattttcgatctttattaaggttttggtctatgttttggaattgtattgaatatatttgtaacttactatgtatctaaatcggctagtctctcttttttaataaatggcgtcattgccgttgtagtttgtttactcggtactattttatcagcagtttcgatttgtttgctcattttttagaagagggttgaaatgtctttgttgtaagataactttaccatatcttatagattagtatagaatgacccgttctgagccggggccaaacttatattattttataaaaagaatggataaatttctttgatatgtatcagtttattgttattaattagagatatagtttgagaagctcaatgcgatgaaagagagtaacatcgctatattaaactaatcacaaaatagcactttgaataagtcttcggatcccatgttgtagtaggacaaacaataactttgtttctatttatttatcgttctattttcacccatcaaacaaaaagtaaatttgtttctatttagtttgaatagtcgcgtagaaaattcggtagatagcagaacaaagaaaataaattcaagccaaaaatagatcatatttactgttgttctttcagcaactactattttgcatcagttacaagaccaatatgctttaccgtataacaaaaatataaatgtatatgccagtaaattacaga

SEQ ID NO: 2 - DNA, 4210 bp, Nicotiana tabacum
  source 1..4210: mol_type = genomic DNA
gaattttgtaaatgctttttttggctcagtgattttgtggagttgggaaaaatcctattggaaagtaggtcgtggttttttcaccttttgaaccaggtatttttcatgtaaaaatacttgtgttctttactttttgcatttactattccacaactgtagtgtaaggaacacgtagaagaaccatgtcctataatctgtgcacgcgaaaaattggacaccacgcaaatcacccttattgtgtgtcattgaaatataaaacatcaagctcaatcatagattaatctttttttagtaccaacatcatatgcaaaaatcaattcaacccccaaaacataatacaaccaatgttaatgcaatatctctgctgctatcacgaaaataattgtagctcacgaaagtaggatacattatgtaggttacatcacatagaggtaatctaaagctcccaataataagatgtgtaatgttgattatgtagaaatttgccaggttatttagaataaacaagaagaggagaaaaaaagtacaatttacctgaactcttgaatgtatcctacaaataacctagacttcatggacgtcagttgtcagtttacttttgttttaatggtacatcatttgtcaaatactttatttggataaaaacagttttgcctaaggagtaaacagatccggagtaagaaagcagacgattaaagcaatttttaaaaaaggagagagaaattaatgagcacacacatatactagtgaaattagggtactaatttactaataattgcaccgagacaaacttatattttagttccaaaatgtcagtctaaccctgcacgttgtaataaatttttaactctattatattatatcgagttgcgccctccactcctcggtgtccaaattgtatttaaatgcatagatgtttaatgggagtgtacagcaagctttcggaaaatacaaaccataatactttctcttcttcaatttgtttagtttaattttgaaaatggaagtcatatctaccaacacaaatggctctaccatcttcaagaatggtgccattcccatgaacggccaccaaaatggcacttctgaacacctcaacggctaccagaatggcacttccaaacaccaaaacgggcaccagaatggcactttcgaacatcggaacggccaccagaatgggacatccgaacaacagaacgggacaatcagccatgacaatggcaacgagctactgggaagctccgactctattaagcctggctggttttcagagtttagcgcattatggccaggttagtattaagaaagaaactcaaatgcatcgtactcttgtattttggtttgtgtataatttataatatggataaattatgacgaagtaaactcgcatacattaatttgaaataatctaattttgcaggtgaagcattctcacttaaggttgagaagttactattccaggggaagtctgattaccaagatgtcatgctctttgaggtaattaatattctgatacacatgctttaatctaaagtgatacttttaatttacttttagtttattgcatgtgcacgtacagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtaagtcaaacttcttttacccacataaagaaaatgatttagattgcaattctttttatttttctaaaagaataaatatattctctctttttttttttaaaacaaaattctctttcttacaggtatccagaaaatttttcccttatctggcagctaattttaacgatcctcgtgtaaccctagttctcggagatggtgcgtatatgatagtctcgttttatattttatttcacttgatttttacctttttttgtggttaattaatcatctaccattggttctctttaccttcaggagctgcatttgtaaaggctgcacaagcgggatattatgatgctattatagtggactcttctgatcccattggtacgctattactatttaataccaagactattcttattatataagctactaagaaactaattgaataattaataaacgtaactgtaattgatttctaaaataatatatataatttcaggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccacgtattctttttctctctctcttcctgtctttttcgatgcaatgtaaatttataaaattggaagtccgttttacttttctatagacgtagatcctaaaattgtcaagaaatggagaattgacttacaagaaaaatcaacttcttttcatttactattctttttggtgacaaactttacttattatttcgttctaaaatgaaaatttatttttatattttaaaataatttagctttaaacttttaattttacttgttatatttttaataaaaaagatttattgtcaaataaatgttgtggccatacaataagtttcaaattatgtcacaaaaattgaaacagagtgagcaaatttttatttcaagtaaactttcaaggaattgtgtttaagttttctcaactgatactgatcaatgatgtcttaaatattttgatttcagcggtgtgatcggttatatgctctgctctactgaagggccagaagttgacttcaagaatccagtaaatccaattgacaaagagacaactcaagtcaagtccaaattaggacctctcaagttctacaactctgatgtaacttcatatctcacaatttctttttccgttttactgtatgttcttcatcaaattttataactaactcttttcatattgtctttttttttcagattcacaaagcagcattcattttaccatctttcgccagaagtatgatcgagtcttaatcaagtgaataatgaacactggtcgtacaatcattggaccaagatcaagtcttaatcaagtgaataaataagtgaaatgcaacgtattgtatgagaattctgcagtaattatcataatttccaattcactaattgttgtaaaattctttctctgtggtgtttggtactttaatataaattttcctgctgaagttttaaaatggacgttccaattcgatcctcgcaaatcagttcattatccttctttcagtgtactaaagccaacaaatctcatagttaccgtggtgttgtttttactatgggatttgcgatcttattaaggttttggtctatgttttggaattgtaatattcatatttttaagttactctgtatctaaatcggcctagtctctcttttttaataaatggcgtcatcgtcgttgcagtttgtttgctcatttttcagaagtatgatggtttgaaatgtctttgtttctatggcctctttactgcagcttaccgatttgcttatgttggatattgcttctgattcatatgggatatgaagtttggtatttgttgaatgattaaggaaaaaatttacaacaataactatgcctcaatcccaacctaattatgatcagcgcttcaaaaaaaaaaaaaaagtagaactaaaatttttaagcttagatgcaaatccataacactgtaaaaaaaagtagggatttctgttcctttcttttatagttaagaaagaactctctgccttgtgaatcttattgcatctctctaattcagcaaaataattgaacaatgactaggataaaatatttcagctaagataaccctaattttctggatggaatcaccttctccaccaagttgttattttcttgcattgtgagcagactagttcaattacaccattagagttttttctcagcactatcctggcaatgatgcgctaagctctgaaaaccaattagccttaatagagttggagctccccaataactctt

SEQ ID NO: 3 - DNA, 3686 bp, Nicotiana tabacum
  source 1..3686: mol_type = genomic DNA
ttgtgagagatttggtgtcctctacaatgattgttgaagtccctatttatagctatacacaggaaacaaaatcctaggatcaagcccctcttaaatgacaataatggggttaatgatgaatatgtagcggcatgacatgaatgccaaaattctccgcaacgactatttatttaatattgaggaatattttttattaaatactatctggtgacaagcattcgtttgcttccgttgattacgttgattttgggatctactctataccaaccgaagccgttgtccttgatcttcgctttcatttaattcatcttccgtctgcctccgatttcacaagtcatgcacccattcaattatttaatggaaaccaattttaccctatacaaatggtacatcattcgtcaaatactttacttggatataaacaattttgcccgaggagtaaacagatgcgaagaaagaaagcagacgattaaagaaatttttaaaaaaggagagagaaatgaacacacacatgtactaataaaattagggtactactttactaataattggacagagactaaattcatattttagttccaaaatgtctcgggcagtccaaccatgcacgttgtaatgattttttaactctattatatcgagttgcgccctccactcctcggtgtccaaattgtatataaatgcatatgtgtctattgggagtgtacatcaagctttcataaagtacaaatcgtaatacttgttgaaacataatactttctcttctccaatttgtttagtttaattttgaaaatggaagtcatatctaccaacacaaatggctctaccatcttcaagagtggtgccattcccatgaatggccaccataatggcacttccaaacaccaaaacggccacaagaatgggacttccgaacaacagaacgggacaatcagccttgataatggcaacgagctactgggaaactccaattgtattaagcctggttggttttcagagtttagcgcattatggccaggttagtactgagaaagaaactcaaatgcatatttaaagttaaaattgttaggctaatataaggagttgatattcttttagtgattaattaaaaaggaaaaagtatcaaataaattcaaaaaatggatagtaacttcgcatattactctacacattaatttgaaataaatcgaattttgcaggtgaagcattctcacttaaggttgagaagttactgttccaggggaagtctgactaccaagatgtcatgctctttgaggtaaataatattttaatacacatgcttccatttaaattgatacttttaatttacttttactttattgcatgtgtacgtacagtcagcaacttatgggaaggttctgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgagatcgatgacgtggtagttgatgtaagtcaaacttcttttactcacataaaaaaatggtttagattgcttcttgttatttttctaaaagaatactatttttttaaaacaaaattttcttttttacaggtatctagaaaatttttcccttatctcgctgctaattttaacgatcctcgtgtaaccctagtccttggagatggtgcgtatttgataatctcgcttttgttttatcttttatttttattgcatttaatttttaccttttggtgtgtggttaattcacctgccattggttctctttcatttcaggggctgcatttgtaaaggctgcacaagcagaatattatgatgctattatagtggactcttctgatcccattggtactctattacttcttaataccaagactaatcttattgaataagctactaataaacggtaattgatttctaaaacaatataatttcaggtccagcaaaagatttgtttgagaggccattctttgaggcagtagctaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaacgtatttttctctctctctctcttcctataaaattggaagttttgattctataattgtcaagaaatggagaatcagttccaagaaaaaccaacttcttttcttttactcttcaaggtattgtgtttaattttttttcaactgatatgatcaattattttgatttcagcggtgtgattggttatatgctctgctctactgaaggaccagaaattgacttcaagaatccagtaaatccaattgacaaagagacagctcaagtcaagtccaaattagcacctctcaagttctacaactctgatgtaacttcatatctcacaatttcttttttcctattgtactttatgttcttcgtcaaattttataattaactcttttcaaattgtctttttttttttcagattcacaaagcagcattcattttgccatctttcgccagaagtatgatcgagtcttaatcaactgattaatgaatactggtggtacaatcattggaccaagatcaataagtgaaagacgtattgtatgagaattctgcagtaattaattatcataatttccaatttaccaattattgtaaaattctttctctgtggtgtttggtactttaatataaattttcctgcttaagttttgaatcgacgtatcaactcaatcctcgcaaatcacttcattaccctcctttcagtgtactaaagtaaactgttgcggaatatcgtgggttaactagaacacaatcacacacaaatcaaatagagaagaatttattaacggggttcggctaagcctaatcctcaggacaaaagcagagagagttttccactatgaatgagaagaaaaacacaatacaatatataaaatcctcaactacaacccctatatatgatcccaaaaggtcccaaacatatatgagaaaagtttcccaatttgacaaagattataggttttcctttcccaaatctattagggcaatgggttttcttaaacctatggggactatggctttcctaaaaatacaaggaaataattcaaaccacaaataataaatcttccccttggcttgaattctcttcatcaacaggaacaatagctctctaccttgccctcaaccctcgcaagggctctattgattgtcgcacacatcaaccaagtctaggcaacgcctaaacttgttatgagacttaatctcttcagccatagcactaatccgtcgatttggttctgtacccgaaatagcttttggatagctataacactgaaacacatcagtggtctctgcaactaccaaagcaaatcccacagtattcgtatatccaagaagatttccatcaactacgtttgcaaacctttgcggtcggttgatcactctcttctctctgtatgttgcaatgctatatggttgttgttgcgcaggtgcatcaacattatcgtcttgatcaatattttgtacctcctcta

SEQ ID NO: 4 - DNA, 4035 bp, Nicotiana tabacum
  source 1..4035: mol_type = genomic DNA
ggaagagtgtggtatgggagatgcctcccagggagtacctaaagctgaatactgatggaagttttaacaaacaaattgggaaagcagggattggagggattctcagagatgaagagggaggctttgtcatggctttttcgatgcctataatctataataacatcagtgaagcagaattgaaagccatcaagtatgggtgtgaatggtgcaaatacaaaggaatatcaaacttcattgtggaaactgactcgaggatgatctatgacatactacagaccaaaaatctaagcaacaacaagttgaaacaagagaccgagaaattaatggagattctggacacctgcaggacacctgttacccattgccttcgcgaagcaaatcaagtggcagactggtttgctaaagaggccaccagagctaacgaaggtatcactcatacagattttagacaggtatcaaaagcggccaagggccctttcttcatggatatgtggcaggtcccttattttagaattagatatgaaaaatctaatttttttttgtaagttaattctgtgtatagtgagaggaaatcgtctaatatgtatttttgcccatagactcttcctctccttaggtaaaaaggtagctccgaggtaaggtttatgttcccctcagtgtaacctttttttgtttatataatagacatggtatgggtccagctaaacccccaacaccacaggggatagatacctgggtgattggtttattttttaaaaaaaaaaactttactaataattgcacggagacaaaacttatattttagttccaaaatgacagtccaaccatgcacgttgtaatgattttttaactctattatatcgagttccgccctccactcctcggtgtccaaattgtatttaaatgcatagatatgtttattgggagtgtacatcaagctttcagaaaatacaaaccataatactttctcttctccaatttgcttagtttaatttggaaaatggaagtcatatctaccaacacaaatggctctactatcttcaagaatggtgccattcccatgaacggttaccagaatggcacttccaaacaccaaaacggccaccagaatggcacttccgaacatcggaacggccaccagaatgggatttccgaacaccaaaacggccaccagaatggcacttccgagcatcagaacggccatcagaatgggacaatcagccatgacaacggcaacgagctacagctactgggaagctccaactctattaagcctggttggttttcagagtttagcgcattatggccaggttagtactaagaaagaaactcaaatgcatcgtactcttgtattctgctttgcgtataatttagatgatggtgtttgactaagcactgagtttaaaaataaaaagtttaaagttaaattgttactatagagagctatatctttaggaactaactaaaaaggaaaaattatcacataaaattgggatgaagtaagcagttaacttcgcatattattcgacacattaatttgaaataaatcgaattttgcaggtgaagcattctcacttaaggttgagaagttactattccaggggaagtctgattaccaagatgtcatgctctttgaggtaattaattaatactaatagtcaagctcatgtatgattatatttaaagtggtatttttcgtttatttttaatttattgcacgtgtacgtacagtcagcaacatatgggaaggttctgactttggatggagcaattcaacacacagagaatggtggatttccatacactgaaatgattgttcatcttccacttggttccatcccaaaccctaaaaaggttttgatcatcggcggaggaattggttttacattattcgaaatgcttcgttatcctacaatcgaaaaaattgacattgttgagatcgatgacgtggtagttgatgtaagtcaaacttcttttactcacataaaaaaatgatttagattcttatttttctaaaagaattaaaacaaaattttccgttttacaggtatctagaaaatttttcccttatcttgctgctaattttagcgatcctcgtgtaaccctagtccttggagatggtgcgtatttgataatctcgtttttattttatcttttacttttattttatttaatttttacctttttgtgtgtggttaattcacctgccattggttctttttatttcaggggctgcatttgtaaaggccgcacaagcaggatattatgatgctattatagtggactcttctgatcccattggtactctattactacttaataccaagactattcttattaaataagctactaataaacgtaactctgatagttttctaaaataatataatttcaggtccagcaaaagacttgtttgagaggccattctttgaggcagtagccaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaacgtatttttctctctctcttcctataaaattggaagttttgattctataattgtcaagaaatggagaatcagttccaagaaaaaccaaattcttttcttttactcttcaaggtgtgtttaagttttttaaactgatactgatcaattattttgatttcagcggtgtgattggttatatgctctgttctactgaaggaccagaagttgacttcaagaatccagtaaatccaattgacaaagagacaactcaagtcaagtccaaattagcacctctcaagttctacaactctgatgtaacttcatatctcaatttcttttttcttattgtactttatgttcttagtcaaattttataattaactcttttcaaattgtctttttttttcagattcacaaagcagcattcattttgccatctttcgccagaagtatgatcgagtcttaatcaagtgactaatgaatactggcggtacaatcattggaccaagatcgagtcttaatcaagtgaataaataagtgaaatgcgacgtattgtataagaattctgcagtagttaattatcataatttccaattcaccaattactgtaaaattctttctctgtggtgtttggtactttactataaattttcccgcttaagttttgaatcgacgtttcaactcagtcctcgcaaatcacttcattacccttctttcggtatactaaagtaaactgttgcagaatatcgtgggttaactagaacacaatcacacacatagcaaatagagaagaaaaatcaacacaagaatttattaacgaggttcggctaagcctaatcctccgggcaaaagcagagagagtttttcactatgaatgagaagtaaaacacaatacaatctatagaatttccaactacaacccctatatatagatttcaaaaggtcccaaacatatatgagaaaggtttcccaatttgacaaggattataggttttcttttccaaaatctattagggtaatgggttttcctaaacctatgaggactatgggtttcctaaaaatacaaggaaataatttaaaccaaaaataacaaatcttccccttggtttgaattctcttcattaacaggaacaacagctctctacctttccctcagccctcgcaatggctctattgattgccgcactcatcaaccaagtctacgcaacgcctaaacttgttaagagacttaatctcttcagccatagcactaatttgtcgatttggttctgtacccgaaatagcttttggataactataacactcaaatgcatcagtggtctctgcaactaccaaagcaaatcccacaagattcgtatatccaagaagatttccatcaactacatttgcaaacctttgcggtcggttgatcactctctctctctctgcatgttgcaatgctatatggttgttgttgtgcatgtgca

SEQ ID NO: 5 - DNA, 4431 bp, Nicotiana tabacum
  source 1..4431: mol_type = genomic DNA
acggtacaattgaatttgttgcgtgacttgtagacaagtgaattgatttgtccaaaatgattaataaatcaaattaaaaataaggcttagcgttaaaatcaaaggaaatggcaagcctgactcccggagcaatgcttctgaggacagtagtaaaaacaatatcagacaaaaagtaaagttatattatttagcttgaggataaagtatgtcatcagttttgttagagatttggtgtcctctacaatgattgttgaagtccctatttatagctatacataggaaacaagatcctagaatcaaacccttcttaaatgacattaatgggagttattgatgaatatgtagcggcatgacatgaatgccaaaattctccgcaacggctatttacttaatattgaggaatatttttcattaaatactatctggtgacaagtattcgtttgcttccgttgattgcattgattttgggatctactatgtaccaaccgaagttgttgtccttgatcttcgctttcatttaattcatctttcgtcgacctctgattccacaagtcatgcacccattcaattatttaatggaaaccaattttaccctgtacaaatggtacaaatactttccttggataaaaacaattttgcctaaggagtaaacagatgcgaagtaagaaagcagacgactaaagaaaattttaaaaaaggagagagaaatgagcacacacacgtactaataaaattagggtactactttactaataattggacagagactaaattcatattttagttccaaaatgtctcgggcagtccaaccatgcacgttgtaatgagtttttaactctattatctcgagttgcgccctccactcctctgtgtccaagttgtatataaatgcatatatgtctattgggagtgtacagcgagctttcataaagtacaaatcataatacttgttgaaacataatactttctcttctccaatttgtttagtttaattttgaaaatggaagtcatatctaccaacacaaatggctcgaccatcttcaagaatggtgccattcccatgaatggccaccagagtggcacttccaaacacctcaacggctaccagaacggcacttccaaacaccaaaacggccaccataatggcacttccgaacatcggaacggccaccagaatgggatttccgaacaccaaaacggccaccagaatgggacttccgaacatcggaacggccaccagaatgggatttccgaacaccaaaacggccaccagaatgggacttccgaacaccaaaacggccaccagaatgggacttccgaacaacagaacgggacaatcagccatgacaatggcaacgagctactgggaaactccaactctattaagcttggttggttttcagagtttagcgcattatggccaggttagtactgagaaagaaactcaaattcatatttaaagttaaaattgttaggctaatataagaagttgattttcttttagtgattaattaaaaaaggaaagagtatcaaataaattccaaaaaatgaccagtaacttcgcatattattctacacattaatttgaaataaatcgaattttgcaggtgaagcattctcccttaaggttgagaagttactatttcaggggaagtctgactaccaagatgtcatgctctttgaggtaaataatattctaatacacatgctttaatatgaataaatacttttaatttacttttagtttattgcacgtgtacgtacagtcagcaacatatgggaaggttttgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgaaatcgatgacgtggtagttgatgtaagtcaaatttcttttactcacataaaaaaatgatttagattgcttctttttatttttctaaaagaataaatatattctctcttagttttaaacaaaattctctttcttacaggtatctagaaaatctttcccttatctcgcagctaattttaatgatcctcgtgtaaccctcgttctcggagatggtgcgtatttataatctcgtttttgttttatcttttatttttatttcatttaatttacctttttgtgtgtggttaatttacccgtcattggttctctttcatttcaggggctgcatttgtaaaggctgcacaagcaggatattatgatgctattatagtggactcttctgatcccattggtactctattactacttaataccaagactaatcttattgaataagctactaataaactgtaattgatttctaaaataatataatttcaggtccagcaaaagatttgtttgagaggccattctttgaggcagtagccaaagccctaaggccaggaggagttgtatgcacacaggccgaaagcatttggcttcatatgcatattattaagcaaatcattgctaactgtcgtcaagtctttaagggctctgtcaactacgcttggactactgttccaacatatcccacgtattttctctctctctctcttcatctttgaaaattgaaaatcctgactactttccttcctttgattcctcggttaaaggggcgtagatcataagattttcaagaaatagataatgacgtccaagaaaaactaacttcttttcatttactattctttttggtgacaaactttatttattatttcgttctaaagagaaaatttatttttatattttaaaataattttgttttaaacttttatttttacttattatatctttaataaaaaaattatagtcaaataaatattatggccacactaaacatccaagtttttgaaaccataagttttagagccaaatgagttaatttgtttttggtatgcgggtgcggagtcaaattatgtcacaaaaattgtaatggagtgagcaaatttttatttcgagtaaactttcaaggtattgtgttaaagttttttcaactgatactaatcaattatgtctcaaccattttgatttcagtggtgtaattgggtatatgctctgctctactgaagggccagaagttgacttcaagaatccaataaatccaattgacaaagagacaactcaagtcaagtccaaattagcacctctcaagttttacaattctgatgtaacttcatatctaacaatttctttttctgttttactgtatcttcattgtcaaaattttataattaactcttctcaaattatctttttttttagattcacaaagcagcattcattttgccatctttcgccagaagtatgatcgagtcttaatcaagtgaataatgaacactggtggtgcaatcattggaccaagatcgagtcttaatcaagtgaataaataagtgaaatgccgacgtattgtatgagaattctacagtaattaattatcataatttccaattcaccaattattgtaaaattctttctctgtggtgtttggtacttcaatataaattttcctgctgaagttttgaatcgatgttccaactcaatcctcgcaaatcagttccttatttttctttcagtgtacttagtaatcttagtgggatcggcgcttccataaaagaatgtaaacttaatattttttttaagcctaaatgcaaatccatcacactgtaaaacatattgatcttcagcctctgaagatgagtagggatttctgctcctttcatttacagtcgagaactttctgtcttgcatctctctaattcagccaaattattgataaccacgactttagttaaacatttgtttaactaaaacaactcccctaaccatggtaaaacacagtagttatcagtgtttaattaacctttcctttttggtaaatatagggtgaagtcttgctgcgaactttgttctcatatgttgaagatagtaatccaactcaagaaacaaacatgattgattaaatgttgtcgatgacacactttcaactgatccaaatggtaacgttccggccggctattttatgtatttgagtcccgttctcctacttgatattttccctatgcttgtttgacgttttgtgagttgtggagatggttggtttggttcgggaaggttttggaatgagtagagacgcttcaactcattttgaaagcttaagttgtacgagttgatcaaggtttgactttggtataaacgatatcaatttgatggtttcagtatgttcatatggtgattttggaattagacgcatgttcggatatgaatttggaggtttctagaatgttttgtctctgagtgccaaaagctgggaatttaaagg

SEQ ID NO: 6 - DNA, 1128 bp, Nicotiana tabacum
  source 1..1128: mol_type = genomic DNA
atggaagtcatatctaccaacacaaatggctctaccatcttcaagaatggtaccattcccatgaacggccaccaaaatggctcttccgaacacctcaacggctaccagaatggcatttccaaacaccaaaacgggcaccagaatggcacttccgaacatcggaacggccaccagaatgggacatccgaacaacagaacgggacaatcagccatgacaatggcaacgagctactgggaagctccaactctattaagcctggttggttttcagagtttagcgcattatggccaggtgaagcattctcacttaaggtcgagaagttactattccaggggaaatctgattaccaagatgtcatgctctttgagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtatccagaaaatttttcccttatctggcagctaattttaacgatcctcgtgtaaccctagttctcggagatggagctgcatttgtaaaggctgcacaagcgggatattatgatgctattatagtggactcttctgatcccattggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccaccggtgtgattggttatatgctctgctctactgaagggccagaagttaacttcaagaatccagtaaatccaattgacaaagagacaactcaagtcaagtccaaattaggacctctcaagttctacaactctgatattcacaaagcagcattcattttgccatctttcgcccgaagtatgatcgagtcttaa

SEQ ID NO: 7 - DNA, 1128 bp, Nicotiana tabacum
  source 1..1128: mol_type = genomic DNA
atggaagtcatatctaccaacacaaatggctctaccatcttcaagaatggtgccattcccatgaacggccaccaaaatggcacttctgaacacctcaacggctaccagaatggcacttccaaacaccaaaacgggcaccagaatggcactttcgaacatcggaacggccaccagaatgggacatccgaacaacagaacgggacaatcagccatgacaatggcaacgagctactgggaagctccgactctattaagcctggctggttttcagagtttagcgcattatggccaggtgaagcattctcacttaaggttgagaagttactattccaggggaagtctgattaccaagatgtcatgctctttgagtcagcaacttatgggaaggttctgactttggatggagcaattcaacatacagagaatggtggatttccatacactgaaatgattgttcatctaccacttggttccatcccaaacccaaaaaaggttttgatcatcggcggaggaattggttttacattattcgaaatgcttcgttatccttcaatcgaaaaaattgacattgttgagatcgatgacgtggtagttgatgtatccagaaaatttttcccttatctggcagctaattttaacgatcctcgtgtaaccctagttctcggagatggagctgcatttgtaaaggctgcacaagcgggatattatgatgctattatagtggactcttctgatcccattggtccagcaaaagatttgtttgagaggccattctttgaggcagtagccaaagcccttaggccaggaggagttgtatgcacacaggctgaaagcatttggcttcatatgcatattattaagcaaatcattgctaactgtcgtcaagtctttaagggttctgtcaactatgcttggacaaccgttccaacatatcccaccggtgtgatcggttatatgctctgctctactgaagggccagaagttgacttcaagaatccagtaaatccaattgacaaagagacaactcaagtcaagtccaaattaggacctctcaagttctacaactctgatattcacaaagcagcattcattttaccatctttcgccagaagtatgatcgagtcttaa

SEQ ID NO: 8 - DNA, 1062 bp, Nicotiana tabacum
  source 1..1062: mol_type = genomic DNA
atggaagtcatatctaccaacacaaatggctctaccatcttcaagagtggtgccattcccatgaatggccaccataatggcacttccaaacaccaaaacggccacaagaatgggacttccgaacaacagaacgggacaatcagccttgataatggcaacgagctactgggaaactccaattgtattaagcctggttggttttcagagtttagcgcattatggccaggtgaagcattctcacttaaggttgagaagttactgttccaggggaagtctgactaccaagatgtcatgctctttgagtcagcaacttatgggaaggttctgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgagatcgatgacgtggtagttgatgtatctagaaaatttttcccttatctcgctgctaattttaacgatcctcgtgtaaccctagtccttggagatggggctgcatttgtaaaggctgcacaagcagaatattatgatgctattatagtggactcttctgatcccattggtccagcaaaagatttgtttgagaggccattctttgaggcagtagctaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaaccggtgtgattggttatatgctctgctctactgaaggaccagaaattgacttcaagaatccagtaaatccaattgacaaagagacagctcaagtcaagtccaaattagcacctctcaagttctacaactctgatattcacaaagcagcattcattttgccatctttcgccagaagtatgatcgagtcttaa

SEQ ID NO: 9 - DNA, 1146 bp, Nicotiana tabacum
  source 1..1146: mol_type = genomic DNA
atggaagtcatatctaccaacacaaatggctctactatcttcaagaatggtgccattcccatgaacggttaccagaatggcacttccaaacaccaaaacggccaccagaatggcacttccgaacatcggaacggccaccagaatgggatttccgaacaccaaaacggccaccagaatggcacttccgagcatcagaacggccatcagaatgggacaatcagccatgacaacggcaacgagctacagctactgggaagctccaactctattaagcctggttggttttcagagtttagcgcattatggccaggtgaagcattctcacttaaggttgagaagttactattccaggggaagtctgattaccaagatgtcatgctctttgagtcagcaacatatgggaaggttctgactttggatggagcaattcaacacacagagaatggtggatttccatacactgaaatgattgttcatcttccacttggttccatcccaaaccctaaaaaggttttgatcatcggcggaggaattggttttacattattcgaaatgcttcgttatcctacaatcgaaaaaattgacattgttgagatcgatgacgtggtagttgatgtatctagaaaatttttcccttatcttgctgctaattttagcgatcctcgtgtaaccctagtccttggagatggggctgcatttgtaaaggccgcacaagcaggatattatgatgctattatagtggactcttctgatcccattggtccagcaaaagacttgtttgagaggccattctttgaggcagtagccaaagccctaaggccaggaggagttgtatgcacacaggctgaaagcatttggcttcatatgcatattattaagcaaatcattgctaactgtcgtcaagtctttaagggctctgtcaactatgcttggactactgttccaacatatccaaccggtgtgattggttatatgctctgttctactgaaggaccagaagttgacttcaagaatccagtaaatccaattgacaaagagacaactcaagtcaagtccaaattagcacctctcaagttctacaactctgatattcacaaagcagcattcattttgccatctttcgccagaagtatgatcgagtcttaa

SEQ ID NO: 10 - DNA, 1260 bp, Nicotiana tabacum
  source 1..1260: mol_type = genomic DNA
atggaagtcatatctaccaacacaaatggctcgaccatcttcaagaatggtgccattcccatgaatggccaccagagtggcacttccaaacacctcaacggctaccagaacggcacttccaaacaccaaaacggccaccataatggcacttccgaacatcggaacggccaccagaatgggatttccgaacaccaaaacggccaccagaatgggacttccgaacatcggaacggccaccagaatgggatttccgaacaccaaaacggccaccagaatgggacttccgaacaccaaaacggccaccagaatgggacttccgaacaacagaacgggacaatcagccatgacaatggcaacgagctactgggaaactccaactctattaagcttggttggttttcagagtttagcgcattatggccaggtgaagcattctcccttaaggttgagaagttactatttcaggggaagtctgactaccaagatgtcatgctctttgagtcagcaacatatgggaaggttttgactttggatggagcaattcaacacacagagaatggtggatttccatacactgaaatgattgttcatcttccacttggttccatcccaaacccaaaaaaggttttgatcatcggcggaggaattggttttacattattcgaaatgcttcgttatcctacaatcgaaaaaattgacattgttgaaatcgatgacgtggtagttgatgtatctagaaaatctttcccttatctcgcagctaattttaatgatcctcgtgtaaccctcgttctcggagatggggctgcatttgtaaaggctgcacaagcaggatattatgatgctattatagtggactcttctgatcccattggtccagcaaaagatttgtttgagaggccattctttgaggcagtagccaaagccctaaggccaggaggagttgtatgcacacaggccgaaagcatttggcttcatatgcatattattaagcaaatcattgctaactgtcgtcaagtctttaagggctctgtcaactacgcttggactactgttccaacatatcccactggtgtaattgggtatatgctctgctctactgaagggccagaagttgacttcaagaatccaataaatccaattgacaaagagacaactcaagtcaagtccaaattagcacctctcaagttttacaattctgatattcacaaagcagcattcattttgccatctttcgccagaagtatgatcgagtcttaa

SEQ ID NO: 11 - protein, 375 aa, Nicotiana tabacum
MEVISTNTNGSTIFKNGTIPMNGHQNGSSEHLNGYQNGISKHQNGHQNGTSEHRNGHQNGTSEQQNGTISHDNGNELLGSSNSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPSIEKIDIVEIDDVVVDVSRKFFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVNFKNPVNPIDKETTQVKSKLGPLKFYNSDIHKAAFILPSFARSMIES

SEQ ID NO: 12 - protein, 375 aa, Nicotiana tabacum
MEVISTNTNGSTIFKNGAIPMNGHQNGTSEHLNGYQNGTSKHQNGHQNGTFEHRNGHQNGTSEQQNGTISHDNGNELLGSSDSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPSIEKIDIVEIDDVVVDVSRKFFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPVNPIDKETTQVKSKLGPLKFYNSDIHKAAFILPSFARSMIES

SEQ ID NO: 13 - protein, 353 aa, Nicotiana tabacum
MEVISTNTNGSTIFKSGAIPMNGHHNGTSKHQNGHKNGTSEQQNGTISLDNGNELLGNSNCIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPTIEKIDIVEIDDVVVDVSRKFFPYLAANFNDPRVTLVLGDGAAFVKAAQAEYYDAIIVDSSDPIGPAKDLFERPFFEAVAKALRPGGVVCTQAESIWLHMHIIKQIIANCRQVFKGSVNYAWTTVPTYPTGVIGYMLCSTEGPEIDFKNPVNPIDKETAQVKSKLAPLKFYNSDIHKAAFILPSFARSMIES

SEQ ID NO: 14 - protein, 381 aa, Nicotiana tabacum
MEVISTNTNGSTIFKNGAIPMNGYQNGTSKHQNGHQNGTSEHRNGHQNGISEHQNGHQNGTSEHQNGHQNGTISHDNGNELQLLGSSNSIKPGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPTIEKIDIVEIDDVVVDVSRKFFPYLAANFSDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPVNPIDKETTQVKSKLAPLKFYNSDIHKAAFILPSFARSMIES

SEQ ID NO: 15 - protein, 419 aa, Nicotiana tabacum
MEVISTNTNGSTIFKNGAIPMNGHQSGTSKHLNGYQNGTSKHQNGHHNGTSEHRNGHQNGISEHQNGHQNGTSEHRNGHQNGISEHQNGHQNGTSEHQNGHQNGTSEQQNGTISHDNGNELLGNSNSIKLGWFSEFSALWPGEAFSLKVEKLLFQGKSDYQDVMLFESATYGKVLTLDGAIQHTENGGFPYTEMIVHLPLGSIPNPKKVLIIGGGIGFTLFEMLRYPTIEKIDIVEIDDVVVDVSRKSFPYLAANFNDPRVTLVLGDGAAFVKAAQAGYYDAIIVDSSDPIGPAKDLFERPFFEAVAKALRPGGVVCTQAESIWLHMHIIKQIIANCRQVFKGSVNYAWTTVPTYPTGVIGYMLCSTEGPEVDFKNPINPIDKETTQVKSKLAPLKFYNSDIHKAAFILPSFARSMIES

SEQ ID NO: 16 - DNA, 20 bp, synthetic construct
  misc_feature 1..20: note = Description of sequence: Synthetic oligonucleotide
  source 1..20: mol_type = other DNA
cccatgaacggccaccaaaa

SEQ ID NO: 17 - DNA, 20 bp, synthetic construct
  misc_feature 1..20: note = Description of sequence: Synthetic oligonucleotide
  source 1..20: mol_type = other DNA
ggcacttccaaacaccaaaa

SEQ ID NO: 18 - DNA, 20 bp, synthetic construct
  misc_feature 1..20: note = Description of sequence: Synthetic oligonucleotide
  source 1..20: mol_type = other DNA
gttgttcggatgtcccattc

SEQ ID NO: 19 - DNA, 20 bp, synthetic construct
  misc_feature 1..20: note = Description of sequence: Synthetic oligonucleotide
  source 1..20: mol_type = other DNA
ctaaactctgaaaaccaacc

SEQ ID NO: 20 - DNA, 20 bp, synthetic construct
  misc_feature 1..20: note = Description of sequence: Synthetic oligonucleotide
  source 1..20: mol_type = other DNA
tttcagagtttagcgcatta

SEQ ID NO: 21 - DNA, 23 bp, synthetic construct
  misc_feature 1..23: note = Description of sequence: Synthetic oligonucleotide
  source 1..23: mol_type = other DNA
gatggagcaattcaacatacaga

SEQ ID NO: 22 - DNA, 23 bp, synthetic construct
  misc_feature 1..23: note = Description of sequence: Synthetic oligonucleotide
  source 1..23: mol_type = other DNA
gatggagcaattcaacacacaga

SEQ ID NO: 23 - DNA, 40 bp, synthetic construct
  misc_feature 1..40: note = Description of sequence: Synthetic oligonucleotide
  source 1..40: mol_type = other DNA
tggcatttccaaacaccaaacgggcaccagaatggcactt

SEQ ID NO: 24 - DNA, 40 bp, synthetic construct
  misc_feature 1..40: note = Description of sequence: Synthetic oligonucleotide
  source 1..40: mol_type = other DNA
ccaactctattaagcctggtggttttcagagtttagcgca

SEQ ID NO: 25 - DNA, 40 bp, synthetic construct
  misc_feature 1..40: note = Description of sequence: Synthetic oligonucleotide
  source 1..40: mol_type = other DNA
ttctgactttggatggagcaatacagagaatggtggattt

SEQ ID NO: 26 - DNA, 40 bp, synthetic construct
  misc_feature 1..40: note = Description of sequence: Synthetic oligonucleotide
  source 1..40: mol_type = other DNA
ctgactttggatggagcaatgagaatggtggatttccata

SEQ ID NO: 27 - DNA, 40 bp, synthetic construct
  misc_feature 1..40: note = Description of sequence: Synthetic oligonucleotide
  source 1..40: mol_type = other DNA
tgactttggatggagcaattcagagaatggtggatttcca

SEQ ID NO: 28 - DNA, 40 bp, synthetic construct
  misc_feature 1..40: note = Description of sequence: Synthetic oligonucleotide
  source 1..40: mol_type = other DNA
tgactttggatggagcaattagaatggtggatttccatac

SEQ ID NO: 29 - DNA, 40 bp, synthetic construct
  misc_feature 1..40: note = Description of sequence: Synthetic oligonucleotide
  source 1..40: mol_type = other DNA
gactttggatggagcaattcagagaatggtggatttccat

SEQ ID NO: 30 - DNA, 40 bp, synthetic construct
  misc_feature 1..40: note = Description of sequence: Synthetic oligonucleotide
  source 1..40: mol_type = other DNA
gactttggatggagcaattcgagaatggtggatttccata

SEQ ID NO: 31 - DNA, 40 bp, synthetic construct
  misc_feature 1..40: note = Description of sequence: Synthetic oligonucleotide
  source 1..40: mol_type = other DNA
gactttggatggagcaattcagaatggtggatttccatac

SEQ ID NO: 32 - DNA, 40 bp, synthetic construct
  misc_feature 1..40: note = Description of sequence: Synthetic oligonucleotide
  source 1..40: mol_type = other DNA
gactttggatggagcaattctggatttccatacactgaaa

SEQ ID NO: 33 - DNA, 41 bp, synthetic construct
  misc_feature 1..41: note = Description of sequence: Synthetic oligonucleotide
  source 1..41: mol_type = other DNA
actttggatggagcaattcatacagagaatggtggatttcc

SEQ ID NO: 34 - DNA, 40 bp, synthetic construct
  misc_feature 1..40: note = Description of sequence: Synthetic oligonucleotide
  source 1..40: mol_type = other DNA
actttggatggagcaattcacagagaatggtggatttcca

SEQ ID NO: 35 - DNA, 40 bp, synthetic construct
  misc_feature 1..40: note = Description of sequence: Synthetic oligonucleotide
  source 1..40: mol_type = other DNA
actttggatggagcaattcaagagaatggtggatttccat

SEQ ID NO: 36 - DNA, 40 bp, synthetic construct
  misc_feature 1..40: note = Description of sequence: Synthetic oligonucleotide
  source 1..40: mol_type = other DNA
actttggatggagcaattcagagaatggtggatttccata

SEQ ID NO: 37 - DNA, 40 bp, synthetic construct
  misc_feature 1..40: note = Description of sequence: Synthetic oligonucleotide
  source 1..40: mol_type = other DNA
actttggatggagcaattcaatacactgaaatgattgttc

SEQ ID NO: 38 - DNA, 40 bp, synthetic construct
  misc_feature 1..40: note = Description of sequence: Synthetic oligonucleotide
  source 1..40: mol_type = other DNA
ctttggatggagcaattcaatacagagaatggtggatttc

SEQ ID NO: 39 - DNA, 40 bp, synthetic construct
  misc_feature 1..40: note = Description of sequence: Synthetic oligonucleotide
  source 1..40: mol_type = other DNA
ctttggatggagcaattcaacagagaatggtggatttcca

SEQ ID NO: 40 - DNA, 40 bp, synthetic construct
  misc_feature 1..40: note = Description of sequence: Synthetic oligonucleotide
  source 1..40: mol_type = other DNA
ctttggatggagcaattcaagagaatggtggatttccata

SEQ ID NO: 41 - DNA, 40 bp, synthetic construct
  misc_feature 1..40: note = Description of sequence: Synthetic oligonucleotide
  source 1..40: mol_type = other DNA
ctttggatggagcaattcaaagaatggtggatttccatac

SEQ ID NO: 42 - DNA, 40 bp, synthetic construct
  misc_feature 1..40: note = Description of sequence: Synthetic oligonucleotide
  source 1..40: mol_type = other DNA
tttggatggagcaattcaactacagagaatggtggatttc

SEQ ID NO: 43 - DNA, 40 bp, synthetic construct
  misc_feature 1..40: note = Description of sequence: Synthetic oligonucleotide
  source 1..40: mol_type = other DNA
tttggatggagcaattcaaccagagaatggtggatttcca

SEQ ID NO: 44 - DNA, 40 bp, synthetic construct
  misc_feature 1..40: note = Description of sequence: Synthetic oligonucleotide
  source 1..40: mol_type = other DNA
tttggatggagcaattcaacagagaatggtggatttccat

SEQ ID NO: 45 - DNA, 40 bp, synthetic construct
  misc_feature 1..40: note = Description of sequence: Synthetic oligonucleotide
  source 1..40: mol_type = other DNA
tttggatggagcaattcaacgagaatggtggatttccata

SEQ ID NO: 46 - DNA, 40 bp, synthetic construct
  misc_feature 1..40: note = Description of sequence: Synthetic oligonucleotide
  source 1..40: mol_type = other DNA
tttggatggagcaattcaacagaatggtggatttccatac

SEQ ID NO: 47 - DNA, 40 bp, synthetic construct
  misc_feature 1..40: note = Description of sequence: Synthetic oligonucleotide
  source 1..40: mol_type = other DNA
tttggatggagcaattcaacaatggtggatttccatacac

SEQ ID NO: 48 - DNA, 40 bp, synthetic construct
  misc_feature 1..40: note = Description of sequence: Synthetic oligonucleotide
  source 1..40: mol_type = other DNA
ttggatggagcaattcaacacagagaatggtggatttcca

SEQ ID NO: 49 - DNA, 40 bp, synthetic construct
  misc_feature 1..40: note = Description of sequence: Synthetic oligonucleotide
  source 1..40: mol_type = other DNA
ttggatggagcaattcaacagagaatggtggatttccata

SEQ ID NO: 50 - DNA, 40 bp, synthetic construct
  misc_feature 1..40: note = Description of sequence: Synthetic oligonucleotide
  source 1..40: mol_type = other DNA
tggatggagcaattcaacatagagaatggtggatttccat

SEQ ID NO: 51 - DNA, 40 bp, synthetic construct
  misc_feature 1..40: note = Description of sequence: Synthetic oligonucleotide
  source 1..40: mol_type = other DNA
tggatggagcaattcaacatgagaatggtggatttccata

SEQ ID NO: 52 - DNA, 40 bp, synthetic construct
  misc_feature 1..40: note = Description of sequence: Synthetic oligonucleotide
  source 1..40: mol_type = other DNA
tggatggagcaattcaacatagaatggtggatttccatac

SEQ ID NO: 53 - DNA, 40 bp, synthetic construct
  misc_feature 1..40: note = Description of sequence: Synthetic oligonucleotide
  source 1..40: mol_type = other DNA
tggatggagcaattcaacataatggtggatttccatacac

SEQ ID NO: 54 - DNA, 40 bp, synthetic construct
  misc_feature 1..40: note = Description of sequence: Synthetic oligonucleotide
  source 1..40: mol_type = other DNA
ggatggagcaattcaacatagagaatggtggatttccata

SEQ ID NO: 55 - DNA, 40 bp, synthetic construct
  misc_feature 1..40: note = Description of sequence: Synthetic oligonucleotide
  source 1..40: mol_type = other DNA
caattcaacatacagagaatgtggatttccatacactgaa

SEQ ID NO: 56 - DNA, 41 bp, synthetic construct
  misc_feature 1..41: note = Description of sequence: Synthetic oligonucleotide
  source 1..41: mol_type = other DNA
gcacttccaaacaccaaaacagggcaccagaatggcacttt

SEQ ID NO: 57 - DNA, 40 bp, synthetic construct; identical to SEQ ID NO: 25
  misc_feature 1..40: note = Description of sequence: Synthetic oligonucleotide
  source 1..40: mol_type = other DNA

SEQ ID NO: 58 - DNA, 40 bp, synthetic construct
  misc_feature 1..40: note = Description of sequence: Synthetic oligonucleotide
  source 1..40: mol_type = other DNA
ttctgactttggatggagcagagaatggtggatttccata

SEQ ID NO: 59 - DNA, 40 bp, synthetic construct
  misc_feature 1..40: note = Description of sequence: Synthetic oligonucleotide
  source 1..40: mol_type = other DNA
ctgactttggatggagcaatacagagaatggtggatttcc

SEQ ID NO: 60 - DNA, 40 bp, synthetic construct; identical to SEQ ID NO: 26
  misc_feature 1..40: note = Description of sequence: Synthetic oligonucleotide
  source 1..40: mol_type = other DNA

SEQ ID NO: 61 - DNA, 40 bp, synthetic construct; identical to SEQ ID NO: 27
  misc_feature 1..40: note = Description of sequence: Synthetic oligonucleotide
  source 1..40: mol_type = other DNA

SEQ ID NO: 62 - DNA, 40 bp, synthetic construct; identical to SEQ ID NO: 28
  misc_feature 1..40: note = Description of sequence: Synthetic oligonucleotide
  source 1..40: mol_type = other DNA

SEQ ID NO: 63 - DNA, 40 bp, synthetic construct; identical to SEQ ID NO: 29
  misc_feature 1..40: note = Description of sequence: Synthetic oligonucleotide
  source 1..40: mol_type = other DNA

SEQ ID NO: 64 - DNA, 40 bp, synthetic construct; identical to SEQ ID NO: 30
  misc_feature 1..40: note = Description of sequence: Synthetic oligonucleotide
  source 1..40: mol_type = other DNA

SEQ ID NO: 65 - DNA, 40 bp, synthetic construct; identical to SEQ ID NO: 31
  misc_feature 1..40: note = Description of sequence: Synthetic oligonucleotide
  source 1..40: mol_type = other DNA

SEQ ID NO: 66 - DNA, 40 bp, synthetic construct
  misc_feature 1..40: note = Description of sequence: Synthetic oligonucleotide
  source 1..40: mol_type = other DNA
gactttggatggagcaattcgaatggtggatttccataca

SEQ ID NO: 67 - DNA, 40 bp, synthetic construct; identical to SEQ ID NO: 32
  misc_feature 1..40: note = Description of sequence: Synthetic oligonucleotide
  source 1..40: mol_type = other DNA

SEQ ID NO: 68 - DNA, 40 bp, synthetic construct
  misc_feature 1..40: note = Description of sequence: Synthetic oligonucleotide
  source 1..40: mol_type = other DNA
actttggatggagcaattcaatacagagaatggtggattt

SEQ ID NO: 69 - DNA, 40 bp, synthetic construct
  misc_feature 1..40: note = Description of sequence: Synthetic oligonucleotide
  source 1..40: mol_type = other DNA
actttggatggagcaattcaacagagaatggtggatttcc

SEQ ID NO: 70 - DNA, 40 bp, synthetic construct; identical to SEQ ID NO: 35
  misc_feature 1..40: note = Description of sequence: Synthetic oligonucleotide
  source 1..40: mol_type = other DNA

SEQ ID NO: 71 - DNA, 40 bp, synthetic construct; identical to SEQ ID NO: 36
  misc_feature 1..40: note = Description of sequence: Synthetic oligonucleotide
  source 1..40: mol_type = other DNA

SEQ ID NO: 72 - DNA, 40 bp, synthetic construct
  misc_feature 1..40: note = Description of sequence: Synthetic oligonucleotide
  source 1..40: mol_type = other DNA
actttggatggagcaattcaagaatggtggatttccatac

SEQ ID NO: 73 - DNA, 40 bp, synthetic construct
  misc_feature 1..40: note = Description of sequence: Synthetic oligonucleotide
  source 1..40: mol_type = other DNA
actttggatggagcaattcaaatggtggatttccatacac

SEQ ID NO: 74 - DNA, 40 bp, synthetic construct
  misc_feature 1..40: note = Description of sequence: Synthetic oligonucleotide
  source 1..40: mol_type = other DNA
ctttggatggagcaattcaaatacagagaatggtggattt

SEQ ID NO: 75 - DNA, 40 bp, synthetic construct; identical to SEQ ID NO: 38
  misc_feature 1..40: note = Description of sequence: Synthetic oligonucleotide
  source 1..40: mol_type = other DNA

SEQ ID NO: 76 - DNA, 40 bp, synthetic construct; identical to SEQ ID NO: 39
  misc_feature 1..40: note = Description of sequence: Synthetic oligonucleotide
  source 1..40: mol_type = other DNA

SEQ ID NO: 77 - DNA, 40 bp, synthetic construct
  misc_feature 1..40: note = Description of sequence: Synthetic oligonucleotide
  source 1..40: mol_type = other DNA
ctttggatggagcaattcaaagagaatggtggatttccat

SEQ ID NO: 78 - DNA, 40 bp, synthetic construct; identical to SEQ ID NO: 41
  misc_feature 1..40: note = Description of sequence: Synthetic oligonucleotide
  source 1..40: mol_type = other DNA

SEQ ID NO: 79 - DNA, 41 bp, synthetic construct
  misc_feature 1..41: note = Description of sequence: Synthetic oligonucleotide
  source 1..41: mol_type = other DNA
tttggatggagcaattcaactacagagaatggtggatttcc

SEQ ID NO: 80 - DNA, 40 bp, synthetic construct; identical to SEQ ID NO: 44
  misc_feature 1..40: note = Description of sequence: Synthetic oligonucleotide
  source 1..40: mol_type = other DNA

SEQ ID NO: 81 - DNA, 40 bp, synthetic construct; identical to SEQ ID NO: 45
  misc_feature 1..40: note = Description of sequence: Synthetic oligonucleotide
  source 1..40: mol_type = other DNA

SEQ ID NO: 82 - DNA, 40 bp, synthetic construct; identical to SEQ ID NO: 46
  misc_feature 1..40: note = Description of sequence: Synthetic oligonucleotide
  source 1..40: mol_type = other DNA

SEQ ID NO: 83 - DNA, 40 bp, synthetic construct
  misc_feature 1..40: note = Description of sequence: Synthetic oligonucleotide
  source 1..40: mol_type = other DNA
tttggatggagcaattcaacgaatggtggatttccataca

SEQ ID NO: 84 - DNA, 40 bp, synthetic construct
  misc_feature 1..40: note = Description of sequence: Synthetic oligonucleotide
  source 1..40: mol_type = other DNA
tttggatggagcaattcaacggatttccatacactgaaat

SEQ ID NO: 85 - DNA, 40 bp, synthetic construct
  misc_feature 1..40: note = Description of sequence: Synthetic oligonucleotide
  source 1..40: mol_type = other DNA
ttggatggagcaattcaacaacagagaatggtggatttcc

SEQ ID NO: 86 - DNA, 40 bp, synthetic construct; identical to SEQ ID NO: 48
  misc_feature 1..40: note = Description of sequence: Synthetic oligonucleotide
  source 1..40: mol_type = other DNA

SEQ ID NO: 87 - DNA, 40 bp, synthetic construct
  misc_feature 1..40: note = Description of sequence: Synthetic oligonucleotide
  source 1..40: mol_type = other DNA
ttggatggagcaattcaacaagagaatggtggatttccat

SEQ ID NO: 88 - DNA, 40 bp, synthetic construct; identical to SEQ ID NO: 49
  misc_feature 1..40: note = Description of sequence: Synthetic oligonucleotide
  source 1..40: mol_type = other DNA

SEQ ID NO: 89 - DNA, 40 bp, synthetic construct
  misc_feature 1..40: note = Description of sequence: Synthetic oligonucleotide
  source 1..40: mol_type = other DNA
ttggatggagcaattcaacaagaatggtggatttccatac

SEQ ID NO: 90 - DNA, 40 bp, synthetic construct
  misc_feature 1..40: note = Description of sequence: Synthetic oligonucleotide
  source 1..40: mol_type = other DNA
ttggatggagcaattcaacagaatggtggatttccataca

SEQ ID NO: 91 - DNA, 40 bp, synthetic construct
  misc_feature 1..40: note = Description of sequence: Synthetic oligonucleotide
  source 1..40: mol_type = other DNA
ttggatggagcaattcaacaaatggtggatttccatacac

SEQ ID NO: 92 - DNA, 40 bp, synthetic construct; identical to SEQ ID NO: 50
  misc_feature 1..40: note = Description of sequence: Synthetic oligonucleotide
  source 1..40: mol_type = other DNA

SEQ ID NO: 93 - DNA, 40 bp, synthetic construct; identical to SEQ ID NO: 52
  misc_feature 1..40: note = Description of sequence: Synthetic oligonucleotide
  source 1..40: mol_type = other DNA

SEQ ID NO: 94 - DNA, 40 bp, synthetic construct
  misc_feature 1..40: note = Description of sequence: Synthetic oligonucleotide
  source 1..40: mol_type = other DNA
tggatggagcaattcaacatatggtggatttccatacact

SEQ ID NO: 95 - DNA, 40 bp, synthetic construct; identical to SEQ ID NO: 54
  misc_feature 1..40: note = Description of sequence: Synthetic oligonucleotide
  source 1..40: mol_type = other DNA

SEQ ID NO: 96 - DNA, 40 bp, synthetic construct
  misc_feature 1..40: note = Description of sequence: Synthetic oligonucleotide
  source 1..40: mol_type = other DNA
ggatggagcaattcaacataagaatggtggatttccatac

SEQ ID NO: 97 - DNA, 40 bp, synthetic construct
  misc_feature 1..40: note = Description of sequence: Synthetic oligonucleotide
  source 1..40: mol_type = other DNA
gagcaattcaacatacagagtggtggatttccatacactg

SEQ ID NO: 98 - DNA, 40 bp, synthetic construct
  misc_feature 1..40: note = Description of sequence: Synthetic oligonucleotide
  source 1..40: mol_type = other DNA
tggcacttccaaacaccaaacggccacaagaatgggactt

SEQ ID NO: 99 - DNA, 40 bp, synthetic construct
  misc_feature 1..40: note = Description of sequence: Synthetic oligonucleotide
  source 1..40: mol_type = other DNA
ccaattgtattaagcctggtggttttcagagtttagcgca

SEQ ID NO: 100 - DNA, 40 bp, synthetic construct
  misc_feature 1..40: note = Description of sequence: Synthetic oligonucleotide
  source 1..40: mol_type = other DNA
tgactttggatggagcaattcacagagaatggtggatttc

SEQ ID NO: 101 - DNA, 40 bp, synthetic construct; identical to SEQ ID NO: 27
  misc_feature 1..40: note = Description of sequence: Synthetic oligonucleotide
  source 1..40: mol_type = other DNA

SEQ ID NO: 102 - DNA, 40 bp, synthetic construct; identical to SEQ ID NO: 28
  misc_feature 1..40: note = Description of sequence: Synthetic oligonucleotide
  source 1..40: mol_type = other DNA

SEQ ID NO: 103 - DNA, 40 bp, synthetic construct; identical to SEQ ID NO: 30
  misc_feature 1..40: note = Description of sequence: Synthetic oligonucleotide
  source 1..40: mol_type = other DNA

SEQ ID NO: 104 - DNA, 40 bp, synthetic construct
  misc_feature 1..40: note = Description of sequence: Synthetic oligonucleotide
  source 1..40: mol_type = other DNA
gactttggatggagcaattcaatggtggatttccatacac

SEQ ID NO: 105 - DNA, 40 bp, synthetic construct
  misc_feature 1..40: note = Description of sequence: Synthetic oligonucleotide
  source 1..40: mol_type = other DNA
actttggatggagcaattcaacacagagaatggtggattt

SEQ ID NO: 106 - DNA, 40 bp, synthetic construct; identical to SEQ ID NO: 69
  misc_feature 1..40: note = Description of sequence: Synthetic oligonucleotide
  source 1..40: mol_type = other DNA

SEQ ID NO: 107 - DNA, 40 bp, synthetic construct; identical to SEQ ID NO: 34
  misc_feature 1..40: note = Description of sequence: Synthetic oligonucleotide
  source 1..40: mol_type = other DNA

SEQ ID NO: 108 - DNA, 40 bp, synthetic construct; identical to SEQ ID NO: 35
  misc_feature 1..40: note = Description of sequence: Synthetic oligonucleotide
  source 1..40: mol_type = other DNA

SEQ ID NO: 109 - DNA, 40 bp, synthetic construct; identical to SEQ ID NO: 36
  misc_feature 1..40: note = Description of sequence: Synthetic oligonucleotide
  source 1..40: mol_type = other DNA

SEQ ID NO: 110 - DNA, 40 bp, synthetic construct; identical to SEQ ID NO: 72
  misc_feature 1..40: note = Description of sequence: Synthetic oligonucleotide
  source 1..40: mol_type = other DNA

SEQ ID NO: 111 - DNA, 40 bp, synthetic construct; identical to SEQ ID NO: 73
  misc_feature 1..40: note = Description of sequence: Synthetic oligonucleotide
  source 1..40: mol_type = other DNA

SEQ ID NO: 112 - DNA, 40 bp, synthetic construct
  misc_feature 1..40: note = Description of sequence: Synthetic oligonucleotide
  source 1..40: mol_type = other DNA
ctttggatggagcaattcaaacacagagaatggtggattt

SEQ ID NO: 113 - DNA, 40 bp, synthetic construct
  misc_feature 1..40: note = Description of sequence: Synthetic oligonucleotide
  source 1..40: mol_type = other DNA
ctttggatggagcaattcaacacagagaatggtggatttc

SEQ ID NO: 114 - DNA, 40 bp, synthetic construct; identical to SEQ ID NO: 39
  misc_feature 1..40: note = Description of sequence: Synthetic oligonucleotide
  source 1..40: mol_type = other DNA

SEQ ID NO: 115 - DNA, 40 bp, synthetic construct; identical to SEQ ID NO: 41
  misc_feature 1..40: note = Description of sequence: Synthetic oligonucleotide
  source 1..40: mol_type = other DNA

SEQ ID NO: 116 - DNA, 40 bp, synthetic construct
  misc_feature 1..40: note = Description of sequence: Synthetic oligonucleotide
  source 1..40: mol_type = other DNA
tttggatggagcaattcaacacagagaatggtggatttcc

SEQ ID NO: 117 - DNA, 40 bp, synthetic construct; identical to SEQ ID NO: 44
  misc_feature 1..40: note = Description of sequence: Synthetic oligonucleotide
  source 1..40: mol_type = other DNA

SEQ ID NO: 118 - DNA, 40 bp, synthetic construct; identical to SEQ ID NO: 45
  misc_feature 1..40: note = Description of sequence: Synthetic oligonucleotide
  source 1..40: mol_type = other DNA

SEQ ID NO: 119 - DNA, 40 bp, synthetic construct; identical to SEQ ID NO: 46
  misc_feature 1..40: note = Description of sequence: Synthetic oligonucleotide
  source 1..40: mol_type = other DNA

SEQ ID NO: 120 - DNA, 40 bp, synthetic construct; identical to SEQ ID NO: 47
  misc_feature 1..40: note = Description of sequence: Synthetic oligonucleotide
  source 1..40: mol_type = other DNA

SEQ ID NO: 121 - DNA, 40 bp, synthetic construct; identical to SEQ ID NO: 84
  misc_feature 1..40: note = Description of sequence: Synthetic oligonucleotide
  source 1..40: mol_type = other DNA

SEQ ID NO: 122 - DNA, 40 bp, synthetic construct; identical to SEQ ID NO: 48
  misc_feature 1..40: note = Description of sequence: Synthetic oligonucleotide
  source 1..40: mol_type = other DNA

SEQ ID NO: 123 - DNA, 40 bp, synthetic construct; identical to SEQ ID NO: 49
  misc_feature 1..40: note = Description of sequence: Synthetic oligonucleotide
  source 1..40: mol_type = other DNA

SEQ ID NO: 124 - DNA, 40 bp, synthetic construct; identical to SEQ ID NO: 90
  misc_feature 1..40: note = Description of sequence: Synthetic oligonucleotide
  source 1..40: mol_type = other DNA

SEQ ID NO: 125 - DNA, 40 bp, synthetic construct; identical to SEQ ID NO: 91
  misc_feature 1..40: note = Description of sequence: Synthetic oligonucleotide
  source 1..40: mol_type = other DNA

SEQ ID NO: 126 - DNA, 40 bp, synthetic construct
  misc_feature 1..40: note = Description of sequence: Synthetic oligonucleotide
  source 1..40: mol_type = other DNA
ttggatggagcaattcaacatggatttccatacactgaaa

SEQ ID NO: 127 - DNA, 40 bp, synthetic construct
  misc_feature 1..40: note = Description of sequence: Synthetic oligonucleotide
  source 1..40: mol_type = other DNA
tggatggagcaattcaacacgagaatggtggatttccata

SEQ ID NO: 128 - DNA, 40 bp, synthetic construct
  misc_feature 1..40: note = Description of sequence: Synthetic oligonucleotide
  source 1..40: mol_type = other DNA
tggatggagcaattcaacacagaatggtggatttccatac

SEQ ID NO: 129 - DNA, 40 bp, synthetic construct
  misc_feature 1..40: note = Description of sequence: Synthetic oligonucleotide
  source 1..40: mol_type = other DNA
ggatggagcaattcaacacagagaatggtggatttccata

SEQ ID NO: 130 - DNA, 40 bp, synthetic construct
  misc_feature 1..40: note = Description of sequence: Synthetic oligonucleotide
  source 1..40: mol_type = other DNA
ggatggagcaattcaacacagaatggtggatttccataca

SEQ ID NO: 131 - DNA, 40 bp, synthetic construct
  misc_feature 1..40: note = Description of sequence: Synthetic oligonucleotide
  source 1..40: mol_type = other DNA
ggatggagcaattcaacacagtggatttccatacactgaa

SEQ ID NO: 132 - DNA, 40 bp, synthetic construct
  misc_feature 1..40: note = Description of sequence: Synthetic oligonucleotide
  source 1..40: mol_type = other DNA
gatggagcaattcaacacactggtggatttccatacactg

SEQ ID NO: 133 - DNA, 40 bp, synthetic construct
  misc_feature 1..40: note = Description of sequence: Synthetic oligonucleotide
  source 1..40: mol_type = other DNA
tggcacttccaaacaccaaacggccaccagaatggcactt

SEQ ID NO: 134 - DNA, 40 bp, synthetic construct; identical to SEQ ID NO: 24
  misc_feature 1..40: note = Description of sequence: Synthetic oligonucleotide
  source 1..40: mol_type = other DNA

SEQ ID NO: 135 - DNA, 40 bp, synthetic construct
  misc_feature 1..40: note = Description of sequence: Synthetic oligonucleotide
  source 1..40: mol_type = other DNA
aacatatgggaaggttctgattggatggagcaattcaaca

SEQ ID NO: 136 - DNA, 40 bp, synthetic construct
  misc_feature 1..40: note = Description of sequence: Synthetic oligonucleotide
  source 1..40: mol_type = other DNA
atgggaaggttctgactttgtggagcaattcaacacacag

SEQ ID NO: 137 - DNA, 40 bp, synthetic construct
  misc_feature 1..40: note = Description of sequence: Synthetic oligonucleotide
  source 1..40: mol_type = other DNA
gttctgactttggatggagcttcaacacacagagaatggt

SEQ ID NO: 138 - DNA, 40 bp, synthetic construct; identical to SEQ ID NO: 59
  misc_feature 1..40: note = Description of sequence: Synthetic oligonucleotide
  source 1..40: mol_type = other DNA

SEQ ID NO: 139 - DNA, 40 bp, synthetic construct; identical to SEQ ID NO: 26
  misc_feature 1..40: note = Description of sequence: Synthetic oligonucleotide
  source 1..40: mol_type = other DNA

SEQ ID NO: 140 - DNA, 40 bp, synthetic construct
  misc_feature 1..40: note = Description of sequence: Synthetic oligonucleotide
  source 1..40: mol_type = other DNA
ctgactttggatggagcaatagaatggtggatttccatac

SEQ ID NO: 141 - DNA, 40 bp, synthetic construct; identical to SEQ ID NO: 27
  misc_feature 1..40: note = Description of sequence: Synthetic oligonucleotide
  source 1..40: mol_type = other DNA

SEQ ID NO: 142 - DNA, 40 bp, synthetic construct
  misc_feature 1..40: note = Description of sequence: Synthetic oligonucleotide
  source 1..40: mol_type = other DNA
gactttggatggagcaattcacacacagagaatggtggat

SEQ ID NO: 143 - DNA, 40 bp, synthetic construct
  misc_feature 1..40: note = Description of sequence: Synthetic oligonucleotide
  source 1..40: mol_type = other DNA
gactttggatggagcaattccacacagagaatggtggatt

SEQ ID NO: 144 - DNA, 40 bp, synthetic construct; identical to SEQ ID NO: 30
  misc_feature 1..40: note = Description of sequence: Synthetic oligonucleotide
  source 1..40: mol_type = other DNA

SEQ ID NO: 145 - DNA, 40 bp, synthetic construct; identical to SEQ ID NO: 104
  misc_feature 1..40: note = Description of sequence: Synthetic oligonucleotide
  source 1..40: mol_type = other DNA

SEQ ID NO: 146 - DNA, 40 bp, synthetic construct; identical to SEQ ID NO: 105
  misc_feature 1..40: note = Description of sequence: Synthetic oligonucleotide
  source 1..40: mol_type = other DNA

SEQ ID NO: 147 - DNA, 40 bp, synthetic construct; identical to SEQ ID NO: 69
  misc_feature 1..40: note = Description of sequence: Synthetic oligonucleotide
  source 1..40: mol_type = other DNA

SEQ ID NO: 148 - DNA, 40 bp, synthetic construct; identical to SEQ ID NO: 35
  misc_feature 1..40: note = Description of sequence: Synthetic oligonucleotide
  source 1..40: mol_type = other DNA

SEQ ID NO: 149 - DNA, 40 bp, synthetic construct; identical to SEQ ID NO: 72
  misc_feature 1..40: note = Description of sequence: Synthetic oligonucleotide
  source 1..40: mol_type = other DNA

SEQ ID NO: 150 - DNA, 40 bp, synthetic construct; identical to SEQ ID NO: 73
  misc_feature 1..40: note = Description of sequence: Synthetic oligonucleotide
  source 1..40: mol_type = other DNA

SEQ ID NO: 151 - DNA, 40 bp, synthetic construct
  misc_feature 1..40: note = Description of sequence: Synthetic oligonucleotide
  source 1..40: mol_type = other DNA
actttggatggagcaattcatggatttccatacactgaaa

SEQ ID NO: 152 - DNA, 40 bp, synthetic construct
  misc_feature 1..40: note = Description of sequence: Synthetic oligonucleotide
  source 1..40: mol_type = other DNA
actttggatggagcaattcagatttccatacactgaaatg

SEQ ID NO: 153 - DNA, 40 bp, synthetic construct; identical to SEQ ID NO: 112
  misc_feature 1..40: note = Description of sequence: Synthetic oligonucleotide
  source 1..40: mol_type = other DNA

SEQ ID NO: 154 - DNA, 40 bp, synthetic construct; identical to SEQ ID NO: 39
  misc_feature 1..40: note = Description of sequence: Synthetic oligonucleotide
  source 1..40: mol_type = other DNA

SEQ ID NO: 155 - DNA, 40 bp, synthetic construct; identical to SEQ ID NO: 77
  misc_feature 1..40: note = Description of sequence: Synthetic oligonucleotide
  source 1..40: mol_type = other DNA

SEQ ID NO: 156 - DNA, 40 bp, synthetic construct; identical to SEQ ID NO: 41
  misc_feature 1..40: note = Description of sequence: Synthetic oligonucleotide
  source 1..40: mol_type = other DNA

SEQ ID NO: 157 - DNA, 40 bp, synthetic construct; identical to SEQ ID NO: 116
  misc_feature 1..40: note = Description of sequence: Synthetic oligonucleotide
  source 1..40: mol_type = other DNA

SEQ ID NO: 158 - DNA, 40 bp, synthetic construct; identical to SEQ ID NO: 44
  misc_feature 1..40: note = Description of sequence: Synthetic oligonucleotide
  source 1..40: mol_type = other DNA

SEQ ID NO: 159 - DNA, 40 bp, synthetic construct; identical to SEQ ID NO: 45
  misc_feature 1..40: note = Description of sequence: Synthetic oligonucleotide
  source 1..40: mol_type = other DNA

SEQ ID NO: 160 - DNA, 40 bp, synthetic construct; identical to SEQ ID NO: 48
  misc_feature 1..40: note = Description of sequence: Synthetic oligonucleotide
  source 1..40: mol_type = other DNA

SEQ ID NO: 161 - DNA, 40 bp, synthetic construct; identical to SEQ ID NO: 49
  misc_feature 1..40: note = Description of sequence: Synthetic oligonucleotide
  source 1..40: mol_type = other DNA

SEQ ID NO: 162 - DNA, 40 bp, synthetic construct; identical to SEQ ID NO: 89
  misc_feature 1..40: note = Description of sequence: Synthetic oligonucleotide
  source 1..40: mol_type = other DNA

SEQ ID NO: 163 - DNA, 40 bp, synthetic construct
  misc_feature 1..40: note = Description of sequence: Synthetic oligonucleotide
  source 1..40: mol_type = other DNA
ttggatggagcaattcaacaggatttccatacactgaaat

SEQ ID NO: 164 - DNA, 40 bp, synthetic construct
  misc_feature 1..40: note = Description of sequence: Synthetic oligonucleotide
  source 1..40: mol_type = other DNA
tggatggagcaattcaacacagagaatggtggatttccat

SEQ ID NO: 165 - DNA, 40 bp, synthetic construct; identical to SEQ ID NO: 128
  misc_feature 1..40: note = Description of sequence: Synthetic oligonucleotide
  source 1..40: mol_type = other DNA

SEQ ID NO: 166 - DNA, 40 bp, synthetic construct
  misc_feature 1..40: note = Description of sequence: Synthetic oligonucleotide
  source 1..40: mol_type = other DNA
tggatggagcaattcaacactggtggatttccatacactg

SEQ ID NO: 167 - DNA, 40 bp, synthetic construct; identical to SEQ ID NO: 129
  misc_feature 1..40: note = Description of sequence: Synthetic oligonucleotide
  source 1..40: mol_type = other DNA

SEQ ID NO: 168 - DNA, 40 bp, synthetic construct
  misc_feature 1..40: note = Description of sequence: Synthetic oligonucleotide
  source 1..40: mol_type = other DNA
tggagcaattcaacacacagatggtggatttccatacact

SEQ ID NO: 169 - DNA, 40 bp, synthetic construct
  misc_feature 1..40: note = Description of sequence: Synthetic oligonucleotide
  source 1..40: mol_type = other DNA
tggagcaattcaacacacagtggtggatttccatacactg

SEQ ID NO: 170 - DNA, 40 bp, synthetic construct
  misc_feature 1..40: note = Description of sequence: Synthetic oligonucleotide
  source 1..40: mol_type = other DNA
ggagcaattcaacacacagaaatggtggatttccatacac

SEQ ID NO: 171 - DNA, 40 bp, synthetic construct
  misc_feature 1..40: note = Description of sequence: Synthetic oligonucleotide
  source 1..40: mol_type = other DNA
caattcaacacacagagaatatttccatacactgaaatga

SEQ ID NO: 172 - DNA, 40 bp, synthetic construct
  misc_feature 1..40: note = Description of sequence: Synthetic oligonucleotide
  source 1..40: mol_type = other DNA
aacacacagagaatggtggaaaatgattgttcatcttcca

SEQ ID NO: 173 - DNA, 40 bp, synthetic construct
  misc_feature 1..40: note = Description of sequence: Synthetic oligonucleotide
  source 1..40: mol_type = other DNA
cggcacttccaaacaccaaacggccaccataatggcactt

SEQ ID NO: 174 - DNA, 40 bp, synthetic construct
  misc_feature 1..40: note = Description of sequence: Synthetic oligonucleotide
  source 1..40: mol_type = other DNA
ttttgactttggatggagcaagagaatggtggatttccat

SEQ ID NO: 175 - DNA, 40 bp, synthetic construct
  misc_feature 1..40: note = Description of sequence: Synthetic oligonucleotide
  source 1..40: mol_type = other DNA
ttgactttggatggagcaatgaatggtggatttccataca

SEQ ID NO: 176 - DNA, 40 bp, synthetic construct; identical to SEQ ID NO: 27
  misc_feature 1..40: note = Description of sequence: Synthetic oligonucleotide
  source 1..40: mol_type = other DNA

SEQ ID NO: 177 - DNA, 40 bp, synthetic construct; identical to SEQ ID NO: 28
  misc_feature 1..40: note = Description of sequence: Synthetic oligonucleotide
  source 1..40: mol_type = other DNA

SEQ ID NO: 178 - DNA, 40 bp, synthetic construct; identical to SEQ ID NO: 104
  misc_feature 1..40: note = Description of sequence: Synthetic oligonucleotide
  source 1..40: mol_type = other DNA

SEQ ID NO: 179 - DNA, 40 bp, synthetic construct
  misc_feature 1..40: note = Description of sequence: Synthetic oligonucleotide
  source 1..40: mol_type = other DNA
gactttggatggagcaattctggtggatttccatacactg

SEQ ID NO: 180 - DNA, 40 bp, synthetic construct; identical to SEQ ID NO: 105
  misc_feature 1..40: note = Description of sequence: Synthetic oligonucleotide
  source 1..40: mol_type = other DNA

SEQ ID NO: 181 - DNA, 40 bp, synthetic construct; identical to SEQ ID NO: 69
  misc_feature 1..40: note = Description of sequence: Synthetic oligonucleotide
  source 1..40: mol_type = other DNA

SEQ ID NO: 182 - DNA, 40 bp, synthetic construct; identical to SEQ ID NO: 35
  misc_feature 1..40: note = Description of sequence: Synthetic oligonucleotide
  source 1..40: mol_type = other DNA

SEQ ID NO: 183 - DNA, 40 bp, synthetic construct; identical to SEQ ID NO: 36
  misc_feature 1..40: note = Description of sequence: Synthetic oligonucleotide
  source 1..40: mol_type = other DNA

SEQ ID NO: 184 - DNA, 40 bp, synthetic construct; identical to SEQ ID NO: 72
  misc_feature 1..40: note = Description of sequence: Synthetic oligonucleotide
  source 1..40: mol_type = other DNA

SEQ ID NO: 185 - DNA, 40 bp, synthetic construct; identical to SEQ ID NO: 39
  misc_feature 1..40: note = Description of sequence: Synthetic oligonucleotide
  source 1..40: mol_type = other DNA

SEQ ID NO: 186 - DNA, 40 bp, synthetic construct; identical to SEQ ID NO: 77
  misc_feature 1..40: note = Description of sequence: Synthetic oligonucleotide
  source 1..40: mol_type = other DNA

SEQ ID NO: 187 - DNA, 40 bp, synthetic construct; identical to SEQ ID NO: 116
  misc_feature 1..40: note = Description of sequence: Synthetic oligonucleotide
  source 1..40: mol_type = other DNA

SEQ ID NO: 188 - DNA, 40 bp, synthetic construct; identical to SEQ ID NO: 44
  misc_feature 1..40: note = Description of sequence: Synthetic oligonucleotide
  source 1..40: mol_type = other DNA

SEQ ID NO: 189 - DNA, 40 bp, synthetic construct; identical to SEQ ID NO: 45
  misc_feature 1..40: note = Description of sequence: Synthetic oligonucleotide
  source 1..40: mol_type = other DNA

SEQ ID NO: 190 - DNA, 40 bp, synthetic construct; identical to SEQ ID NO: 49
  misc_feature 1..40: note = Description of sequence: Synthetic oligonucleotide
  source 1..40: mol_type = other DNA

SEQ ID NO: 191 - DNA, 40 bp, synthetic construct; identical to SEQ ID NO: 89
  misc_feature 1..40: note = Description of sequence: Synthetic oligonucleotide
  source 1..40: mol_type = other DNA

SEQ ID NO: 192 - DNA, 40 bp, synthetic construct; identical to SEQ ID NO: 164
  misc_feature 1..40: note = Description of sequence: Synthetic oligonucleotide
  source 1..40: mol_type = other DNA

SEQ ID NO: 193 - DNA, 40 bp, synthetic construct; identical to SEQ ID NO: 127
  misc_feature 1..40: note = Description of sequence: Synthetic oligonucleotide
  source 1..40: mol_type = other DNA

SEQ ID NO: 194 - DNA, 40 bp, synthetic construct; identical to SEQ ID NO: 128
  misc_feature 1..40: note = Description of sequence: Synthetic oligonucleotide
  source 1..40: mol_type = other DNA

SEQ ID NO: 195 - DNA, 40 bp, synthetic construct
  misc_feature 1..40: note = Description of sequence: Synthetic oligonucleotide
  source 1..40: mol_type = other DNA
tggatggagcaattcaacacatggtggatttccatacact

SEQ ID NO: 196 - DNA, 40 bp, synthetic construct; identical to SEQ ID NO: 129
  misc_feature 1..40: note = Description of sequence: Synthetic oligonucleotide
  source 1..40: mol_type = other DNA

SEQ ID NO: 197 - DNA, 40 bp, synthetic construct
  misc_feature 1..40: note = Description of sequence: Synthetic oligonucleotide
  source 1..40: mol_type = other DNA
tggagcaattcaacacacaggaatggtggatttccataca

SEQ ID NO: 198 - DNA, 40 bp, synthetic construct
  misc_feature 1..40: note = Description of sequence: Synthetic oligonucleotide
  source 1..40: mol_type = other DNA
gcaattcaacacacagagaaatttccatacactgaaatga

SEQ ID NO: 199 - DNA, 40 bp, synthetic construct
  misc_feature 1..40: note = Description of sequence: Synthetic oligonucleotide
  source 1..40: mol_type = other DNA
aacacacagagaatggtggatccatacactgaaatgattg

SEQ ID NO: 200 - DNA, 40 bp, synthetic construct
  misc_feature 1..40: note = Description of sequence: Synthetic oligonucleotide
  source 1..40: mol_type = other DNA
cacagagaatggtggatttccactgaaatgattgttcatc

SEQ ID NO: 201 - DNA, 41 bp, Nicotiana tabacum
  source 1..41: mol_type = genomic DNA
tggcatttccaaacaccaaaacgggcaccagaatggcactt

SEQ ID NO: 202 - DNA, 41 bp, Nicotiana tabacum
  source 1..41: mol_type = genomic DNA
ccaactctattaagcctggttggttttcagagtttagcgca

SEQ ID NO: 203 - DNA, 47 bp, Nicotiana tabacum
  source 1..47: mol_type = genomic DNA
ttctgactttggatggagcaattcaacatacagagaatggtggattt

SEQ ID NO: 204 - DNA, 50 bp, Nicotiana tabacum
  source 1..50: mol_type = genomic DNA
ctgactttggatggagcaattcaacatacagagaatggtggatttccata

SEQ ID NO: 205 - DNA, 47 bp, Nicotiana tabacum
  source 1..47: mol_type = genomic DNA
tgactttggatggagcaattcaacatacagagaatggtggatttcca

SEQ ID NO: 206 - DNA, 50 bp, Nicotiana tabacum
  source 1..50: mol_type = genomic DNA
tgactttggatggagcaattcaacatacagagaatggtggatttccatac

SEQ ID NO: 207 - DNA, 47 bp, Nicotiana tabacum
  source 1..47: mol_type = genomic DNA
gactttggatggagcaattcaacatacagagaatggtggatttccat

SEQ ID NO: 208 - DNA, 48 bp, Nicotiana tabacum
  source 1..48: mol_type = genomic DNA
gactttggatggagcaattcaacatacagagaatggtggatttccata

SEQ ID NO: 209 - DNA, 49 bp, Nicotiana tabacum
  source 1..49: mol_type = genomic DNA
gactttggatggagcaattcaacatacagagaatggtggatttccatac

SEQ ID NO: 210 - DNA, 56 bp, Nicotiana tabacum
  source 1..56: mol_type = genomic DNA
gactttggatggagcaattcaacatacagagaatggtggatttccatacactgaaa

SEQ ID NO: 211 - DNA, 44 bp, Nicotiana tabacum
  source 1..44: mol_type = genomic DNA
actttggatggagcaattcaacatacagagaatggtggatttcc

SEQ ID NO: 212 - DNA, 45 bp, Nicotiana tabacum
  source 1..45: mol_type = genomic DNA
actttggatggagcaattcaacatacagagaatggtggatttcca

SEQ ID NO: 213 - DNA, 46 bp, Nicotiana tabacum
  source 1..46: mol_type = genomic DNA
actttggatggagcaattcaacatacagagaatggtggatttccat

SEQ ID NO: 214 - DNA, 47 bp, Nicotiana tabacum
  source 1..47: mol_type = genomic DNA
actttggatggagcaattcaacatacagagaatggtggatttccata

SEQ ID NO: 215 - DNA, 64 bp, Nicotiana tabacum
  source 1..64: mol_type = genomic DNA
actttggatggagcaattcaacatacagagaatggtggatttccatacactgaaatgattgttc

SEQ ID NO: 216 - DNA, 42 bp, Nicotiana tabacum
  source 1..42: mol_type = genomic DNA
ctttggatggagcaattcaacatacagagaatggtggatttc

SEQ ID NO: 217 - DNA, 44 bp, Nicotiana tabacum
  source 1..44: mol_type = genomic DNA
ctttggatggagcaattcaacatacagagaatggtggatttcca

SEQ ID NO: 218 - DNA, 46 bp, Nicotiana tabacum
  source 1..46: mol_type = genomic DNA
ctttggatggagcaattcaacatacagagaatggtggatttccata

SEQ ID NO: 219 - DNA, 47 bp, Nicotiana tabacum
  source 1..47: mol_type = genomic DNA
ctttggatggagcaattcaacatacagagaatggtggatttccatac

SEQ ID NO: 220 - DNA, 41 bp, Nicotiana tabacum
  source 1..41: mol_type = genomic DNA
tttggatggagcaattcaacatacagagaatggtggatttc

SEQ ID NO: 221 - DNA, 43 bp, Nicotiana tabacum
  source 1..43: mol_type = genomic DNA
tttggatggagcaattcaacatacagagaatggtggatttcca

SEQ ID NO: 222 - DNA, 44 bp, Nicotiana tabacum
  source 1..44: mol_type = genomic DNA
tttggatggagcaattcaacatacagagaatggtggatttccat

SEQ ID NO: 223 - DNA, 45 bp, Nicotiana tabacum
  source 1..45: mol_type = genomic DNA
tttggatggagcaattcaacatacagagaatggtggatttccata

SEQ ID NO: 224 - DNA, 46 bp, Nicotiana tabacum
  source 1..46: mol_type = genomic DNA
tttggatggagcaattcaacatacagagaatggtggatttccatac

SEQ ID NO: 225 - DNA, 48 bp, Nicotiana tabacum
  source 1..48: mol_type = genomic DNA
tttggatggagcaattcaacatacagagaatggtggatttccatacac

SEQ ID NO: 226 - DNA, 42 bp, Nicotiana tabacum
  source 1..42: mol_type = genomic DNA
ttggatggagcaattcaacatacagagaatggtggatttcca

SEQ ID NO: 227 - DNA, 44 bp, Nicotiana tabacum
  source 1..44: mol_type = genomic DNA
ttggatggagcaattcaacatacagagaatggtggatttccata

SEQ ID NO: 228 - DNA, 42 bp, Nicotiana tabacum
  source 1..42: mol_type = genomic DNA
tggatggagcaattcaacatacagagaatggtggatttccat

SEQ ID NO: 229 - DNA, 43 bp, Nicotiana tabacum
  source 1..43: mol_type = genomic DNA
tggatggagcaattcaacatacagagaatggtggatttccata

SEQ ID NO: 230 - DNA, 44 bp, Nicotiana tabacum
  source 1..44: mol_type = genomic DNA
tggatggagcaattcaacatacagagaatggtggatttccatac

SEQ ID NO: 231 - DNA, 46 bp, Nicotiana tabacum
  source 1..46: mol_type = genomic DNA
tggatggagcaattcaacatacagagaatggtggatttccatacac

SEQ ID NO: 232 - DNA, 42 bp, Nicotiana tabacum
  source 1..42: mol_type = genomic DNA
ggatggagcaattcaacatacagagaatggtggatttccata

SEQ ID NO: 233 - DNA, 41 bp, Nicotiana tabacum
  source 1..41: mol_type = genomic DNA
caattcaacatacagagaatggtggatttccatacactgaa

SEQ ID NO: 234 - DNA, 40 bp, Nicotiana tabacum
  source 1..40: mol_type = genomic DNA
gcacttccaaacaccaaaacgggcaccagaatggcacttt

SEQ ID NO: 235 - DNA, 47 bp, Nicotiana tabacum; identical to SEQ ID NO: 203
  source 1..47: mol_type = genomic DNA

SEQ ID NO: 236 - DNA, 52 bp, Nicotiana tabacum
  source 1..52: mol_type = genomic DNA
ttctgactttggatggagcaattcaacatacagagaatggtggatttccata

SEQ ID NO: 237 - DNA, 47 bp, Nicotiana tabacum
  source 1..47: mol_type = genomic DNA
ctgactttggatggagcaattcaacatacagagaatggtggatttcc

SEQ ID NO: 238 - DNA, 50 bp, Nicotiana tabacum; identical to SEQ ID NO: 204
  source 1..50: mol_type = genomic DNA

SEQ ID NO: 239 - DNA, 47 bp, Nicotiana tabacum; identical to SEQ ID NO: 205
  source 1..47: mol_type = genomic DNA

SEQ ID NO: 240 - DNA, 50 bp, Nicotiana tabacum; identical to SEQ ID NO: 206
  source 1..50: mol_type = genomic DNA

SEQ ID NO: 241 - DNA, 47 bp, Nicotiana tabacum; identical to SEQ ID NO: 207
  source 1..47: mol_type = genomic DNA

SEQ ID NO: 242 - DNA, 48 bp, Nicotiana tabacum; identical to SEQ ID NO: 208
  source 1..48: mol_type = genomic DNA

SEQ ID NO: 243 - DNA, 49 bp, Nicotiana tabacum; identical to SEQ ID NO: 209
  source 1..49: mol_type = genomic DNA

SEQ ID NO: 244 - DNA, 50 bp, Nicotiana tabacum
  source 1..50: mol_type = genomic DNA
gactttggatggagcaattcaacatacagagaatggtggatttccataca

SEQ ID NO: 245 - DNA, 56 bp, Nicotiana tabacum; identical to SEQ ID NO: 210
  source 1..56: mol_type = genomic DNA

SEQ ID NO: 246 - DNA, 42 bp, Nicotiana tabacum
  source 1..42: mol_type = genomic DNA
actttggatggagcaattcaacatacagagaatggtggattt

SEQ ID NO: 247 - DNA, 44 bp, Nicotiana tabacum; identical to SEQ ID NO: 211
  source 1..44: mol_type = genomic DNA

SEQ ID NO: 248 - DNA, 46 bp, Nicotiana tabacum; identical to SEQ ID NO: 213
  source 1..46: mol_type = genomic DNA

SEQ ID NO: 249 - DNA, 47 bp, Nicotiana tabacum; identical to SEQ ID NO: 214
  source 1..47: mol_type = genomic DNA

SEQ ID NO: 250 - DNA, 48 bp, Nicotiana tabacum
  source 1..48: mol_type = genomic DNA
actttggatggagcaattcaacatacagagaatggtggatttccatac

SEQ ID NO: 251 - DNA, 50 bp, Nicotiana tabacum
  source 1..50: mol_type = genomic DNA
actttggatggagcaattcaacatacagagaatggtggatttccatacac

SEQ ID NO: 252 - DNA, 41 bp, Nicotiana tabacum
  source 1..41: mol_type = genomic DNA
ctttggatggagcaattcaacatacagagaatggtggattt

SEQ ID NO: 253 - DNA, 42 bp, Nicotiana tabacum; identical to SEQ ID NO: 216
  source 1..42: mol_type = genomic DNA

SEQ ID NO: 254 - DNA, 44 bp, Nicotiana tabacum; identical to SEQ ID NO: 217
  source 1..44: mol_type = genomic DNA

SEQ ID NO: 255 - DNA, 45 bp, Nicotiana tabacum
  source 1..45: mol_type = genomic DNA
ctttggatggagcaattcaacatacagagaatggtggatttccat

SEQ ID NO: 256 - DNA, 47 bp, Nicotiana tabacum; identical to SEQ ID NO: 219
  source 1..47: mol_type = genomic DNA

SEQ ID NO: 257 - DNA, 42 bp, Nicotiana tabacum
  source 1..42: mol_type = genomic DNA
tttggatggagcaattcaacatacagagaatggtggatttcc

SEQ ID NO: 258 - DNA, 44 bp, Nicotiana tabacum; identical to SEQ ID NO: 222
  source 1..44: mol_type = genomic DNA

SEQ ID NO: 259 - DNA, 45 bp, Nicotiana tabacum; identical to SEQ ID NO: 223
  source 1..45: mol_type = genomic DNA

SEQ ID NO: 260 - DNA, 46 bp, Nicotiana tabacum; identical to SEQ ID NO: 224
  source 1..46: mol_type = genomic DNA

SEQ ID NO: 261 - DNA, 47 bp, Nicotiana tabacum
  source 1..47: mol_type = genomic DNA
tttggatggagcaattcaacatacagagaatggtggatttccataca

SEQ ID NO: 262 - DNA, 54 bp, Nicotiana tabacum
  source 1..54: mol_type = genomic DNA
tttggatggagcaattcaacatacagagaatggtggatttccatacactgaaat

SEQ ID NO: 263 - DNA, 41 bp, Nicotiana tabacum
  source 1..41: mol_type = genomic DNA
ttggatggagcaattcaacatacagagaatggtggatttcc

SEQ ID NO: 264 - DNA, 42 bp, Nicotiana tabacum; identical to SEQ ID NO: 226
  source 1..42: mol_type = genomic DNA

SEQ ID NO: 265 - DNA, 43 bp, Nicotiana tabacum
  source 1..43: mol_type = genomic DNA
ttggatggagcaattcaacatacagagaatggtggatttccat

SEQ ID NO: 266 - DNA, 44 bp, Nicotiana tabacum; identical to SEQ ID NO: 227
  source 1..44: mol_type = genomic DNA

SEQ ID NO: 267 - DNA, 45 bp, Nicotiana tabacum
  source 1..45: mol_type = genomic DNA
ttggatggagcaattcaacatacagagaatggtggatttccatac

SEQ ID NO: 268 - DNA, 46 bp, Nicotiana tabacum
  source 1..46: mol_type = genomic DNA
ttggatggagcaattcaacatacagagaatggtggatttccataca

SEQ ID NO: 269 - DNA, 47 bp, Nicotiana tabacum
  source 1..47: mol_type = genomic DNA
ttggatggagcaattcaacatacagagaatggtggatttccatacac

SEQ ID NO: 270 - DNA, 42 bp, Nicotiana tabacum; identical to SEQ ID NO: 228
  source 1..42: mol_type = genomic DNA

SEQ ID NO: 271 - DNA, 44 bp, Nicotiana tabacum; identical to SEQ ID NO: 230
  source 1..44: mol_type = genomic DNA

SEQ ID NO: 272 - DNA, 47 bp, Nicotiana tabacum
  source 1..47: mol_type = genomic DNA
tggatggagcaattcaacatacagagaatggtggatttccatacact

SEQ ID NO: 273 - DNA, 42 bp, Nicotiana tabacum; identical to SEQ ID NO: 232
  source 1..42: mol_type = genomic DNA

SEQ ID NO: 274 - DNA, 43 bp, Nicotiana tabacum
  source 1..43: mol_type = genomic DNA
ggatggagcaattcaacatacagagaatggtggatttccatac

SEQ ID NO: 275 - DNA, 42 bp, Nicotiana tabacum
  source 1..42: mol_type = genomic DNA
gagcaattcaacatacagagaatggtggatttccatacactg

SEQ ID NO: 276 - DNA, 41 bp, Nicotiana tabacum
  source 1..41: mol_type = genomic DNA
tggcacttccaaacaccaaaacggccacaagaatgggactt

SEQ ID NO: 277 - DNA, 41 bp, Nicotiana tabacum
  source 1..41: mol_type = genomic DNA
ccaattgtattaagcctggttggttttcagagtttagcgca

SEQ ID NO: 278 - DNA, 45 bp, Nicotiana tabacum
  source 1..45: mol_type = genomic DNA
tgactttggatggagcaattcaacacacagagaatggtggatttc

SEQ ID NO: 279 - DNA, 47 bp, Nicotiana tabacum
  source 1..47: mol_type = genomic DNA
tgactttggatggagcaattcaacacacagagaatggtggatttcca

SEQ ID NO: 280 - DNA, 50 bp, Nicotiana tabacum
  source 1..50: mol_type = genomic DNA
tgactttggatggagcaattcaacacacagagaatggtggatttccatac

SEQ ID NO: 281 - DNA, 48 bp, Nicotiana tabacum
  source 1..48: mol_type = genomic DNA
gactttggatggagcaattcaacacacagagaatggtggatttccata

SEQ ID NO: 282 - DNA, 51 bp, Nicotiana tabacum
  source 1..51: mol_type = genomic DNA
gactttggatggagcaattcaacacacagagaatggtggatttccatacac

SEQ ID NO: 283 - DNA, 42 bp, Nicotiana tabacum
  source 1..42: mol_type = genomic DNA
actttggatggagcaattcaacacacagagaatggtggattt

SEQ ID NO: 284 - DNA, 44 bp, Nicotiana tabacum
  source 1..44: mol_type = genomic DNA
actttggatggagcaattcaacacacagagaatggtggatttcc

SEQ ID NO: 285 - DNA, 45 bp, Nicotiana tabacum
  source 1..45: mol_type = genomic DNA
actttggatggagcaattcaacacacagagaatggtggatttcca

SEQ ID NO: 286 - DNA, 46 bp, Nicotiana tabacum
  source 1..46: mol_type = genomic DNA
actttggatggagcaattcaacacacagagaatggtggatttccat

SEQ ID NO: 287 - DNA, 47 bp, Nicotiana tabacum
  source 1..47: mol_type = genomic DNA
actttggatggagcaattcaacacacagagaatggtggatttccata

SEQ ID NO: 288 - DNA, 48 bp, Nicotiana tabacum
  source 1..48: mol_type = genomic DNA
actttggatggagcaattcaacacacagagaatggtggatttccatac

SEQ ID NO: 289 - DNA, 50 bp, Nicotiana tabacum
  source 1..50: mol_type = genomic DNA
actttggatggagcaattcaacacacagagaatggtggatttccatacac

SEQ ID NO: 290 - DNA, 41 bp, Nicotiana tabacum
  source 1..41: mol_type = genomic DNA
ctttggatggagcaattcaacacacagagaatggtggattt

SEQ ID NO: 291 - DNA, 42 bp, Nicotiana tabacum
  source 1..42: mol_type = genomic DNA
ctttggatggagcaattcaacacacagagaatggtggatttc

SEQ ID NO: 292 - DNA, 44 bp, Nicotiana tabacum
  source 1..44: mol_type = genomic DNA
ctttggatggagcaattcaacacacagagaatggtggatttcca

SEQ ID NO: 293 - DNA, 47 bp, Nicotiana tabacum
  source 1..47: mol_type = genomic DNA
ctttggatggagcaattcaacacacagagaatggtggatttccatac

SEQ ID NO: 294 - DNA, 42 bp, Nicotiana tabacum
  source 1..42: mol_type = genomic DNA
tttggatggagcaattcaacacacagagaatggtggatttcc

SEQ ID NO: 295 - DNA, 44 bp, Nicotiana tabacum
  source 1..44: mol_type = genomic DNA
tttggatggagcaattcaacacacagagaatggtggatttccat

SEQ ID NO: 296 - DNA, 45 bp, Nicotiana tabacum
  source 1..45: mol_type = genomic DNA
tttggatggagcaattcaacacacagagaatggtggatttccata

SEQ ID NO: 297 - DNA, 46 bp, Nicotiana tabacum
  source 1..46: mol_type = genomic DNA
tttggatggagcaattcaacacacagagaatggtggatttccatac

SEQ ID NO: 298 - DNA, 48 bp, Nicotiana tabacum
  source 1..48: mol_type = genomic DNA
tttggatggagcaattcaacacacagagaatggtggatttccatacac

SEQ ID NO: 299 - DNA, 54 bp, Nicotiana tabacum
  source 1..54: mol_type = genomic DNA
tttggatggagcaattcaacacacagagaatggtggatttccatacactgaaat

SEQ ID NO: 300 - DNA, 42 bp, Nicotiana tabacum
  source 1..42: mol_type = genomic DNA
ttggatggagcaattcaacacacagagaatggtggatttcca

SEQ ID NO: 301 - DNA, 44 bp, Nicotiana tabacum
  source 1..44: mol_type = genomic DNA
ttggatggagcaattcaacacacagagaatggtggatttccata

SEQ ID NO: 302 - DNA, 46 bp, Nicotiana tabacum
  source 1..46: mol_type = genomic DNA
ttggatggagcaattcaacacacagagaatggtggatttccataca

SEQ ID NO: 303 - DNA, 47 bp, Nicotiana tabacum
  source 1..47: mol_type = genomic DNA
ttggatggagcaattcaacacacagagaatggtggatttccatacac

SEQ ID NO: 304 - DNA, 52 bp, Nicotiana tabacum
  source 1..52: mol_type = genomic DNA
ttggatggagcaattcaacacacagagaatggtggatttccatacactgaaa

SEQ ID NO: 305 - DNA, 43 bp, Nicotiana tabacum
  source 1..43: mol_type = genomic DNA
tggatggagcaattcaacacacagagaatggtggatttccata

SEQ ID NO: 306 - DNA, 44 bp, Nicotiana tabacum
  source 1..44: mol_type = genomic DNA
tggatggagcaattcaacacacagagaatggtggatttccatac

SEQ ID NO: 307 - DNA, 42 bp, Nicotiana tabacum
  source 1..42: mol_type = genomic DNA
ggatggagcaattcaacacacagagaatggtggatttccata

SEQ ID NO: 308 - DNA, 44 bp, Nicotiana tabacum
  source 1..44: mol_type = genomic DNA
ggatggagcaattcaacacacagagaatggtggatttccataca

SEQ ID NO: 309 - DNA, 49 bp, Nicotiana tabacum
  source 1..49: mol_type = genomic DNA
ggatggagcaattcaacacacagagaatggtggatttccatacactgaa

SEQ ID NO: 310 - DNA, 46 bp, Nicotiana tabacum
  source 1..46: mol_type = genomic DNA
gatggagcaattcaacacacagagaatggtggatttccatacactg

SEQ ID NO: 311 - DNA, 41 bp, Nicotiana tabacum
  source 1..41: mol_type = genomic DNA
tggcacttccaaacaccaaaacggccaccagaatggcactt

SEQ ID NO: 312 - DNA, 41 bp, Nicotiana tabacum; identical to SEQ ID NO: 202
  source 1..41: mol_type = genomic DNA

SEQ ID NO: 313 - DNA, 42 bp, Nicotiana tabacum
  source 1..42: mol_type = genomic DNA
aacatatgggaaggttctgactttggatggagcaattcaaca

SEQ ID NO: 314 - DNA, 42 bp, Nicotiana tabacum
  source 1..42: mol_type = genomic DNA
atgggaaggttctgactttggatggagcaattcaacacacag

SEQ ID NO: 315 - DNA, 42 bp, Nicotiana tabacum
  source 1..42: mol_type = genomic DNA
gttctgactttggatggagcaattcaacacacagagaatggt

SEQ ID NO: 316 - DNA, 47 bp, Nicotiana tabacum
  source 1..47: mol_type = genomic DNA
ctgactttggatggagcaattcaacacacagagaatggtggatttcc

SEQ ID NO: 317 - DNA, 50 bp, Nicotiana tabacum
  source 1..50: mol_type = genomic DNA
ctgactttggatggagcaattcaacacacagagaatggtggatttccata

SEQ ID NO: 318 - DNA, 51 bp, Nicotiana tabacum
  source 1..51: mol_type = genomic DNA
ctgactttggatggagcaattcaacacacagagaatggtggatttccatac

SEQ ID NO: 319 - DNA, 47 bp, Nicotiana tabacum; identical to SEQ ID NO: 279
  source 1..47: mol_type = genomic DNA

SEQ ID NO: 320 - DNA, 41 bp, Nicotiana tabacum
  source 1..41: mol_type = genomic DNA
gactttggatggagcaattcaacacacagagaatggtggat

SEQ ID NO: 321 - DNA, 42 bp, Nicotiana tabacum
  source 1..42: mol_type = genomic DNA
gactttggatggagcaattcaacacacagagaatggtggatt

SEQ ID NO: 322 - DNA, 48 bp, Nicotiana tabacum; identical to SEQ ID NO: 281
  source 1..48: mol_type = genomic DNA

SEQ ID NO: 323 - DNA, 51 bp, Nicotiana tabacum; identical to SEQ ID NO: 282
  source 1..51: mol_type = genomic DNA

SEQ ID NO: 324 - DNA, 42 bp, Nicotiana tabacum; identical to SEQ ID NO: 283
  source 1..42: mol_type = genomic DNA

SEQ ID NO: 325 - DNA, 44 bp, Nicotiana tabacum; identical to SEQ ID NO: 284
  source 1..44: mol_type = genomic DNA

SEQ ID NO: 326 - DNA, 46 bp, Nicotiana tabacum; identical to SEQ ID NO: 286
  source 1..46: mol_type = genomic DNA

SEQ ID NO: 327 - DNA, 48 bp, Nicotiana tabacum; identical to SEQ ID NO: 288
  source 1..48: mol_type = genomic DNA

SEQ ID NO: 328 - DNA, 50 bp, Nicotiana tabacum; identical to SEQ ID NO: 289
  source 1..50: mol_type = genomic DNA

SEQ ID NO: 329 - DNA, 55 bp, Nicotiana tabacum
  source 1..55: mol_type = genomic DNA
actttggatggagcaattcaacacacagagaatggtggatttccatacactgaaa

SEQ ID NO: 330 - DNA, 57 bp, Nicotiana tabacum
  source 1..57: mol_type = genomic DNA
actttggatggagcaattcaacacacagagaatggtggatttccatacactgaaatg

SEQ ID NO: 331 - DNA, 41 bp, Nicotiana tabacum; identical to SEQ ID NO: 290
  source 1..41: mol_type = genomic DNA

SEQ ID NO: 332 - DNA, 44 bp, Nicotiana tabacum; identical to SEQ ID NO: 292
  source 1..44: mol_type = genomic DNA

SEQ ID NO: 333 - DNA, 45 bp, Nicotiana tabacum
  source 1..45: mol_type = genomic DNA
ctttggatggagcaattcaacacacagagaatggtggatttccat

SEQ ID NO: 334 - DNA, 47 bp, Nicotiana tabacum; identical to SEQ ID NO: 293
  source 1..47: mol_type = genomic DNA

SEQ ID NO: 335 - DNA, 42 bp, Nicotiana tabacum; identical to SEQ ID NO: 294
  source 1..42: mol_type = genomic DNA

SEQ ID NO: 336 - DNA, 44 bp, Nicotiana tabacum; identical to SEQ ID NO: 295
  source 1..44: mol_type = genomic DNA

SEQ ID NO: 337 - DNA, 45 bp, Nicotiana tabacum; identical to SEQ ID NO: 296
  source 1..45: mol_type = genomic DNA

SEQ ID NO: 338 - DNA, 42 bp, Nicotiana tabacum; identical to SEQ ID NO: 300
  source 1..42: mol_type = genomic DNA

SEQ ID NO: 339 - DNA, 44 bp, Nicotiana tabacum; identical to SEQ ID NO: 301
  source 1..44: mol_type = genomic DNA

SEQ ID NO: 340 - DNA, 45 bp, Nicotiana tabacum
  source 1..45: mol_type = genomic DNA
ttggatggagcaattcaacacacagagaatggtggatttccatac

SEQ ID NO: 341 - DNA, 53 bp, Nicotiana tabacum
  source 1..53: mol_type = genomic DNA
ttggatggagcaattcaacacacagagaatggtggatttccatacactgaaat

SEQ ID NO: 342 - DNA, 42 bp, Nicotiana tabacum
  source 1..42: mol_type = genomic DNA
tggatggagcaattcaacacacagagaatggtggatttccat

SEQ ID NO: 343 - DNA, 44 bp, Nicotiana tabacum; identical to SEQ ID NO: 306
  source 1..44: mol_type = genomic DNA

SEQ ID NO: 344 - DNA, 48 bp, Nicotiana tabacum
  source 1..48: mol_type = genomic DNA
tggatggagcaattcaacacacagagaatggtggatttccatacactg

SEQ ID NO: 345 - DNA, 42 bp, Nicotiana tabacum; identical to SEQ ID NO: 307
  source 1..42: mol_type = genomic DNA

SEQ ID NO: 346 - DNA, 43 bp, Nicotiana tabacum
  source 1..43: mol_type = genomic DNA
tggagcaattcaacacacagagaatggtggatttccatacact

SEQ ID NO: 347 - DNA, 44 bp, Nicotiana tabacum
  source 1..44: mol_type = genomic DNA
tggagcaattcaacacacagagaatggtggatttccatacactg

SEQ ID NO: 348 - DNA, 41 bp, Nicotiana tabacum
  source 1..41: mol_type = genomic DNA
ggagcaattcaacacacagagaatggtggatttccatacac

SEQ ID NO: 349 - DNA, 45 bp, Nicotiana tabacum
  source 1..45: mol_type = genomic DNA
caattcaacacacagagaatggtggatttccatacactgaaatga

SEQ ID NO: 350 - DNA, 53 bp, Nicotiana tabacum
  source 1..53: mol_type = genomic DNA
aacacacagagaatggtggatttccatacactgaaatgattgttcatcttcca

SEQ ID NO: 351 - DNA, 41 bp, Nicotiana tabacum
  source 1..41: mol_type = genomic DNA
cggcacttccaaacaccaaaacggccaccataatggcactt

SEQ ID NO: 352 - DNA, 51 bp, Nicotiana tabacum
  source 1..51: mol_type = genomic DNA
ttttgactttggatggagcaattcaacacacagagaatggtggatttccat

SEQ ID NO: 353 - DNA, 52 bp, Nicotiana tabacum
  source 1..52: mol_type = genomic DNA
ttgactttggatggagcaattcaacacacagagaatggtggatttccataca

SEQ ID NO: 354 - DNA, 47 bp, Nicotiana tabacum; identical to SEQ ID NO: 279
  source 1..47: mol_type = genomic DNA

SEQ ID NO: 355 - DNA, 50 bp, Nicotiana tabacum; identical to SEQ ID NO: 280
  source 1..50: mol_type = genomic DNA

SEQ ID NO: 356 - DNA, 51 bp, Nicotiana tabacum; identical to SEQ ID NO: 282
  source 1..51: mol_type = genomic DNA

SEQ ID NO: 357 - DNA, 53 bp, Nicotiana tabacum
  source 1..53: mol_type = genomic DNA
gactttggatggagcaattcaacacacagagaatggtggatttccatacactg

SEQ ID NO: 358 - DNA, 42 bp, Nicotiana tabacum; identical to SEQ ID NO: 283
  source 1..42: mol_type = genomic DNA

SEQ ID NO: 359 - DNA, 44 bp, Nicotiana tabacum; identical to SEQ ID NO: 284
  source 1..44: mol_type = genomic DNA

SEQ ID NO: 360 - DNA, 46 bp, Nicotiana tabacum; identical to SEQ ID NO: 286
  source 1..46: mol_type = genomic DNA

SEQ ID NO: 361 - DNA, 47 bp, Nicotiana tabacum; identical to SEQ ID NO: 287
  source 1..47: mol_type = genomic DNA

SEQ ID NO: 362 - DNA, 48 bp, Nicotiana tabacum; identical to SEQ ID NO: 288
  source 1..48: mol_type = genomic DNA

SEQ ID NO: 363 - DNA, 44 bp, Nicotiana tabacum; identical to SEQ ID NO: 292
  source 1..44: mol_type = genomic DNA

SEQ ID NO: 364 - DNA, 45 bp, Nicotiana tabacum; identical to SEQ ID NO: 333
  source 1..45: mol_type = genomic DNA

SEQ ID NO: 365 - DNA, 42 bp, Nicotiana tabacum; identical to SEQ ID NO: 294
  source 1..42: mol_type = genomic DNA

SEQ ID NO: 366 - DNA, 44 bp, Nicotiana tabacum; identical to SEQ ID NO: 295
  source 1..44: mol_type = genomic DNA

SEQ ID NO: 367 - DNA, 45 bp, Nicotiana tabacum; identical to SEQ ID NO: 296
  source 1..45: mol_type = genomic DNA

SEQ ID NO: 368 - DNA, 44 bp, Nicotiana tabacum; identical to SEQ ID NO: 301
  source 1..44: mol_type = genomic DNA

SEQ ID NO: 369 - DNA, 45 bp, Nicotiana tabacum; identical to SEQ ID NO: 340
  source 1..45: mol_type = genomic DNA

SEQ ID NO: 370 - DNA, 42 bp, Nicotiana tabacum; identical to SEQ ID NO: 342
  source 1..42: mol_type = genomic DNA

SEQ ID NO: 371 - DNA, 43 bp, Nicotiana tabacum; identical to SEQ ID NO: 305
  source 1..43: mol_type = genomic DNA

SEQ ID NO: 372 - DNA, 44 bp, Nicotiana tabacum; identical to SEQ ID NO: 306
  source 1..44: mol_type = genomic DNA

SEQ ID NO: 373 - DNA, 47 bp, Nicotiana tabacum
  source 1..47: mol_type = genomic DNA
tggatggagcaattcaacacacagagaatggtggatttccatacact

SEQ ID NO: 374 - DNA, 42 bp, Nicotiana tabacum; identical to SEQ ID NO: 307
  source 1..42: mol_type = genomic DNA

SEQ ID NO: 375 - DNA, 41 bp, Nicotiana tabacum
  source 1..41: mol_type = genomic DNA
tggagcaattcaacacacagagaatggtggatttccataca

SEQ ID NO: 376 - DNA, 46 bp, Nicotiana tabacum
  source 1..46: mol_type = genomic DNA
gcaattcaacacacagagaatggtggatttccatacactgaaatga

SEQ ID NO: 377 - DNA, 42 bp, Nicotiana tabacum
  source 1..42: mol_type = genomic DNA
aacacacagagaatggtggatttccatacactgaaatgattg

SEQ ID NO: 378 - DNA, 44 bp, Nicotiana tabacum
  source 1..44: mol_type = genomic DNA
cacagagaatggtggatttccatacactgaaatgattgttcatc

SEQ ID NO: 379 - DNA, 10 bp, Nicotiana tabacum
  source 1..10: mol_type = genomic DNA
tcaacataca

SEQ ID NO: 380 - DNA, 10 bp, Nicotiana tabacum
  source 1..10: mol_type = genomic DNA
caacatacag

SEQ ID NO: 381 - DNA, 16 bp, Nicotiana tabacum
  source 1..16: mol_type = genomic DNA
aacatacagagaatgg

SEQ ID NO: 382 - DNA, 24 bp, Nicotiana tabacum
  source 1..24: mol_type = genomic DNA
acatacagagaatggtggatttcc

SEQ ID NO: 383 - DNA, 12 bp, Nicotiana tabacum
  source 1..12: mol_type = genomic DNA
attcaacataca

SEQ ID NO: 384 - DNA, 10 bp, Nicotiana tabacum
  source 1..10: mol_type = genomic DNA
tcaacataca

SEQ ID NO: 385 - DNA, 10 bp, Nicotiana tabacum
  source 1..10: mol_type = genomic DNA
caacatacag

SEQ ID NO: 386 - DNA, 10 bp, Nicotiana tabacum
  source 1..10: mol_type = genomic DNA
aacatacaga

SEQ ID NO: 387 - DNA, 16 bp, Nicotiana tabacum
  source 1..16: mol_type = genomic DNA
aacatacagagaatgg

SEQ ID NO: 388 - DNA, 10 bp, Nicotiana tabacum
  source 1..10: mol_type = genomic DNA
acatacagag

SEQ ID NO: 389 - DNA, 14 bp, Nicotiana tabacum
  source 1..14: mol_type = genomic DNA
atacagagaatggt

SEQ ID NO: 390 - DNA, 10 bp, Nicotiana tabacum
  source 1..10: mol_type = genomic DNA
caacacacag

SEQ ID NO: 391 - DNA, 11 bp, Nicotiana tabacum
  source 1..11: mol_type = genomic DNA
aacacacagag

SEQ ID NO: 392 - DNA, 10 bp, Nicotiana tabacum
  source 1..10: mol_type = genomic DNA
acacacagag

SEQ ID NO: 393 - DNA, 14 bp, Nicotiana tabacum
  source 1..14: mol_type = genomic DNA
acacagagaatggt

SEQ ID NO: 394 - DNA, 12 bp, Nicotiana tabacum
  source 1..12: mol_type = genomic DNA
cacagagaatgg

SEQ ID NO: 395 - DNA, 10 bp, Nicotiana tabacum
  source 1..10: mol_type = genomic DNA
tcaacacaca

SEQ ID NO: 396 - DNA, 11 bp, Nicotiana tabacum
  source 1..11: mol_type = genomic DNA
tcaacacacag

SEQ ID NO: 397 - DNA, 11 bp, Nicotiana tabacum
  source 1..11: mol_type = genomic DNA
aacacacagag

SEQ ID NO: 398 - DNA, 10 bp, Nicotiana tabacum
  source 1..10: mol_type = genomic DNA
acacacagag

SEQ ID NO: 399 - DNA, 15 bp, Nicotiana tabacum
  source 1..15: mol_type = genomic DNA
acacacagagaatgg

SEQ ID NO: 400 - DNA, 17 bp, Nicotiana tabacum
  source 1..17: mol_type = genomic DNA
acacacagagaatggtg

SEQ ID NO: 401 - DNA, 13 bp, Nicotiana tabacum
  source 1..13: mol_type = genomic DNA
cacagagaatggt

SEQ ID NO: 402 - DNA, 13 bp, Nicotiana tabacum
  source 1..13: mol_type = genomic DNA
tttccatacactg

SEQ ID NO: 403 - DNA, 11 bp, Nicotiana tabacum
  source 1..11: mol_type = genomic DNA
attcaacacac

SEQ ID NO: 404 - DNA, 12 bp, Nicotiana tabacum
  source 1..12: mol_type = genomic DNA
tcaacacacaga

SEQ ID NO: 405 - DNA, 10 bp, Nicotiana tabacum
  source 1..10: mol_type = genomic DNA
caacacacag

SEQ ID NO: 406 - DNA, 11 bp, Nicotiana tabacum
  source 1..11: mol_type = genomic DNA
aacacacagag

SEQ ID NO: 407 - DNA, 13 bp, Nicotiana tabacum
  source 1..13: mol_type = genomic DNA
aacacacagagaa

SEQ ID NO: 408 - DNA, 19 bp, Nicotiana tabacum
  source 1..19: mol_type = genomic DNA
gagcaattcaacatacaga

SEQ ID NO: 409 - DNA, 13 bp, Nicotiana tabacum
  source 1..13: mol_type = genomic DNA
ggagcaattcaac

SEQ ID NO: 410 - DNA, 86 bp, Nicotiana tabacum
  source 1..86: mol_type = genomic DNA
tcagcaacttatgggaaggttctgactttggatggagcaattcagagaatggtggatttccatacactgaaatgattgttcatcta

SEQ ID NO: 411 - DNA, 89 bp, Nicotiana tabacum
  source 1..89: mol_type = genomic DNA
tcagcaacttatgggaaggttctgactttggatggagcaattcaacagagaatggtggatttccatacactgaaatgattgttcatcta

SEQ ID NO: 412 - DNA, 83 bp, Nicotiana tabacum
  source 1..83: mol_type = genomic DNA
tcagcaacttatgggaaggttctgactttggatggagcaatgagaatggtggatttccatacactgaaatgattgttcatcta

SEQ ID NO: 413 - DNA, 89 bp, Nicotiana tabacum; identical to SEQ ID NO: 411
  source 1..89: mol_type = genomic DNA

SEQ ID NO: 414 - DNA, 89 bp, Nicotiana tabacum; identical to SEQ ID NO: 411
  source 1..89: mol_type = genomic DNA

SEQ ID NO: 415 - DNA, 86 bp, Nicotiana tabacum
  source 1..86: mol_type = genomic DNA
tcagcaacttatgggaaggttctgactttggatggagcaattcaaagaatggtggatttccatacactgaaatgattgttcatcta

SEQ ID NO: 416 - DNA, 86 bp, Nicotiana tabacum; identical to SEQ ID NO: 415
  source 1..86: mol_type = genomic DNA

SEQ ID NO: 417 - DNA, 89 bp, Nicotiana tabacum; identical to SEQ ID NO: 411
  source 1..89: mol_type = genomic DNA

SEQ ID NO: 418 - DNA, 91 bp, Nicotiana tabacum
  source 1..91: mol_type = genomic DNA
tcagcaacttatgggaaggttctgactttggatggagcaattcaacacagagaatggtggatttccatacactgaaatgattgttcatcta

SEQ ID NO: 419 - DNA, 89 bp, Nicotiana tabacum; identical to SEQ ID NO: 411
  source 1..89: mol_type = genomic DNA

SEQ ID NO: 420 - DNA, 74 bp, Nicotiana tabacum
  source 1..74: mol_type = genomic DNA
tcagcaacttatgggaaggttctgactttggatggaatggtggatttccatacactgaaatgattgttcatcta

SEQ ID NO: 421 - DNA, 89 bp, Nicotiana tabacum; identical to SEQ ID NO: 411
  source 1..89: mol_type = genomic DNA

SEQ ID NO: 422 - DNA, 91 bp, Nicotiana tabacum; identical to SEQ ID NO: 418
  source 1..91: mol_type = genomic DNA

SEQ ID NO: 423 - DNA, 91 bp, Nicotiana tabacum; identical to SEQ ID NO: 418
  source 1..91: mol_type = genomic DNA

SEQ ID NO: 424 - DNA, 89 bp, Nicotiana tabacum; identical to SEQ ID NO: 411
  source 1..89: mol_type = genomic DNA

SEQ ID NO: 425 - DNA, 89 bp, Nicotiana tabacum; identical to SEQ ID NO: 411
  source 1..89: mol_type = genomic DNA

SEQ ID NO: 426 - DNA, 91 bp, Nicotiana tabacum
  source 1..91: mol_type = genomic DNA
tcagcaacttatgggaaggttctgactttggatggagcaattcaacatagagaatggtggatttccatacactgaaatgattgttcatcta

SEQ ID NO: 427 - DNA, 86 bp, Nicotiana tabacum; identical to SEQ ID NO: 410
  source 1..86: mol_type = genomic DNA

SEQ ID NO: 428 - DNA, 89 bp, Nicotiana tabacum; identical to SEQ ID NO: 411
  source 1..89: mol_type = genomic DNA

SEQ ID NO: 429 - DNA, 91 bp, Nicotiana tabacum; identical to SEQ ID NO: 426
  source 1..91: mol_type = genomic DNA

SEQ ID NO: 430 - DNA, 86 bp, Nicotiana tabacum
  source 1..86: mol_type = genomic DNA
tcagcaacttatgggaaggttctgactttggatggagcaattcaacaaatggtggatttccatacactgaaatgattgttcatcta

SEQ ID NO: 431 - DNA, 91 bp, Nicotiana tabacum; identical to SEQ ID NO: 426
  source 1..91: mol_type = genomic DNA

SEQ ID NO: 432 - DNA, 89 bp, Nicotiana tabacum; identical to SEQ ID NO: 411
  source 1..89: mol_type = genomic DNA

SEQ ID NO: 433 - DNA, 89 bp, Nicotiana tabacum; identical to SEQ ID NO: 411
  source 1..89: mol_type = genomic DNA

SEQ ID NO: 434 - DNA, 89 bp, Nicotiana tabacum; identical to SEQ ID NO: 411
  source 1..89: mol_type = genomic DNA

SEQ ID NO: 435 - DNA, 89 bp, Nicotiana tabacum; identical to SEQ ID NO: 411
  source 1..89: mol_type = genomic DNA

SEQ ID NO: 436 - DNA, 89 bp, Nicotiana tabacum; identical to SEQ ID NO: 411
  source 1..89: mol_type = genomic DNA

SEQ ID NO: 437 - DNA, 89 bp, Nicotiana tabacum; identical to SEQ ID NO: 411
  source 1..89: mol_type = genomic DNA

SEQ ID NO: 438 - DNA, 80 bp, Nicotiana tabacum
  source 1..80: mol_type = genomic DNA
tcagcaacttatgggaaggttctgactttggatatacagagaatggtggatttccatacactgaaatgattgttcatcta

SEQ ID NO: 439 - DNA, 89 bp, Nicotiana tabacum; identical to SEQ ID NO: 411
  source 1..89: mol_type = genomic DNA

SEQ ID NO: 440 - DNA, 91 bp, Nicotiana tabacum
  source 1..91: mol_type = genomic DNA
tcagcaacttatgggaaggttctgactttggatggagcaattcaatacagagaatggtggatttccatacactgaaatgattgttcatcta

SEQ ID NO: 441 - DNA, 86 bp, Nicotiana tabacum; identical to SEQ ID NO: 410
  source 1..86: mol_type = genomic DNA

SEQ ID NO: 442 - DNA, 93 bp, Nicotiana tabacum
  source 1..93: mol_type = genomic DNA
tcagcaacttatgggaaggttctgactttggatggagcaattcaacatacagagaatggtggatttccatacactgaaatgattgttcatcta

SEQ ID NO: 443 - DNA, 93 bp, Nicotiana tabacum; identical to SEQ ID NO: 442
  source 1..93: mol_type = genomic DNA

SEQ ID NO: 444 - DNA, 93 bp, Nicotiana tabacum; identical to SEQ ID NO: 442
  source 1..93: mol_type = genomic DNA

SEQ ID NO: 445 - DNA, 93 bp, Nicotiana tabacum; identical to SEQ ID NO: 442
  source 1..93: mol_type = genomic DNA

SEQ ID NO: 446 - DNA, 93 bp, Nicotiana tabacum; identical to SEQ ID NO: 442
  source 1..93: mol_type = genomic DNA

SEQ ID NO: 447 - DNA, 104 bp, Nicotiana tabacum
  source 1..104: mol_type = genomic DNA
tcagcaacttatgggaaggttctgactttggatggagcaattcaacatacagagaatggtggatttccatacactgaaatgattgttcatctatcaaagaatgg

SEQ ID NO: 448 - DNA, 104 bp, Nicotiana tabacum; identical to SEQ ID NO: 447
  source 1..104: mol_type = genomic DNA

SEQ ID NO: 449 - DNA, 93 bp, Nicotiana tabacum; identical to SEQ ID NO: 442
  source 1..93: mol_type = genomic DNA

SEQ ID NO: 450 - DNA, 93 bp, Nicotiana tabacum; identical to SEQ ID NO: 442
  source 1..93: mol_type = genomic DNA

SEQ ID NO: 451 - DNA, 93 bp, Nicotiana tabacum; identical to SEQ ID NO: 442
  source 1..93: mol_type = genomic DNA

SEQ ID NO: 452 - DNA, 93 bp, Nicotiana tabacum; identical to SEQ ID NO: 442
  source 1..93: mol_type = genomic DNA

SEQ ID NO: 453 - DNA, 93 bp, Nicotiana tabacum; identical to SEQ ID NO: 442
  source 1..93: mol_type = genomic DNA

SEQ ID NO: 454 - DNA, 93 bp, Nicotiana tabacum; identical to SEQ ID NO: 442
  source 1..93: mol_type = genomic DNA

SEQ ID NO: 455 - DNA, 93 bp, Nicotiana tabacum; identical to SEQ ID NO: 442
  source 1..93: mol_type = genomic DNA

SEQ ID NO: 456 - DNA, 93 bp, Nicotiana tabacum; identical to SEQ ID NO: 442
  source 1..93: mol_type = genomic DNA

SEQ ID NO: 457 - DNA, 93 bp, Nicotiana tabacum; identical to SEQ ID NO: 442
  source 1..93: mol_type = genomic DNA

SEQ ID NO: 458 - DNA, 93 bp, Nicotiana tabacum; identical to SEQ ID NO: 442
  source 1..93: mol_type = genomic DNA

SEQ ID NO: 459 - DNA, 93 bp, Nicotiana tabacum; identical to SEQ ID NO: 442
  source 1..93: mol_type = genomic DNA

SEQ ID NO: 460 - DNA, 93 bp, Nicotiana tabacum; identical to SEQ ID NO: 442
  source 1..93: mol_type = genomic DNA

SEQ ID NO: 461 - DNA, 93 bp, Nicotiana tabacum; identical to SEQ ID NO: 442
  source 1..93: mol_type = genomic DNA

SEQ ID NO: 462 - DNA, 93 bp, Nicotiana tabacum; identical to SEQ ID NO: 442
  source 1..93: mol_type = genomic DNA

SEQ ID NO: 463 - DNA, 93 bp, Nicotiana tabacum; identical to SEQ ID NO: 442
  source 1..93: mol_type = genomic DNA

SEQ ID NO: 464 - DNA, 93 bp, Nicotiana tabacum; identical to SEQ ID NO: 442
  source 1..93: mol_type = genomic DNA

SEQ ID NO: 465 - DNA, 93 bp, Nicotiana tabacum; identical to SEQ ID NO: 442
  source 1..93: mol_type = genomic DNA

SEQ ID NO: 466 - DNA, 93 bp, Nicotiana tabacum; identical to SEQ ID NO: 442
  source 1..93: mol_type = genomic DNA

SEQ ID NO: 467 - DNA, 93 bp, Nicotiana tabacum; identical to SEQ ID NO: 442
  source 1..93: mol_type = genomic DNA

SEQ ID NO: 468 - DNA, 93 bp, Nicotiana tabacum; identical to SEQ ID NO: 442
  source 1..93: mol_type = genomic DNA

SEQ ID NO: 469 - DNA, 93 bp, Nicotiana tabacum; identical to SEQ ID NO: 442
  source 1..93: mol_type = genomic DNA

SEQ ID NO: 470 - DNA, 93 bp, Nicotiana tabacum; identical to SEQ ID NO: 442
  source 1..93: mol_type = genomic DNA

SEQ ID NO: 471 - DNA, 93 bp, Nicotiana tabacum; identical to SEQ ID NO: 442
  source 1..93: mol_type = genomic DNA

SEQ ID NO: 472 - DNA, 93 bp, Nicotiana tabacum; identical to SEQ ID NO: 442
  source 1..93: mol_type = genomic DNA

SEQ ID NO: 473 - DNA, 93 bp, Nicotiana tabacum; identical to SEQ ID NO: 442
  source 1..93: mol_type = genomic DNA

SEQ ID NO: 474 - DNA, 89 bp, Nicotiana tabacum; identical to SEQ ID NO: 411
  source 1..89: mol_type = genomic DNA

SEQ ID NO: 475 - DNA, 89 bp, Nicotiana tabacum; identical to SEQ ID NO: 411
  source 1..89: mol_type = genomic DNA

SEQ ID NO: 476 - DNA, 89 bp, Nicotiana tabacum; identical to SEQ ID NO: 411
  source 1..89: mol_type = genomic DNA

SEQ ID NO: 477 - DNA, 89 bp, Nicotiana tabacum; identical to SEQ ID NO: 411
  source 1..89: mol_type = genomic DNA

SEQ ID NO: 478 - DNA, 89 bp, Nicotiana tabacum; identical to SEQ ID NO: 411
  source 1..89: mol_type = genomic DNA

SEQ ID NO: 479 - DNA, 91 bp, Nicotiana tabacum; identical to SEQ ID NO: 426
  source 1..91: mol_type = genomic DNA

SEQ ID NO: 480 - DNA, 91 bp, Nicotiana tabacum; identical to SEQ ID NO: 426
  source 1..91: mol_type = genomic DNA

SEQ ID NO: 481 - DNA, 89 bp, Nicotiana tabacum; identical to SEQ ID NO: 411
  source 1..89: mol_type = genomic DNA

SEQ ID NO: 482 - DNA, 86 bp, Nicotiana tabacum; identical to SEQ ID NO: 410
  source 1..86: mol_type = genomic DNA

SEQ ID NO: 483 - DNA, 91 bp, Nicotiana tabacum; identical to SEQ ID NO: 418
  source 1..91: mol_type = genomic DNA

SEQ ID NO: 484 - DNA, 89 bp, Nicotiana tabacum; identical to SEQ ID NO: 411
  source 1..89: mol_type = genomic DNA

SEQ ID NO: 485 - DNA, 86 bp, Nicotiana tabacum; identical to SEQ ID NO: 410
  source 1..86: mol_type = genomic DNA

SEQ ID NO: 486 - DNA, 89 bp, Nicotiana tabacum; identical to SEQ ID NO: 411
  source 1..89: mol_type = genomic DNA

SEQ ID NO: 487 - DNA, 89 bp, Nicotiana tabacum; identical to SEQ ID NO: 411
  source 1..89: mol_type = genomic DNA

SEQ ID NO: 488 - DNA, 89 bp, Nicotiana tabacum; identical to SEQ ID NO: 411
  source 1..89: mol_type = genomic DNA

SEQ ID NO: 489 - DNA, 89 bp, Nicotiana tabacum; identical to SEQ ID NO: 411
  source 1..89: mol_type = genomic DNA

SEQ ID NO: 490 - DNA, 89 bp, Nicotiana tabacum; identical to SEQ ID NO: 411
  source 1..89: mol_type = genomic DNA

SEQ ID NO: 491 - DNA, 91 bp, Nicotiana tabacum; identical to SEQ ID NO: 440
  source 1..91: mol_type = genomic DNA

SEQ ID NO: 492 - DNA, 88 bp, Nicotiana tabacum
  source 1..88: mol_type = genomic DNA
tcagcaacttatgggaaggttctgactttggatggagcaattcacagagaatggtggatttccatacactgaaatgattgttcatcta

SEQ ID NO: 493 - DNA, 86 bp, Nicotiana tabacum; identical to SEQ ID NO: 410
  source 1..86: mol_type = genomic DNA

SEQ ID NO: 494 - DNA, 89 bp, Nicotiana tabacum; identical to SEQ ID NO: 411
  source 1..89: mol_type = genomic DNA

SEQ ID NO: 495 - DNA, 89 bp, Nicotiana tabacum; identical to SEQ ID NO: 411
  source 1..89: mol_type = genomic DNA

SEQ ID NO: 496 - DNA, 89 bp, Nicotiana tabacum
  source 1..89: mol_type = genomic DNA
tcagcaacttatgggaaggttctgactttggatggagcaattcaacatagaatggtggatttccatacactgaaatgattgttcatcta

SEQ ID NO: 497 - DNA, 89 bp, Nicotiana tabacum; identical to SEQ ID NO: 411
  source 1..89: mol_type = genomic DNA

SEQ ID NO: 498 - DNA, 88 bp, Nicotiana tabacum; identical to SEQ ID NO: 492
  source 1..88: mol_type = genomic DNA

SEQ ID NO: 499 - DNA, 89 bp, Nicotiana tabacum; identical to SEQ ID NO: 411
  source 1..89: mol_type = genomic DNA

SEQ ID NO: 500 - DNA, 89 bp, Nicotiana tabacum; identical to SEQ ID NO: 411
  source 1..89: mol_type = genomic DNA

SEQ ID NO: 501 - DNA, 89 bp, Nicotiana tabacum; identical to SEQ ID NO: 411
  source 1..89: mol_type = genomic DNA

SEQ ID NO: 502 - DNA, 83 bp, Nicotiana tabacum
  source 1..83: mol_type = genomic DNA
tcagcaacttatgggaaggttctgactttggatggagcaattagaatggtggatttccatacactgaaatgattgttcatcta

SEQ ID NO: 503 - DNA, 89 bp, Nicotiana tabacum; identical to SEQ ID NO: 496
  source 1..89: mol_type = genomic DNA

SEQ ID NO: 504 - DNA, 89 bp, Nicotiana tabacum; identical to SEQ ID NO: 411
  source 1..89: mol_type = genomic DNA

SEQ ID NO: 505 - DNA, 85 bp, Nicotiana tabacum
  source 1..85: mol_type = genomic DNA
tcagcaacttatgggaaggttctgactttggatggagcaattcaacactggtggatttccatacactgaaatgattgttcatcta

SEQ ID NO: 506 - DNA, 93 bp, Nicotiana tabacum; identical to SEQ ID NO: 442
  source 1..93: mol_type = genomic DNA

SEQ ID NO: 507 - DNA, 93 bp, Nicotiana tabacum; identical to SEQ ID NO: 442
  source 1..93: mol_type = genomic DNA

SEQ ID NO: 508 - DNA, 93 bp, Nicotiana tabacum; identical to SEQ ID NO: 442
  source 1..93: mol_type = genomic DNA

SEQ ID NO: 509 - DNA, 93 bp, Nicotiana tabacum; identical to SEQ ID NO: 442
  source 1..93: mol_type = genomic DNA

SEQ ID NO: 510 - DNA, 93 bp, Nicotiana tabacum; identical to SEQ ID NO: 442
  source 1..93: mol_type = genomic DNA

SEQ ID NO: 511 - DNA, 93 bp, Nicotiana tabacum; identical to SEQ ID NO: 442
  source 1..93: mol_type = genomic DNA

SEQ ID NO: 512 - DNA, 93 bp, Nicotiana tabacum; identical to SEQ ID NO: 442
  source 1..93: mol_type = genomic DNA

SEQ ID NO: 513 - DNA, 93 bp, Nicotiana tabacum; identical to SEQ ID NO: 442
  source 1..93: mol_type = genomic DNA

SEQ ID NO: 514 - DNA, 93 bp, Nicotiana tabacum; identical to SEQ ID NO: 442
  source 1..93: mol_type = genomic DNA

SEQ ID NO: 515 - DNA, 93 bp, Nicotiana tabacum; identical to SEQ ID NO: 442
  source 1..93: mol_type = genomic DNA

SEQ ID NO: 516 - DNA, 93 bp, Nicotiana tabacum; identical to SEQ ID NO: 442
  source 1..93: mol_type = genomic DNA

SEQ ID NO: 517 - DNA, 93 bp, Nicotiana tabacum; identical to SEQ ID NO: 442
  source 1..93: mol_type = genomic DNA

SEQ ID NO: 518 - DNA, 93 bp, Nicotiana tabacum; identical to SEQ ID NO: 442
  source 1..93: mol_type = genomic DNA

SEQ ID NO: 519 - DNA, 93 bp, Nicotiana tabacum; identical to SEQ ID NO: 442
  source 1..93: mol_type = genomic DNA

SEQ ID NO: 520 - DNA, 93 bp, Nicotiana tabacum; identical to SEQ ID NO: 442
  source 1..93: mol_type = genomic DNA

SEQ ID NO: 521 - DNA, 93 bp, Nicotiana tabacum; identical to SEQ ID NO: 442
  source 1..93: mol_type = genomic DNA

SEQ ID NO: 522 - DNA, 93 bp, Nicotiana tabacum; identical to SEQ ID NO: 442
  source 1..93: mol_type = genomic DNA

SEQ ID NO: 523 - DNA, 93 bp, Nicotiana tabacum; identical to SEQ ID NO: 442
  source 1..93: mol_type = genomic DNA

SEQ ID NO: 524 - DNA, 93 bp, Nicotiana tabacum; identical to SEQ ID NO: 442
  source 1..93: mol_type = genomic DNA

SEQ ID NO: 525 - DNA, 93 bp, Nicotiana tabacum; identical to SEQ ID NO: 442
  source 1..93: mol_type = genomic DNA

SEQ ID NO: 526 - DNA, 93 bp, Nicotiana tabacum; identical to SEQ ID NO: 442
  source 1..93: mol_type = genomic DNA

SEQ ID NO: 527 - DNA, 93 bp, Nicotiana tabacum; identical to SEQ ID NO: 442
  source 1..93: mol_type = genomic DNA

SEQ ID NO: 528 - DNA, 93 bp, Nicotiana tabacum; identical to SEQ ID NO: 442
  source 1..93: mol_type = genomic DNA

SEQ ID NO: 529 - DNA, 93 bp, Nicotiana tabacum; identical to SEQ ID NO: 442
  source 1..93: mol_type = genomic DNA

SEQ ID NO: 530 - DNA, 93 bp, Nicotiana tabacum; identical to SEQ ID NO: 442
  source 1..93: mol_type = genomic DNA

SEQ ID NO: 531 - DNA, 93 bp, Nicotiana tabacum; identical to SEQ ID NO: 442
  source 1..93: mol_type = genomic DNA

SEQ ID NO: 532 - DNA, 93 bp, Nicotiana tabacum; identical to SEQ ID NO: 442
  source 1..93: mol_type = genomic DNA

SEQ ID NO: 533 - DNA, 93 bp, Nicotiana tabacum; identical to SEQ ID NO: 442
  source 1..93: mol_type = genomic DNA

SEQ ID NO: 534 - DNA, 93 bp, Nicotiana tabacum; identical to SEQ ID NO: 442
  source 1..93: mol_type = genomic DNA

SEQ ID NO: 535 - DNA, 93 bp, Nicotiana tabacum; identical to SEQ ID NO: 442
  source 1..93: mol_type = genomic DNA

SEQ ID NO: 536 - DNA, 93 bp, Nicotiana tabacum; identical to SEQ ID NO: 442
  source 1..93: mol_type = genomic DNA

SEQ ID NO: 537 - DNA, 93 bp, Nicotiana tabacum
  source 1..93: mol_type = genomic DNA
tcagcaacttatgggaaggttctgactttggatggagcaattcaacatacagagactggtggatttccatacactgaaatgattgttcatcta

SEQ ID NO: 538 - DNA, 91 bp, Nicotiana tabacum
  source 1..91: mol_type = genomic DNA
tcagcaacttatgggaaggttctgactttggatggagcaattcaacacagagaatggtggatttccatacactgaaatgattgttcatctt

SEQ ID NO: 539 - DNA, 91 bp, Nicotiana tabacum; identical to SEQ ID NO: 538
  source 1..91: mol_type = genomic DNA

SEQ ID NO: 540 - DNA, 91 bp, Nicotiana tabacum; identical to SEQ ID NO: 538
  source 1..91: mol_type = genomic DNA

SEQ ID NO: 541 - DNA, 85 bp, Nicotiana tabacum
  source 1..85: mol_type = genomic DNA
tcagcaacttatgggaaggttctgactttggatggagcaattcaagaatggtggatttccatacactgaaatgattgttcatctt

SEQ ID NO: 542 - DNA, 91 bp, Nicotiana tabacum; identical to SEQ ID NO: 538
  source 1..91: mol_type = genomic DNA

SEQ ID NO: 543 - DNA, 91 bp, Nicotiana tabacum; identical to SEQ ID NO: 538
  source 1..91: mol_type = genomic DNA

SEQ ID NO: 544 - DNA, 91 bp, Nicotiana tabacum; identical to SEQ ID NO: 538
  source 1..91: mol_type = genomic DNA

SEQ ID NO: 545 - DNA, 89 bp, Nicotiana tabacum
  source 1..89: mol_type = genomic DNA
tcagcaacttatgggaaggttctgactttggatggagcaattcaacagagaatggtggatttccatacactgaaatgattgttcatctt

SEQ ID NO: 546 - DNA, 91 bp, Nicotiana tabacum; identical to SEQ ID NO: 538
  source 1..91: mol_type = genomic DNA

SEQ ID NO: 547 - DNA, 91 bp, Nicotiana tabacum; identical to SEQ ID NO: 538
  source 1..91: mol_type = genomic DNA

SEQ ID NO: 548 - DNA, 91 bp, Nicotiana tabacum; identical to SEQ ID NO: 538
  source 1..91: mol_type = genomic DNA

SEQ ID NO: 549 - DNA, 91 bp, Nicotiana tabacum; identical to SEQ ID NO: 538
  source 1..91: mol_type = genomic DNA

SEQ ID NO: 550 - DNA, 91 bp, Nicotiana tabacum; identical to SEQ ID NO: 538
  source 1..91: mol_type = genomic DNA

SEQ ID NO: 551 - DNA, 91 bp, Nicotiana tabacum; identical to SEQ ID NO: 538
  source 1..91: mol_type = genomic DNA

SEQ ID NO: 552 - DNA, 91 bp, Nicotiana tabacum; identical to SEQ ID NO: 538
  source 1..91: mol_type = genomic DNA

SEQ ID NO: 553 - DNA, 91 bp, Nicotiana tabacum; identical to SEQ ID NO: 538
  source 1..91: mol_type = genomic DNA

SEQ ID NO: 554 - DNA, 91 bp, Nicotiana tabacum; identical to SEQ ID NO: 538
  source 1..91: mol_type = genomic DNA

SEQ ID NO: 555 - DNA, 91 bp, Nicotiana tabacum; identical to SEQ ID NO: 538
  source 1..91: mol_type = genomic DNA

SEQ ID NO: 556 - DNA, 85 bp, Nicotiana tabacum; identical to SEQ ID NO: 541
  source 1..85: mol_type = genomic DNA

SEQ ID NO: 557 - DNA, 85 bp, Nicotiana tabacum; identical to SEQ ID NO: 541
  source 1..85: mol_type = genomic DNA

SEQ ID NO: 558 - DNA, 91 bp, Nicotiana tabacum; identical to SEQ ID NO: 538
  source 1..91: mol_type = genomic DNA

SEQ ID NO: 559 - DNA, 89 bp, Nicotiana tabacum; identical to SEQ ID NO: 545
  source 1..89: mol_type = genomic DNA

SEQ ID NO: 560 - DNA, 89 bp, Nicotiana tabacum; identical to SEQ ID NO: 545
  source 1..89: mol_type = genomic DNA

SEQ ID NO: 561 - DNA, 91 bp, Nicotiana tabacum; identical to SEQ ID NO: 538
  source 1..91: mol_type = genomic DNA

SEQ ID NO: 562 - DNA, 91 bp, Nicotiana tabacum; identical to SEQ ID NO: 538
  source 1..91: mol_type = genomic DNA

SEQ ID NO: 563 - DNA, 91 bp, Nicotiana tabacum; identical to SEQ ID NO: 538
  source 1..91: mol_type = genomic DNA

SEQ ID NO: 564 - DNA, 89 bp, Nicotiana tabacum; identical to SEQ ID NO: 545
  source 1..89: mol_type = genomic DNA

SEQ ID NO: 565 - DNA, 89 bp, Nicotiana tabacum; identical to SEQ ID NO: 545
  source 1..89: mol_type = genomic DNA

SEQ ID NO: 566 - DNA, 89 bp, Nicotiana tabacum; identical to SEQ ID NO: 545
  source 1..89: mol_type = genomic DNA

SEQ ID NO: 567 - DNA, 89 bp, Nicotiana tabacum; identical to SEQ ID NO: 545
  source 1..89: mol_type = genomic DNA

SEQ ID NO: 568 - DNA, 89 bp, Nicotiana tabacum; identical to SEQ ID NO: 545
  source 1..89: mol_type = genomic DNA

SEQ ID NO: 569 - DNA, 89 bp, Nicotiana tabacum; identical to SEQ ID NO: 545
  source 1..89: mol_type = genomic DNA

SEQ ID NO: 570 - DNA, 93 bp, Nicotiana tabacum
  source 1..93: mol_type = genomic DNA
tcagcaacttatgggaaggttctgactttggatggagcaattcaacacacagagaatggtggatttccatacactgaaatgattgttcatctt

SEQ ID NO: 571 - DNA, 93 bp, Nicotiana tabacum; identical to SEQ ID NO: 570
  source 1..93: mol_type = genomic DNA

SEQ ID NO: 572 - DNA, 93 bp, Nicotiana tabacum; identical to SEQ ID NO: 570
  source 1..93: mol_type = genomic DNA

SEQ ID NO: 573 - DNA, 93 bp, Nicotiana tabacum; identical to SEQ ID NO: 570
  source 1..93: mol_type = genomic DNA

SEQ ID NO: 574 - DNA, 93 bp, Nicotiana tabacum; identical to SEQ ID NO: 570
  source 1..93: mol_type = genomic DNA

SEQ ID NO: 575 - DNA, 93 bp, Nicotiana tabacum; identical to SEQ ID NO: 570
  source 1..93: mol_type = genomic DNA

SEQ ID NO: 576 - DNA, 93 bp, Nicotiana tabacum; identical to SEQ ID NO: 570
  source 1..93: mol_type = genomic DNA

SEQ ID NO: 577 - DNA, 93 bp, Nicotiana tabacum; identical to SEQ ID NO: 570
  source 1..93: mol_type = genomic DNA

SEQ ID NO: 578 - DNA, 93 bp, Nicotiana tabacum; identical to SEQ ID NO: 570
  source 1..93: mol_type = genomic DNA

SEQ ID NO: 579 - DNA, 93 bp, Nicotiana tabacum; identical to SEQ ID NO: 570
  source 1..93: mol_type = genomic DNA

SEQ ID NO: 580 - DNA, 93 bp, Nicotiana tabacum; identical to SEQ ID NO: 570
  source 1..93: mol_type = genomic DNA

SEQ ID NO: 581 - DNA, 93 bp, Nicotiana tabacum; identical to SEQ ID NO: 570
  source 1..93: mol_type = genomic DNA

SEQ ID NO: 582 - DNA, 93 bp, Nicotiana tabacum; identical to SEQ ID NO: 570
  source 1..93: mol_type = genomic DNA

SEQ ID NO: 583 - DNA, 93 bp, Nicotiana tabacum; identical to SEQ ID NO: 570
  source 1..93: mol_type = genomic DNA

SEQ ID NO: 584 - DNA, 93 bp, Nicotiana tabacum; identical to SEQ ID NO: 570
  source 1..93: mol_type = genomic DNA

SEQ ID NO: 585 - DNA, 93 bp, Nicotiana tabacum; identical to SEQ ID NO: 570
  source 1..93: mol_type = genomic DNA

SEQ ID NO: 586 - DNA, 93 bp, Nicotiana tabacum; identical to SEQ ID NO: 570
  source 1..93: mol_type = genomic DNA

SEQ ID NO: 587 - DNA, 93 bp, Nicotiana tabacum; identical to SEQ ID NO: 570
  source 1..93: mol_type = genomic DNA

SEQ ID NO: 588 - DNA, 93 bp, Nicotiana tabacum; identical to SEQ ID NO: 570
  source 1..93: mol_type = genomic DNA

SEQ ID NO: 589 - DNA, 93 bp, Nicotiana tabacum; identical to SEQ ID NO: 570
  source 1..93: mol_type = genomic DNA

SEQ ID NO: 590 - DNA, 93 bp, Nicotiana tabacum; identical to SEQ ID NO: 570
  source 1..93: mol_type = genomic DNA

SEQ ID NO: 591 - DNA, 93 bp, Nicotiana tabacum; identical to SEQ ID NO: 570
  source 1..93: mol_type = genomic DNA

SEQ ID NO: 592 - DNA, 93 bp, Nicotiana tabacum; identical to SEQ ID NO: 570
  source 1..93: mol_type = genomic DNA

SEQ ID NO: 593 - DNA, 93 bp, Nicotiana tabacum; identical to SEQ ID NO: 570
  source 1..93: mol_type = genomic DNA

SEQ ID NO: 594 - DNA, 93 bp, Nicotiana tabacum; identical to SEQ ID NO: 570
  source 1..93: mol_type = genomic DNA

SEQ ID NO: 595 - DNA, 93 bp, Nicotiana tabacum; identical to SEQ ID NO: 570
  source 1..93: mol_type = genomic DNA

SEQ ID NO: 596 - DNA, 93 bp, Nicotiana tabacum; identical to SEQ ID NO: 570
  source 1..93: mol_type = genomic DNA

SEQ ID NO: 597 - DNA, 93 bp, Nicotiana tabacum; identical to SEQ ID NO: 570
  source 1..93: mol_type = genomic DNA

SEQ ID NO: 598 - DNA, 93 bp, Nicotiana tabacum; identical to SEQ ID NO: 570
  source 1..93: mol_type = genomic DNA

SEQ ID NO: 599 - DNA, 93 bp, Nicotiana tabacum; identical to SEQ ID NO: 570
  source 1..93: mol_type = genomic DNA

SEQ ID NO: 600 - DNA, 93 bp, Nicotiana tabacum; identical to SEQ ID NO: 570
  source 1..93: mol_type = genomic DNA

SEQ ID NO: 601 - DNA, 93 bp, Nicotiana tabacum; identical to SEQ ID NO: 570
  source 1..93: mol_type = genomic DNA

SEQ ID NO: 602 - DNA, 89 bp, Nicotiana tabacum
  source 1..89: mol_type = genomic DNA
tcagcaacatatgggaaggttctgactttggatggagcaattcaacagagaatggtggatttccatacactgaaatgattgttcatctt

SEQ ID NO: 603 - DNA, 89 bp, Nicotiana tabacum; identical to SEQ ID NO: 602
  source 1..89: mol_type = genomic DNA

SEQ ID NO: 604 - DNA, 88 bp, Nicotiana tabacum
  source 1..88: mol_type = genomic DNA
tcagcaacatatgggaaggttctgactttggatggagcaattcaaagagaatggtggatttccatacactgaaatgattgttcatctt

SEQ ID NO: 605 - DNA, 91 bp, Nicotiana tabacum
  source 1..91: mol_type = genomic DNA
tcagcaacatatgggaaggttctgactttggatggagcaattcaacacagagaatggtggatttccatacactgaaatgattgttcatctt

SEQ ID NO: 606 - DNA, 89 bp, Nicotiana tabacum; identical to SEQ ID NO: 602
  source 1..89: mol_type = genomic DNA

SEQ ID NO: 607 - DNA, 89 bp, Nicotiana tabacum; identical to SEQ ID NO: 602
  source 1..89: mol_type = genomic DNA

SEQ ID NO: 608 - DNA, 89 bp, Nicotiana tabacum; identical to SEQ ID NO: 602
  source 1..89: mol_type = genomic DNA

SEQ ID NO: 609 - DNA, 84 bp, Nicotiana tabacum
  source 1..84: mol_type = genomic DNA
tcagcaacatatgggaaggttctgactttggatggagcaattcagaatggtggatttccatacactgaaatgattgttcatctt

SEQ ID NO: 610 - DNA, 89 bp, Nicotiana tabacum; identical to SEQ ID NO: 602
  source 1..89: mol_type = genomic DNA

SEQ ID NO: 611 - DNA, 89 bp, Nicotiana tabacum; identical to SEQ ID NO: 602
  source 1..89: mol_type = genomic DNA

SEQ ID NO: 612 - DNA, 86 bp, Nicotiana tabacum
  source 1..86: mol_type = genomic DNA
tcagcaacatatgggaaggttctgactttggatggagcaattcagagaatggtggatttccatacactgaaatgattgttcatctt

SEQ ID NO: 613 - DNA, 86 bp, Nicotiana tabacum; identical to SEQ ID NO: 612
  source 1..86: mol_type = genomic DNA

SEQ ID NO: 614 - DNA, 86 bp, Nicotiana tabacum
  source 1..86: mol_type = genomic DNA
tcagcaacatatgggaaggttctgactttggatggagcaattcaaagaatggtggatttccatacactgaaatgattgttcatctt

SEQ ID NO: 615 - DNA, 89 bp, Nicotiana tabacum; identical to SEQ ID NO: 602
  source 1..89: mol_type = genomic DNA

SEQ ID NO: 616 - DNA, 86 bp, Nicotiana tabacum; identical to SEQ ID NO: 614
  source 1..86: mol_type = genomic DNA

SEQ ID NO: 617 - DNA, 89 bp, Nicotiana tabacum; identical to SEQ ID NO: 602
  source 1..89: mol_type = genomic DNA

SEQ ID NO: 618 - DNA, 89 bp, Nicotiana tabacum; identical to SEQ ID NO: 602
  source 1..89: mol_type = genomic DNA

SEQ ID NO: 619 - DNA, 89 bp, Nicotiana tabacum; identical to SEQ ID NO: 602
  source 1..89: mol_type = genomic DNA

SEQ ID NO: 620 - DNA, 91 bp, Nicotiana tabacum; identical to SEQ ID NO: 605
  source 1..91: mol_type = genomic DNA

SEQ ID NO: 621 - DNA, 91 bp, Nicotiana tabacum; identical to SEQ ID NO: 605
  source 1..91: mol_type = genomic DNA

SEQ ID NO: 622 - DNA, 91 bp, Nicotiana tabacum; identical to SEQ ID NO: 605
  source 1..91: mol_type = genomic DNA

SEQ ID NO: 623 - DNA, 89 bp, Nicotiana tabacum; identical to SEQ ID NO: 602
  source 1..89: mol_type = genomic DNA

SEQ ID NO: 624 - DNA, 91 bp, Nicotiana tabacum; identical to SEQ ID NO: 605
  source 1..91: mol_type = genomic DNA

SEQ ID NO: 625 - DNA, 89 bp, Nicotiana tabacum
  source 1..89: mol_type = genomic DNA
tcagcaacatatgggaaggttctgactttggatggagcaattcaacacagaatggtggatttccatacactgaaatgattgttcatctt

SEQ ID NO: 626 - DNA, 91 bp, Nicotiana tabacum; identical to SEQ ID NO: 605
  source 1..91: mol_type = genomic DNA

SEQ ID NO: 627 - DNA, 91 bp, Nicotiana tabacum; identical to SEQ ID NO: 605
  source 1..91: mol_type = genomic DNA

SEQ ID NO: 628 - DNA, 86 bp, Nicotiana tabacum; identical to SEQ ID NO: 612
  source 1..86: mol_type = genomic DNA

SEQ ID NO: 629 - DNA, 89 bp, Nicotiana tabacum; identical to SEQ ID NO: 602
  source 1..89: mol_type = genomic DNA

SEQ ID NO: 630 - DNA, 91 bp, Nicotiana tabacum; identical to SEQ ID NO: 605
  source 1..91: mol_type = genomic DNA

SEQ ID NO: 631 - DNA, 91 bp, Nicotiana tabacum; identical to SEQ ID NO: 605
  source 1..91: mol_type = genomic DNA

SEQ ID NO: 632 - DNA, 91 bp, Nicotiana tabacum; identical to SEQ ID NO: 605
  source 1..91: mol_type = genomic DNA

SEQ ID NO: 633 - DNA, 89 bp, Nicotiana tabacum; identical to SEQ ID NO: 602
  source 1..89: mol_type = genomic DNA

SEQ ID NO: 634 - DNA, 93 bp, Nicotiana tabacum
  source 1..93: mol_type = genomic DNA
tcagcaacatatgggaaggttctgactttggatggagcaattcaacacacagagaatggtggatttccatacactgaaatgattgttcatctt

SEQ ID NO: 635 - DNA, 93 bp, Nicotiana tabacum; identical to SEQ ID NO: 634
  source 1..93: mol_type = genomic DNA

SEQ ID NO: 636 - DNA, 93 bp, Nicotiana tabacum; identical to SEQ ID NO: 634
  source 1..93: mol_type = genomic DNA

SEQ ID NO: 637 - DNA, 93 bp, Nicotiana tabacum; identical to SEQ ID NO: 634
  source 1..93: mol_type = genomic DNA

SEQ ID NO: 638 - DNA, 93 bp, Nicotiana tabacum; identical to SEQ ID NO: 634
  source 1..93: mol_type = genomic DNA

SEQ ID NO: 639 - DNA, 93 bp, Nicotiana tabacum; identical to SEQ ID NO: 634
  source 1..93: mol_type = genomic DNA

SEQ ID NO: 640 - DNA, 93 bp, Nicotiana tabacum; identical to SEQ ID NO: 634
  source 1..93: mol_type = genomic DNA

SEQ ID NO: 641 - DNA, 93 bp, Nicotiana tabacum; identical to SEQ ID NO: 634
  source 1..93: mol_type = genomic DNA

SEQ ID NO: 642 - DNA, 93 bp, Nicotiana tabacum; identical to SEQ ID NO: 634
  source 1..93: mol_type = genomic DNA

SEQ ID NO: 643 - DNA, 93 bp, Nicotiana tabacum; identical to SEQ ID NO: 634
  source 1..93: mol_type = genomic DNA

SEQ ID NO: 644 - DNA, 93 bp, Nicotiana tabacum; identical to SEQ ID NO: 634
  source 1..93: mol_type = genomic DNA

SEQ ID NO: 645 - DNA, 93 bp, Nicotiana tabacum; identical to SEQ ID NO: 634
  source 1..93: mol_type = genomic DNA

SEQ ID NO: 646 - DNA, 93 bp, Nicotiana tabacum; identical to SEQ ID NO: 634
  source 1..93: mol_type = genomic DNA

SEQ ID NO: 647 - DNA, 93 bp, Nicotiana tabacum; identical to SEQ ID NO: 634
  source 1..93: mol_type = genomic DNA

SEQ ID NO: 648 - DNA, 93 bp, Nicotiana tabacum; identical to SEQ ID NO: 634
  source 1..93: mol_type = genomic DNA

SEQ ID NO: 649 - DNA, 93 bp, Nicotiana tabacum; identical to SEQ ID NO: 634
  source 1..93: mol_type = genomic DNA

SEQ ID NO: 650 - DNA, 93 bp, Nicotiana tabacum; identical to SEQ ID NO: 634
  source 1..93: mol_type = genomic DNA

SEQ ID NO: 651 - DNA, 93 bp, Nicotiana tabacum; identical to SEQ ID NO: 634
  source 1..93: mol_type = genomic DNA

SEQ ID NO: 652 - DNA, 93 bp, Nicotiana tabacum; identical to SEQ ID NO: 634
  source 1..93: mol_type = genomic DNA

SEQ ID NO: 653 - DNA, 93 bp, Nicotiana tabacum; identical to SEQ ID NO: 634
  source 1..93: mol_type = genomic DNA

SEQ ID NO: 654 - DNA, 93 bp, Nicotiana tabacum; identical to SEQ ID NO: 634
  source 1..93: mol_type = genomic DNA

SEQ ID NO: 655 - DNA, 93 bp, Nicotiana tabacum; identical to SEQ ID NO: 634
  source 1..93: mol_type = genomic DNA

SEQ ID NO: 656 - DNA, 93 bp, Nicotiana tabacum; identical to SEQ ID NO: 634
  source 1..93: mol_type = genomic DNA

SEQ ID NO: 657 - DNA, 93 bp, Nicotiana tabacum; identical to SEQ ID NO: 634
  source 1..93: mol_type = genomic DNA

SEQ ID NO: 658 - DNA, 93 bp, Nicotiana tabacum; identical to SEQ ID NO: 634
  source 1..93: mol_type = genomic DNA

SEQ ID NO: 659 - DNA, 93 bp, Nicotiana tabacum; identical to SEQ ID NO: 634
  source 1..93: mol_type = genomic DNA

SEQ ID NO: 660 - DNA, 93 bp, Nicotiana tabacum; identical to SEQ ID NO: 634
  source 1..93: mol_type = genomic DNA

SEQ ID NO: 661 - DNA, 93 bp, Nicotiana tabacum; identical to SEQ ID NO: 634
  source 1..93: mol_type = genomic DNA

SEQ ID NO: 662 - DNA, 93 bp, Nicotiana tabacum; identical to SEQ ID NO: 634
  source 1..93: mol_type = genomic DNA

SEQ ID NO: 663 - DNA, 93 bp, Nicotiana tabacum; identical to SEQ ID NO: 634
  source 1..93: mol_type = genomic DNA

SEQ ID NO: 664 - DNA, 93 bp, Nicotiana tabacum; identical to SEQ ID NO: 634
  source 1..93: mol_type = genomic DNA

SEQ ID NO: 665 - DNA, 93 bp, Nicotiana tabacum; identical to SEQ ID NO: 634
  source 1..93: mol_type = genomic DNA

SEQ ID NO: 666 - DNA, 89 bp, Nicotiana tabacum
  source 1..89: mol_type = genomic DNA
tcagcaacatatgggaaggttttgactttggatggagcaattcaacagagaatggtggatttccatacactgaaatgattgttcatctt

SEQ ID NO: 667 - DNA, 89 bp, Nicotiana tabacum; identical to SEQ ID NO: 666
  source 1..89: mol_type = genomic DNA

SEQ ID NO: 668 - DNA, 88 bp, Nicotiana tabacum
  source 1..88: mol_type = genomic DNA
tcagcaacatatgggaaggttttgactttggatggagcaattcaacgagaatggtggatttccatacactgaaatgattgttcatctt

SEQ ID NO: 669 - DNA, 88 bp, Nicotiana tabacum; identical to SEQ ID NO: 668
  source 1..88: mol_type = genomic DNA

SEQ ID NO: 670 - DNA, 88 bp, Nicotiana tabacum
  source 1..88: mol_type = genomic DNA
tcagcaacatatgggaaggttttgactttggatggagcaattcaaagagaatggtggatttccatacactgaaatgattgttcatctt

SEQ ID NO: 671 - DNA, 88 bp, Nicotiana tabacum; identical to SEQ ID NO: 670
  source 1..88: mol_type = genomic DNA

SEQ ID NO: 672 - DNA, 89 bp, Nicotiana tabacum; identical to SEQ ID NO: 666
  source 1..89: mol_type = genomic DNA

SEQ ID NO: 673 - DNA, 89 bp, Nicotiana tabacum; identical to SEQ ID NO: 666
  source 1..89: mol_type = genomic DNA

SEQ ID NO: 674 - DNA, 91 bp, Nicotiana tabacum
  source 1..91: mol_type = genomic DNA
tcagcaacatatgggaaggttttgactttggatggagcaattcaacacagagaatggtggatttccatacactgaaatgattgttcatctt

SEQ ID NO: 675 - DNA, 91 bp, Nicotiana tabacum; identical to SEQ ID NO: 674
  source 1..91: mol_type = genomic DNA

SEQ ID NO: 676 - DNA, 91 bp, Nicotiana tabacum; identical to SEQ ID NO: 674
  source 1..91: mol_type = genomic DNA

SEQ ID NO: 677 - DNA, 91 bp, Nicotiana tabacum; identical to SEQ ID NO: 674
  source 1..91: mol_type = genomic DNA

SEQ ID NO: 678 - DNA, 91 bp, Nicotiana tabacum; identical to SEQ ID NO: 674
  source 1..91: mol_type = genomic DNA

SEQ ID NO: 679 - DNA, 83 bp, Nicotiana tabacum
  source 1..83: mol_type = genomic DNA
tcagcaacatatgggaaggttttgactttggatggagcaattagaatggtggatttccatacactgaaatgattgttcatctt

SEQ ID NO: 680 - DNA, 91 bp, Nicotiana tabacum; identical to SEQ ID NO: 674
  source 1..91: mol_type = genomic DNA

SEQ ID NO: 681 - DNA, 83 bp, Nicotiana tabacum; identical to SEQ ID NO: 679
  source 1..83: mol_type = genomic DNA

SEQ ID NO: 682 - DNA, 89 bp, Nicotiana tabacum; identical to SEQ ID NO: 666
  source 1..89: mol_type = genomic DNA

SEQ ID NO: 683 - DNA, 89 bp, Nicotiana tabacum; identical to SEQ ID NO: 666
  source 1..89: mol_type = genomic DNA

SEQ ID NO: 684 - DNA, 91 bp, Nicotiana tabacum; identical to SEQ ID NO: 674
  source 1..91: mol_type = genomic DNA

SEQ ID NO: 685 - DNA, 91 bp, Nicotiana tabacum; identical to SEQ ID NO: 674
  source 1..91: mol_type = genomic DNA

SEQ ID NO: 686 - DNA, 89 bp, Nicotiana tabacum; identical to SEQ ID NO: 666
  source 1..89: mol_type = genomic DNA

SEQ ID NO: 687 - DNA, 91 bp, Nicotiana tabacum; identical to SEQ ID NO: 674
  source 1..91: mol_type = genomic DNA

SEQ ID NO: 688 - DNA, 91 bp, Nicotiana tabacum; identical to SEQ ID NO: 674
  source 1..91: mol_type = genomic DNA

SEQ ID NO: 689 - DNA, 91 bp, Nicotiana tabacum; identical to SEQ ID NO: 674
  source 1..91: mol_type = genomic DNA

SEQ ID NO: 690 - DNA, 89 bp, Nicotiana tabacum; identical to SEQ ID NO: 666
  source 1..89: mol_type = genomic DNA

SEQ ID NO: 691 - DNA, 88 bp, Nicotiana tabacum
  source 1..88: mol_type = genomic DNA
tcagcaacatatgggaaggttttgactttggatggagcaattcaacaagaatggtggatttccatacactgaaatgattgttcatctt

SEQ ID NO: 692 - DNA, 91 bp, Nicotiana tabacum; identical to SEQ ID NO: 674
  source 1..91: mol_type = genomic DNA

SEQ ID NO: 693 - DNA, 89 bp, Nicotiana tabacum; identical to SEQ ID NO: 666
  source 1..89: mol_type = genomic DNA

SEQ ID NO: 694 - DNA, 91 bp, Nicotiana tabacum; identical to SEQ ID NO: 674
  source 1..91: mol_type = genomic DNA

SEQ ID NO: 695 - DNA, 91 bp, Nicotiana tabacum; identical to SEQ ID NO: 674
  source 1..91: mol_type = genomic DNA

SEQ ID NO: 696 - DNA, 91 bp, Nicotiana tabacum; identical to SEQ ID NO: 674
  source 1..91: mol_type = genomic DNA

SEQ ID NO: 697 - DNA, 85 bp, Nicotiana tabacum
  source 1..85: mol_type = genomic DNA
tcagcaacatatgggaaggttttgactttggatggagcaattcaagaatggtggatttccatacactgaaatgattgttcatctt

SEQ ID NO: 698 - DNA, 93 bp, Nicotiana tabacum
  source 1..93: mol_type = genomic DNA
tcagcaacatatgggaaggttttgactttggatggagcaattcaacacacagagaatggtggatttccatacactgaaatgattgttcatctt

SEQ ID NO: 699 - DNA, 93 bp, Nicotiana tabacum; identical to SEQ ID NO: 698
  source 1..93: mol_type = genomic DNA

SEQ ID NO: 700 - DNA, 93 bp, Nicotiana tabacum; identical to SEQ ID NO: 698
  source 1..93: mol_type = genomic DNA

SEQ ID NO: 701 - DNA, 93 bp, Nicotiana tabacum; identical to SEQ ID NO: 698
  source 1..93: mol_type = genomic DNA

SEQ ID NO: 702 - DNA, 93 bp, Nicotiana tabacum; identical to SEQ ID NO: 698
  source 1..93: mol_type = genomic DNA

SEQ ID NO: 703 - DNA, 93 bp, Nicotiana tabacum; identical to SEQ ID NO: 698
  source 1..93: mol_type = genomic DNA

SEQ ID NO: 704 - DNA, 93 bp, Nicotiana tabacum; identical to SEQ ID NO: 698
  source 1..93: mol_type = genomic DNA

SEQ ID NO: 705 - DNA, 93 bp, Nicotiana tabacum; identical to SEQ ID NO: 698
  source 1..93: mol_type = genomic DNA

SEQ ID NO: 706 - DNA, 93 bp, Nicotiana tabacum; identical to SEQ ID NO: 698
  source 1..93: mol_type = genomic DNA

SEQ ID NO: 707 - DNA, 93 bp, Nicotiana tabacum; identical to SEQ ID NO: 698
  source 1..93: mol_type = genomic DNA

SEQ ID NO: 708 - DNA, 93 bp, Nicotiana tabacum; identical to SEQ ID NO: 698
  source 1..93: mol_type = genomic DNA

SEQ ID NO: 709 - DNA, 93 bp, Nicotiana tabacum; identical to SEQ ID NO: 698
  source 1..93: mol_type = genomic DNA

SEQ ID NO: 710 - DNA, 93 bp, Nicotiana tabacum; identical to SEQ ID NO: 698
  source 1..93: mol_type = genomic DNA

SEQ ID NO: 711 - DNA, 93 bp, Nicotiana tabacum; identical to SEQ ID NO: 698
  source 1..93: mol_type = genomic DNA

SEQ ID NO: 712 - DNA, 93 bp, Nicotiana tabacum; identical to SEQ ID NO: 698
  source 1..93: mol_type = genomic DNA

SEQ ID NO: 713 - DNA, 93 bp, Nicotiana tabacum; identical to SEQ ID NO: 698
  source 1..93: mol_type = genomic DNA

SEQ ID NO: 714 - DNA, 93 bp, Nicotiana tabacum; identical to SEQ ID NO: 698
  source 1..93: mol_type = genomic DNA

SEQ ID NO: 715 - DNA, 93 bp, Nicotiana tabacum; identical to SEQ ID NO: 698
  source 1..93: mol_type = genomic DNA

SEQ ID NO: 716 - DNA, 93 bp, Nicotiana tabacum; identical to SEQ ID NO: 698
  source 1..93: mol_type = genomic DNA

SEQ ID NO: 717 - DNA, 93 bp, Nicotiana tabacum; identical to SEQ ID NO: 698
  source 1..93: mol_type = genomic DNA

SEQ ID NO: 718 - DNA, 93 bp, Nicotiana tabacum; identical to SEQ ID NO: 698
  source 1..93: mol_type = genomic DNA

SEQ ID NO: 719 - DNA, 93 bp, Nicotiana tabacum; identical to SEQ ID NO: 698
  source 1..93: mol_type = genomic DNA

SEQ ID NO: 720 - DNA, 93 bp, Nicotiana tabacum; identical to SEQ ID NO: 698
  source 1..93: mol_type = genomic DNA

SEQ ID NO: 721 - DNA, 93 bp, Nicotiana tabacum; identical to SEQ ID NO: 698
  source 1..93: mol_type = genomic DNA

SEQ ID NO: 722 - DNA, 93 bp, Nicotiana tabacum; identical to SEQ ID NO: 698
  source 1..93: mol_type = genomic DNA

SEQ ID NO: 723 - DNA, 93 bp, Nicotiana tabacum; identical to SEQ ID NO: 698
  source 1..93: mol_type = genomic DNA

SEQ ID NO: 724 - DNA, 93 bp, Nicotiana tabacum; identical to SEQ ID NO: 698
  source 1..93: mol_type = genomic DNA

SEQ ID NO: 725 - DNA, 93 bp, Nicotiana tabacum; identical to SEQ ID NO: 698
  source 1..93: mol_type = genomic DNA

SEQ ID NO: 726 - DNA, 93 bp, Nicotiana tabacum; identical to SEQ ID NO: 698
  source 1..93: mol_type = genomic DNA

SEQ ID NO: 727 - DNA, 93 bp, Nicotiana tabacum; identical to SEQ ID NO: 698
  source 1..93: mol_type = genomic DNA

SEQ ID NO: 728 - DNA, 93 bp, Nicotiana tabacum; identical to SEQ ID NO: 698
  source 1..93: mol_type = genomic DNA

SEQ ID NO: 729 - DNA, 93 bp, Nicotiana tabacum; identical to SEQ ID NO: 698
  source 1..93: mol_type = genomic DNA

(19) United States
(12) Patent Application Publication
(10) Pub. No.: US 2026/0347865 A1
(43) Pub. Date: Nov. 5, 2026

(54) COMPOSITIONS AND METHODS BASED ON PMT ENGINEERING FOR PRODUCING TOBACCO PLANTS AND PRODUCTS HAVING ALTERED ALKALOID LEVELS

(71) Applicant: Altria Client Services LLC, Richmond, VA (US)

(72) Inventors: Chengalrayan KUDITHIPUDI, Midlothian, VA (US); Dong QI, Henrico, VA (US); Yanxin SHEN, Henrico, VA (US); Ujwala WAREK, Richmond, VA (US); James STRICKLAND, Richmond, VA (US); Dongmei XU, Glen Allen, VA (US)

(73) Assignee: Altria Client Services LLC, Richmond, VA (US)

(21) Appl. No.: 19/555,356

(22) Filed: Mar. 3, 2026

Related U.S. Application Data
(63) Continuation of application No. 17/795,409, filed on Jan. 26, 2021, now abandoned.
(60) Provisional application No. 62/966,259, filed on Jan. 27, 2020.

(51) Int. Cl. C12N 15/82; C12N 9/10

(52) U.S. Cl. CPC C12N 15/8242; C12N 9/1007; C12N 15/8213

(57) ABSTRACT

The present disclosure provides compositions and methods related to tobacco plants with altered total alkaloid and nicotine levels and commercially acceptable leaf grade, their development via breeding or transgenic approaches, and production of tobacco products from these tobacco plants.

### CROSS-REFERENCE TO RELATED APPLICATIONS

This application is a continuation of U.S. patent application Ser. No. 17/795,409, filed Jul. 26, 2022, which is a U.S. National Stage of International Application No. PCT/US2021/015105, filed Jan. 26, 2021, which in turn claims the benefit of U.S. Provisional Application No. 62/966,259, filed Jan. 27, 2020, each of which are incorporated by reference herein in their entireties.

### INCORPORATION OF SEQUENCE LISTING

A sequence listing contained in the file named “P34801US02.xml” which is 779,166 bytes (measured in operating system MS-Windows®), produced on Feb. 4, 2026, containing a total number of 729 sequences, starting from SEQ ID NO:1 to SEQ ID NO: 729, is filed electronically herewith and incorporated by reference in its entirety.

### FIELD

The present disclosure provides tobacco genetic engineering for modulating alkaloid and nicotine levels.

### BACKGROUND

Nicotine is the predominant alkaloid, usually accounting for more than 90-95% of the total alkaloids in commercial tobacco cultivars. The remaining alkaloid fraction is primarily comprised of three additional alkaloids: nornicotine, anabasine, and anatabine. Tobacco plants with reduced nicotine levels have been achieved with varying and inconsistent results by modulating different nicotine biosynthetic genes and transcriptional regulators through traditional plant breeding and other biotechnological techniques. There is a need for new technologies to reduce nicotine levels in tobacco leaves.

### SUMMARY

The present disclosure provides tobacco plants with altered total alkaloid and nicotine levels and commercially acceptable leaf grade, their development via breeding or transgenic approaches, and production of tobacco products from these tobacco plants.

In an aspect, the present disclosure provides a tobacco plant, or part thereof, comprising one or more mutant alleles in at least one PMT gene selected from the group consisting of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions.

In an aspect, the present disclosure provides a tobacco plant, or part thereof, comprising one or more mutant alleles in at least one PMT gene selected from the group consisting of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions.

In another aspect, a tobacco plant comprises one or more mutant alleles in at least two PMT genes selected from the group consisting of PMT1a, PMT1b, PMT2, PMT3, and PMT4.

In a further aspect, a tobacco plant comprises one or more mutant alleles in at least three PMT genes selected from the group consisting of PMT1a, PMT1b, PMT2, PMT3, and PMT4.

In another aspect, a tobacco plant comprises one or more mutant alleles in at least four PMT genes selected from the group consisting of PMT1a, PMT1b, PMT2, PMT3, and PMT4.

In a further aspect, a tobacco plant comprises one or more mutant alleles in five PMT genes selected from the group consisting of PMT1a, PMT1b, PMT2, PMT3, and PMT4.

In an aspect, the present disclosure provides a tobacco plant selected from the group consisting of a single pmt mutant, a double pmt mutant, a triple mutant, a quadruple mutant, and a quintuple mutant, as listed in Tables 8A to 8E.

In an aspect, the present disclosure provides a tobacco plant as listed in Tables 4A to 4E or Table 10. In another aspect, the present disclosure provides a progeny plant of a tobacco plant in Tables 4A to 4E or Table 10, from either self-pollinating or a cross with another plant in Tables 4A to 4E or Table 10.

In another aspect, the present disclosure provides a tobacco plant comprising various combinations of the pmt mutant alleles listed in Tables 5A to 5E or Tables 12A to 12E or a quintuple mutant. In an aspect, the present disclosure provides a tobacco plant comprising a pmt mutant allele sequence selected from the group consisting of SEQ ID Nos. 21 to 200, 410 to 441, 474 to 505, 538 to 569, 602 to 633, and 666 to 697.

The present disclosure further provides cured tobacco, tobacco blends, tobacco products comprising plant material from tobacco plants, lines, varieties or hybrids disclosed.

### BRIEF DESCRIPTION OF THE SEQUENCES

SEQ ID Nos: 1 to 5 set forth exemplary genomic sequences of PMT1b, PMT1a, PMT2, PMT3 and PMT4, respectively, from a TN90 reference genome.

SEQ ID Nos: 6 to 10 set forth exemplary cDNA sequences of PMT1b, PMT1a, PMT2, PMT3 and PMT4, respectively, from TN90.

SEQ ID Nos: 11 to 15 set forth exemplary polypeptide sequences of PMT1b, PMT1a, PMT2, PMT3 and PMT4, respectively, from TN90.

SEQ ID Nos: 16 to 22 set forth exemplary guide RNA sequences.

SEQ ID Nos: 23 to 200, 410 to 441, 474 to 505, 538 to 569, 602 to 633, and 666 to 697 set forth exemplary edited pmt mutant sequences.

### BRIEF DESCRIPTION OF THE DRAWINGS

FIG. 1: RNA expression of five PMT genes in TN90 roots

FIG. 2: Nicotine levels in various low-alkaloid lines: CS15 (a quintuple pmt knock-out mutant line CS15 in the NLM (Ph Ph) background), a PMT RNAi transgenic line in the VA359 background) and a low-nicotine KY171 (“LN KY171”) variety (the KY 171 background harboring nic1 and nic2 double mutations), in comparison to their respective normal-alkaloid control line: NLM (Ph Ph), VA359, and KY171 background.

FIG. 3: Nornicotine levels in various low-alkaloid lines: CS15, PMT RNAi, and LN KY171, in comparison to their respective normal-alkaloid control line: NLM (Ph Ph), VA359, and KY171 background.

FIG. 4: Anabasine levels in various low-alkaloid lines: CS15, PMT RNAi, and LN KY171, in comparison to their respective normal-alkaloid control line: NLM (Ph Ph), VA359, and KY171 background.

FIG. 5: Anatabine levels in various low-alkaloid lines: CS15, PMT RNAi, and LN KY171, in comparison to their respective normal-alkaloid control line: NLM (Ph Ph), VA359, and KY171 background.

FIG. 6: Total alkaloid levels in various low-alkaloid lines: CS15, PMT RNAi, and LN KY171, in comparison to their respective normal-alkaloid control line: NLM (Ph Ph), VA359, and KY171 background.

FIG. 7: N-nitrosonornicotine (NNN) levels in various low-alkaloid lines: CS15, PMT RNAi, and LN KY171, in comparison to their respective normal-alkaloid control line: NLM (Ph Ph), VA359, and KY171 background.

FIG. 8: Nicotine-derived nitrosamine ketone (NNK) levels in various low-alkaloid lines: CS15, PMT RNAi, and LN KY171, in comparison to their respective normal-alkaloid control line: NLM (Ph Ph), VA359, and KY171 background.

FIG. 9: N-nitrosoanabasine (NAB) levels in various low-alkaloid lines: CS15, PMT RNAi, and LN KY171, in comparison to their respective normal-alkaloid control line: NLM (Ph Ph), VA359, and KY171 background.

FIG. 10: N-nitrosoanatabine (NAT) levels in various low-alkaloid lines: CS15, PMT RNAi, and LN KY171, in comparison to their respective normal-alkaloid control line: NLM (Ph Ph), VA359, and KY171 background.

FIG. 11: Total tobacco-specific nitrosamine (TSNA) levels in various low-alkaloid lines: CS15, PMT RNAi, and LN KY171, in comparison to their respective normal-alkaloid control line: NLM (Ph Ph), VA359, and KY171 background.

FIG. 12: Leaf yield in various low-alkaloid lines: CS15, PMT RNAi, and LN KY171, in comparison to their respective normal-alkaloid control line: NLM (Ph Ph), VA359, and KY171 background.

FIG. 13: Leaf quality in various low-alkaloid lines: CS15, PMT RNAi, and LN KY171, in comparison to their respective normal-alkaloid control line: NLM (Ph Ph), VA359, and KY171 background.

FIGS. 14A to 14E: Photographs depicting mold growth on cured tobacco, including TN90 LC (FIG. 14A), LA BU 21 (FIG. 14B), TN90 comprising an RNAi construct to downregulate PR50 (FIG. 14C), TN90 comprising an RNAi construct to downregulate PMT genes (FIG. 14D), and TN90 comprising edits to all five PMT genes (FIG. 14E).

FIG. 15: Depiction of mold infection observed in the lines examined in FIGS. 14A-14E.

FIG. 16: Nicotine levels in the lamina of various low-alkaloid burley lines: CS47, CS59, CS63, CS64, and LA Burley 21, in comparison to a normal-alkaloid control line TN 90 LC. Levels are measured two-weeks post-topping, at harvest, and after curing. All plants are field grown.

FIG. 17: Nicotine levels in the lamina of various low-alkaloid flue-cured lines: CS69, CS70, CS72, CS73, and LA FC 53, in comparison to a normal-alkaloid control line K326. Levels are measured two-weeks post-topping, at harvest, and after curing. All plants are field grown.

FIG. 18: Nornicotine levels in the lamina of various low-alkaloid burley lines: CS47, CS59, CS63, CS64, and LA Burley 21, in comparison to a normal-alkaloid control line TN 90 LC. Levels are measured two-weeks post-topping, at harvest, and after curing. All plants are field grown.

FIG. 19: Nornicotine levels in the lamina of various low-alkaloid flue-cured lines: CS69, CS70, CS72, CS73, and LA FC 53, in comparison to a normal-alkaloid control line K326. Levels are measured two-weeks post-topping, at harvest, and after curing. All plants are field grown.

FIG. 20: Anabasine levels in the lamina of various low-alkaloid burley lines: CS47, CS59, CS63, CS64, and LA Burley 21, in comparison to a normal-alkaloid control line TN 90 LC. Levels are measured two-weeks post-topping, at harvest, and after curing. All plants are field grown.

FIG. 21: Anabasine levels in the lamina of various low-alkaloid flue-cured lines: CS69, CS70, CS72, CS73, and LA FC 53, in comparison to a normal-alkaloid control line K326. Levels are measured two-weeks post-topping, at harvest, and after curing. All plants are field grown.

FIG. 22: Anatabine levels in the lamina of various low-alkaloid burley lines: CS47, CS59, CS63, CS64, and LA Burley 21, in comparison to a normal-alkaloid control line TN 90 LC. Levels are measured two-weeks post-topping, at harvest, and after curing. All plants are field grown.

FIG. 23: Anatabine levels in the lamina of various low-alkaloid flue-cured lines: CS69, CS70, CS72, CS73, and LA FC 53, in comparison to a normal-alkaloid control line K326. Levels are measured two-weeks post-topping, at harvest, and after curing. All plants are field grown.

FIG. 24: Total alkaloid levels in the lamina of various low-alkaloid burley lines: CS47, CS59, CS63, CS64, and LA Burley 21, in comparison to a normal-alkaloid control line TN 90 LC. Levels are measured two-weeks post-topping, at harvest, and after curing. All plants are field grown.

FIG. 25: Total alkaloid levels in the lamina of various low-alkaloid flue-cured lines: CS69, CS70, CS72, CS73, and LA FC 53, in comparison to a normal-alkaloid control line K326. Levels are measured two-weeks post-topping, at harvest, and after curing. All plants are field grown.

FIG. 26: Nitrite analysis of cured burley lamina from low-alkaloid lines CS47, CS59, CS64, and LA Burley 21, and the normal-alkaloid control line TN 90 LC. All plants are field grown.

FIG. 27: Nitrate analysis of cured burley lamina from low-alkaloid lines CS47, CS59, CS64, and LA Burley 21, and the normal-alkaloid control line TN 90 LC. All plants are field grown.

FIG. 28: NNN analysis of cured burley lamina from low-alkaloid lines CS47, CS59, CS64, and LA Burley 21, and the normal-alkaloid control line TN 90 LC. All plants are field grown.

FIG. 29: NNN analysis of cured flue-cured lamina from low-alkaloid lines CS70, CS72, CS73, LA FC 53, and the normal-alkaloid control line K326. All plants are field grown.

FIG. 30: NNK analysis of cured burley lamina from low-alkaloid lines CS47, CS59, CS64, and LA Burley 21, and the normal-alkaloid control line TN 90 LC. All plants are field grown.

FIG. 31: NNK analysis of cured flue-cured lamina from low-alkaloid lines CS70, CS72, CS73, LA FC 53, and the normal-alkaloid control line K326. All plants are field grown.

FIG. 32: NAB analysis of cured burley lamina from low-alkaloid lines CS47, CS59, CS64, and LA Burley 21, and the normal-alkaloid control line TN 90 LC. All plants are field grown.

FIG. 33: NAT analysis of cured burley lamina from low-alkaloid lines CS47, CS59, CS64, and LA Burley 21, and the normal-alkaloid control line TN 90 LC. All plants are field grown.

FIG. 34: NAT analysis of cured flue-cured lamina from low-alkaloid lines CS70, CS72, CS73, LA FC 53, and the normal-alkaloid control line K326. All plants are field grown.

FIG. 35: Yield analysis of cured burley lamina from low-alkaloid lines CS47, CS59, CS64, and LA Burley 21, and the normal-alkaloid control line TN 90 LC. All plants are field grown.

FIG. 36: Yield analysis of cured flue-cured lamina from low-alkaloid lines CS70, CS72, CS73, LA FC 53, and the normal-alkaloid control line K326. All plants are field grown.

FIG. 37: Reducing sugars analysis of cured flue-cured lamina from low-alkaloid lines CS70, CS72, CS73, LA FC 53, and the normal-alkaloid control line K326. All plants are field grown.

FIG. 38: Leaf quality analysis of cured burley lamina from low-alkaloid lines CS47, CS59, CS64, and LA Burley 21, and the normal-alkaloid control line TN 90 LC. All plants are field grown.

FIG. 39: Leaf quality analysis of cured flue-cured lamina from low-alkaloid lines CS70, CS72, CS73, LA FC 53, and the normal-alkaloid control line K326. All plants are field grown.

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. Where a term is provided in the singular, the inventors also contemplate aspects of the disclosure described by the plural of that term, and vice versa. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). For purposes of the present disclosure, the following terms are defined below.

Any references cited herein, including, e.g., all patents and publications are incorporated by reference in their entirety and to the same extent as if each individual publication, patent, or patent application is specifically and individually indicated to be incorporated by reference.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof.

When the term “about” is used in conjunction with a numerical range, it modifies that range by extending the boundaries above and below the numerical values set forth by 10%.

As used herein, phrases such as “less than”, “more than”, “at least”, “at most”, “approximately”, “below”, “above”, and “about”, when used in conjunction with a series of numerical values, modify each and every value within the series. For example, an expression of “less than 1%, 2%, or 3%” is equivalent to “less than 1%, less than 2%, or less than 3%.” Similarly, to avoid any doubt, used herein, terms or phrases such as “less than”, “more than about”, “at least”, “at least about”, “at most”, “approximately”, “below”, “above”, and “about”, when used in conjunction with a series of numerical values, such terms or phrases are deemed to modify each and every value within the series.

As used herein, a tobacco plant refers to a plant from the species Nicotiana tabacum.

As used herein, a “low alkaloid variety” (also referred to as “LA variety”) of tobacco refers to tobacco variety comprising one or more genetic modifications reducing the total alkaloids (measured via dry weight) to a level less than 25% of the total alkaloid level in a control tobacco variety of a substantially similar genetic background except for the one or more genetic modifications. As a non-limiting example, KY171 can serve as a control for a low-alkaloid variety LA KY171. Without being limiting, low-alkaloid tobacco varieties include LA Burley 21, LAFC53, LN B&W, and LN KY171. Similarly, a “low nicotine variety” (also referred to as “LN variety”) of tobacco refers to tobacco variety comprising one or more genetic modifications reducing nicotine (measured via dry weight) to a level less than 25% of the nicotine level in a control tobacco variety of a substantially similar genetic background except for the one or more genetic modifications.

Nicotine biosynthesis in tobacco starts with the methylation of the polyamine, putrescine, to N-methylputrescine by the enzyme, putrescine N-methyltransferase (PMT), using S-adenosyl-methionine as the co-factor. This is a step that commits precursor metabolites to nicotine biosynthesis. PMT enzymes are classified under the enzyme classification system as EC 2.1.1.53. In Nicotiana tabacum, five genes encode putrescine N-methyltransferases, designated PMT1a, PMT1b, PMT2, PMT3, and PMT4. Table 1A lists genomic DNA sequences, cDNA sequences, and protein sequences of these five PMT genes in a TN90 plant. The present disclosure describes compositions and methods that are used to edit PMT genes to produce pmt mutant plants having reduced nicotine levels while maintaining leaf quality.

As used herein, “PMT1b” or the “PMT1b gene” refers to a genic locus in tobacco encoding a polypeptide having an exemplary amino acid sequence in TN90 as set forth in SEQ ID No. 11.

As used herein, “PMT1a” or the “PMT1a gene” refers to a genic locus in tobacco encoding a polypeptide having an exemplary amino acid sequence in TN90 as set forth in SEQ ID No. 12.

As used herein, “PMT2” or the “PMT2 gene” refers to a genic locus in tobacco encoding a polypeptide having an exemplary amino acid sequence in TN90 as set forth in SEQ ID No. 13.

As used herein, “PMT3” or the “PMT3 gene” refers to a genic locus in tobacco encoding a polypeptide having an exemplary amino acid sequence in TN90 as set forth in SEQ ID No. 14.

As used herein, “PMT4” or the “PMT4 gene” refers to a genic locus in tobacco encoding a polypeptide having an exemplary amino acid sequence in TN90 as set forth in SEQ ID No. 15.

As used herein, a mutation refers to an inheritable genetic modification introduced into a gene to reduce, inhibit, or eliminate the expression or activity of a product encoded by the gene. Such a modification can be in any sequence region of a gene, for example, in a promoter, 5′ UTR, exon, intron, 3′ UTR, or terminator region. In an aspect, mutations are not natural polymorphisms that exist in a particular tobacco variety or cultivar. As used herein, a “mutant allele” refers to an allele from a locus where the allele comprises a mutation. It will be appreciated that, when identifying a mutation, the reference sequence should be from the same tobacco variety or background. For example, if a modified tobacco plant comprising a mutation is from the variety TN90, then the corresponding reference sequence should be the endogenous TN90 sequence, not a homologous sequence from a different tobacco variety (e.g., K326). In an aspect, a mutation is a “non-natural” or “non-naturally occurring” mutation. As used herein, a “non-natural” or “non-naturally occurring” mutation refers to a mutation that is not, and does not correspond to, a spontaneous mutation generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations and non-naturally occurring mutations do not include spontaneous mutations that arise naturally (e.g., via aberrant DNA replication in a germ line of a plant.

As used herein, a “genetic modification” refers to a change in the genetic makeup of a plant or plant genome. A genetic modification can be introduced by methods including, but not limited to, mutagenesis, genome editing, genetic transformation, or a combination thereof. A genetic modification includes, for example, a mutation (e.g., a non-natural mutation) in a gene or a transgene targeting a gene. As used here, “targeting” refers to either directly upregulating or directly downregulating the expression or activity of a gene. As used here, “directly”, in the context of a transgene impacting the expression or activity of a gene, refers to the impact being exerted over the gene via a physical contact or chemical interaction between the gene (e.g., a promoter region or a UTR region) or a product encoded therein (e.g., a mRNA molecule or a polypeptide) and a product encoded by the transgene (e.g., a small non-coding RNA molecule or a protein such as a transcription factor or a dominant negative polypeptide variant). In an aspect, a transgene impacts the expression or activity of a target gene without involving a transcription factor (e.g., the transgene does not encode a transcription factor and/or does not suppress the expression or activity of a transcription factor that in turn regulates the target gene).

As used herein, a “pmt mutant” refers to a tobacco plant comprising one or more mutations in one or more PMT genes. A pmt mutant can be a single mutant, a double mutant, a triple mutant, a quadruple mutant, or a quintuple mutant. As used herein, a single, double, triple, quadruple, or quintuple pmt mutant refers to a mutant having modifications in one, two, three, four, or five PMT genes, respectively. A pmt mutant can also be a homozygous mutant, a heterozygous mutant, or a heteroallelic mutant combination in one or more PMT genes.

As used herein, a gene name or a genic locus name is capitalized and shown in italic, e.g., PMT1a, PMT1b, PMT2, PMT3, and PMT4. A protein or polypeptide name is capitalized without being italicized, e.g., PMT1a, PMT1b, PMT2, PMT3, and PMT4. A mutant name (for either referencing to a general mutation in a gene or a group of genes, or referencing to a specific mutant allele) is shown in lower case and italic, e.g., pmt, pmt1a, pmt1b, pmt2, pmt3, and pmt4.

In an aspect, the present disclosure provides a tobacco plant, or part thereof, comprising one or more mutant alleles in at least one PMT gene selected from the group consisting of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions. In an aspect, the present disclosure provides a tobacco plant, or part thereof, comprising one or more mutant alleles in at least two PMT genes selected from the group consisting of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions. In an aspect, the present disclosure provides a tobacco plant, or part thereof, comprising one or more mutant alleles in at least three PMT genes selected from the group consisting of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions. In an aspect, the present disclosure provides a tobacco plant, or part thereof, comprising one or more mutant alleles in at least four PMT genes selected from the group consisting of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions. In an aspect, the present disclosure provides a tobacco plant, or part thereof, comprising one or more mutant alleles in each of PMT1a, PMT1b, PMT2, PMT3, and PMT4, wherein said tobacco plant is capable of producing a leaf comprising an anatabine level greater than the anatabine level of a leaf from a control tobacco plant not having said one or more mutant alleles when grown and processed under comparable conditions.

In an aspect, a single pmt mutant tobacco plant is capable of producing a leaf comprising an anatabine level at least 1%, at least 2%, at least 5%, at least 10%, at least 25%, at least 50%, at least 75%, at least 100%, at least 150%, at least 200%, at least 300%, at least 400%, at least 500%, at least 600%, at least 700%, at least 800%, or at least 900% greater than the anatabine level of a leaf from a control tobacco plant grown and processed under comparable conditions. In an aspect, a double pmt mutant tobacco plant is capable of producing a leaf comprising an anatabine level at least 1%, at least 2%, at least 5%, at least 10%, at least 25%, at least 50%, at least 75%, at least 100%, at least 150%, at least 200%, at least 300%, at least 400%, at least 500%, at least 600%, at least 700%, at least 800%, or at least 900% greater than the anatabine level of a leaf from a control tobacco plant grown and processed under comparable conditions. In an aspect, a triple pmt mutant tobacco plant is capable of producing a leaf comprising an anatabine level at least 1%, at least 2%, at least 5%, at least 10%, at least 25%, at least 50%, at least 75%, at least 100%, at least 150%, at least 200%, at least 300%, at least 400%, at least 500%, at least 600%, at least 700%, at least 800%, or at least 900% greater than the anatabine level of a leaf from a control tobacco plant grown and processed under comparable conditions. In an aspect, a quadruple pmt mutant tobacco plant is capable of producing a leaf comprising an anatabine level at least 1%, at least 2%, at least 5%, at least 10%, at least 25%, at least 50%, at least 75%, at least 100%, at least 150%, at least 200%, at least 300%, at least 400%, at least 500%, at least 600%, at least 700%, at least 800%, or at least 900% greater than the anatabine level of a leaf from a control tobacco plant grown and processed under comparable conditions. In an aspect, a quintuple pmt mutant tobacco plant is capable of producing a leaf comprising an anatabine level at least 1%, at least 2%, at least 5%, at least 10%, at least 25%, at least 50%, at least 75%, at least 100%, at least 150%, at least 200%, at least 300%, at least 400%, at least 500%, at least 600%, at least 700%, at least 800%, or at least 900% greater than the anatabine level of a leaf from a control tobacco plant grown and processed under comparable conditions.

In an aspect, a single pmt mutant tobacco plant is capable of producing a leaf comprising an anatabine level of at least 0.13%, at least 0.2%, at least 0.3%, at least 0.4%, at least 0.5%, at least 0.6%, at least 0.7%, at least 0.8%, at least 0.9%, or at least 1% dry weight per gram of leaf lamina. In an aspect, a double pmt mutant tobacco plant is capable of producing a leaf comprising an anatabine level of at least 0.13%, at least 0.2%, at least 0.3%, at least 0.4%, at least 0.5%, at least 0.6%, at least 0.7%, at least 0.8%, at least 0.9%, or at least 1% dry weight per gram of leaf lamina. In an aspect, a triple pmt mutant tobacco plant is capable of producing a leaf comprising an anatabine level of at least 0.13%, at least 0.2%, at least 0.3%, at least 0.4%, at least 0.5%, at least 0.6%, at least 0.7%, at least 0.8%, at least 0.9%, or at least 1% dry weight per gram of leaf lamina. In an aspect, a quadruple pmt mutant tobacco plant is capable of producing a leaf comprising an anatabine level of at least 0.13%, at least 0.2%, at least 0.3%, at least 0.4%, at least 0.5%, at least 0.6%, at least 0.7%, at least 0.8%, at least 0.9%, or at least 1% dry weight per gram of leaf lamina. In an aspect, a quintuple pmt mutant tobacco plant is capable of producing a leaf comprising an anatabine level of at least 0.13%, at least 0.2%, at least 0.3%, at least 0.4%, at least 0.5%, at least 0.6%, at least 0.7%, at least 0.8%, at least 0.9%, or at least 1% dry weight per gram of leaf lamina.

In an aspect, the present disclosure provides a tobacco plant, or part thereof, comprising one or more mutant alleles in at least one PMT gene selected from the group consisting of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions. In an aspect, the present disclosure provides a tobacco plant, or part thereof, comprising one or more mutant alleles in at least two PMT genes selected from the group consisting of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions. In an aspect, the present disclosure provides a tobacco plant, or part thereof, comprising one or more mutant alleles in at least three PMT genes selected from the group consisting of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions. In an aspect, the present disclosure provides a tobacco plant, or part thereof, comprising one or more mutant alleles in at least four PMT genes selected from the group consisting of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions. In an aspect, the present disclosure provides a tobacco plant, or part thereof, comprising one or more mutant alleles in each of PMT1a, PMT1b, PMT2, PMT3, and PMT4, wherein said tobacco plant is capable of producing a leaf comprising an anabasine level greater than the anabasine level of a leaf from a control tobacco plant not having said one or more mutant alleles when grown and processed under comparable conditions.

In an aspect, a single pmt mutant tobacco plant is capable of producing a leaf comprising an anabasine level at least 1%, at least 2%, at least 5%, at least 10%, at least 20% at least 25%, at least 30%, at least 40%, at least 50%, at least 60%, at least 70%, at least 75%, at least 80%, at least 90%, at least 100%, at least 125%, at least 150%, at least 175%, at least 200%, at least 225%, at least 250%, or at least 300% greater than the anabasine level of a leaf from a control tobacco plant grown and processed under comparable conditions. In an aspect, a double pmt mutant tobacco plant is capable of producing a leaf comprising an anabasine level at least 1%, at least 2%, at least 5%, at least 10%, at least 20% at least 25%, at least 30%, at least 40%, at least 50%, at least 60%, at least 70%, at least 75%, at least 80%, at least 90%, at least 100%, at least 125%, at least 150%, at least 175%, at least 200%, at least 225%, at least 250%, or at least 300% greater than the anabasine level of a leaf from a control tobacco plant grown and processed under comparable conditions. In an aspect, a triple pmt mutant tobacco plant is capable of producing a leaf comprising an anabasine level at least 1%, at least 2%, at least 5%, at least 10%, at least 20% at least 25%, at least 30%, at least 40%, at least 50%, at least 60%, at least 70%, at least 75%, at least 80%, at least 90%, at least 100%, at least 125%, at least 150%, at least 175%, at least 200%, at least 225%, at least 250%, or at least 300% greater than the anabasine level of a leaf from a control tobacco plant grown and processed under comparable conditions. In an aspect, a quadruple pmt mutant tobacco plant is capable of producing a leaf comprising an anabasine level at least 1%, at least 2%, at least 5%, at least 10%, at least 20% at least 25%, at least 30%, at least 40%, at least 50%, at least 60%, at least 70%, at least 75%, at least 80%, at least 90%, at least 100%, at least 125%, at least 150%, at least 175%, at least 200%, at least 225%, at least 250%, or at least 300% greater than the anabasine level of a leaf from a control tobacco plant grown and processed under comparable conditions. In an aspect, a quintuple pmt mutant tobacco plant is capable of producing a leaf comprising an anabasine level at least 1%, at least 2%, at least 5%, at least 10%, at least 20% at least 25%, at least 30%, at least 40%, at least 50%, at least 60%, at least 70%, at least 75%, at least 80%, at least 90%, at least 100%, at least 125%, at least 150%, at least 175%, at least 200%, at least 225%, at least 250%, or at least 300% greater than the anabasine level of a leaf from a control tobacco plant grown and processed under comparable conditions.

In an aspect, a single pmt mutant tobacco plant is capable of producing a leaf comprising an anabasine level of at least 0.017%, at least 0.02%, at least 0.025%, at least 0.03%, at least 0.035%, at least 0.04%, at least 0.05%, at least 0.06%, at least 0.07%, at least 0.08%, at least 0.09% or at least 0.1% dry weight per gram of leaf lamina. In an aspect, a double pmt mutant tobacco plant is capable of producing a leaf comprising an anabasine level of at least 0.017%, at least 0.02%, at least 0.025%, at least 0.03%, at least 0.035%, at least 0.04%, at least 0.05%, at least 0.06%, at least 0.07%, at least 0.08%, at least 0.09% or at least 0.1% dry weight per gram of leaf lamina. In an aspect, a triple pmt mutant tobacco plant is capable of producing a leaf comprising an anabasine level of at least 0.017%, at least 0.02%, at least 0.025%, at least 0.03%, at least 0.035%, at least 0.04%, at least 0.05%, at least 0.06%, at least 0.07%, at least 0.08%, at least 0.09% or at least 0.1% dry weight per gram of leaf lamina. In an aspect, a quadruple pmt mutant tobacco plant is capable of producing a leaf comprising an anabasine level of at least 0.017%, at least 0.02%, at least 0.025%, at least 0.03%, at least 0.035%, at least 0.04%, at least 0.05%, at least 0.06%, at least 0.07%, at least 0.08%, at least 0.09% or at least 0.1% dry weight per gram of leaf lamina. In an aspect, a quintuple pmt mutant tobacco plant is capable of producing a leaf comprising an anabasine level of at least 0.017%, at least 0.02%, at least 0.025%, at least 0.03%, at least 0.035%, at least 0.04%, at least 0.05%, at least 0.06%, at least 0.07%, at least 0.08%, at least 0.09% or at least 0.1% dry weight per gram of leaf lamina.

In an aspect, a single pmt mutant tobacco plant is capable of producing a leaf comprising a reduced level of nornicotine as compared to a control plant grown under comparable conditions. In an aspect, a double pmt mutant tobacco plant is capable of producing a leaf comprising a reduced level of nornicotine as compared to a control plant grown under comparable conditions. In an aspect, a triple pmt mutant tobacco plant is capable of producing a leaf comprising a reduced level of nornicotine as compared to a control plant grown under comparable conditions. In an aspect, a quadruple pmt mutant tobacco plant is capable of producing a leaf comprising a reduced level of nornicotine as compared to a control plant grown under comparable conditions. In an aspect, a quintuple pmt mutant tobacco plant is capable of producing a leaf comprising a reduced level of nornicotine as compared to a control plant grown under comparable conditions.

In an aspect, a reduced level of nornicotine comprises a reduction of at least 1%, at least 2%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% as compared to a control tobacco plant when grown and processed under comparable conditions.

In an aspect, a single pmt mutant tobacco plant is capable of producing a leaf comprising an increased level of nornicotine as compared to a control plant grown under comparable conditions. In an aspect, a double pmt mutant tobacco plant is capable of producing a leaf comprising an increased level of nornicotine as compared to a control plant grown under comparable conditions. In an aspect, a triple pmt mutant tobacco plant is capable of producing a leaf comprising an increased level of nornicotine as compared to a control plant grown under comparable conditions. In an aspect, a quadruple pmt mutant tobacco plant is capable of producing a leaf comprising an increased level of nornicotine as compared to a control plant grown under comparable conditions. In an aspect, a quintuple pmt mutant tobacco plant is capable of producing a leaf comprising an increased level of nornicotine as compared to a control plant grown under comparable conditions.

In an aspect, an increased level of nornicotine comprises an increase of at least 1%, at least 2%, at least 5%, at least 10%, at least 25%, at least 50%, at least 75%, at least 100%, at least 200%, at least 300%, at least 400%, at least 500%, or at least 600% as compared to the control tobacco plant.

In an aspect, the present disclosure provides a tobacco plant, or part thereof, comprising one or more mutant alleles in at least one PMT gene selected from the group consisting of PMT1a, PMT1b, PMT2, PMT3, and PMT4, wherein the tobacco plant is capable of producing a leaf comprising a nicotine level less than the nicotine level of a leaf from a control tobacco plant not having the one or more mutant alleles when grown and processed under comparable conditions. In an aspect, a single pmt mutant tobacco plant is provided. In another aspect, a single pmt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the single pmt mutation when grown in similar growth conditions. In a further aspect, a single pmt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the single pmt mutation when grown in similar growth conditions.

In another aspect, a tobacco plant comprises one or more mutant alleles in at least two PMT genes selected from the group consisting of PMT1a, PMT1b, PMT2, PMT3, and PMT4. In an aspect, a double pmt mutant tobacco plant is provided. In another aspect, a double pmt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the double pmt mutations when grown in similar growth conditions. In a further aspect, a double pmt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the double pmt mutations when grown in similar growth conditions.

In a further aspect, a tobacco plant comprises one or more mutant alleles in at least three PMT genes selected from the group consisting of PMT1a, PMT1b, PMT2, PMT3, and PMT4. In an aspect, a triple pmt mutant tobacco plant is provided. In another aspect, a triple pmt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the triple pmt mutations when grown in similar growth conditions. In a further aspect, a triple pmt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the triple pmt mutations when grown in similar growth conditions.

In another aspect, a tobacco plant comprises one or more mutant alleles in at least four PMT genes selected from the group consisting of PMT1a, PMT1b, PMT2, PMT3, and PMT4. In an aspect, a quadruple pmt mutant tobacco plant is provided. In another aspect, a quadruple pmt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the quadruple pmt mutations when grown in similar growth conditions. In a further aspect, a quadruple pmt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the quadruple pmt mutations when grown in similar growth conditions.

In a further aspect, a tobacco plant comprises one or more mutant alleles in five PMT genes selected from the group consisting of PMT1a, PMT1b, PMT2, PMT3, and PMT4. In an aspect, a quintuple pmt mutant tobacco plant is provided. In another aspect, a quintuple pmt mutant tobacco plant comprises nicotine at a level below 1%, below 2%, below 5%, below 8%, below 10%, below 12%, below 15%, below 20%, below 25%, below 30%, below 40%, below 50%, below 60%, below 70%, below 80%, below 90%, or below 95% of the nicotine level of a control plant not having the quintuple pmt mutations when grown in similar growth conditions. In a further aspect, a quintuple pmt mutant tobacco plant comprises nicotine at a level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control plant not having the quintuple pmt mutations when grown in similar growth conditions.

In an aspect, a single pmt mutant tobacco plant comprises a nicotine level of less than 1%, less than 0.75%, less than 0.5%, less than 0.25%, less than 0.1%, less than 0.05%, less than 0.01%, less than 0.005%, or less than 0.002% dry weight per gram of leaf lamina. In an aspect, a double pmt mutant tobacco plant comprises a nicotine level of less than 1%, less than 0.75%, less than 0.5%, less than 0.25%, less than 0.1%, less than 0.05%, less than 0.01%, less than 0.005%, or less than 0.002% dry weight per gram of leaf lamina. In an aspect, a triple pmt mutant tobacco plant comprises a nicotine level of less than 1%, less than 0.75%, less than 0.5%, less than 0.25%, less than 0.1%, less than 0.05%, less than 0.01%, less than 0.005%, or less than 0.002% dry weight per gram of leaf lamina. In an aspect, a quadruple pmt mutant tobacco plant comprises a nicotine level of less than 1%, less than 0.75%, less than 0.5%, less than 0.25%, less than 0.1%, less than 0.05%, less than 0.01%, less than 0.005%, or less than 0.002% dry weight per gram of leaf lamina. In an aspect, a quintuple pmt mutant tobacco plant comprises a nicotine level of less than 1%, less than 0.75%, less than 0.5%, less than 0.25%, less than 0.1%, less than 0.05%, less than 0.01%, less than 0.005%, or less than 0.002% dry weight per gram of leaf lamina.

In an aspect, a tobacco plant is a single pmt mutant, a double pmt mutant, a triple mutant, a quadruple mutant, or a quintuple mutant as listed in Tables 8A to 8E. In another aspect, a tobacco plant comprises one or more pmt mutant alleles listed in Tables 5A to 5E and Tables 12A to 12E. Each and every combination of the pmt mutant alleles listed in Tables 5A to 5E and Tables 12A to 12E is also provided to give rise to a single pmt mutant, a double pmt mutant, a triple mutant, a quadruple mutant, or a quintuple mutant. Each of the mutated loci can be either homozygous or heterozygous, or comprises a heteroallelic combination. In another aspect, a tobacco plant comprises a pmt mutant genotype combination as shown for each individual line listed in Tables 4A to 4E and Table 10. In an aspect, a tobacco plant comprises a pmt mutant allele sequence selected from the group consisting of SEQ ID Nos. 21 to 200, 410 to 441, 474 to 505, 538 to 569, 602 to 633, and 666 to 697. In another aspect, the present disclosure provides a double pmt mutant, a triple mutant, a quadruple mutant, or a quintuple mutant comprising pmt mutant allele sequences selected from the group consisting of SEQ ID Nos. 21 to 200, 410 to 441, 474 to 505, 538 to 569, 602 to 633, and 666 to 697.

In an aspect, a tobacco plant is capable of producing a leaf comprising a nicotine level less than 90%, 80%, 70%, 60%, 50%, 40%, 30%, 20%, 15%, 10%, 9%, 8%, 7%, 6%, 5%, 4%, 3%, 2%, 1%, 0.5%, or 0.25% of the nicotine level of a leaf from a control tobacco plant when grown and processed under comparable conditions. In another aspect, a tobacco plant is capable of producing a leaf comprising a nicotine level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the nicotine level of a control tobacco plant when grown and processed under comparable conditions.

In another aspect, a tobacco plant is capable of producing a leaf comprising a total alkaloid level less than 90%, 80%, 70%, 60%, 50%, 40%, 30%, 20%, 15%, 10%, 9%, 8%, 7%, 6%, 5%, 4%, 3%, 2%, 1%, 0.5%, or 0.25% of the total alkaloid level of a leaf from a control tobacco plant when grown and processed under comparable conditions. In another aspect, a tobacco plant is capable of producing a leaf comprising a total alkaloid level between 1% and 5%, between 5% and 10%, between 10% and 20%, between 20% and 30%, between 30% and 40%, between 40% and 50%, between 50% and 60%, between 60% and 70%, between 70% and 80%, between 80% and 90%, or between 90% and 95% of the total alkaloid level of a control tobacco plant when grown and processed under comparable conditions.

In a further aspect, a tobacco plant is capable of producing a leaf comprising a total alkaloid level less than 40%, 35%, 30%, 25%, 20%, 15%, 10%, or 5% of the total alkaloid level of a leaf from a control tobacco plant when grown and processed under comparable conditions.

In an aspect, a mutant pmt allele comprises a mutation in a PMT sequence region selected from the group consisting of a promoter, 5′ UTR, first exon, first intron, second exon, second intron, third exon, third intron, fourth exon, fourth intron, fifth exon, fifth intron, sixth exon, sixth intron, seventh exon, seventh intron, eighth exon, 3′ UTR, terminator, and any combination thereof. In another aspect, a mutant pmt allele comprises a mutation in a PMT genomic sequence region listed in Tables 1D to 1H.

In another aspect, a mutant pmt allele comprises one or more mutation types selected from the group consisting of a nonsense mutation, a missense mutation, a frameshift mutation, a splice-site mutation, and any combination thereof. In an aspect, a mutant pmt allele is a null allele or a knock-out allele.

In an aspect, a mutant pmt allele results in one or more of the following: a PMT protein truncation, a non-translatable PMT gene transcript, a non-functional PMT protein, a premature stop codon in a PMT gene, and any combination thereof.

In another aspect, a mutant pmt allele comprises a mutation selected from the group consisting of a substitution, a deletion, an insertion, a duplication, and an inversion of one or more nucleotides relative to a wild-type PMT gene.

In an aspect, a pmt mutant comprises a zygosity status selected from the group consisting of homozygous, heterozygous, and heteroallelic. In another aspect, a pmt mutant is homozygous or heteroallelic in at least 1, 2, 3, 4, or 5 PMT genes. In an aspect, a pmt mutant is homozygous or heteroallelic in at least 4 PMT genes. In another aspect, a pmt mutant is homozygous or heteroallelic in all five PMT genes. In another aspect, a pmt mutant comprises mutations in PMT1a and PMT3.

In an aspect, a tobacco plant is capable of producing a leaf comprising a nicotine level selected from the group consisting of less than 0.15%, less than 0.125%, less than 0.1%, less than 0.08%, less than 0.06%, less than 0.05%, less than 0.04%, less than 0.03%, less than 0.02%, and less than 0.01% dry weight.

In another aspect, a tobacco plant is capable of producing a leaf comprising a total alkaloid level selected from the group consisting of less than 1%, less than 0.8%, less than 0.7%, less than 0.6%, less than 0.5%, less than 0.4%, less than 0.3%, and less than 0.2% dry weight.

In a further aspect, a tobacco plant is capable of producing a cured leaf comprising a total tobacco-specific nitrosamine (TSNA) level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm.

In an aspect, a tobacco plant is capable of producing leaves, when cured, having a USDA grade index value selected from the group consisting of 50 or more, 55 or more, 60 or more, 65 or more, 70 or more, 75 or more, 80 or more, 85 or more, 90 or more, and 95 or more. In another aspect, a tobacco plant is capable of producing leaves, when cured, having a USDA grade index value comparable to that of a control plant when grown and cured in similar conditions, where the control plant shares an essentially identical genetic background with the tobacco plant except for the modification. In a further aspect, a tobacco plant is capable of producing leaves, when cured, having a USDA grade index value of at least about 65%, at least about 70%, at least about 75%, at least about 80%, at least about 85%, at least about 90%, at least about 95%, or at least about 98% of the USDA grade index value of a control plant when grown in similar conditions, where the control plant shares an essentially identical genetic background with the tobacco plant except the modification. In a further aspect, a tobacco plant is capable of producing leaves, when cured, having a USDA grade index value of between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the USDA grade index value of a control plant. In a further aspect, a tobacco plant is capable of producing leaves, when cured, having a USDA grade index value of between 70% and 125%, between 75% and 120%, between 80% and 115%, between 85% and 110%, or between 90% and 100% of the USDA grade index value of a control plant.

In an aspect, a tobacco plant comprises nicotine at a level below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the control plant shares an essentially identical genetic background with the tobacco plant except for the modification.

In a further aspect, a tobacco plant comprises one or more pmt mutant alleles and further comprises a transgene or mutation directly suppressing the expression or activity of one or more genes encoding a product selected from the group consisting of MPO, QPT, BBL, A622, aspartate oxidase, agmatine deiminase (AIC), arginase, diamine oxidase, ornithine decarboxylase, arginine decarboxylase, nicotine uptake permease (NUP), and MATE transporter.

In an aspect, a tobacco plant comprises one or more pmt mutant alleles and further comprises a mutation in an ERF gene of Nic2 locus. In an aspect, a tobacco plant further comprises one or more mutations in two or more, three or more, four or more, five or more, six or more, or all seven genes selected from the group consisting of ERF189, ERF115, ERF221, ERF104, ERF179, ERF17, and ERF168. See Shoji et al., Plant Cell, (10):3390-409 (2010); and Kajikawa et al., Plant physiol. 2017, 174:999-1011. In an aspect, a tobacco plant further comprises one or more mutations in ERF189, ERF115, or both.

In an aspect, a tobacco plant comprises one or more qpt mutant alleles and further comprises a mutation in an ERF gene of Nic1 locus (or Nic1b locus as in PCT/US2019/013345 filed on Jan. 11, 2019, published as WO/2019/140297). See also WO/2018/237107. In an aspect, a tobacco plant further comprises one or more mutations in two or more, three or more, four or more, five or more, six or more, or seven or more genes selected from the group consisting of ERF101, ERF110, ERFnew, ERF199, ERF19, ERF130, ERF16, ERF29, ERF210, and ERF91L2. See Kajikawa et al., Plant physiol. 2017, 174:999-1011. In an aspect, a tobacco plant further comprises one or more mutations in one or more, two or more, three or more, four or more, five or more, or all six genes selected from the group consisting of ERFnew, ERF199, ERF19, ERF29, ERF210, and ERF91L2.

In an aspect, a low-nicotine tobacco plant (e.g., having one or more qpt mutant alleles) further comprises one of more genetic modifications providing a reduced level of anatabine. Exemplary genetic modifications that provide reduce anatabine can be found in US20160010103A1 and U.S. Pat. No. 10,375,910B2. In an aspect, a anatabine-reducing genetic modification comprising a mutation in a Quinolinate Synthase (QS) gene. In another aspect, a QS gene mutation comprising a mutation resulting in an amino acid substitution at a position corresponding to the cysteine residue at position 487 and/or the valine residue at position 516 of SEQ ID No: 8 of US20160010103A1. In another aspect, a anatabine-reducing genetic modification is present in, introgressed from or originates from tobacco plant line dMS932, wherein a representative sample of seed of said tobacco plant is deposited under ATCC Accession Number PTA-124990. In another aspect, a anatabine-reducing genetic modification is present in, introgressed from or originates from a tobacco plant line selected from the group consisting of MS108, MS445, MS170, and MS3908 from U.S. Pat. No. 10,375,910B2.

In an aspect, the present disclosure further provides a pmt mutant tobacco plant, or part thereof, comprising a nicotine or total alkaloid level selected from the group consisting of less than 3%, less than 2.75%, less than 2.5%, less than 2.25%, less than 2.0%, less than 1.75%, less than 1.5%, less than 1.25%, less than 1%, less than 0.9%, less than 0.8%, less than 0.7%, less than 0.6%, less than 0.5%, less than 0.4%, less than 0.3%, less than 0.2%, less than 0.1%, less than 0.05%, less than 0.025%, less than 0.01%, and less than 0.005%, where the tobacco plant is capable of producing leaves, when cured, having a USDA grade index value of 50 or more 55 or more, 60 or more, 65 or more, 70 or more, 75 or more, 80 or more, 85 or more, 90 or more, and 95 or more. In another aspect, such pmt mutant tobacco plant comprises a nicotine level of less than 0.02% and are capable of producing leaves, when cured, having a USDA grade index value of 70 or more. In a further aspect, such tobacco plant comprises a nicotine level of less than 0.01% and are capable of producing leaves, when cured, having a USDA grade index value of 70 or more.

In an aspect, the present disclosure also provides a tobacco plant, or part thereof, comprising a non-transgenic mutation, where the non-transgenic mutation reduces the nicotine or total alkaloid level of the tobacco plant to below 1%, below 2%, below 5%, below 8%, below 10%, below 12%, below 15%, below 20%, below 25%, below 30%, below 40%, below 50%, below 60%, below 70%, or below 80% of the nicotine level of a control plant when grown in similar growth conditions, where the tobacco plant is capable of producing leaves, when cured, having a USDA grade index value comparable to the USDA grade index value of the control plant, and where the control plant shares an essentially identical genetic background with the tobacco plant except the non-transgenic mutation.

In an aspect, a tobacco plant comprises a pmt mutation introduced by an approach selected from the group consisting of random mutagenesis and targeted mutagenesis. In another aspect, a pmt mutation is introduced by a targeted mutagenesis approach selected from the group consisting of meganuclease, zinc finger nuclease, TALEN, and CRISPR.

Unless specified otherwise, measurements of alkaloid or nicotine levels (or another leaf chemistry or property characterization) or leaf grade index values mentioned herein for a tobacco plant, variety, cultivar, or line refer to average measurements, including, for example, an average of multiple leaves of a single plant or an average measurement from a population of tobacco plants from a single variety, cultivar, or line.

Unless specified otherwise, the nicotine or alkaloid level (or another leaf chemistry or property characterization) of a tobacco plant is measured after topping in a pooled leaf sample collected from leaf number 3, 4, and 5 after topping. As used herein, whenever a comparison between leaves from two plants (e.g., a mutant plant versus a control plant) is mentioned, leaves from the same or comparable stalk position(s) and developmental stage(s) are intended so that the comparison can demonstrate effects due to genotype differences, not from other factors. As an illustration, leaf 3 of a wild-type control plant is intended as a reference point for comparing with leaf 3 of a pmt mutant plant. In an aspect, a tobacco plant comprising at least one pmt mutation is compared to a control tobacco plant of the same tobacco variety.

Nicotine or alkaloid level (or another leaf chemistry or property characterization) of a tobacco plant can also be measured in alternative ways. In an aspect, the nicotine or alkaloid level (or another leaf chemistry or property characterization) of a tobacco plant is measured after topping in a leaf having the highest level of nicotine or alkaloid (or another leaf chemistry or property characterization). In an aspect, the nicotine or alkaloid level of a tobacco plant is measured after topping in leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In another aspect, the nicotine or alkaloid level (or another leaf chemistry or property characterization) of a tobacco plant is measured after topping in a pool of two or more leaves with consecutive leaf numbers selected from the group consisting of leaf number 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30. In another aspect, the nicotine or alkaloid level (or another leaf chemistry or property characterization) of a tobacco plant is measured after topping in a leaf with a leaf number selected from the group consisting of between 1 and 5, between 6 and 10, between 11 and 15, between 16 and 20, between 21 and 25, and between 26 and 30. In another aspect, the nicotine or alkaloid level (or another leaf chemistry or property characterization) of a tobacco plant is measured after topping in a pool of two or more leaves with leaf numbers selected from the group consisting of between 1 and 5, between 6 and 10, between 11 and 15, between 16 and 20, between 21 and 25, and between 26 and 30. In another aspect, the nicotine or alkaloid level (or another leaf chemistry or property characterization) of a tobacco plant is measured after topping in a pool of three or more leaves with leaf numbers selected from the group consisting of between 1 and 5, between 6 and 10, between 11 and 15, between 16 and 20, between 21 and 25, and between 26 and 30.

As used herein, leaf numbering is based on the leaf position on a tobacco stalk with leaf number 1 being the youngest leaf (at the top) after topping and the highest leaf number assigned to the oldest leaf (at the bottom).

A population of tobacco plants or a collection of tobacco leaves for determining an average measurement (e.g., alkaloid or nicotine level or leaf grading) can be of any size, for example, 5, 10, 15, 20, 25, 30, 35, 40, or 50. Industry-accepted standard protocols are followed for determining average measurements or grad index values.

As used herein, “topping” refers to the removal of the stalk apex, including the SAM, flowers, and up to several adjacent leaves, when a tobacco plant is near vegetative maturity and around the start of reproductive growth. Typically, tobacco plants are topped in the button stage (soon after the flower begins to appear). For example, greenhouse or field-grown tobacco plants can be topped when 50% of the plants have at least one open flower. Topping a tobacco plant results in the loss of apical dominance and also induces increased alkaloid production.

Unless indicated otherwise, the nicotine or alkaloid level (or another leaf chemistry or property characterization) of a tobacco plant is measured 2 weeks after topping. Alternatively, other time points can be used. In an aspect, the nicotine or alkaloid level (or another leaf chemistry or property characterization) of a tobacco plant is measured about 1, 2, 3, 4, or 5 weeks after topping. In another aspect, the nicotine, alkaloid, or polyamine level (or another leaf chemistry or property characterization) of a tobacco plant is measured about 3, 5, 7, 10, 12, 14, 17, 19 or 21 days after topping.

As used herein, “similar growth conditions” or “comparable growth conditions” refer to similar environmental conditions and/or agronomic practices for growing and making meaningful comparisons between two or more plant genotypes so that neither environmental conditions nor agronomic practices would contribute to or explain any difference observed between the two or more plant genotypes. Environmental conditions include, for example, light, temperature, water (humidity), and nutrition (e.g., nitrogen and phosphorus). Agronomic practices include, for example, seeding, clipping, undercutting, transplanting, topping, and suckering. See Chapters 4B and 4C of Tobacco, Production, Chemistry and Technology, Davis & Nielsen, eds., Blackwell Publishing, Oxford (1999), pp 70-103.

“Alkaloids” are complex, nitrogen-containing compounds that naturally occur in plants, and have pharmacological effects in humans and animals. “Nicotine” is the primary natural alkaloid in commercialized cigarette tobacco and accounts for about 90 percent of the alkaloid content in Nicotiana tabacum. Other major alkaloids in tobacco include cotinine, nornicotine, myosmine, nicotyrine, anabasine and anatabine. Minor tobacco alkaloids include nicotine-n-oxide, N-methyl anatabine, N-methyl anabasine, pseudooxynicotine, 2,3 dipyridyl and others.

As used herein, “comparable leaves” refer to leaves having similar size, shape, age, and/or stalk position.

Alkaloid levels can be assayed by methods known in the art, for example by quantification based on gas-liquid chromatography, high performance liquid chromatography, radio-immunoassays, and enzyme-linked immunosorbent assays. For example, nicotinic alkaloid levels can be measured by a GC-FID method based on CORESTA Recommended Method No. 7, 1987 and ISO Standards (ISO TC 126N 394 E. See also Hibi et al., Plant Physiology 100:826-35 (1992) for a method using gas-liquid chromatography equipped with a capillary column and an FID detector.

Unless specifically indicated otherwise, alkaloids and nicotine levels are measured using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009). Alternatively, tobacco total alkaloids can be measured using a segmented-flow colorimetric method developed for analysis of tobacco samples as adapted by Skalar Instrument Co (West Chester, PA) and described by Collins et al., Tobacco Science 13:79-81 (1969). In short, samples of tobacco can be dried, ground, and extracted prior to analysis of total alkaloids and reducing sugars. The method then employs an acetic acid/methanol/water extraction and charcoal for decolorization. Determination of total alkaloids is based on the reaction of cyanogen chloride with nicotine alkaloids in the presence of an aromatic amine to form a colored complex which is measured at 460 nm. Unless specified otherwise, total alkaloid levels or nicotine levels shown herein are on a dry weight basis (e.g., percent total alkaloid or percent nicotine).

In an aspect, a tobacco plant comprises an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, a tobacco plant comprises an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, a tobacco plant comprises an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

The present disclosure also provides a tobacco plant having an altered nicotine level without negative impacts over other tobacco traits, e.g., leaf grade index value. In an aspect, a low-nicotine or nicotine-free tobacco variety provides cured tobacco of commercially acceptable grade. Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaves to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40 (1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by inference in their entirety). In an aspect, a USDA grade index is a 0-100 numerical representation of federal grade received and is a weighted average of all stalk positions. A higher grade index indicates higher quality. Alternatively, leaf grade can be determined via hyper-spectral imaging. See e.g., WO 2011/027315 (published on Mar. 10, 2011, and incorporated by inference in its entirety).

In an aspect, a tobacco plant provided herein comprises a similar level of one or more tobacco aroma compounds compared to a control tobacco plant when grown in similar growth conditions. In another aspect, a tobacco plant provided herein comprise a similar level of one or more tobacco aroma compounds selected from the group consisting of 3-methylvaleric acid, valeric acid, isovaleric acid, a labdenoid, a cembrenoid, a sugar ester, and a reducing sugar, compared to a control tobacco plant when grown in similar growth conditions.

As used herein, tobacco aroma compounds are compounds associated with the flavor and aroma of tobacco smoke. These compounds include, but are not limited to, 3-methylvaleric acid, valeric acid, isovaleric acid, cembrenoid and labdenoid diterpenes, and sugar esters. Concentrations of tobacco aroma compounds can be measured by any known metabolite profiling methods in the art including, without limitation, gas chromatography mass spectrometry (GC-MS), Nuclear Magnetic Resonance Spectroscopy, liquid chromatography-linked mass spectrometry. See The Handbook of Plant Metabolomics, edited by Weckwerth and Kahl, (Wiley-Blackwell) (May 28, 2013).

As used herein, “reducing sugar(s)” are any sugar (monosaccharide or polysaccharide) that has a free or potentially free aldehdye or ketone group. Glucose and fructose act as nicotine buffers in cigarette smoke by reducing smoke pH and effectively reducing the amount of “free” unprotonated nicotine. Reducing sugars balances smoke flavor, for example, by modifying the sensory impact of nicotine and other tobacco alkaloids. An inverse relationship between sugar content and alkaloid content has been reported across tobacco varieties, within the same variety, and within the same plant line caused by planting conditions. Reducing sugar levels can be measured using a segmented-flow colorimetric method developed for analysis of tobacco samples as adapted by Skalar Instrument Co (West Chester, PA) and described by Davis, Tobacco Science 20:139-144 (1976). For example, a sample is dialyzed against a sodium carbonate solution. Copper neocuproin is added to the sample and the solution is heated. The copper neocuproin chelate is reduced in the presence of sugars resulting in a colored complex which is measured at 460 nm.

In an aspect, a tobacco plant comprises one or more non-naturally existing mutant alleles in one or more PMT gene loci which reduce or eliminate PMT enzymatic activity from the one or more PMT gene loci. In an aspect, these mutant alleles result in lower nicotine levels. Mutant pmt alleles can be introduced by any method known in the art including random or targeted mutagenesis approaches.

Such mutagenesis methods include, without limitation, treatment of seeds with ethyl methylsulfate (EMS) (Hildering and Verkerk, In, The use of induced mutations in plant breeding. Pergamon press, pp 317-320, 1965) or UV-irradiation, X-rays, and fast neutron irradiation (see, for example, Verkerk, Neth. J. Agric. Sci. 19:197-203, 1971; and Poehlman, Breeding Field Crops, Van Nostrand Reinhold, New York (3.sup.rd ed), 1987), transposon tagging (Fedoroff et al., 1984; U.S. Pat. Nos. 4,732,856 and 5,013,658), as well as T-DNA insertion methodologies (Hoekema et al., 1983; U.S. Pat. No. 5,149,645). EMS-induced mutagenesis consists of chemically inducing random point mutations over the length of the genome. Fast neutron mutagenesis consists of exposing seeds to neutron bombardment which causes large deletions through double stranded DNA breakage. Transposon tagging comprises inserting a transposon within an endogenous gene to reduce or eliminate expression of the gene. The types of mutations that may be present in a tobacco gene include, for example, point mutations, deletions, insertions, duplications, and inversions. Such mutations desirably are present in the coding region of a tobacco gene; however mutations in the promoter region, and intron, or an untranslated region of a tobacco gene may also be desirable.

In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See, McCallum et al. (2000) Nat. Biotechnol. 18:455-457. Mutations that impact gene expression or that interfere with the function of genes can be determined using methods that are well known in the art. Insertional mutations in gene exons usually result in null-mutants. Mutations in conserved residues can be particularly effective in inhibiting the function of a protein. In an aspect, tobacco plants comprise a nonsense (e.g., stop codon) mutation in one or more PMT genes described herein.

It will be appreciated that, when identifying a mutation, the endogenous reference DNA sequence should be from the same variety of tobacco. For example, if a modified tobacco plant comprising a mutation is from the variety TN90, then the endogenous reference sequence must be the endogenous TN90 sequence, not a homologous sequence from a different tobacco variety (e.g., K326). Similarly, if a modified tobacco cell comprising a mutation is a TN90 cell, then the endogenous reference sequence must be the endogenous TN90 sequence, not a homologous sequence from a tobacco cell from a different tobacco variety (e.g., K326).

In an aspect, the present disclosure also provides a tobacco line with altered nicotine levels while maintaining commercially acceptable leaf quality. This line can be produced by introducing mutations into one or more PMT genes via precise genome engineering technologies, for example, Transcription activator-like effector nucleases (TALENs), meganuclease, zinc finger nuclease, and a clustered regularly-interspaced short palindromic repeats (CRISPR)/Cas9 system, a CRISPR/Cpf1 system, a CRISPR/Csm1 system, and a combination thereof (see, for example, U.S. Patent Application publication 2017/0233756). See, e.g., Gaj et al., Trends in Biotechnology, 31 (7): 397-405 (2013).

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454), enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In an aspect, a tobacco plant or plant genome provided herein is mutated or edited by a nuclease selected from the group consisting of a meganuclease, a zinc-finger nuclease (ZFN), a transcription activator-like effector nuclease (TALEN), a CRISPR/Cas9 nuclease, a CRISPR/Cpf1 nuclease, or a CRISPR/Csm1 nuclease.

As used herein, “editing” or “genome editing” refers to targeted mutagenesis of at least 1, at least 2, at least 3, at least 4, at least 5, at least 6, at least 7, at least 8, at least 9, or at least 10 nucleotides of an endogenous plant genome nucleic acid sequence, or removal or replacement of an endogenous plant genome nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with an endogenous nucleic acid sequence. In an aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide selected from the group consisting of SEQ ID NOs: 1 to 10, and fragments thereof. In another aspect, an edited nucleic acid sequence provided has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide encoding a polypeptide selected from the group consisting of SEQ ID NOs: 11 to 15.

Meganucleases, ZFNs, TALENS, CRISPR/Cas9, CRISPR/Csm1 and CRISPR/Cpf1 induce a double-strand DNA break at a target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of donor nucleic acid sequences by HR. In an aspect, a method provided comprises editing a plant genome with a nuclease provided to mutate at least 1, at least 2, at least 3, at least 4, at least 5, at least 6, at least 7, at least 8, at least 9, at least 10, or more than 10 nucleotides in the plant genome via HR with a donor polynucleotide. In an aspect, a mutation provided is caused by genome editing using a nuclease. In another aspect, a mutation provided is caused by non-homologous end-joining or homologous recombination.

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

ZFNs are synthetic proteins consisting of an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger co-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

TALENs are artificial restriction enzymes generated by fusing the transcription activator-like effector (TALE) DNA binding domain to a FokI nuclease domain. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

Transcription activator-like effectors (TALEs) can be engineered to bind practically any DNA sequence. TALE proteins are DNA-binding domains derived from various plant bacterial pathogens of the genus Xanthomonas. The Xanthomonas pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

A relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about.

A CRISPR/Cas9 system, CRISPR/Csm1, or a CRISPR/Cpf1 system are alternatives to the FokI-based methods ZFN and TALEN. The CRISPR systems are based on RNA-guided engineered nucleases that use complementary base pairing to recognize DNA sequences at target sites.

CRISPR/Cas9, CRISPR/Csm1, and a CRISPR/Cpf1 systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5-NGG-3 but less frequently NAG. Specificity is provided by the so-called “seed sequence” approximately 12 bases upstream of the PAM, which must match between the RNA and target DNA. Cpf1 and Csm1 act in a similar manner to Cas9, but Cpf1 and Csm1 do not require a tracrRNA.

In still another aspect, a tobacco plant provided here comprises one or more pmt mutations and further comprises one or more mutations in one or more loci encoding a nicotine demethylase (e.g., CYP82E4, CYP82E5, CYP82E10) that confer reduced amounts of nornicotine (See U.S. Pat. Nos. 8,319,011; 8,124,851; 9,187,759; 9,228,194; 9,228,195; 9,247,706) compared to a control plant lacking one or more mutations in one or more loci encoding a nicotine demethylase. In an aspect, a tobacco plant described further comprises reduced nicotine demethylase activity compared to a control plant when grown and cured under comparable conditions.

In an aspect, a pmt mutant tobacco plant is capable of producing a leaf comprising an anabasine level more than 150% of the anabasine level of a leaf from a wild-type control tobacco plant when grown and processed under comparable conditions. In another aspect, a pmt mutant tobacco plant is capable of producing a leaf comprising an anabasine level more than 175%, 200%, 250%, 300%, 350%, 400%, 500%, or 600% of the anabasine level of a leaf from a wild-type control tobacco plant when grown and processed under comparable conditions.

In an aspect, a pmt mutant tobacco plant is capable of producing a leaf comprising an anatabine level more than 2 folds of the anatabine level of a leaf from a control tobacco plant when grown and processed under comparable conditions. In another aspect, a pmt mutant tobacco plant is capable of producing a leaf comprising an anatabine level more than 3, 4, 5, 6, 7, 8, 9, 10, 11, 12, or 13 folds of the anatabine level of a leaf from a wild-type control tobacco plant when grown and processed under comparable conditions. In another aspect, a tobacco plant provided here comprises one or more genetic modifications providing an increased anatabine levels and at least one genetic modifications providing a commercially acceptable leaf grade. In an aspect, one or more genetic modifications providing an increased anatabine levels are the same or overlap with at least one genetic modifications providing a commercially acceptable leaf grade. In an aspect, leaves with a commercially acceptable leaf grade refer to leaves, when cured, having a USDA grade index value of 50 or more 55 or more, 60 or more, 65 or more, 70 or more, 75 or more, 80 or more, 85 or more, 90 or more, and 95 or more.

In an aspect, a pmt mutant tobacco plant is capable of producing a leaf comprising a nornicotine level more than 2, 3, 4, 5, 6, 7, 8, 9, 10, 11, 12, or 13 folds of the nornicotine level of a leaf from a wild-type control tobacco plant when grown and processed under comparable conditions.

In an aspect, a pmt mutant tobacco plant further comprises a mutation capable of producing a leaf comprising an anabasine level less than the anabasine level of a leaf from a wild-type control tobacco plant when grown and processed under comparable conditions. In another aspect, a pmt mutant tobacco plant further comprises a mutation capable of producing a leaf comprising an anabasine level less than 5%, 10%, 20%, 25%, 30%, 40%, 50%, 60%, 70%, or 80% of the anabasine level of a leaf from a wild-type control tobacco plant when grown and processed under comparable conditions.

In an aspect, a pmt mutant tobacco plant comprises a further mutation capable of producing a leaf comprising a more than 2 fold reduction of the anatabine level compared to a leaf from a control tobacco plant when grown and processed under comparable conditions. In another aspect, a pmt mutant tobacco plant comprises a further mutation capable of producing a leaf comprising a more than 3, 4, 5, 6, 7, 8, 9, 10, 11, 12, or 13 fold reduction of the anatabine level compared to a leaf from a wild-type control tobacco plant when grown and processed under comparable conditions. In an aspect, a mutation providing lower level of anatabine is a mutation described in US Application Publication No. 2014/0283165 and US Application Publication No. 2016/0010103. In another aspect, a pmt mutant further comprises a mutation in a quinolate phosphoribosyl transferase (QPT) or quinolinate synthase (QS) gene. In a further aspect, a pmt mutant plant further comprises a transgene or mutation suppressing the expression or activity of a QPT or QS gene.

In an aspect, a pmt mutant tobacco plant further comprises a mutation capable of providing a nornicotine level less than 80%, 75%, 70%, 65%, 60%, 55%, 50%, 45%, 40%, or 35% of the nornicotine level of a leaf from a wild-type control tobacco plant when grown and processed under comparable conditions.

In an aspect, a pmt mutant tobacco plant is capable of producing a cured leaf comprising a total N-nitrosonornicotine (NNN) level of less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm.

In another aspect, a pmt mutant tobacco plant is capable of producing a cured leaf comprising a total NNN level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 parts per million (ppm).

In an aspect, a pmt mutant tobacco plant is capable of producing a cured leaf comprising a total nicotine-derived nitrosamine ketone (NNK) level of less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm.

In another aspect, a pmt mutant tobacco plant is capable of producing a cured leaf comprising a total NNK level of between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm.

In an aspect, a pmt mutant tobacco plant further comprises a mutation or transgene providing an increased level of one or more antioxidants. In another aspect, a pmt mutant tobacco plant further comprises a genetic modification in an endogenous gene and further comprises an increased level of one or more antioxidants in a cured leaf compared to a control cured tobacco leaf lacking the genetic modification, where the endogenous gene encodes an antioxidant biosynthetic enzyme, a regulatory transcription factor of an antioxidant, an antioxidant transporter, an antioxidant metabolic enzyme, or a combination thereof. In a further aspect, a pmt mutant tobacco plant further comprises a transgene and further comprises an increased level of one or more antioxidants in a cured leaf compared to a control cured tobacco leaf lacking the transgene, where the transgene encodes or directly modulates an antioxidant biosynthetic enzyme, a regulatory transcription factor of an antioxidant, an antioxidant transporter, an antioxidant metabolic enzyme, or a combination thereof. In an aspect, a pmt mutant tobacco plant further comprises a transgene or a cisgenic construct expressing one or more genes selected from the group consisting of AtPAP1, NtAN2, NtAN1, NtJAF13, NtMyb3, chorismate mutase, and arogenate dehydrotase (ADT). In another aspect, a pmt mutant tobacco plant further comprises one or more transgenes or genetic modification for increasing antioxidants or decreasing one or more TSNAs as described in WIPO Publication No. 2018/067985 or US Publication No. 2018/0119163.

In an aspect, a tobacco plant described is a modified tobacco plant. As used herein, “modified”, in the context of a plant, refers to a plant comprising a genetic alteration introduced for certain purposes and beyond natural polymorphisms.

In an aspect, a tobacco plant described is a cisgenic plant. As used herein, “cisgenesis” or “cisgenic” refers to genetic modification of a plant, plant cell, or plant genome in which all components (e.g., promoter, donor nucleic acid, selection gene) have only plant origins (i.e., no non-plant origin components are used). In an aspect, a plant, plant cell, or plant genome provided is cisgenic. Cisgenic plants, plant cells, and plant genomes provided can lead to ready-to-use tobacco lines. In another aspect, a tobacco plant provided comprises no non-tobacco genetic material or sequences.

As used herein, “gene expression” or expression of a gene refers to the biosynthesis or production of a gene product, including the transcription and/or translation of the gene product.

In an aspect, a tobacco plant provided comprises one or more pmt mutations and further comprises reduced expression or activity of one or more genes involved in nicotine biosynthesis or transport. Genes involved in nicotine biosynthesis include, but are not limited to, arginine decarboxylase (ADC), methylputrescine oxidase (MPO), NADH dehydrogenase, ornithine decarboxylase (ODC), phosphoribosylanthranilate isomerase (PRAI), quinolate phosphoribosyl transferase (QPT), and S-adenosyl-methionine synthetase (SAMS). Nicotine Synthase, which catalyzes the condensation step between a nicotinic acid derivative and methylpyrrolinium cation, has not been elucidated although two candidate genes (A622 and NBB1) have been proposed. See US 2007/0240728 A1 and US 2008/0120737A1. A622 encodes an isoflavone reductase-like protein. In addition, several transporters may be involved in the translocation of nicotine. A transporter gene, named MATE, has been cloned and characterized (Morita et al., PNAS 106:2447-52 (2009)).

In an aspect, a tobacco plant provided comprises one or more pmt mutations and further comprises a reduced level of mRNA, protein, or both of one or more genes encoding a product selected from the group consisting of MPO, QPT, ADC, ODC, PRAI, SAMS, BBL, MATE, A622, and NBB1, compared to a control tobacco plant. In another aspect, a tobacco plants provided comprises one or more pmt mutations and further comprises a transgene directly suppressing the expression of one or more genes encoding a product selected from the group consisting of MPO, QPT, ADC, ODC, PRAI, SAMS, BBL, MATE, A622, and NBB1. In another aspect, a tobacco plant provided comprises one or more pmt mutations and further comprises a transgene or mutation suppressing the expression or activity of one or more genes encoding a product selected from the group consisting of MPO, QPT, ADC, ODC, PRAI, SAMS, BBL, MATE, A622, and NBB1.

In an aspect, a tobacco plant provided is from a tobacco type selected from the group consisting of flue-cured tobacco, air-cured tobacco, dark air-cured tobacco, dark fire-cured tobacco, Galpao tobacco, and Oriental tobacco. In another aspect, a tobacco plant provided is from a tobacco type selected from the group consisting of Burley tobacco, Maryland tobacco, and dark tobacco. In an aspect, tobacco plants or seeds or modified tobacco plants or seeds provided here are of a tobacco variety selected from the group consisting of the tobacco varieties listed in Tables 16 to 22, and any variety essentially derived from any one of the foregoing varieties. See WO 2004/041006 A1.

In an aspect, a tobacco plant provided is in a flue-cured tobacco background or exhibits one or more flue-cured tobacco characteristic described here. Flue-cured tobaccos (also called Virginia or bright tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the U.S. In an aspect, a low-alkaloid or low-nicotine tobacco plant or seed provided is in a flue-cured tobacco background selected from the group consisting of CC 13, CC 27, CC 33, CC 37, CC 65, CC 67, CC 700, GF 318, GL 338, GL 368, GL 939, K 346, K 399, K326, NC 102, NC 196, NC 291, NC 297, NC 299, NC 471, NC 55, NC 606, NC 71, NC 72, NC 92, PVH 1118, PVH 1452, PVH 2110, SPEIGHT 168, SPEIGHT 220, SPEIGHT 225, SPEIGHT 227, SPEIGHT 236, and any variety essentially derived from any one of the foregoing varieties. In another aspect, a low-alkaloid or low-nicotine tobacco plant or seed provided is in a flue-cured tobacco background selected from the group consisting of Coker 48, Coker 176, Coker 371-Gold, Coker 319, Coker 347, GL 939, K 149, K326, K 340, K 346, K 358, K 394, K 399, K 730, NC 27NF, NC 37NF, NC 55, NC 60, NC 71, NC 72, NC 82, NC 95, NC 297, NC 606, NC 729, NC 2326, McNair 373, McNair 944, Ox 207, Ox 414 NF, Reams 126, Reams 713, Reams 744, RG 8, RG 11, RG 13, RG 17, RG 22, RG 81, RG H4, RG H51, Speight H-20, Speight G-28, Speight G-58, Speight G-70, Speight G-108, Speight G-111, Speight G-117, Speight 168, Speight 179, Speight NF-3, Va 116, Va 182, and any variety essentially derived from any one of the foregoing varieties. See WO 2004/041006 A1. In a further aspect, low-alkaloid or low-nicotine tobacco plants, seeds, hybrids, varieties, or lines are in any flue cured background selected from the group consisting of K326, K346, and NC196.

In an aspect, a tobacco plant provided is in an air-cured tobacco background or exhibits one or more air-cured tobacco characteristic described here. Air-cured tobaccos include Burley, Maryland, and dark tobaccos. The common factor is that curing is primarily without artificial sources of heat and humidity. Burley tobaccos are light to dark brown in color, high in oil, and low in sugar. Burley tobaccos are air-cured in barns. Major Burley growing countries are Argentina, Brazil, Italy, Malawi, and the U.S. Maryland tobaccos are extremely fluffy, have good burning properties, low nicotine and a neutral aroma. Major Maryland growing countries include the U.S. and Italy. In an aspect, a low-alkaloid or low-nicotine tobacco plant or seed provided is in a Burley tobacco background selected from the group consisting of Clay 402, Clay 403, Clay 502, Ky 14, Ky 907, Ky 910, Ky 8959, NC 2, NC 3, NC 4, NC 5, NC 2000, TN 86, TN 90, TN 97, R 610, R 630, R 711, R 712, NCBH 129, Bu 21×Ky 10, HB04P, Ky 14×L 8, Kt 200, Newton 98, Pedigo 561, Pf561 and Va 509. In a further aspect, low-alkaloid or low-nicotine tobacco plants, seeds, hybrids, varieties, or lines are in any Burley background selected from the group consisting of TN 90, KT 209, KT 206, KT212, and HB 4488. In another aspect, a low-alkaloid or low-nicotine tobacco plant or seed provided is in a Maryland tobacco background selected from the group consisting of Md 10, Md 40, Md 201, Md 609, Md 872 and Md 341.

In an aspect, a tobacco plant provided is in a dark air-cured tobacco background or exhibits one or more dark air-cured tobacco characteristic described here. Dark air-cured tobaccos are distinguished from other types primarily by its curing process which gives dark air-cured tobacco its medium- to dark-brown color and distinct aroma. Dark air-cured tobaccos are mainly used in the production of chewing tobacco and snuff. In an aspect, a low-alkaloid or low-nicotine tobacco plant or seed provided is in a dark air-cured tobacco background selected from the group consisting of Sumatra, Jatim, Dominican Cubano, Besuki, One sucker, Green River, Virginia sun-cured, and Paraguan Passado.

In an aspect, a tobacco plant provided is in a dark fire-cured tobacco background or exhibits one or more dark fire-cured tobacco characteristic described here. Dark fire-cured tobaccos are generally cured with low-burning wood fires on the floors of closed curing barns. Their leaves have low sugar content but high nicotine content. Dark fire-cured tobaccos are used for making pipe blends, cigarettes, chewing tobacco, snuff and strong-tasting cigars. Major growing regions for dark fire-cured tobaccos are Tennessee, Kentucky, and Virginia, USA. In an aspect, a low-alkaloid or low-nicotine tobacco plant or seed provided is in a dark fire-cured tobacco background selected from the group consisting of Narrow Leaf Madole, Improved Madole, Tom Rosson Madole, Newton's VH Madole, Little Crittenden, Green Wood, Little Wood, Small Stalk Black Mammoth, DT 508, DT 518, DT 592, KY 171, DF 911, DF 485, TN D94, TN D950, VA 309, and VA 359.

In an aspect, a tobacco plant provided is in an Oriental tobacco background or exhibits one or more Oriental tobacco characteristic described here. Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant and leaf size, characteristic of today's Oriental varieties, as well as its unique aroma properties are a result of the plant's adaptation to the poor soil and stressful climatic conditions in which it develop over many past centuries. In an aspect, a low-alkaloid or low-nicotine tobacco plant or seed provided is in an Oriental tobacco background selected from the group consisting of Izmir, Katerini, Samsun, Basma and Krumovgrad, Trabzon, Thesalian, Tasova, Sinop, Izmit, Hendek, Edirne, Semdinli, Adiyanman, Yayladag, Iskenderun, Duzce, Macedonian, Mavra, Prilep, Bafra, Bursa, Bucak, Bitlis, Balikesir, and any variety essentially derived from any one of the foregoing varieties.

In an aspect, low-alkaloid or low-nicotine tobacco plants, seeds, hybrids, varieties, or lines are essentially derived from or in the genetic background of BU 64, CC 101, CC 200, CC 27, CC 301, CC 400, CC 500, CC 600, CC 700, CC 800, CC 900, Coker 176, Coker 319, Coker 371 Gold, Coker 48, CU 263, DF911, Galpao tobacco, GL 26H, GL 350, GL 600, GL 737, GL 939, GL 973, HB 04P, K 149, K 326, K 346, K 358, K394, K 399, K 730, KDH 959, KT 200, KT204LC, KY 10, KY 14, KY 160, KY 17, KY 171, KY 907, KY907LC, KTY14×L8 LC, Little Crittenden, McNair 373, McNair 944, msKY 14×L8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, or VA359, Maryland 609, HB3307PLC, HB4488PLC, KT206LC, KT209LC, KT210LC, KT212LC, R610LC, PVH2310, NC196, KTD14LC, KTD6LC, KTD8LC, PD7302LC, PD7305LC, PD7309LC, PD7318LC, PD7319LC, PD7312LC, ShireyLC, or any commercial tobacco variety according to standard tobacco breeding techniques known in the art.

All foregoing mentioned specific varieties of dark air-cured, Burley, Maryland, dark fire-cured, or Oriental type are listed only for exemplary purposes. Any additional dark air-cured, Burley, Maryland, dark fire-cured, Oriental varieties are also contemplated in the present application.

Also provided are populations of tobacco plants described. In an aspect, a population of tobacco plants has a planting density of between about 5,000 and about 8000, between about 5,000 and about 7,600, between about 5,000 and about 7,200, between about 5,000 and about 6,800, between about 5,000 and about 6,400, between about 5,000 and about 6,000, between about 5,000 and about 5,600, between about 5,000 and about 5,200, between about 5,200 and about 8,000, between about 5,600 and about 8,000, between about 6,000 and about 8,000, between about 6,400 and about 8,000, between about 6,800 and about 8,000, between about 7,200 and about 8,000, or between about 7,600 and about 8,000 plants per acre. In another aspect, a population of tobacco plants is in a soil type with low to medium fertility.

Also provided are containers of seeds from tobacco plants described. A container of tobacco seeds of the present disclosure may contain any number, weight, or volume of seeds. For example, a container can contain at least, or greater than, about 100, 200, 300, 400, 500, 600, 700, 800, 900, 1000, 1500, 2000, 2500, 3000, 3500, 4000 or more seeds. Alternatively, the container can contain at least, or greater than, about 1 ounce, 5 ounces, 10 ounces, 1 pound, 2 pounds, 3 pounds, 4 pounds, 5 pounds or more seeds. Containers of tobacco seeds may be any container available in the art. By way of non-limiting example, a container may be a box, a bag, a packet, a pouch, a tape roll, a tube, or a bottle.

Also provided is cured tobacco material made from a low-alkaloid or low-nicotine tobacco plant described. Further provided is cured tobacco material made from a tobacco plant described with higher levels of total alkaloid or nicotine.

“Curing” is the aging process that reduces moisture and brings about the destruction of chlorophyll giving tobacco leaves a golden color and by which starch is converted to sugar. Cured tobacco therefore has a higher reducing sugar content and a lower starch content compared to harvested green leaf. In an aspect, green leaf tobacco provided can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation. Fermentation typically is characterized by high initial moisture content, heat generation, and a 10 to 20% loss of dry weight. See, e.g., U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cure, aged, and fermented tobacco can be further processed (e.g., cut, shredded, expanded, or blended). See, for example, U.S. Pat. Nos. 4,528,993; 4,660,577; and 4,987,907. In an aspect, the cured tobacco material of the present disclosure is sun-cured. In another aspect, the cured tobacco material of the present disclosure is flue-cured, air-cured, or fire-cured.

The presence of mold on cured tobacco can significantly reduce the quality and marketability (e.g., leaf grade) of the cured leaves. Mold growth is a common problem that can occur during extended periods of high humidity (e.g., greater than 70% relative humidity) at temperatures between approximately 10 °C (50 °F) and 32.2 °C (90 °F). Mold tends to be more prevalent at higher temperatures.

Tobacco plants, varieties, and lines provided herein comprising a mutant allele in one or more PMT genes, two or more PMT genes, three or more PMT genes, four or more PMT genes, or five PMT genes exhibit reduced mold infection as compared to the low alkaloid tobacco variety LA Burley 21 (LA BU 21). Similarly, tobacco plants, varieties, and lines provided herein comprising an RNAi construct that downregulates expression or translation of one or more PMT genes, two or more PMT genes, three or more PMT genes, four or more PMT genes, or five PMT genes exhibit reduced mold infection as compared to the low alkaloid tobacco variety LA Burley 21 (LA BU 21).

LA BU 21 is a low total alkaloid tobacco line produced by incorporation of a low alkaloid gene(s) from a Cuban cigar variety into Burley 21 through several backcrosses (Legg et al., Crop Science, 10:212 (1970)). It has approximately 0.2% total alkaloids (dry weight) compared to the about 3.5% (dry weight) of its parent, Burley 21. LA BU 21 has a leaf grade well below commercially acceptable standards.

In an aspect, a cured tobacco leaf comprising a mutant allele of pmt1a comprises no observable mold infection. In another aspect, a cured tobacco leaf comprising a mutant allele of pmt1b comprises no observable mold infection. In another aspect, a cured tobacco leaf comprising a mutant allele of pmt2 comprises no observable mold infection. In another aspect, a cured tobacco leaf comprising a mutant allele of pmt3 comprises no observable mold infection. In another aspect, a cured tobacco leaf comprising a mutant allele of pmt4 comprises no observable mold infection. In another aspect, a cured tobacco leaf comprising a mutant allele of pmt1a, a mutant allele of pmt1b, a mutant allele of pmt2, a mutant allele of pmt3, and a mutant allele of pmt4 comprises no observable mold infection.

In an aspect, a cured tobacco leaf comprising a mutant allele of pmt1a comprises a reduced mold infection as compared to a control cured tobacco leaf from the variety LA BU 21. In another aspect, a cured tobacco leaf comprising a mutant allele of pmt1b comprises a reduced mold infection as compared to a control cured tobacco leaf from the variety LA BU 21. In another aspect, a cured tobacco leaf comprising a mutant allele of pmt2 comprises a reduced mold infection as compared to a control cured tobacco leaf from the variety LA BU 21. In another aspect, a cured tobacco leaf comprising a mutant allele of pmt3 comprises a reduced mold infection as compared to a control cured tobacco leaf from the variety LA BU 21. In another aspect, a cured tobacco leaf comprising a mutant allele of pmt4 comprises a reduced mold infection as compared to a control cured tobacco leaf from the variety LA BU 21. In another aspect, a cured tobacco leaf comprising a mutant allele of pmt1a, a mutant allele of pmt1b, a mutant allele of pmt2, a mutant allele of pmt3, and a mutant allele of pmt4 comprises a reduced mold infection as compared to a control cured tobacco leaf from the variety LA BU 21.

In an aspect, a cured leaf from a tobacco plant, variety, or line provided in any one of Tables 4A to 4E, Table 10, or Table 14 comprises no observable mold infection. In another aspect, a cured leaf from a tobacco plant, variety, or line provided in any one of Tables 4A to 4E, Table 10, or Table 14 comprises a reduced mold infection as compared to a control cured tobacco leaf from the variety LA BU 21.

In an aspect, a cured leaf from a tobacco plant, variety, or line comprising one or more pmt mutations provided in any one of Tables 5A to 5E and Tables 12A to 12E comprises no observable mold infection. In another aspect, a cured leaf from a tobacco plant, variety, or line comprising one or more pmt mutations provided in any one of Tables 5A to 5E and Tables 12A to 12E comprises a reduced mold infection as compared to a control cured leaf from the variety LA BU 21.

In an aspect, a cured leaf from a tobacco plant, variety, or line comprising a mutant allele of pmt1a comprises a higher leaf grade than a control cured leaf from the variety LA BU 21. In an aspect, a cured leaf from a tobacco plant, variety, or line comprising a mutant allele of pmt1b comprises a higher leaf grade than a control cured leaf from the variety LA BU 21. In an aspect, a cured leaf from a tobacco plant, variety, or line comprising a mutant allele of pmt2 comprises a higher leaf grade than a control cured leaf from the variety LA BU 21. In an aspect, a cured leaf from a tobacco plant, variety, or line comprising a mutant allele of pmt3 comprises a higher leaf grade than a control cured leaf from the variety LA BU 21. In an aspect, a cured leaf from a tobacco plant, variety, or line comprising a mutant allele of pmt4 comprises a higher leaf grade than a control cured leaf from the variety LA BU 21. In another aspect, a cured tobacco leaf from a plant, variety, or line comprising a mutant allele of pmt1a, a mutant allele of pmt1b, a mutant allele of pmt2, a mutant allele of pmt3, and a mutant allele of pmt4 comprises a higher leaf grade than a control cured leaf from the variety LA BU 21.

In an aspect, a cured leaf from a tobacco plant, variety, or line provided in any one of Tables 4A to 4E, Table 10, or Table 14 comprises a higher leaf grade than a control cured leaf from the variety LA BU 21.

In an aspect, a cured leaf from a tobacco plant, variety, or line comprising one or more pmt mutations provided in any one of Tables 5A to 5E and Tables 12A to 12E comprises a higher leaf grade than a control cured leaf from the variety LA BU 21.

In an aspect, a “reduced mold infection” refers to a reduced area of infected leaf. In another aspect, a “reduced mold infection” refers to a reduced number of viable mold spores on an infected leaf. Standard methods of detecting and counting viable mold spores are known and available in the art.

In an aspect, a reduced mold infection comprises a reduction of infected leaf area of at least 1% as compared to a control leaf. In an aspect, a reduced mold infection comprises a reduction of infected leaf area of at least 2% as compared to a control leaf. In an aspect, a reduced mold infection comprises a reduction of infected leaf area of at least 3% as compared to a control leaf. In an aspect, a reduced mold infection comprises a reduction of infected leaf area of at least 4% as compared to a control leaf. In an aspect, a reduced mold infection comprises a reduction of infected leaf area of at least 5% as compared to a control leaf. In an aspect, a reduced mold infection comprises a reduction of infected leaf area of at least 10% as compared to a control leaf. In an aspect, a reduced mold infection comprises a reduction of infected leaf area of at least 15% as compared to a control leaf. In an aspect, a reduced mold infection comprises a reduction of infected leaf area of at least 20% as compared to a control leaf. In an aspect, a reduced mold infection comprises a reduction of infected leaf area of at least 25% as compared to a control leaf. In an aspect, a reduced mold infection comprises a reduction of infected leaf area of at least 30% as compared to a control leaf. In an aspect, a reduced mold infection comprises a reduction of infected leaf area of at least 35% as compared to a control leaf. In an aspect, a reduced mold infection comprises a reduction of infected leaf area of at least 40% as compared to a control leaf. In an aspect, a reduced mold infection comprises a reduction of infected leaf area of at least 50% as compared to a control leaf. In an aspect, a reduced mold infection comprises a reduction of infected leaf area of at least 60% as compared to a control leaf. In an aspect, a reduced mold infection comprises a reduction of infected leaf area of at least 70% as compared to a control leaf. In an aspect, a reduced mold infection comprises a reduction of infected leaf area of at least 75% as compared to a control leaf. In an aspect, a reduced mold infection comprises a reduction of infected leaf area of at least 80% as compared to a control leaf. In an aspect, a reduced mold infection comprises a reduction of infected leaf area of at least 90% as compared to a control leaf. In an aspect, a reduced mold infection comprises a reduction of infected leaf area of at least 95% as compared to a control leaf. In an aspect, a reduced mold infection comprises a reduction of infected leaf area of 100% as compared to a control leaf.

In an aspect, a reduced mold infection comprises a reduction of infected leaf area of between 1% and 100% as compared to a control leaf. In an aspect, a reduced mold infection comprises a reduction of infected leaf area of between 1% and 90% as compared to a control leaf. In an aspect, a reduced mold infection comprises a reduction of infected leaf area of between 1% and 80% as compared to a control leaf. In an aspect, a reduced mold infection comprises a reduction of infected leaf area of between 1% and 70% as compared to a control leaf. In an aspect, a reduced mold infection comprises a reduction of infected leaf area of between 1% and 60% as compared to a control leaf. In an aspect, a reduced mold infection comprises a reduction of infected leaf area of between 1% and 50% as compared to a control leaf. In an aspect, a reduced mold infection comprises a reduction of infected leaf area of between 1% and 40% as compared to a control leaf. In an aspect, a reduced mold infection comprises a reduction of infected leaf area of between 1% and 30% as compared to a control leaf. In an aspect, a reduced mold infection comprises a reduction of infected leaf area of between 1% and 20% as compared to a control leaf. In an aspect, a reduced mold infection comprises a reduction of infected leaf area of between 1% and 10% as compared to a control leaf. In an aspect, a reduced mold infection comprises a reduction of infected leaf area of between 10% and 100% as compared to a control leaf. In an aspect, a reduced mold infection comprises a reduction of infected leaf area of between 20% and 100% as compared to a control leaf. In an aspect, a reduced mold infection comprises a reduction of infected leaf area of between 30% and 100% as compared to a control leaf. In an aspect, a reduced mold infection comprises a reduction of infected leaf area of between 40% and 100% as compared to a control leaf. In an aspect, a reduced mold infection comprises a reduction of infected leaf area of between 50% and 100% as compared to a control leaf. In an aspect, a reduced mold infection comprises a reduction of infected leaf area of between 60% and 100% as compared to a control leaf. In an aspect, a reduced mold infection comprises a reduction of infected leaf area of between 70% and 100% as compared to a control leaf. In an aspect, a reduced mold infection comprises a reduction of infected leaf area of between 80% and 100% as compared to a control leaf. In an aspect, a reduced mold infection comprises a reduction of infected leaf area of between 90% and 100% as compared to a control leaf. In an aspect, a reduced mold infection comprises a reduction of infected leaf area of between 10% and 75% as compared to a control leaf. In an aspect, a reduced mold infection comprises a reduction of infected leaf area of between 25% and 75% as compared to a control leaf. In an aspect, a reduced mold infection comprises a reduction of infected leaf area of between 25% and 50% as compared to a control leaf.

In an aspect, mold infecting cured tobacco is of a genus selected from the group consisting of Cladosporium, Penicillium, Alternaria, Aspergillus, and Mucor.

Tobacco material obtained from the tobacco lines, varieties or hybrids of the present disclosure can be used to make tobacco products. As used herein, “tobacco product” is defined as any product made or derived from tobacco that is intended for human use or consumption.

Tobacco products provided include, without limitation, cigarette products (e.g., cigarettes and bidi cigarettes), cigar products (e.g., cigar wrapping tobacco and cigarillos), pipe tobacco products, products derived from tobacco, tobacco-derived nicotine products, smokeless tobacco products (e.g., moist snuff, dry snuff, and chewing tobacco), films, chewables, tabs, shaped parts, gels, consumable units, insoluble matrices, hollow shapes, reconstituted tobacco, expanded tobacco, and the like. See, e.g., U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokable material includes cured tobacco within a tobacco blend. In an aspect, a tobacco product of the present disclosure is selected from the group consisting of a cigarillo, a non-ventilated recess filter cigarette, a vented recess filter cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff. In yet another aspect, a tobacco product of the present disclosure is selected from the group consisting of an electronically heated cigarette, an e-cigarette, an electronic vaporing device.

In an aspect, a tobacco product of the present disclosure can be a blended tobacco product. In another aspect, a tobacco product of the present disclosure can be a low nicotine tobacco product. In a further aspect, a tobacco product of the present disclosure may comprise nornicotine at a level of less than about 3 mg/g. For example, the nornicotine content in such a product can be 3.0 mg/g, 2.5 mg/g, 2.0 mg/g, 1.5 mg/g, 1.0 mg/g, 750 pg/g, 500 pg/g, 250 pg/g, 100 pg/g, 75 pg/g, 50 pg/g, 25 pg/g, 10 pg/g, 7.0 pg/g, 5.0 pg/g, 4.0 pg/g, 2.0 pg/g, 1.0 pg/g, 0.5 pg/g, 0.4 pg/g, 0.2 pg/g, 0.1 pg/g, 0.05 pg/g, 0.01 pg/g, or undetectable.

In an aspect, cured tobacco material or tobacco products provided comprise an average nicotine or total alkaloid level selected from the group consisting of about 0.01%, 0.02%, 0.05%, 0.75%, 0.1%, 0.15%, 0.2%, 0.3%, 0.35%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 5%, 6%, 7%, 8%, and 9% on a dry weight basis. In another aspect, cured tobacco material or tobacco products provided comprise an average nicotine or total alkaloid level selected from the group consisting of about between 0.01% and 0.02%, between 0.02% and 0.05%, between 0.05% and 0.75%, between 0.75% and 0.1%, between 0.1% and 0.15%, between 0.15% and 0.2%, between 0.2% and 0.3%, between 0.3% and 0.35%, between 0.35% and 0.4%, between 0.4% and 0.5%, between 0.5% and 0.6%, between 0.6% and 0.7%, between 0.7% and 0.8%, between 0.8% and 0.9%, between 0.9% and 1%, between 1% and 1.1%, between 1.1% and 1.2%, between 1.2% and 1.3%, between 1.3% and 1.4%, between 1.4% and 1.5%, between 1.5% and 1.6%, between 1.6% and 1.7%, between 1.7% and 1.8%, between 1.8% and 1.9%, between 1.9% and 2%, between 2% and 2.1%, between 2.1% and 2.2%, between 2.2% and 2.3%, between 2.3% and 2.4%, between 2.4% and 2.5%, between 2.5% and 2.6%, between 2.6% and 2.7%, between 2.7% and 2.8%, between 2.8% and 2.9%, between 2.9% and 3%, between 3% and 3.1%, between 3.1% and 3.2%, between 3.2% and 3.3%, between 3.3% and 3.4%, between 3.4% and 3.5%, and between 3.5% and 3.6% on a dry weight basis. In a further aspect, cured tobacco material or tobacco products provided comprise an average nicotine or total alkaloid level selected from the group consisting of about between 0.01% and 0.1%, between 0.02% and 0.2%, between 0.03% and 0.3%, between 0.04% and 0.4%, between 0.05% and 0.5%, between 0.75% and 1%, between 0.1% and 1.5%, between 0.15% and 2%, between 0.2% and 3%, and between 0.3% and 3.5% on a dry weight basis.

The present disclosure also provides methods for breeding tobacco lines, cultivars, or varieties comprising a desirable level of total alkaloid or nicotine, e.g., low nicotine or nicotine free. Breeding can be carried out via any known procedures. DNA fingerprinting, SNP mapping, haplotype mapping or similar technologies may be used in a marker-assisted selection (MAS) breeding program to transfer or breed a desirable trait or allele into a tobacco plant. For example, a breeder can create segregating populations in a F₂ or backcross generation using F1 hybrid plants or further crossing the F1 hybrid plants with other donor plants with an agronomically desirable genotype. Plants in the F₂ or backcross generations can be screened for a desired agronomic trait or a desirable chemical profile using one of the techniques known in the art or listed herein. Depending on the expected inheritance pattern or the MAS technology used, self-pollination of selected plants before each cycle of backcrossing to aid identification of the desired individual plants can be performed. Backcrossing or other breeding procedure can be repeated until the desired phenotype of the recurrent parent is recovered. A recurrent parent in the present disclosure can be a flue-cured variety, a Burley variety, a dark air-cured variety, a dark fire-cured variety, or an Oriental variety. Other breeding techniques can be found, for example, in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y., incorporated herein by reference in their entirety.

Results of a plant breeding program using the tobacco plants described includes useful lines, cultivars, varieties, progeny, inbreds, and hybrids of the present disclosure. As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

In an aspect, this disclosure provides a tobacco plant, variety, line, or cell comprising one or more pmt mutations provided in any one of Tables 5A to 5E and Tables 12A to 12E.

In another aspect, this disclosure provides a tobacco plant, variety, line, or cell derived from any tobacco plant, variety, or line provided in any one of Tables 4A to 4E, Table 10, or Table 14.

In an aspect, this disclosure provides the tobacco line 18GH203 and F₁ or F₂ tobacco plants, or male sterile tobacco plants, derived therefrom. In an aspect, this disclosure provides the tobacco line 18GH341 and F₁ or F₂ tobacco plants, or male sterile tobacco plants, derived therefrom. In an aspect, this disclosure provides the tobacco line 17GH1678 and F₁ or F₂ tobacco plants, or male sterile tobacco plants, derived therefrom. In an aspect, this disclosure provides the tobacco line 17GH1680 and F₁ or F₂ tobacco plants, or male sterile tobacco plants, derived therefrom. In an aspect, this disclosure provides the tobacco line 17GH1804 and F₁ or F₂ tobacco plants, or male sterile tobacco plants, derived therefrom. In an aspect, this disclosure provides the tobacco line 17GH1898 and F₁ or F₂ tobacco plants, or male sterile tobacco plants, derived therefrom. In an aspect, this disclosure provides the tobacco line 18GH207 and F₁ or F₂ tobacco plants, or male sterile tobacco plants, derived therefrom. In an aspect, this disclosure provides the tobacco line 18GH342 and F₁ or F₂ tobacco plants, or male sterile tobacco plants, derived therefrom. In an aspect, this disclosure provides the tobacco line 18GH343 and F₁ or F₂ tobacco plants, or male sterile tobacco plants, derived therefrom. In an aspect, this disclosure provides the tobacco line 18GH348 and F₁ or F₂ tobacco plants, or male sterile tobacco plants, derived therefrom. In an aspect, this disclosure provides the tobacco line 18GH349 and F₁ or F₂ tobacco plants, or male sterile tobacco plants, derived therefrom. In an aspect, this disclosure provides the tobacco line 18GH355 and F₁ or F₂ tobacco plants, or male sterile tobacco plants, derived therefrom. In an aspect, this disclosure provides the tobacco line 18GH359 and F₁ or F₂ tobacco plants, or male sterile tobacco plants, derived therefrom. In an aspect, this disclosure provides the tobacco line 18GH64 and F₁ or F₂ tobacco plants, or male sterile tobacco plants, derived therefrom. In an aspect, this disclosure provides the tobacco line 18GH682 and F₁ or F₂ tobacco plants, or male sterile tobacco plants, derived therefrom. In an aspect, this disclosure provides the tobacco line 18GH692 and F₁ or F₂ tobacco plants, or male sterile tobacco plants, derived therefrom. In an aspect, this disclosure provides the tobacco line 18GH697 and F₁ or F₂ tobacco plants, or male sterile tobacco plants, derived therefrom. In an aspect, this disclosure provides the tobacco line 18GH922 and F₁ or F₂ tobacco plants, or male sterile tobacco plants, derived therefrom. In an aspect, this disclosure provides the tobacco line 18GH957 and F₁ or F₂ tobacco plants, or male sterile tobacco plants, derived therefrom. In an aspect, this disclosure provides the tobacco line 17GH1808 and F₁ or F₂ tobacco plants, or male sterile tobacco plants, derived therefrom. In an aspect, this disclosure provides the tobacco line 17GH1810 and F₁ or F₂ tobacco plants, or male sterile tobacco plants, derived therefrom. In an aspect, this disclosure provides the tobacco line 17GH1886 and F₁ or F₂ tobacco plants, or male sterile tobacco plants, derived therefrom. In an aspect, this disclosure provides the tobacco line 17GH1888 and F₁ or F₂ tobacco plants, or male sterile tobacco plants, derived therefrom. In an aspect, this disclosure provides the tobacco line 17GH1889 and F₁ or F₂ tobacco plants, or male sterile tobacco plants, derived therefrom. In an aspect, this disclosure provides the tobacco line 17GH189 and F₁ or F₂ tobacco plants, or male sterile tobacco plants, derived therefrom. In an aspect, this disclosure provides the tobacco line 17GH1893 and F₁ or F₂ tobacco plants, or male sterile tobacco plants, derived therefrom. In an aspect, this disclosure provides the tobacco line 17GH1901 and F₁ or F₂ tobacco plants, or male sterile tobacco plants, derived therefrom. In an aspect, this disclosure provides the tobacco line 17GH1902 and F₁ or F₂ tobacco plants, or male sterile tobacco plants, derived therefrom. In an aspect, this disclosure provides the tobacco line 18GH3 and F₁ or F₂ tobacco plants, or male sterile tobacco plants, derived therefrom. In an aspect, this disclosure provides the tobacco line 18GH125 and F₁ or F₂ tobacco plants, or male sterile tobacco plants, derived therefrom. In an aspect, this disclosure provides the tobacco line 18GH208 and F₁ or F₂ tobacco plants, or male sterile tobacco plants, derived therefrom. In an aspect, this disclosure provides the tobacco line 18GH403 and F₁ or F₂ tobacco plants, or male sterile tobacco plants, derived therefrom. In an aspect, this disclosure provides the tobacco line 18GH414 and F₁ or F₂ tobacco plants, or male sterile tobacco plants, derived therefrom. In an aspect, this disclosure provides the tobacco line 18GH434 and F₁ or F₂ tobacco plants, or male sterile tobacco plants, derived therefrom. In an aspect, this disclosure provides the tobacco line 18GH436 and F₁ or F₂ tobacco plants, or male sterile tobacco plants, derived therefrom. In an aspect, this disclosure provides the tobacco line 18GH437 and F₁ or F₂ tobacco plants, or male sterile tobacco plants, derived therefrom. In an aspect, this disclosure provides the tobacco line 18GH449 and F₁ or F₂ tobacco plants, or male sterile tobacco plants, derived therefrom. In an aspect, this disclosure provides the tobacco line 18GH706 and F₁ or F₂ tobacco plants, or male sterile tobacco plants, derived therefrom. In an aspect, this disclosure provides the tobacco line 18GH709 and F₁ or F₂ tobacco plants, or male sterile tobacco plants, derived therefrom. In an aspect, this disclosure provides the tobacco line 18GH710 and F₁ or F₂ tobacco plants, or male sterile tobacco plants, derived therefrom. In an aspect, this disclosure provides the tobacco line 18GH716 and F₁ or F₂ tobacco plants, or male sterile tobacco plants, derived therefrom. In an aspect, this disclosure provides the tobacco line 18GH729 and F₁ or F₂ tobacco plants, or male sterile tobacco plants, derived therefrom. In an aspect, this disclosure provides the tobacco line 18GH731 and F₁ or F₂ tobacco plants, or male sterile tobacco plants, derived therefrom. In an aspect, this disclosure provides the tobacco line 18GH752 and F₁ or F₂ tobacco plants, or male sterile tobacco plants, derived therefrom. In an aspect, this disclosure provides the tobacco line 18GH756 and F₁ or F₂ tobacco plants, or male sterile tobacco plants, derived therefrom. In an aspect, this disclosure provides the tobacco line 18GH768 and F₁ or F₂ tobacco plants, or male sterile tobacco plants, derived therefrom. In an aspect, this disclosure provides the tobacco line 18GH771 and F₁ or F₂ tobacco plants, or male sterile tobacco plants, derived therefrom. In an aspect, this disclosure provides the tobacco line 18GH776 and F₁ or F₂ tobacco plants, or male sterile tobacco plants, derived therefrom. In an aspect, this disclosure provides the tobacco line 18GH800 and F₁ or F₂ tobacco plants, or male sterile tobacco plants, derived therefrom. In an aspect, this disclosure provides the tobacco line 18GH818 and F₁ or F₂ tobacco plants, or male sterile tobacco plants, derived therefrom. In an aspect, this disclosure provides the tobacco line 18GH10 and F₁ or F₂ tobacco plants, or male sterile tobacco plants, derived therefrom. In an aspect, this disclosure provides the tobacco line 18GH1004 and F₁ or F₂ tobacco plants, or male sterile tobacco plants, derived therefrom. In an aspect, this disclosure provides the tobacco line 18GH1033 and F₁ or F₂ tobacco plants, or male sterile tobacco plants, derived therefrom. In an aspect, this disclosure provides the tobacco line 18GH132 and F₁ or F₂ tobacco plants, or male sterile tobacco plants, derived therefrom. In an aspect, this disclosure provides the tobacco line 18GH134 and F₁ or F₂ tobacco plants, or male sterile tobacco plants, derived therefrom. In an aspect, this disclosure provides the tobacco line 18GH217 and F₁ or F₂ tobacco plants, or male sterile tobacco plants, derived therefrom. In an aspect, this disclosure provides the tobacco line 18GH456 and F₁ or F₂ tobacco plants, or male sterile tobacco plants, derived therefrom. In an aspect, this disclosure provides the tobacco line 18GH457 and F₁ or F₂ tobacco plants, or male sterile tobacco plants, derived therefrom. In an aspect, this disclosure provides the tobacco line 18GH460 and F₁ or F₂ tobacco plants, or male sterile tobacco plants, derived therefrom. In an aspect, this disclosure provides the tobacco line 18GH465 and F₁ or F₂ tobacco plants, or male sterile tobacco plants, derived therefrom. In an aspect, this disclosure provides the tobacco line 18GH71 and F₁ or F₂ tobacco plants, or male sterile tobacco plants, derived therefrom. In an aspect, this disclosure provides the tobacco line 18GH830 and F₁ or F₂ tobacco plants, or male sterile tobacco plants, derived therefrom. In an aspect, this disclosure provides the tobacco line 18GH831 and F₁ or F₂ tobacco plants, or male sterile tobacco plants, derived therefrom. In an aspect, this disclosure provides the tobacco line 18GH836 and F₁ or F₂ tobacco plants, or male sterile tobacco plants, derived therefrom. In an aspect, this disclosure provides the tobacco line 18GH841 and F₁ or F₂ tobacco plants, or male sterile tobacco plants, derived therefrom. In an aspect, this disclosure provides the tobacco line 18GH974 and F₁ or F₂ tobacco plants, or male sterile tobacco plants, derived therefrom. In an aspect, this disclosure provides the tobacco line 18GH981 and F₁ or F₂ tobacco plants, or male sterile tobacco plants, derived therefrom. In an aspect, this disclosure provides the tobacco line 18GH994 and F₁ or F₂ tobacco plants, or male sterile tobacco plants, derived therefrom. In an aspect, this disclosure provides the tobacco line 17GH1905 and F₁ or F₂ tobacco plants, or male sterile tobacco plants, derived therefrom. In an aspect, this disclosure provides the tobacco line 18GH128 and F₁ or F₂ tobacco plants, or male sterile tobacco plants, derived therefrom. In an aspect, this disclosure provides the tobacco line 18GH130 and F₁ or F₂ tobacco plants, or male sterile tobacco plants, derived therefrom. In an aspect, this disclosure provides the tobacco line 18GH131 and F₁ or F₂ tobacco plants, or male sterile tobacco plants, derived therefrom. In an aspect, this disclosure provides the tobacco line 18GH133 and F₁ or F₂ tobacco plants, or male sterile tobacco plants, derived therefrom. In an aspect, this disclosure provides the tobacco line 18GH136 and F₁ or F₂ tobacco plants, or male sterile tobacco plants, derived therefrom. In an aspect, this disclosure provides the tobacco line 18GH216 and F₁ or F₂ tobacco plants, or male sterile tobacco plants, derived therefrom. In an aspect, this disclosure provides the tobacco line 18GH227 and F₁ or F₂ tobacco plants, or male sterile tobacco plants, derived therefrom. In an aspect, this disclosure provides the tobacco line 18GH5 and F₁ or F₂ tobacco plants, or male sterile tobacco plants, derived therefrom. In an aspect, this disclosure provides the tobacco line 18GH6 and F₁ or F₂ tobacco plants, or male sterile tobacco plants, derived therefrom. In an aspect, this disclosure provides the tobacco line 18GH65 and F₁ or F₂ tobacco plants, or male sterile tobacco plants, derived therefrom. In an aspect, this disclosure provides the tobacco line 18GH66 and F₁ or F₂ tobacco plants, or male sterile tobacco plants, derived therefrom. In an aspect, this disclosure provides the tobacco line 18GH69 and F₁ or F₂ tobacco plants, or male sterile tobacco plants, derived therefrom. In an aspect, this disclosure provides the tobacco line 18GH72 and F₁ or F₂ tobacco plants, or male sterile tobacco plants, derived therefrom. In an aspect, this disclosure provides the tobacco line 18GH73 and F₁ or F₂ tobacco plants, or male sterile tobacco plants, derived therefrom. In an aspect, this disclosure provides the tobacco line 18GH74 and F₁ or F₂ tobacco plants, or male sterile tobacco plants, derived therefrom. In an aspect, this disclosure provides the tobacco line 18GH78 and F₁ or F₂ tobacco plants, or male sterile tobacco plants, derived therefrom. In an aspect, this disclosure provides the tobacco line 18GH79 and F₁ or F₂ tobacco plants, or male sterile tobacco plants, derived therefrom. In an aspect, this disclosure provides the tobacco line 18GH8 and F₁ or F₂ tobacco plants, or male sterile tobacco plants, derived therefrom. In an aspect, this disclosure provides the tobacco line 18GH9 and F₁ or F₂ tobacco plants, or male sterile tobacco plants, derived therefrom. In an aspect, this disclosure provides the tobacco line 17GH1696 and F₁ or F₂ tobacco plants, or male sterile tobacco plants, derived therefrom. In an aspect, this disclosure provides the tobacco line 17GH1717 and F₁ or F₂ tobacco plants, or male sterile tobacco plants, derived therefrom. In an aspect, this disclosure provides the tobacco line 17GH1719 and F₁ or F₂ tobacco plants, or male sterile tobacco plants, derived therefrom. In an aspect, this disclosure provides the tobacco line 17GH1729 and F₁ or F₂ tobacco plants, or male sterile tobacco plants, derived therefrom. In an aspect, this disclosure provides the tobacco line 17GH1736 and F₁ or F₂ tobacco plants, or male sterile tobacco plants, derived therefrom. In an aspect, this disclosure provides the tobacco line 17GH1737 and F₁ or F₂ tobacco plants, or male sterile tobacco plants, derived therefrom. In an aspect, this disclosure provides the tobacco line 17GH1739 and F₁ or F₂ tobacco plants, or male sterile tobacco plants, derived therefrom. In an aspect, this disclosure provides the tobacco line 17GH1740 and F₁ or F₂ tobacco plants, or male sterile tobacco plants, derived therefrom. In an aspect, this disclosure provides the tobacco line 17GH1835 and F₁ or F₂ tobacco plants, or male sterile tobacco plants, derived therefrom. In an aspect, this disclosure provides the tobacco line 17GH1848 and F₁ or F₂ tobacco plants, or male sterile tobacco plants, derived therefrom. In an aspect, this disclosure provides the tobacco line 17GH1849 and F₁ or F₂ tobacco plants, or male sterile tobacco plants, derived therefrom. In an aspect, this disclosure provides the tobacco line 17GH1912 and F₁ or F₂ tobacco plants, or male sterile tobacco plants, derived therefrom. In an aspect, this disclosure provides the tobacco line 17GH1937 and F₁ or F₂ tobacco plants, or male sterile tobacco plants, derived therefrom. In an aspect, this disclosure provides the tobacco line 17GH1940 and F₁ or F₂ tobacco plants, or male sterile tobacco plants, derived therefrom. In an aspect, this disclosure provides the tobacco line 17GH1943 and F₁ or F₂ tobacco plants, or male sterile tobacco plants, derived therefrom. In an aspect, this disclosure provides the tobacco line 17GH1944 and F₁ or F₂ tobacco plants, or male sterile tobacco plants, derived therefrom. In an aspect, this disclosure provides the tobacco line 18GH1051 and F₁ or F₂ tobacco plants, or male sterile tobacco plants, derived therefrom. In an aspect, this disclosure provides the tobacco line 18GH22 and F₁ or F₂ tobacco plants, or male sterile tobacco plants, derived therefrom. In an aspect, this disclosure provides the tobacco line 18GH34 and F₁ or F₂ tobacco plants, or male sterile tobacco plants, derived therefrom. In an aspect, this disclosure provides the tobacco line 18GH473 and F₁ or F₂ tobacco plants, or male sterile tobacco plants, derived therefrom. In an aspect, this disclosure provides the tobacco line 18GH49 and F₁ or F₂ tobacco plants, or male sterile tobacco plants, derived therefrom. In an aspect, this disclosure provides the tobacco line 18GH50 and F₁ or F₂ tobacco plants, or male sterile tobacco plants, derived therefrom. In an aspect, this disclosure provides the tobacco line 18GH848 and F₁ or F₂ tobacco plants, or male sterile tobacco plants, derived therefrom. In an aspect, this disclosure provides the tobacco line 18GH850 and F₁ or F₂ tobacco plants, or male sterile tobacco plants, derived therefrom. In an aspect, this disclosure provides the tobacco line 18GH851 and F₁ or F₂ tobacco plants, or male sterile tobacco plants, derived therefrom. In an aspect, this disclosure provides the tobacco line 17GH1699 and F₁ or F₂ tobacco plants, or male sterile tobacco plants, derived therefrom. In an aspect, this disclosure provides the tobacco line 17GH1708 and F₁ or F₂ tobacco plants, or male sterile tobacco plants, derived therefrom. In an aspect, this disclosure provides the tobacco line 17GH1722 and F₁ or F₂ tobacco plants, or male sterile tobacco plants, derived therefrom. In an aspect, this disclosure provides the tobacco line 17GH1724 and F₁ or F₂ tobacco plants, or male sterile tobacco plants, derived therefrom. In an aspect, this disclosure provides the tobacco line 17GH1725 and F₁ or F₂ tobacco plants, or male sterile tobacco plants, derived therefrom. In an aspect, this disclosure provides the tobacco line 17GH1845 and F₁ or F₂ tobacco plants, or male sterile tobacco plants, derived therefrom. In an aspect, this disclosure provides the tobacco line 17GH1846 and F₁ or F₂ tobacco plants, or male sterile tobacco plants, derived therefrom. In an aspect, this disclosure provides the tobacco line 17GH1847 and F₁ or F₂ tobacco plants, or male sterile tobacco plants, derived therefrom. In an aspect, this disclosure provides the tobacco line 17GH1911 and F₁ or F₂ tobacco plants, or male sterile tobacco plants, derived therefrom. In an aspect, this disclosure provides the tobacco line 17GH1912 and F₁ or F₂ tobacco plants, or male sterile tobacco plants, derived therefrom. In an aspect, this disclosure provides the tobacco line 17GH1915 and F₁ or F₂ tobacco plants, or male sterile tobacco plants, derived therefrom. In an aspect, this disclosure provides the tobacco line 17GH1918 and F₁ or F₂ tobacco plants, or male sterile tobacco plants, derived therefrom. In an aspect, this disclosure provides the tobacco line 17GH1928 and F₁ or F₂ tobacco plants, or male sterile tobacco plants, derived therefrom. In an aspect, this disclosure provides the tobacco line 17GH1932 and F₁ or F₂ tobacco plants, or male sterile tobacco plants, derived therefrom. In an aspect, this disclosure provides the tobacco line 17GH1933 and F₁ or F₂ tobacco plants, or male sterile tobacco plants, derived therefrom. In an aspect, this disclosure provides the tobacco line 17GH1936 and F₁ or F₂ tobacco plants, or male sterile tobacco plants, derived therefrom. In an aspect, this disclosure provides the tobacco line 18GH20 and F₁ or F₂ tobacco plants, or male sterile tobacco plants, derived therefrom. In an aspect, this disclosure provides the tobacco line 18GH28 and F₁ or F₂ tobacco plants, or male sterile tobacco plants, derived therefrom. In an aspect, this disclosure provides the tobacco line 18GH31 and F₁ or F₂ tobacco plants, or male sterile tobacco plants, derived therefrom. In an aspect, this disclosure provides the tobacco line 18GH47 and F₁ or F₂ tobacco plants, or male sterile tobacco plants, derived therefrom. In an aspect, this disclosure provides the tobacco line 18GH51 and F₁ or F₂ tobacco plants, or male sterile tobacco plants, derived therefrom. In an aspect, this disclosure provides the tobacco line 18GH52 and F₁ or F₂ tobacco plants, or male sterile tobacco plants, derived therefrom. In an aspect, this disclosure provides the tobacco line CS107 and F₁ or F₂ tobacco plants, or male sterile tobacco plants, derived therefrom. In an aspect, this disclosure provides the tobacco line CS106 and F₁ or F₂ tobacco plants, or male sterile tobacco plants, derived therefrom. In an aspect, this disclosure provides the tobacco line CS115 and F₁ or F₂ tobacco plants, or male sterile tobacco plants, derived therefrom. In an aspect, this disclosure provides the tobacco line 17GH1809-13 and F₁ or F₂ tobacco plants, or male sterile tobacco plants, derived therefrom. In an aspect, this disclosure provides the tobacco line CS111 and F₁ or F₂ tobacco plants, or male sterile tobacco plants, derived therefrom. In an aspect, this disclosure provides the tobacco line CS112 and F₁ or F₂ tobacco plants, or male sterile tobacco plants, derived therefrom. In an aspect, this disclosure provides the tobacco line 17GH1678-60 and F₁ or F₂ tobacco plants, or male sterile tobacco plants, derived therefrom. In an aspect, this disclosure provides the tobacco line CS131 and F₁ or F₂ tobacco plants, or male sterile tobacco plants, derived therefrom. In an aspect, this disclosure provides the tobacco line 18GH709-01 and F₁ or F₂ tobacco plants, or male sterile tobacco plants, derived therefrom. In an aspect, this disclosure provides the tobacco line 18GH709-08 and F₁ or F₂ tobacco plants, or male sterile tobacco plants, derived therefrom. In an aspect, this disclosure provides the tobacco line 18GH414-11 and F₁ or F₂ tobacco plants, or male sterile tobacco plants, derived therefrom. In an aspect, this disclosure provides the tobacco line 18GH414-19 and F₁ or F₂ tobacco plants, or male sterile tobacco plants, derived therefrom. In an aspect, this disclosure provides the tobacco line 18GH437-04 and F₁ or F₂ tobacco plants, or male sterile tobacco plants, derived therefrom. In an aspect, this disclosure provides the tobacco line 18GH437-08 and F₁ or F₂ tobacco plants, or male sterile tobacco plants, derived therefrom. In an aspect, this disclosure provides the tobacco line 18GH437-32 and F₁ or F₂ tobacco plants, or male sterile tobacco plants, derived therefrom. In an aspect, this disclosure provides the tobacco line 18GH437-39 and F₁ or F₂ tobacco plants, or male sterile tobacco plants, derived therefrom. In an aspect, this disclosure provides the tobacco line 18GH449-26 and F₁ or F₂ tobacco plants, or male sterile tobacco plants, derived therefrom. In an aspect, this disclosure provides the tobacco line 18GH449-33 and F₁ or F₂ tobacco plants, or male sterile tobacco plants, derived therefrom. In an aspect, this disclosure provides the tobacco line 18GH125-48 and F₁ or F₂ tobacco plants, or male sterile tobacco plants, derived therefrom. In an aspect, this disclosure provides the tobacco line CS102 and F₁ or F₂ tobacco plants, or male sterile tobacco plants, derived therefrom. In an aspect, this disclosure provides the tobacco line CS103 and F₁ or F₂ tobacco plants, or male sterile tobacco plants, derived therefrom. In an aspect, this disclosure provides the tobacco line 17GH1719-30 and F₁ or F₂ tobacco plants, or male sterile tobacco plants, derived therefrom. In an aspect, this disclosure provides the tobacco line 17GH1740-36 and F₁ or F₂ tobacco plants, or male sterile tobacco plants, derived therefrom. In an aspect, this disclosure provides the tobacco line 17GH1698-22 and F₁ or F₂ tobacco plants, or male sterile tobacco plants, derived therefrom. In an aspect, this disclosure provides the tobacco line 17GH1700-13 and F₁ or F₂ tobacco plants, or male sterile tobacco plants, derived therefrom. In an aspect, this disclosure provides the tobacco line 17GH1702-17 and F₁ or F₂ tobacco plants, or male sterile tobacco plants, derived therefrom. In an aspect, this disclosure provides the tobacco line 17GH1849-01 and F₁ or F₂ tobacco plants, or male sterile tobacco plants, derived therefrom. In an aspect, this disclosure provides the tobacco line 17GH1849-48 and F₁ or F₂ tobacco plants, or male sterile tobacco plants, derived therefrom. In an aspect, this disclosure provides the tobacco line 17GH1737-24 and F₁ or F₂ tobacco plants, or male sterile tobacco plants, derived therefrom. In an aspect, this disclosure provides the tobacco line CS118 and F₁ or F₂ tobacco plants, or male sterile tobacco plants, derived therefrom. In an aspect, this disclosure provides the tobacco line CS133 and F₁ or F₂ tobacco plants, or male sterile tobacco plants, derived therefrom. In an aspect, this disclosure provides the tobacco line CS120 and F₁ or F₂ tobacco plants, or male sterile tobacco plants, derived therefrom. In an aspect, this disclosure provides the tobacco line 18GH1108-07 and F₁ or F₂ tobacco plants, or male sterile tobacco plants, derived therefrom. In an aspect, this disclosure provides the tobacco line 18GH2162 and F₁ or F₂ tobacco plants, or male sterile tobacco plants, derived therefrom. In an aspect, this disclosure provides the tobacco line CS164 and F₁ or F₂ tobacco plants, or male sterile tobacco plants, derived therefrom. In an aspect, this disclosure provides the tobacco line CS163 and F₁ or F₂ tobacco plants, or male sterile tobacco plants, derived therefrom. In an aspect, this disclosure provides the tobacco line CS146 and F₁ or F₂ tobacco plants, or male sterile tobacco plants, derived therefrom. In an aspect, this disclosure provides the tobacco line CS147 and F₁ or F₂ tobacco plants, or male sterile tobacco plants, derived therefrom. In an aspect, this disclosure provides the tobacco line CS150 and F₁ or F₂ tobacco plants, or male sterile tobacco plants, derived therefrom. In an aspect, this disclosure provides the tobacco line CS151 and F₁ or F₂ tobacco plants, or male sterile tobacco plants, derived therefrom. In an aspect, this disclosure provides the tobacco line CS148 and F₁ or F₂ tobacco plants, or male sterile tobacco plants, derived therefrom. In an aspect, this disclosure provides the tobacco line CS149 and F₁ or F₂ tobacco plants, or male sterile tobacco plants, derived therefrom. In an aspect, this disclosure provides the tobacco line CS152 and F₁ or F₂ tobacco plants, or male sterile tobacco plants, derived therefrom. In an aspect, this disclosure provides the tobacco line CS153 and F₁ or F₂ tobacco plants, or male sterile tobacco plants, derived therefrom. In an aspect, this disclosure provides the tobacco line CS143 and F₁ or F₂ tobacco plants, or male sterile tobacco plants, derived therefrom. In an aspect, this disclosure provides the tobacco line 18GH2169 and F₁ or F₂ tobacco plants, or male sterile tobacco plants, derived therefrom. In an aspect, this disclosure provides the tobacco line 18GH2171 and F₁ or F₂ tobacco plants, or male sterile tobacco plants, derived therefrom. In an aspect, this disclosure provides the tobacco line CS165 and F₁ or F₂ tobacco plants, or male sterile tobacco plants, derived therefrom. In an aspect, this disclosure provides the tobacco line CS118 and F₁ or F₂ tobacco plants, or male sterile tobacco plants, derived therefrom. In an aspect, this disclosure provides the tobacco line 18GH2254-7 and F₁ or F₂ tobacco plants, or male sterile tobacco plants, derived therefrom.

In an aspect, the present disclosure provides a method of introgressing a low nicotine trait into a tobacco variety, the method comprising: (a) crossing a first tobacco variety comprising a low nicotine trait with a second tobacco variety without the low nicotine trait to produce one or more progeny tobacco plants; (b) genotyping the one or more progeny tobacco plants for a pmt mutant allele selected from those listed in Tables 4A to 4E, Tables 5A to 5E, Table 10, and Tables 12A to 12E; and (c) selecting a progeny tobacco plant comprising the pmt mutant allele. In another aspect, these methods further comprise backcrossing the selected progeny tobacco plant with the second tobacco variety. In a further aspect, these methods further comprise: (d) crossing the selected progeny plant with itself or with the second tobacco variety to produce one or more further progeny tobacco plants; and (e) selecting a further progeny tobacco plant comprising a low nicotine trait. In an aspect, the step (e) of selecting comprises marker-assisted selection. In an aspect, these methods produce a single gene conversion comprising a low nicotine trait. In an aspect, these methods produce a single gene conversion comprising a pmt mutant allele. In an aspect, the second tobacco variety is an elite variety. In another aspect, the genotyping step of these methods involve one or more molecular marker assays. In another aspect, the genotyping may involve a polymorphic marker comprising a polymorphism selected from the group consisting of single nucleotide polymorphisms (SNPs), insertions or deletions in DNA sequence (Indels), simple sequence repeats of DNA sequence (SSRs), a restriction fragment length polymorphism (RFLP), and a tag SNP.

As used herein, “locus” is a chromosomal locus or region where a polymorphic nucleic acid, trait determinant, gene, or marker is located. A “locus” can be shared by two homologous chromosomes to refer to their corresponding locus or region. As used herein, “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant allele for a gene may have a reduced or eliminated activity or expression level for the gene relative to the wild-type allele. For diploid organisms such as tobacco, a first allele can occur on one chromosome, and a second allele can occur at the same locus on a second homologous chromosome. If one allele at a locus on one chromosome of a plant is a mutant allele and the other corresponding allele on the homologous chromosome of the plant is wild-type, then the plant is described as being heterozygous for the mutant allele. However, if both alleles at a locus are mutant alleles, then the plant is described as being homozygous for the mutant alleles. A plant homozygous for mutant alleles at a locus may comprise the same mutant allele or different mutant alleles if heteroallelic or biallelic.

As used herein, “introgression” or “introgress” refers to the transmission of a desired allele of a genetic locus from one genetic background to another.

As used herein, “crossed” or “cross” means to produce progeny via fertilization (e.g. cells, seeds or plants) and includes crosses between plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F1 generation. The term “BC1” refers to the second use of the recurrent parent, “BC2” refers to the third use of the recurrent parent, and so on. In an aspect, a backcross is performed repeatedly, with a progeny individual of each successive backcross generation being itself backcrossed to the same parental genotype.

As used herein, “single gene converted” or “single gene conversion” refers to plants that are developed using a plant breeding technique known as backcrossing, or via genetic engineering, where essentially all of the desired morphological and physiological characteristics of a variety are recovered in addition to the single gene transferred into the variety via the backcrossing technique or via genetic engineering.

As used herein, “elite variety” means any variety that has resulted from breeding and selection for superior agronomic performance.

As used herein, “selecting” or “selection” in the context of marker-assisted selection or breeding refer to the act of picking or choosing desired individuals, normally from a population, based on certain pre-determined criteria.

As used herein, the term “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes.

As used herein, “marker assay” means a method for detecting a polymorphism at a particular locus using a particular method, e.g., measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, etc.

As used herein, “marker assisted selection” (MAS) is a process by which phenotypes are selected based on marker genotypes. “Marker assisted selection breeding” refers to the process of selecting a desired trait or traits in a plant or plants by detecting one or more nucleic acids from the plant, where the nucleic acid is linked to the desired trait, and then selecting the plant or germplasm possessing those one or more nucleic acids.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism (RFLP), and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable. “SNP markers” exist when SNPs are mapped to sites on the genome.

As used herein, “marker” or “molecular marker” or “marker locus” is a term used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed.

It is understood that any tobacco plant of the present disclosure can further comprise additional agronomically desirable traits, for example, by transformation with a genetic construct or transgene using a technique known in the art. Without limitation, an example of a desired trait is herbicide resistance, pest resistance, disease resistance; high yield; high grade index value; curability; curing quality; mechanical harvestability; holding ability; leaf quality; height, plant maturation (e.g., early maturing, early to medium maturing, medium maturing, medium to late maturing, or late maturing); stalk size (e.g., a small, medium, or a large stalk); or leaf number per plant (e.g., a small (e.g., 5-10 leaves), medium (e.g., 11-15 leaves), or large (e.g., 16-21) number of leaves), or any combination. In an aspect, low-nicotine or nicotine-free tobacco plants or seeds disclosed comprise one or more transgenes expressing one or more insecticidal proteins, such as, for example, a crystal protein of Bacillus thuringiensis or a vegetative insecticidal protein from Bacillus cereus, such as VIP3 (see, for example, Estruch et al. (1997) Nat. Biotechnol. 15:137). In another aspect, tobacco plants further comprise an introgressed trait conferring resistance to brown stem rot (U.S. Pat. No. 5,689,035) or resistance to cyst nematodes (U.S. Pat. No. 5,491,081).

The present disclosure also provides pmt mutant tobacco plants comprising an altered nicotine or total alkaloid level but having a yield comparable to the yield of corresponding initial tobacco plants without such a nicotine level alternation. In an aspect, a pmt mutant variety provides a yield selected from the group consisting of about between 1200 and 3500, between 1300 and 3400, between 1400 and 3300, between 1500 and 3200, between 1600 and 3100, between 1700 and 3000, between 1800 and 2900, between 1900 and 2800, between 2000 and 2700, between 2100 and 2600, between 2200 and 2500, and between 2300 and 2400 lbs/acre. In another aspect, a pmt mutant tobacco variety provides a yield selected from the group consisting of about between 1200 and 3500, between 1300 and 3500, between 1400 and 3500, between 1500 and 3500, between 1600 and 3500, between 1700 and 3500, between 1800 and 3500, between 1900 and 3500, between 2000 and 3500, between 2100 and 3500, between 2200 and 3500, between 2300 and 3500, between 2400 and 3500, between 2500 and 3500, between 2600 and 3500, between 2700 and 3500, between 2800 and 3500, between 2900 and 3500, between 3000 and 3500, and between 3100 and 3500 lbs/acre. In a further aspect, pmt mutant tobacco plants provide a yield between 65% and 130%, between 70% and 130%, between 75% and 130%, between 80% and 130%, between 85% and 130%, between 90% and 130%, between 95% and 130%, between 100% and 130%, between 105% and 130%, between 110% and 130%, between 115% and 130%, or between 120% and 130% of the yield of a control plant having essentially identical genetic background except for pmt mutation(s). In a further aspect, pmt mutant tobacco plants provide a yield between 70% and 125%, between 75% and 120%, between 80% and 115%, between 85% and 110%, or between 90% and 100% of the yield of a control plant having essentially identical genetic background except for pmt mutations.

In an aspect, a tobacco plant disclosed (e.g., a low-nicotine, nicotine-free, or low-alkaloid tobacco variety) comprises a modification conferring a desired trait (e.g., low-nicotine, nicotine-free, or low-alkaloid) without substantially impacting a trait selected from the group consisting of yield, ripening and senescence, susceptibility to insect herbivory, polyamine content after topping, chlorophyll level, mesophyll cell number per unit leaf area, and end-product quality after curing.

In an aspect, a tobacco plant disclosed comprises a modification conferring a desired trait (e.g., low-nicotine, nicotine-free, or low-alkaloid) and further comprises a trait substantially comparable to an unmodified control plant, where the trait is selected from the group consisting of yield, ripening and senescence, susceptibility to insect herbivory, polyamine content after topping, chlorophyll level, mesophyll cell number per unit leaf area, and end-product quality after curing.

In an aspect, a tobacco plant disclosed comprises a modification conferring a desired trait (e.g., low-nicotine, nicotine-free, or low-alkaloid) and further comprises a yield which is more than 80%, more than 85%, more than 90%, more than 95%, more than 100%, more than 105%, more than 110%, more than 115%, more than 120%, more than 125%, more than 130%, more than 135%, or more than 140% relative to the yield of an unmodified control plant. In an aspect, a tobacco plant disclosed comprises a modification conferring a desired trait (e.g., low-nicotine, nicotine-free, or low-alkaloid) and further comprises a yield which is between 70% and 140%, between 75% and 135%, between 80% and 130%, between 85% and 125%, between 90% and 120%, between 95% and 115%, or between 100% and 110% relative to the yield of an unmodified control plant. In an aspect, a tobacco plant disclosed comprises a modification conferring a desired trait (e.g., low-nicotine, nicotine-free, or low-alkaloid) and further comprises a yield which is between 70% and 80%, between 75% and 85%, between 80% and 90%, between 85% and 95%, between 90% and 100%, between 95% and 105%, between 105% and 115%, between 110% and 120%, between 115% to 125%, between 120% and 130%, between 125 and 135%, or between 130% and 140% relative to the yield of an unmodified control plant.

In an aspect, a low-nicotine or nicotine-free tobacco variety disclosed is adapted for machine harvesting. In another aspect, a low-nicotine or nicotine-free tobacco variety disclosed is harvested mechanically.

In an aspect, tobacco plants provided are hybrid plants. Hybrids can be produced by preventing self-pollination of female parent plants (e.g., seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by male sterility (MS), or transgenic male sterility where a transgene inhibits microsporogenesis and/or pollen formation, or self-incompatibility. Female parent plants containing MS are particularly useful. In aspects in which the female parent plants are MS, pollen may be harvested from male fertile plants and applied manually to the stigmas of MS female parent plants, and the resulting F1 seed is harvested.

Plants can be used to form single-cross tobacco F1 hybrids. Pollen from a male parent plant is manually transferred to an emasculated female parent plant or a female parent plant that is male sterile to form F1 seed. Alternatively, three-way crosses can be carried out where a single-cross F1 hybrid is used as a female parent and is crossed with a different male parent. As another alternative, double-cross hybrids can be created where the F1 progeny of two different single-crosses are themselves crossed. Self-incompatibility can be used to particular advantage to prevent self-pollination of female parents when forming a double-cross hybrid.

In an aspect, a low-nicotine or nicotine-free tobacco variety is male sterile. In another aspect, a low-nicotine or nicotine-free tobacco variety is cytoplasmic male sterile. Male sterile tobacco plants may be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fehr (ed.), MacMillan Publishing Go., Inc., New York, N.Y. 761 pp.

In an aspect, this disclosure provides a male sterile tobacco plant, variety, or line comprising one or more pmt mutations provided in any one of Tables 5A to 5E and Tables 12A to 12E.

In another aspect, this disclosure provides a male sterile tobacco plant, variety, or line derived from any tobacco plant, variety, or line provided in any one of Tables 4A to 4E, Table 10, or Table 14.

In an aspect, this disclosure provides the male sterile line dCS11. In another aspect, this disclosure provides the male sterile line dCS12. In another aspect, this disclosure provides the male sterile line dCS13. In another aspect, this disclosure provides the male sterile line dCS14. In another aspect, this disclosure provides the male sterile line dCS15. In another aspect, this disclosure provides the male sterile line dCS16. In another aspect, this disclosure provides the male sterile line dCS17. In another aspect, this disclosure provides the male sterile line dCS18. In another aspect, this disclosure provides the male sterile line dS697.

In a further aspect, tobacco parts provided include, but are not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In an aspect, tobacco part provided does not include seed. In an aspect, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Cells, tissues and organs can be from seed, fruit, leaf, cotyledon, hypocotyl, meristem, embryos, endosperm, root, shoot, stem, pod, flower, infloresence, stalk, pedicel, style, stigma, receptacle, petal, sepal, pollen, anther, filament, ovary, ovule, pericarp, phloem, vascular tissue. In another aspect, this disclosure provides a tobacco plant chloroplast. In a further aspect, this disclosure provides epidermal cells, stomata cell, leaf or root hairs, a storage root, or a tuber. In another aspect, this disclosure provides a tobacco protoplast.

Skilled artisans understand that tobacco plants naturally reproduce via seeds, not via asexual reproduction or vegetative propagation. In an aspect, this disclosure provides tobacco endosperm. In another aspect, this disclosure provides tobacco endosperm cells. In a further aspect, this disclosure provides a male or female sterile tobacco plant, which cannot reproduce without human intervention.

In an aspect, the present disclosure provides a nucleic acid molecule comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity to a sequence selected from the group consisting of SEQ ID NOs: 1 to 10, and fragments thereof. In an aspect, the present disclosure provides a polypeptide or protein comprising at least about 40%, 45%, 50%, 55%, 60%, 65%, 70%, 75%, 80%, 85%, 90%, 91%, 92%, 93%, 94%, 95%, 96%, 97%, 98%, 99%, or 100% identity to an amino acid sequence selected from the group consisting of SEQ ID NOs: 11 to 15.

As used herein, the term “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution.

The present disclosure further provides a method manufacturing a tobacco product comprising tobacco material from tobacco plants disclosed. In an aspect, methods comprise conditioning aged tobacco material made from tobacco plants to increase its moisture content from between about 12.5% and about 13.5% to about 21%, blending the conditioned tobacco material to produce a desirable blend. In an aspect, the method of manufacturing a tobacco product further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

Tobacco material provided can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, burley, flue cured, and cigar filler or wrapper, as well as the products from the whole leaf stemming operation. In an aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with the copolymer and optionally flavorants and other additives.

In an aspect, tobacco material provided can be processed to a desired size. In an aspect, tobacco fibers can be processed to have an average fiber size of less than 200 micrometers. In an aspect, tobacco fibers are between 75 and 125 micrometers. In another aspect, tobacco fibers are processed to have a size of 75 micrometers or less. In an aspect, tobacco fibers include long cut tobacco, which can be cut or shredded into widths of about 10 cuts/inch up to about 110 cuts/inch and lengths of about 0.1 inches up to about 1 inch. Double cut tobacco fibers can have a range of particle sizes such that about 70% of the double cut tobacco fibers falls between the mesh sizes of −20 mesh and 80 mesh.

Tobacco material provided can be processed to have a total oven volatiles content of about 10% by weight or greater; about 20% by weight or greater; about 40% by weight or greater; about 15% by weight to about 25% by weight; about 20% by weight to about 30% by weight; about 30% by weight to about 50% by weight; about 45% by weight to about 65% by weight; or about 50% by weight to about 60% by weight. Those of skill in the art will appreciate that “moist” tobacco typically refers to tobacco that has an oven volatiles content of between about 40% by weight and about 60% by weight (e.g., about 45% by weight to about 55% by weight, or about 50% by weight). As used herein, “oven volatiles” are determined by calculating the percentage of weight loss for a sample after drying the sample in a pre-warmed forced draft oven at 110 °C for 3.25 hours. The oral product can have a different overall oven volatiles content than the oven volatiles content of the tobacco fibers used to make the oral product. The processing steps described can reduce or increase the oven volatiles content.

Having now generally described the disclosure, the same will be more readily understood through reference to the following examples that are provided by way of illustration, and are not intended to be limiting of the present disclosure, unless specified.

### EXAMPLES

### Example 1: Expression Profiling of Five PMT Genes

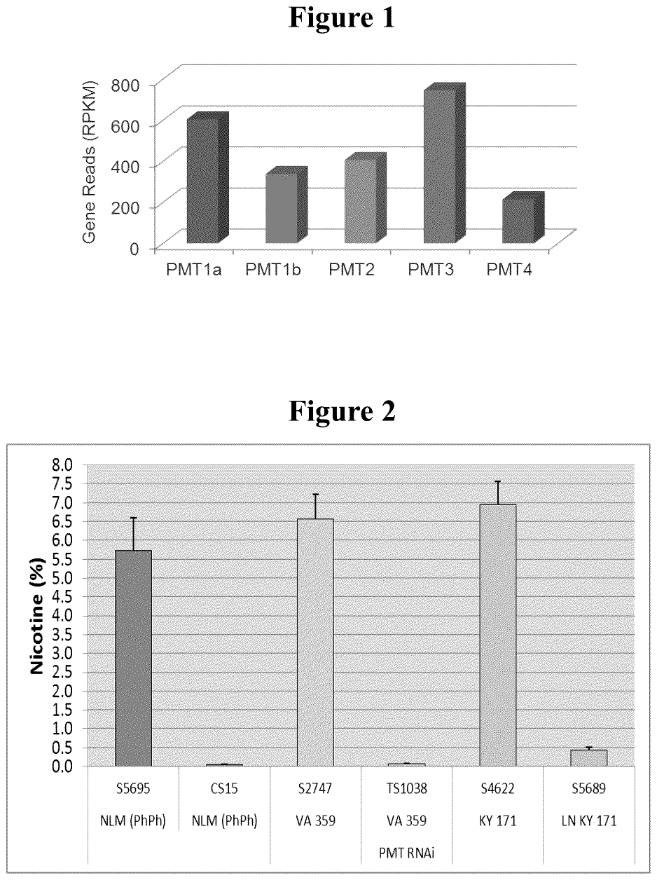

Nicotine biosynthesis starts with conversion of polyamine putrescine to N-methylputrescine by the enzyme putrescine N-methyl transferase (PMT). This is a step that commits precursor metabolites to nicotine biosynthesis. Genes encoding PMT (PMT1a, PMT1b, PMT2, PMT3 and PMT4) are present in the tobacco (Nicotiana tabacum) genome. Table 1A lists genomic DNA sequences, cDNA sequences, and protein sequences of five PMT genes. Tables 1B and 1C provide sequence identities among five PMT genes. Pooled expression levels from before topping to harvest provide support that, without being limited by any particular theory, PMT1a and PMT3 represent two major PMT genes (FIG. 1).

TABLE 1A
Sequences of five tobacco PMT genes.

| | Genomic DNA Sequence | | |
|---|---|---|---|
| | (including regions such | | |
| | as promoter, 5′ UTR, | cDNA | Protein |
| | introns, 3′ UTR, and | Sequence | Sequence |
| | terminator) | (SEQ ID | (SEQ ID |
| Gene Name | (SEQ ID No.) | No.) | No.) |
| PMT1b | 1 | 6 | 11 |
| PMT1a | 2 | 7 | 12 |
| PMT2 | 3 | 8 | 13 |
| PMT3 | 4 | 9 | 14 |
| PMT4 | 5 | 10 | 15 |

TABLE 1B
cDNA sequence identity among five tobacco PMT genes
determined by Clustal2.1.

| cDNA % | | | | | |
|---|---|---|---|---|---|
| identity | PMT1a | PMT1b | PMT2 | PMT3 | PMT4 |
| PMT1a | 100 | | | | |
| PMT1b | 98.85 | 100 | | | |
| PMT2 | 91.81 | 91.71 | 100 | | |
| PMT3 | 93.71 | 93.53 | 91.79 | 100 | |
| PMT4 | 94.24 | 94.06 | 92.75 | 94.59 | 100 |

TABLE 1C
Protein sequence identity among five tobacco PMT
genes determined by Clustal2.1.

| Protein % | | | | | |
|---|---|---|---|---|---|
| identity | PMT1a | PMT1b | PMT2 | PMT3 | PMT4 |
| PMT1a | 100 | | | | |
| PMT1b | 98.4 | 100 | | | |
| PMT2 | 95.42 | 95.75 | 100 | | |
| PMT3 | 97.48 | 97.76 | 96.23 | 100 | |
| PMT4 | 96.27 | 96.8 | 96.32 | 97.63 | 100 |

TABLE 1D
PMT1b genomic sequence (SEQ ID No. 1) annotation.

| Element | location |
|---|---|
| 5′ sequence | 1 . . . 1000 |
| exon 1 | 1001 . . . 1292 |
| intron 1 | 1293 . . . 1464 |
| exon 2 | 1465 . . . 1541 |
| intron 2 | 1542 . . . 1623 |
| exon 3 | 1624 . . . 1851 |
| intron 3 | 1852 . . . 1971 |
| exon 4 | 1972 . . . 2044 |
| intron 4 | 2045 . . . 2143 |
| exon 5 | 2144 . . . 2215 |
| intron 5 | 2216 . . . 2333 |
| exon 6 | 2334 . . . 2529 |
| intron 6 | 2530 . . . 3033 |
| exon 7 | 3034 . . . 3166 |
| intron 7 | 3167 . . . 3260 |
| exon 8 | 3261 . . . 3317 |
| 3′ sequence | 3318 . . . 4317 |

TABLE 1E
PMT1b genomic sequence (SEQ ID No. 2) annotation.

| Element | location |
|---|---|
| 5′ sequence | 1 . . . 1000 |
| exon 1 | 1001 . . . 1294 |
| intron 1 | 1295 . . . 1422 |
| exon 2 | 1423 . . . 1497 |
| intron 2 | 1498 . . . 1579 |
| exon 3 | 1580 . . . 1810 |
| intron 3 | 1811 . . . 1932 |
| exon 4 | 1933 . . . 2003 |
| intron 4 | 2004 . . . 2102 |
| exon 5 | 2103 . . . 2175 |
| intron 5 | 2176 . . . 2293 |
| exon 6 | 2294 . . . 2487 |
| intron 6 | 2488 . . . 2925 |
| exon 7 | 2926 . . . 3058 |
| intron 7 | 3059 . . . 3153 |
| exon 8 | 3154 . . . 3210 |
| 3′ sequence | 3211 . . . 4210 |

TABLE 1F
PMT2 genomic sequence (SEQ ID No. 3) annotation.

| Element | location |
|---|---|
| 5′ sequence | 1 . . . 792 |
| exon 1 | 793 . . . 1020 |
| intron 1 | 1021 . . . 1201 |
| exon 2 | 1202 . . . 1276 |
| intron 2 | 1277 . . . 1358 |
| exon 3 | 1359 . . . 1589 |
| intron 3 | 1590 . . . 1694 |
| exon 4 | 1695 . . . 1765 |
| intron 4 | 1766 . . . 1875 |
| exon 5 | 1876 . . . 1948 |
| intron 5 | 1949 . . . 2037 |
| exon 6 | 2038 . . . 2231 |
| intron 6 | 2232 . . . 2397 |
| exon 7 | 2398 . . . 2530 |
| intron 7 | 2531 . . . 2629 |
| exon 8 | 2630 . . . 2686 |
| 3′ sequence | 2687 . . . 3686 |

TABLE 1G
PMT3 genomic sequence (SEQ ID No. 4) annotation.

| Element | location |
|---|---|
| 5′ sequence | 1 . . . 1000 |
| exon 1 | 1001 . . . 1312 |
| intron 1 | 1313 . . . 1562 |
| exon 2 | 1563 . . . 1637 |
| intron 2 | 1638 . . . 1731 |
| exon 3 | 1732 . . . 1962 |
| intron 3 | 1963 . . . 2050 |
| exon 4 | 2051 . . . 2121 |
| intron 4 | 2122 . . . 2230 |
| exon 5 | 2231 . . . 2303 |
| intron 5 | 2304 . . . 2397 |
| exon 6 | 2398 . . . 2591 |
| intron 6 | 2592 . . . 2750 |
| exon 7 | 2751 . . . 2883 |
| intron 7 | 2884 . . . 2978 |
| exon 8 | 2979 . . . 3035 |
| 3′ sequence | 3036 . . . 4035 |

TABLE 1H
PMT4 genomic sequence (SEQ ID No. 5) annotation.

| Element | location |
|---|---|
| 5′ sequence | 1 . . . 1000 |
| exon 1 | 1001 . . . 1426 |
| intron 1 | 1427 . . . 1609 |
| exon 2 | 1610 . . . 1684 |
| intron 2 | 1685 . . . 1766 |
| exon 3 | 1767 . . . 1997 |
| intron 3 | 1998 . . . 2112 |
| exon 4 | 2113 . . . 2183 |
| intron 4 | 2184 . . . 2290 |
| exon 5 | 2291 . . . 2363 |
| intron 5 | 2364 . . . 2452 |
| exon 6 | 2453 . . . 2646 |
| intron 6 | 2647 . . . 3146 |
| exon 7 | 3147 . . . 3279 |
| intron 7 | 3280 . . . 3374 |
| exon 8 | 3375 . . . 3431 |
| 3′ sequence | 3432 . . . 4431 |

### Example 2: PMT Genome Editing and Tobacco Line Development

PMT knockout mutants are produced by editing various PMT genes. Tobacco protoplasts are transfected using polyethylene glycol (PEG) with plasmids encoding a genome editing technology 1 (GET 1) protein or a genome editing technology (GET) 2 protein and specific guide RNAs (gRNAs) targeting PMT genes at desired positions. Table 2 lists gRNA sequences used for PMT editing. Some gRNAs (e.g., Nos. 6 and 7) are pooled together for targeting multiple PMT genes in a single transfection.

Transfected protoplasts are then immobilized in 1% agarose bead and subjected to tissue culture. When calli grow up to ~1 mm in diameter, they are spread on TOM2 plates. Calli are screened for insertions or deletions (indels) at the target positions using fragment analysis. Candidates, showing size shifts compared to wildtype control, are selected for further culture and the consequent shoots are tested by fragment analysis again to confirm the presence of indels. Rooted shoots are potted and sequenced for the target positions to determine the exact sequences deleted. Young leaf from each plant is harvested and PCR amplified for PMT fragments using phirekit. PMT Libraries for each line is indexed and 384 lines are pooled and sequenced using Miseq.

SNP analysis is carried out to determine both the exact edited pmt mutant allele sequences and the zygosity state at each PMT gene locus. Table 3 provides the zygosity information of representative edited plants. Tables 4A to 4E provide indels sequence information in each edited line of various tobacco varieties (e.g., K326, TN90, NLM, oriental). Tables 5A to 5E provide genomic sequences of about 40 nucleotides from each pmt mutant allele with the edited site in the middle of the genomic sequence (e.g., 20 nucleotides on each side of the deleted or inserted sequence site).

TABLE 2
gRNA sequences used in 2 genome editing
technologies and their target genes.
“Y” represents that a gRNA targets that
PMT gene, while “—” represents that a gRNA
does not target that PMT gene.

| | Genome | | | | | | |
|---|---|---|---|---|---|---|---|
| | Editing | | | | | | |
| | Tech- | | | | | | |
| gRNA | nology | gRNA | Target genes | | | | |
| No. | (GET) | sequence | PMT1b | PMT1a | PMT2 | PMT3 | PMT4 |
| 1 | GET 1 | CCCATGAACG | Y | Y | — | — | — |
| | | GCCACCAAAA | | | | | |
| | | (SEQ ID | | | | | |
| | | NO: 16) | | | | | |
| 2 | GET 1 | GGCACTTCCA | Y | Y | Y | Y | Y |
| | | AACACCAAAA | | | | | |
| | | (SEQ ID | | | | | |
| | | NO: 17) | | | | | |
| 3 | GET 1 | GTTGTTCGGA | Y | Y | — | — | — |
| | | TGTCCCATTC | | | | | |
| | | (SEQ ID | | | | | |
| | | NO: 18) | | | | | |
| 4 | GET 1 | CTAAACTCTG | Y | — | Y | Y | — |
| | | AAAACCAACC | | | | | |
| | | (SEQ ID | | | | | |
| | | NO: 19) | | | | | |
| 5 | GET 1 | TTTCAGAGTT | Y | Y | Y | Y | Y |
| | | TAGCGCATTA | | | | | |
| | | (SEQ ID | | | | | |
| | | NO: 20) | | | | | |
| 6 | GET 2 | GATGGAGCAA | Y | Y | — | — | — |
| | | TTCAACATAC | | | | | |
| | | AGA | | | | | |
| | | (SEQ ID | | | | | |
| | | NO: 21) | | | | | |
| 7 | GET 2 | GATGGAGCAA | — | — | Y | Y | Y |
| | | TTCAACACAC | | | | | |
| | | AGA | | | | | |
| | | (SEQ ID | | | | | |
| | | NO: 22) | | | | | |

TABLE 3
Zygosity of individual PMT genic locus in selected pmt mutants
in various background produced by genome editing using
GET2. Number one (1) represents homozygous for a
single mutant allele. Numbers 2 to 5 represent a heteroallelic
combination having 2 to 5 Indels.
Hyphens indicate no data. Detailed genotype
information is shown in Tables 4A to 4D.

| Variety | Line | PMT1b | PMT1a | PMT2 | PMT3 | PMT4 |
|---|---|---|---|---|---|---|
| Basma | 18GH203 | 1 | 2 | 2 | 2 | 1 |
| Basma | 18GH341 | 1 | 2 | 2 | 2 | 1 |
| K326 | 17GH1678 | 2 | 2 | 1 | 1 | 2 |
| K326 | 17GH1680 | 1 | 2 | 1 | 1 | 1 |
| K326 | 17GH1804 | 1 | 2 | 1 | 1 | 1 |
| K326 | 17GH1898 | 1 | 2 | 1 | 1 | 1 |
| K326 | 18GH207 | 1 | 2 | 1 | 1 | 1 |
| K326 | 18GH342 | 1 | 2 | 1 | 1 | 1 |
| K326 | 18GH343 | 1 | 2 | 1 | 1 | 1 |
| K326 | 18GH348 | 1 | 1 | 1 | 1 | 1 |
| K326 | 18GH349 | 1 | 1 | 1 | 1 | 1 |
| K326 | 18GH355 | 1 | 2 | 2 | 3 | 2 |
| K326 | 18GH359 | 1 | 2 | 1 | 1 | 1 |
| K326 | 18GH64 | 2 | 1 | 2 | 1 | 1 |
| K326 | 18GH682 | 1 | 2 | 2 | 2 | 2 |
| K326 | 18GH692 | 1 | 2 | 2 | 2 | 1 |
| K326 | 18GH697 | 1 | 1 | 1 | 1 | 1 |
| K326 | 18GH922 | 1 | 1 | 1 | 1 | 1 |
| K326 | 18GH957 | 1 | 1 | 2 | 1 | 1 |
| K326 | 17GH1808 | 1 | 2 | 1 | 2 | 1 |
| K326 | 17GH1810 | 1 | 1 | 1 | 2 | 2 |
| K326 | 17GH1886 | — | — | 2 | 1 | — |
| K326 | 17GH1888 | — | 1 | 1 | — | — |
| K326 | 17GH1889 | — | 1 | 1 | — | — |
| K326 | 17GH1892 | 3 | 1 | 2 | 2 | — |
| K326 | 17GH1893 | 1 | 1 | 1 | 1 | 1 |
| K326 | 17GH1901 | 1 | 1 | 1 | 2 | 2 |
| K326 | 17GH1902 | 1 | 1 | 1 | 2 | 2 |
| K326 | 18GH3 | — | 1 | 1 | 1 | — |
| Katerini | 18GH125 | 2 | 2 | 1 | 2 | 1 |
| Katerini | 18GH208 | 2 | 1 | 1 | 2 | 1 |
| Katerini | 18GH403 | 1 | 1 | 1 | 1 | — |
| Katerini | 18GH414 | 2 | 1 | 1 | 1 | 1 |
| Katerini | 18GH434 | 2 | 1 | 1 | 2 | 1 |
| Katerini | 18GH436 | 2 | 1 | 1 | 4 | 1 |
| Katerini | 18GH437 | 1 | 2 | 1 | 2 | 2 |
| Katerini | 18GH449 | 2 | 2 | 1 | 1 | 1 |
| Katerini | 18GH706 | 2 | 1 | 1 | 2 | 1 |
| Katerini | 18GH709 | 2 | 2 | 1 | 1 | 1 |
| Katerini | 18GH710 | 1 | 1 | 2 | 2 | 1 |
| Katerini | 18GH716 | 2 | 2 | 1 | 2 | 2 |
| Katerini | 18GH729 | 1 | 1 | 2 | 1 | 1 |
| Katerini | 18GH731 | 1 | 1 | 1 | 2 | 1 |
| Katerini | 18GH752 | 2 | 1 | 1 | 1 | 1 |
| Katerini | 18GH756 | 1 | 1 | 1 | 2 | 2 |
| Katerini | 18GH768 | 1 | 1 | 1 | 1 | 2 |
| Katerini | 18GH771 | 1 | 1 | 2 | 2 | 1 |
| Katerini | 18GH776 | 2 | 2 | 1 | 1 | 2 |
| Katerini | 18GH800 | 2 | 2 | 1 | 1 | 2 |
| Katerini | 18GH818 | 1 | 1 | 1 | 2 | — |
| NLMz | 18GH10 | 1 | 1 | 1 | 1 | 1 |
| NLMz | 18GH1004 | 2 | 1 | 1 | 2 | 2 |
| NLMz | 18GH1033 | 2 | 1 | 1 | 2 | 2 |
| NLMz | 18GH132 | 1 | 2 | 2 | 3 | 1 |
| NLMz | 18GH134 | 1 | 2 | 1 | 1 | 1 |
| NLMz | 18GH217 | 1 | 2 | 2 | 1 | 2 |
| NLMz | 18GH456 | 2 | 1 | 1 | 1 | 1 |
| NLMz | 18GH457 | 1 | 1 | 1 | 1 | 1 |
| NLMz | 18GH460 | 1 | 2 | 3 | 1 | 1 |
| NLMz | 18GH465 | 2 | 1 | 1 | 2 | 2 |
| NLMz | 18GH71 | 1 | 1 | 1 | 1 | 1 |
| NLMz | 18GH830 | 1 | 1 | 1 | 1 | 1 |
| NLMz | 18GH831 | 1 | 2 | 1 | — | 1 |
| NLMz | 18GH836 | 1 | 1 | 1 | 1 | 1 |
| NLMz | 18GH841 | 2 | 2 | 1 | — | 1 |
| NLMz | 18GH974 | 2 | 1 | 2 | 2 | 1 |
| NLMz | 18GH981 | 1 | 1 | 2 | 2 | 2 |
| NLMz | 18GH994 | 2 | 1 | 1 | 2 | 2 |
| NLMz | 17GH1905 | 1 | 2 | 1 | 2 | 2 |
| NLMz | 18GH128 | — | 2 | 2 | — | 1 |
| NLMz | 18GH130 | 2 | 2 | 1 | 1 | 1 |
| NLMz | 18GH131 | 1 | 3 | 2 | — | 1 |
| NLMz | 18GH133 | 2 | 3 | 2 | — | 1 |
| NLMz | 18GH136 | — | — | 1 | — | — |
| NLMz | 18GH216 | 2 | 2 | 1 | 1 | 2 |
| NLMz | 18GH227 | 1 | 1 | 1 | — | 1 |
| NLMz | 18GH5 | 1 | 2 | 1 | 2 | 2 |
| NLMz | 18GH6 | 1 | 2 | 3 | 1 | 1 |
| NLMz | 18GH65 | 2 | 2 | 2 | 2 | 1 |
| NLMz | 18GH66 | 1 | 2 | 1 | 2 | 2 |
| NLMz | 18GH69 | — | 1 | — | 2 | 1 |
| NLMz | 18GH72 | 2 | 2 | 2 | 2 | 1 |
| NLMz | 18GH73 | — | 2 | 2 | — | 1 |
| NLMz | 18GH74 | — | 1 | — | — | — |
| NLMz | 18GH78 | 1 | 1 | 1 | 3 | 2 |
| NLMz | 18GH79 | — | 2 | 2 | — | 1 |
| NLMz | 18GH8 | 1 | 2 | 2 | 1 | 2 |
| NLMz | 18GH9 | 1 | 2 | 1 | — | 1 |
| TN90 | 17GH1696 | 1 | 1 | 1 | 1 | 1 |
| TN90 | 17GH1717 | 1 | 2 | 2 | 2 | 1 |
| TN90 | 17GH1719 | 1 | 2 | 1 | 1 | 1 |
| TN90 | 17GH1729 | 2 | 1 | 1 | 1 | 1 |
| TN90 | 17GH1736 | 1 | 1 | 2 | 1 | 1 |
| TN90 | 17GH1737 | 1 | 2 | 2 | 2 | 1 |
| TN90 | 17GH1739 | 1 | 1 | 1 | 1 | 2 |
| TN90 | 17GH1740 | 1 | 2 | 1 | 2 | 1 |
| TN90 | 17GH1835 | 2 | 2 | 2 | 1 | 1 |
| TN90 | 17GH1848 | 1 | 2 | 1 | 1 | 1 |
| TN90 | 17GH1849 | 1 | 1 | 1 | 2 | 2 |
| TN90 | 17GH1912 | 1 | 2 | 1 | 1 | 1 |
| TN90 | 17GH1937 | 1 | 2 | 1 | 2 | 1 |
| TN90 | 17GH1940 | 1 | 2 | 1 | 1 | 1 |
| TN90 | 17GH1943 | 1 | 1 | 1 | 1 | 2 |
| TN90 | 17GH1944 | 1 | 1 | 1 | 1 | 2 |
| TN90 | 18GH1051 | 2 | 2 | 2 | 1 | 2 |
| TN90 | 18GH22 | 1 | 2 | 1 | 2 | 1 |
| TN90 | 18GH34 | 1 | 1 | 1 | 1 | 2 |
| TN90 | 18GH473 | 1 | 1 | 1 | 2 | 2 |
| TN90 | 18GH49 | 1 | 1 | 1 | 1 | 1 |
| TN90 | 18GH50 | 2 | 1 | 1 | 1 | 1 |
| TN90 | 18GH848 | 2 | 2 | 2 | 1 | 2 |
| TN90 | 18GH850 | 1 | 2 | 1 | 2 | 2 |
| TN90 | 18GH851 | 1 | 2 | 1 | 2 | 2 |
| TN90 | 17GH1699 | 3 | 2 | 2 | 2 | 1 |
| TN90 | 17GH1708 | 1 | 3 | 1 | 2 | — |
| TN90 | 17GH1722 | 2 | 1 | 2 | 2 | 1 |
| TN90 | 17GH1724 | 2 | 1 | 1 | 2 | 1 |
| TN90 | 17GH1725 | 2 | 1 | 1 | 2 | 1 |
| TN90 | 17GH1845 | 2 | 2 | 1 | 2 | 2 |
| TN90 | 17GH1846 | 2 | 1 | 2 | 2 | 1 |
| TN90 | 17GH1847 | 2 | 1 | 2 | 2 | 1 |
| TN90 | 17GH1911 | 1 | 2 | 1 | 1 | 1 |
| TN90 | 17GH1912 | 1 | 2 | 1 | 1 | 1 |
| TN90 | 17GH1915 | — | 1 | — | 1 | — |
| TN90 | 17GH1918 | 2 | 2 | 1 | 1 | 5 |
| TN90 | 17GH1928 | 2 | 2 | — | 2 | 1 |
| TN90 | 17GH1932 | 2 | 2 | — | — | 1 |
| TN90 | 17GH1933 | 2 | 2 | 2 | 5 | 1 |
| TN90 | 17GH1936 | 2 | 2 | 2 | 1 | 2 |
| TN90 | 18GH20 | — | 1 | 2 | 1 | 2 |
| TN90 | 18GH28 | 2 | 1 | 2 | 1 | 2 |
| TN90 | 18GH31 | 1 | 3 | 1 | 1 | 1 |
| TN90 | 18GH47 | 1 | 3 | 1 | 1 | 1 |
| TN90 | 18GH51 | — | — | — | 1 | — |
| TN90 | 18GH52 | — | — | — | 1 | — |

TABLE 4A
Mutant pmt alleles in K326 produced by genome editing using GET2. The position
of each edited site (e.g.,, indels) is relative to the nucleotide number on the
corresponding cDNA sequence of each PMT gene. For example, line 17GH1678 has bi-
allelic mutations in PMT1b. One of the two alleles has a four-nucleotide
deletion which corresponds to nucleotides 416 to 419 of the PMT1b cDNA sequence.
The other allele has a two-nucleotide deletion which corresponds to nucleotides
418 to 419 of the PMT1b cDNA sequence.
SEQ ID Numbers are assigned and shown for sequences of more than 10 nucleotides.

| | | PMT1b | | PMT1a | | PMT2 | | PMT3 | | PMT4 | |
|---|---|---|---|---|---|---|---|---|---|---|---|
| | | | Deleted | | Deleted | | Deleted | | Deleted | | Deleted |
| VARI- | | | se- | | se- | | se- | | se- | | se- |
| ETY | LINE | Position | quence | Position | quence | Position | quence | Position | quence | Position | quence |
| K326 | 17GH | 416 . . . | ATAC | 415 . . . | CATACAG | 348 . . . | AC | 432 . . . | ACAC | 547 . . . | CACAC |
| | 1678 | 419 | | 421 | | 349 | | 435 | | 551 | |
| | | 418 . . . | AC | 417 . . . | TACA | | | | | 548 . . . | ACAC |
| | | 419 | | 420 | | | | | | 551 | |
| K326 | 17GH | 414 . . . | ACAT | 414 . . . | ACAT | 348 . . . | AC | 432 . . . | ACAC | 546 . . . | AC |
| | 1680 | 417 | | 417 | | 349 | | 435 | | 547 | |
| | | | | 416 . . . | AT | | | | | | |
| | | | | 417 | | | | | | | |
| K326 | 17GH | 414 . . . | ACAT | 411 . . . | TCAACAT | 348 . . . | AC | 433 . . . | CACAC | 548 . . . | ACACA |
| | 1804 | 417 | | 420 | ACA | 349 | | 437 | | 552 | |
| | | | | | (379) | | | | | | |
| | | | | 417 . . . | TACA | | | | | | |
| | | | | 420 | | | | | | | |
| K326 | 17GH | 414 . . . | ACAT | 411 . . . | TCAACAT | 348 . . . | AC | 433 . . . | CACAC | 548 . . . | ACACA |
| | 1898 | 417 | | 420 | ACA | 349 | | 437 | | 552 | |
| | | | | | (379) | | | | | | |
| | | | | 417 . . . | TACA | | | | | | |
| | | | | 420 | | | | | | | |
| K326 | 18GH | 414 . . . | ACAT | 415 . . . | C | 348 . . . | ACAC | 432 . . . | AC | 546 . . . | ACAC |
| | 207 | 417 | | 415 | | 351 | | 433 | | 549 | |
| | | | | 417 . . . | T | | | | | | |
| | | | | 417 | | | | | | | |
| K326 | 18GH | 414 . . . | ACAT | 414 . . . | ACAT | 348 . . . | AC | 432 . . . | ACAC | 546 . . . | AC |
| | 343 | 417 | | 417 | | 349 | | 435 | | 547 | |
| | | | | 416 . . . | AT | | | | | | |
| | | | | 417 | | | | | | | |
| K326 | 18GH | 414 . . . | ACAT | 414 . . . | ACAT | 348 . . . | AC | 429 . . . | TCAACAC | 546 . . . | AC |
| | 348 | 417 | | 417 | | 349 | | 439 | ACAG | 547 | |
| | | | | | | | | | (396) | | |
| K326 | 18GH | 414 . . . | ACAT | 414 . . . | ACAT | 348 . . . | AC | 429 . . . | TCAACAC | 546 . . . | AC |
| | 349 | 417 | | 417 | | 349 | | 439 | ACAG | 547 | |
| | | | | | | | | | (396) | | |
| K326 | 18GH | 414 . . . | ACAT | 414 . . . | ACAT | 348 . . . | ACAC | 431 . . . | AACACAC | 546 . . . | AC |
| | 355 | 417 | | 417 | | 351 | | 438 | A | 547 | |
| | | | | 416 . . . | AT | 350 . . . | AC | 435 . . . | CACA | 550 . . . | ACAG |
| | | | | 417 | | 351 | | 438 | | 553 | |
| | | | | | | | | 440 . . . | AGA | | |
| | | | | | | | | 442 | | | |
| K326 | 18GH | 414 . . . | ACAT | 411 . . . | TCAACAT | 348 . . . | AC | 433 . . . | CACAC | 548 . . . | ACACA |
| | 359 | 417 | | 420 | ACA | 349 | | 437 | | 552 | |
| | | | | | (379) | | | | | | |
| | | | | 417 . . . | TACA | | | | | | |
| | | | | 420 | | | | | | | |
| K326 | 18GH | 413 . . . | AACATACA | 414 . . . | ACAT | 349 . . . | CACA | 432 . . . | AC | 546 . . . | ACAC |
| | 64 | 420 | | 417 | | 352 | | 433 | | 549 | |
| | | 417 . . . | TACA | | | 354 . . . | AGAGAA | | | | |
| | | 420 | | | | 359 | | | | | |
| K326 | 18GH | 415 . . . | CATACAG | 413 . . . | AACATAC | 348 . . . | ACAC | 432 . . . | ACACACA | 543 . . . | TCAACA |
| | 682 | 421 | | 422 | AGA | 351 | | 439 | G | 554 | CACAGA |
| | | | | | (386) | | | | | | (404) |
| | | | | 417 . . . | TACA | 350 . . . | AC | 437 . . . | CA | 549 . . . | CACA |
| | | | | 420 | | 351 | | 438 | | 552 | |
| K326 | 18GH | 414 . . . | ACAT | 414 . . . | ACATACA | 348 . . . | ACAC | 432 . . . | ACACACA | 546 . . . | ACAC |
| | 692 | 417 | | 420 | | 351 | | 439 | G | 549 | |
| | | | | 416 . . . | ATACA | 350 . . . | AC | 437 . . . | CA | | |
| | | | | 420 | | 351 | | 438 | | | |
| K326 | 18GH | 414 . . . | ACAT | 414 . . . | ACAT | 348 . . . | ACAC | 430 . . . | CAACACA | 546 . . . | ACAC |
| | 697 | 417 | | 417 | | 351 | | 436 | | 549 | |
| K326 | 18GH | 414 . . . | ACAT | 414 . . . | ACAT | 348 . . . | ACAC | 430 . . . | CAACACA | 546 . . . | ACAC |
| | 922 | 417 | | 417 | | 351 | | 436 | | 549 | |
| K326 | 18GH | 414 . . . | ACAT | 414 . . . | ACAT | 349 . . . | CACACA | 431 . . . | AACACAC | 546 . . . | AC |
| | 957 | 417 | | 417 | | 355 | G | 438 | A | 547 | |
| | | | | | | 351 . . . | CACA | | | | |
| | | | | | | 354 | | | | | |
| K326 | 17GH | — | — | — | — | 346 . . . | CAACA | 432 . . . | AC | — | — |
| | 1886 | | | | | 350 | | 433 | | | |
| | | | | | | 349 . . . | CA | | | | |
| | | | | | | 350 | | | | | |
| K326 | 17GH | — | — | 418 . . . | AC | 348 . . . | AC | — | — | — | — |
| | 1888 | | | 419 | | 349 | | | | | |
| K326 | 17GH | — | — | 418 . . . | AC | 348 . . . | AC | — | — | — | — |
| | 1889 | | | 419 | | 349 | | | | | |
| K326 | 17GH | 413 . . . | AACATAC | 414 . . . | ACAT | 348 . . . | ACAC | 432 . . . | ACAC | — | — |
| | 1892 | 419 | | 417 | | 351 | | 435 | | | |
| | | 414 . . . | ACATAC | | | 350 . . . | AC | 434 . . . | AC | | |
| | | 419 | | | | 351 | | 435 | | | |
| | | 416 . . . | ATAC | | | | | | | | |
| | | 419 | | | | | | | | | |
| K326 | 17GH | 416 . . . | ATACAG | 416 . . . | ATACA | 348 . . . | AC | 430 . . . | CAACACA | 546 . . . | AC |
| | 1893 | 421 | | 420 | | 349 | | 436 | | 547 | |
| K326 | 17GH | 414 . . . | ACAT | 417 . . . | TACA | 348 . . . | | 432 . . . | ACAC | 548 . . . | ACACA |
| | 1901 | 417 | | 420 | | 355 | | 435 | | 552 | |
| | | | | | | | ACACAC | 434 . . . | AC | 550 . . . | ACA |
| | | | | | | | AG | 435 | | 552 | |
| K326 | 17GH | 414 . . . | ACAT | 417 . . . | TACA | 348 . . . | ACACAC | 432 . . . | ACAC | 548 . . . | ACACA |
| | 1902 | 417 | | 420 | | 355 | AG | 435 | | 552 | |
| | | | | | | | | 434 . . . | AC | 550 . . . | ACA |
| | | | | | | | | 435 | | 552 | |
| K326 | 17GH | 414 . . . | ACAT | 413 . . . | AACATAC | 352 . . . | ACA | 432 . . . | ACAC | 546 . . . | AC |
| | 1808 | 417 | | 421 | AG | 354 | | 435 | | 547 | |
| | | | | 418 . . . | ACAG | | | 434 . . . | AC | | |
| | | | | 421 | | | | 435 | | | |
| K326 | 17GH | 414 . . . | ACAT | 417 . . . | TACA | 348 . . . | ACACAC | 432 . . . | ACAC | 548 . . . | ACACA |
| | 1810 | 417 | | 420 | | 355 | AG | 435 | | 552 | |
| | | | | | | | | 434 . . . | AC | 550 . . . | ACA |
| | | | | | | | | 435 | | 552 | |
| K326 | 18GH | — | — | 414 . . . | ACAT | 348 . . . | AC | 432 . . . | ACAC | — | — |
| | 3 | | | 417 | | 349 | | 435 | | | |
| K326 | 18GH | — | — | 414 . . . | ACAT | 348 . . . | AC | 429 . . . | TCAACAC | 546 . . . | AC |
| | 4 | | | 417 | | 349 | | 439 | ACAG | 547 | |
| | | | | | | | | | (396) | | |

TABLE 4B
Mutant pmt alleles in TN90 produced by genome editing using GET2.

| | | PMT1b | | PMT1a | | PMT2 | | PMT3 | | PMT4 | |
|---|---|---|---|---|---|---|---|---|---|---|---|
| | | | Deleted | | Deleted | | Deleted | | Deleted | | Deleted |
| VARIETY | LINE | Position | sequence | Position | sequence | Position | sequence | Position | sequence | Position | sequence |
| TN90 | 17GH | 414 . . . 417 | ACAT | 414 . . . 417 | ACAT | 348 . . . 349 | AC | 436 . . . 439 | ACAG | 546 . . . 547 | AC |
| | 1696 | | | | | | | | | | |
| TN90 | 17GH | 414 . . . 417 | ACAT | 414 . . . 417 | ACAT | 346 . . . 352 | CAACACA | 432 . . . 435 | ACAC | 546 . . . 547 | AC |
| | 1717 | | | 415 . . . 416 | CA | 349 . . . 352 | CACA | 434 . . . 435 | AC | | |
| TN90 | 17GH | 414 . . . 417 | ACAT | 417 . . . 420 | TACA | 348 . . . 349 | AC | 432 . . . 433 | AC | 546 . . . 549 | ACAC |
| | 1719 | | | 417 . . . 423 | TACAGA | | | | | | |
| | | | | | G | | | | | | |
| TN90 | 17GH | 412 . . . 421 | CAACATA | 412 . . . 418 | CAACAT | 348 . . . 351 | ACAC | 432 . . . 433 | AC | 546 . . . 547 | AC |
| | 1729 | | CAG (380) | | A | | | | | | |
| | | 414 . . . 420 | ACATACA | | | | | | | | |
| TN90 | 17GH | 418 . . . 421 | ACAG | 414 . . . 417 | ACAT | 347 . . . 357 | AACACACA | 432 . . . 433 | AC | 546 . . . 547 | AC |
| | 1736 | | | | | | GAG (391) | | | | |
| | | | | | | 351 . . . 354 | CACA | | | | |
| TN90 | 17GH | 414 . . . 417 | ACAT | 414 . . . 417 | ACAT | 346 . . . 352 | CAACACA | 432 . . . 435 | ACAC | 546 . . . 547 | AC |
| | 1737 | | | 415 . . . 416 | CA | 349 . . . 352 | CACA | 434 . . . 435 | AC | | |
| TN90 | 17GH | 414 . . . 417 | ACAT | 414 . . . 417 | ACAT | 348 . . . 349 | AC | 432 . . . 433 | AC | 546 . . . 549 | ACAC |
| | 1739 | | | | | | | | | 548 . . . 549 | AC |
| TN90 | 17GH | 414 . . . 417 | ACAT | 416 . . . 419 | ATAC | 348 . . . 351 | ACAC | 435 . . . 438 | CACA | 546 . . . 547 | AC |
| | 1740 | | | 418 . . . 419 | AC | | | 436 . . . 439 | ACAG | | |
| TN90 | 17GH | 413 . . . 421 | AACATAC | 417 . . . 420 | TACA | 350 . . . 354 | ACACA | 432 . . . 435 | ACAC | 546 . . . 547 | AC |
| | 1835 | | AG | | | | | | | | |
| | | 417 . . . 420 | TACA | 418 . . . 421 | ACAG | 351 . . . 362 | CACAGAGA | | | | |
| | | | | | | | ATGG (394) | | | | |
| TN90 | 17GH | 414 . . . 417 | ACAT | 417 . . . 420 | TACA | 348 . . . 349 | AC | 432 . . . 433 | AC | 546 . . . 549 | ACAC |
| | 1848 | | | 417 . . . 423 | TACAGA | | | | | | |
| | | | | | G | | | | | | |
| TN90 | 17GH | 414 . . . 417 | ACAT | 414 . . . 417 | ACAT | 348 . . . 351 | ACAC | 430 . . . 436 | CAACACA | 546 . . . 549 | ACAC |
| | 1849 | | | | | | | 433 . . . 436 | CACA | 548 . . . 549 | AC |
| TN90 | 17GH | 414 . . . 417 | ACAT | 417 . . . 420 | TACA | 348 . . . 349 | AC | 432 . . . 433 | AC | 546 . . . 549 | ACAC |
| | 1912 | | | 417 . . . 423 | TACAGA | | | | | | |
| | | | | | G | | | | | | |
| TN90 | 17GH | 416 . . . 419 | ATAC | 412 . . . 421 | CAACAT | 348 . . . 351 | ACAC | 432 . . . 435 | ACAC | 546 . . . 547 | AC |
| | 1937 | | | | ACAG | | | | | | |
| | | | | | (380) | | | | | | |
| | | | | 417 . . . 420 | TACA | | | 434 . . . 435 | AC | | |
| TN90 | 17GH | 414 . . . 417 | ACAT | 417 . . . 420 | TACA | 348 . . . 349 | AC | 432 . . . 433 | AC | 546 . . . 549 | ACAC |
| | 1940 | | | 417 . . . 423 | TACAGA | | | | | | |
| | | | | | G | | | | | | |
| TN90 | 17GH | 414 . . . 417 | ACAT | 414 . . . 417 | ACAT | 348 . . . 349 | AC | 432 . . . 433 | AC | 546 . . . 549 | ACAC |
| | 1943 | | | | | | | | | 548 . . . 549 | AC |
| TN90 | 17GH | 414 . . . 417 | ACAT | 414 . . . 417 | ACAT | 348 . . . 349 | AC | 432 . . . 433 | AC | 546 . . . 549 | ACAC |
| | 1944 | | | | | | | | | 548 . . . 549 | AC |
| TN90 | 18GH | 414 . . . 418 | ACATA | 412 . . . 418 | CAACAT | 348 . . . 351 | ACAC | 432 . . . 433 | AC | 546 . . . 549 | ACAC |
| | 1051 | | | | A | | | | | | |
| | | 415 . . . 421 | CATACAG | 415 . . . 418 | CATA | 350 . . . 351 | AC | | | 548 . . . 549 | AC |
| TN90 | 18GH | 416 . . . 419 | ATAC | 412 . . . 421 | CAACAT | 348 . . . 351 | ACAC | 432 . . . 435 | ACAC | 546 . . . 547 | AC |
| | 22 | | | | ACAG | | | | | | |
| | | | | | (380) | | | | | | |
| | | | | 417 . . . 420 | TACA | | | 434 . . . 435 | AC | | |
| TN90 | 18GH | 414 . . . 417 | ACAT | 414 . . . 417 | ACAT | 348 . . . 349 | AC | 432 . . . 433 | AC | 546 . . . 549 | ACAC |
| | 34 | | | | | | | | | 548 . . . 549 | AC |
| TN90 | 18GH | 414 . . . 417 | ACAT | 414 . . . 417 | ACAT | 348 . . . 351 | ACAC | 430 . . . 436 | CAACACA | 546 . . . 549 | ACAC |
| | 473 | | | | | | | 433 . . . 436 | CACA | 548 . . . 549 | AC |
| TN90 | 18GH | 412 . . . 421 | CAACATA | 412 . . . 418 | CAACAT | 348 . . . 351 | ACAC | 432 . . . 433 | AC | 546 . . . 547 | AC |
| | 49 | | CAG (380) | | A | | | | | | |
| TN90 | 18GH | 412 . . . 421 | CAACATA | 412 . . . 418 | CAACAT | 348 . . . 351 | ACAC | 432 . . . 433 | AC | 546 . . . 547 | AC |
| | 50 | | CAG (380) | | A | | | | | | |
| | | 414 . . . 420 | ACATACA | | | | | | | | |
| TN90 | 18GH | 414 . . . 418 | ACATA | 412 . . . 418 | CAACAT | 348 . . . 351 | ACAC | 432 . . . 433 | AC | 546 . . . 549 | ACAC |
| | 848 | | | | A | | | | | | |
| | | 415 . . . 421 | CATACAG | 415 . . . 418 | CATA | 350 . . . 351 | AC | | | 548 . . . 549 | AC |
| TN90 | 18GH | 414 . . . 417 | ACAT | 416 . . . 422 | ATACAG | 348 . . . 349 | AC | 435 . . . 438 | CACA | 546 . . . 547 | AC |
| | 850 | | | | A | | | | | | |
| | | | | 417 . . . 420 | TACA | | | 436 . . . 439 | ACAG | 550 . . . 553 | ACAG |
| TN90 | 18GH | 414 . . . 417 | ACAT | 416 . . . 422 | ATACAG | 348 . . . 349 | AC | 435 . . . 438 | CACA | 546 . . . 547 | AC |
| | 851 | | | | A | | | | | | |
| | | | | 417 . . . 420 | TACA | | | 436 . . . 439 | ACAG | 550 . . . 553 | ACAG |
| TN90 | 17GH | 419 . . . 420 | CA | 413 . . . 420 | AACATA | 348 . . . 351 | ACAC | 429 . . . 438 | TCAACACAC | 546 . . . 547 | AC |
| | 1699 | | | | CA | | | | A (395) | | |
| | | 418 . . . 423 | ACAGAG | 419 . . . 420 | CA | 349 . . . 349 | C | 432 . . . 446 | ACACACAG | | |
| | | | | | | | | | (399) | | |
| | | 427 . . . 427 | G | | | | | | AGAATGG | | |
| TN90 | 17GH | 414 . . . 417 | ACAT | 414 . . . 415 | AC | 346 . . . 355 | CAACACAC | 432 . . . 437 | ACACAC | — | — |
| | 1708 | | | 418 . . . 424 | ACAGAG | | AG (390) | 440 . . . 443 | AGAA | | |
| | | | | | A | | | | | | |
| | | | | 419 . . . 420 | CA | | | | | | |
| TN90 | 17GH | 415 . . . 420 | CATACA | 414 . . . 417 | ACAT | 348 . . . 351 | ACAC | 432 . . . 433 | AC | 546 . . . 549 | ACAC |
| | 1722 | 418 . . . 421 | ACAG | | | 350 . . . 351 | AC | 435 . . . 439 | CACAG | | |
| TN90 | 17GH | 416 . . . 421 | ATACAG | 414 . . . 417 | ACAT | 348 . . . 351 | ACAC | 432 . . . 435 | ACAC | 550 . . . 553 | ACAG |
| | 1724 | 417 . . . 420 | TACA | | | | | 434 . . . 435 | AC | | |
| TN90 | 17GH | 416 . . . 421 | ATACAG | 414 . . . 417 | ACAT | 348 . . . 351 | ACAC | 432 . . . 435 | ACAC | 550 . . . 553 | ACAG |
| | 1725 | 417 . . . 420 | TACA | | | | | 434 . . . 435 | AC | | |
| TN90 | 17GH | 416 . . . 418 | ATA | 412 . . . 418 | CAACAT | 348 . . . 351 | ACAC | 433 . . . 437 | CACAC | 546 . . . 551 | ACACAC |
| | 1845 | | | | A | | | | | | |
| | | 418 . . . 419 | AC | 415 . . . 418 | CATA | | | 436 . . . 437 | AC | 550 . . . 551 | AC |
| TN90 | 17GH | 415 . . . 420 | CATACA | 414 . . . 417 | ACAT | 348 . . . 351 | ACAC | 432 . . . 433 | AC | 546 . . . 549 | ACAC |
| | 1846 | 418 . . . 421 | ACAG | | | 350 . . . 351 | AC | 435 . . . 439 | CACAG | | |
| TN90 | 17GH | 415 . . . 420 | CATACA | 414 . . . 417 | ACAT | 348 . . . 351 | ACAC | 432 . . . 433 | AC | 546 . . . 549 | ACAC |
| | 1847 | 418 . . . 421 | ACAG | | | 350 . . . 351 | AC | 435 . . . 439 | CACAG | | |
| TN90 | 17GH | 414 . . . 417 | ACAT | 417 . . . 420 | TACA | 348 . . . 349 | AC | 432 . . . 433 | AC | 546 . . . 549 | ACAC |
| | 1911 | | | 417 . . . 423 | TACAGA | | | | | | |
| | | | | | G | | | | | | |
| TN90 | 17GH | 414 . . . 417 | ACAT | 417 . . . 420 | TACA | 348 . . . 349 | AC | 432 . . . 433 | AC | 546 . . . 549 | ACAC |
| | 1912 | | | 417 . . . 423 | TACAGA | | | | | | |
| | | | | | G | | | | | | |
| TN90 | 17GH | — | — | 414 . . . 417 | ACAT | — | — | 432 . . . 435 | ACAC | — | — |
| | 1915 | | | | | | | | | | |
| TN90 | 17GH | 414 . . . 419 | ACATAC | 417 . . . 420 | TACA | 353 . . . 361 | CAGAGAAT | 432 . . . 435 | ACAC | 544 . . . 550 | CAACAC |
| | 1918 | | | | | | | | | | A |
| | | | | | | | G | | | 554 . . . 554 | A |
| | | 416 . . . 419 | ATAC | 418 . . . 421 | ACAG | | | | | 558 . . . 563 | TGGTGG |
| | | | | | | | | | | 565 . . . 566 | TT |
| | | | | | | | | | | 569 . . . 572 | CATA |
| TN90 | 17GH | 412 . . . 418 | CAACATA | 414 . . . 417 | ACAT | | | 432 . . . 448 | ACACACAG | 546 . . . 547 | AC |
| | 1928 | | | | | | | | AGAATGGT | | |
| | | | | | | | | | G (400) | | |
| | | 415 . . . 418 | CATA | 419 . . . 421 | CAG | | | 437 . . . 438 | CA | | |
| TN90 | 17GH | 414 . . . 419 | ACATAC | 416 . . . 419 | ATAC | — | — | — | — | 546 . . . 551 | ACACAC |
| | 1932 | 416 . . . 419 | ATAC | 418 . . . 419 | AC | | | | | | |
| TN90 | 17GH | 414 . . . 419 | ACATAC | 416 . . . 419 | ATAC | 350 . . . 355 | ACACAG | 413 . . . 414 | CT | 546 . . . 551 | ACACAC |
| | 1933 | | | | | | | 418 . . . 419 | GA | | |
| | | 416 . . . 419 | ATAC | 418 . . . 419 | AC | 351 . . . 354 | CACA | 426 . . . 427 | AA | | |
| | | | | | | | | 431 . . . 432 | AA | | |
| | | | | | | | | 432 . . . 435 | ACAC | | |
| TN90 | 17GH | 413 . . . 421 | AACATAC | 416 . . . 419 | ATAC | 348 . . . 351 | ACAC | 432 . . . 433 | AC | 544 . . . 550 | CAACAC |
| | 1936 | | AG | | | | | | | | A |
| | | 417 . . . 420 | TACA | 418 . . . 419 | AC | 350 . . . 351 | AC | | | 547 . . . 550 | CACA |
| TN90 | 18GH | | | 414 . . . 419 | ACATAC | 348 . . . 349 | AC | 432 . . . 433 | AC | 546 . . . 549 | ACAC |
| | 20 | | | | | 352 . . . 355 | ACAG | | | 548 . . . 549 | AC |
| TN90 | 18GH | 414 . . . 419 | ACATAC | 416 . . . 419 | ATAC | 348 . . . 349 | AC | 436 . . . 439 | ACAG | 546 . . . 549 | ACAC |
| | 47 | | | 418 . . . 419 | AC | | | | | | |
| | | | | 424 . . . 425 | AA | | | | | | |
| TN90 | 18GH | 414 . . . 419 | ACATAC | 414 . . . 417 | ACAT | 348 . . . 351 | ACAC | 433 . . . 437 | CACAC | 546 . . . 549 | ACAC |
| | 28 | 416 . . . 419 | ATAC | | | 350 . . . 351 | AC | | | 548 . . . 549 | AC |
| TN90 | 18GH | 414 . . . 419 | ACATAC | 416 . . . 419 | ATAC | 348 . . . 349 | AC | 436 . . . 439 | ACAG | 546 . . . 549 | ACAC |
| | 31 | | | 418 . . . 419 | AC | | | | | | |
| | | | | 424 . . . 425 | AA | | | | | | |

TABLE 4C
Mutant pmt alleles in NLMz produced by genome editing using GET2. NLMz refers to the Narrow Leaf Madole variety
containing triple loss-of-function mutations in three nicotine demethylase genes (CYP82E4, CYP82E5v2, and CYP82E10).

| | | PMT1b | | PMT1a | | PMT2 | | PMT3 | | PMT4 | |
|---|---|---|---|---|---|---|---|---|---|---|---|
| | | | Deleted | | Deleted | | Deleted | | Deleted | | Deleted |
| VARIETY | LINE | Position | sequence | Position | sequence | Position | sequence | Position | sequence | Position | sequence |
| NLMz | 18GH | 414 . . . 417 | ACAT | 414 . . . 417 | ACAT | 350 . . . 351 | AC | 431 . . . 441 | AACACACA | 546 . . . 549 | ACAC |
| | 10 | | | | | | | | GAG (391) | | |
| NLMz | 18GH | 414 . . . 417 | ACAT | 412 . . . 418 | CAACATA | 348 . . . 349 | AC | 430 . . . 436 | CAACACA | 546 . . . 553 | ACACACAG |
| | 1004 | 416 . . . 416 | A | | | | | 435 . . . 436 | CA | 551 . . . 552 | CA |
| NLMz | 18GH | 414 . . . 417 | ACAT | 412 . . . 418 | CAACATA | 348 . . . 349 | AC | 430 . . . 436 | CAACACA | 546 . . . 553 | ACACACAG |
| | 1033 | 416 . . . 416 | A | | | | | 435 . . . 436 | CA | 551 . . . 552 | CA |
| NLMz | 18GH | 417 . . . 418 | TA | 416 . . . 419 | ATAC | 348 . . . 352 | ACACA | 432 . . . 437 | ACACAC | 546 . . . 547 | AC |
| | 132 | | | 418 . . . 419 | AC | 348 . . . 353 | ACACAC | 434 . . . 437 | ACAC | | |
| | | | | | | | | 436 . . . 437 | AC | | |
| NLMz | 18GH | 414 . . . 417 | ACAT | 414 . . . 423 | ACATACAG | 348 . . . 351 | ACAC | 433 . . . 439 | CACACAG | 550 . . . 556 | ACAGAGA |
| | 134 | | | | AG (388) | | | | | | |
| | | | | 419 . . . 420 | CA | | | | | | |
| NLMz | 18GH | 414 . . . 417 | ACAT | 415 . . . 419 | CATAC | 346 . . . 352 | CAACACA | 432 . . . 435 | ACAC | 545 . . . 557 | AACACACA |
| | 217 | | | | | | | | | | GAGAA (407) |
| | | | | 417 . . . 418 | TA | 351 . . . 352 | CA | | | 551 . . . 552 | CA |
| NLMz | 18GH | 416 . . . 419 | ATAC | 414 . . . 417 | ACAT | 348 . . . 349 | AC | 432 . . . 433 | AC | 546 . . . 549 | ACAC |
| | 456 | 418 . . . 419 | AC | | | | | | | | |
| NLMz | 18GH | 414 . . . 417 | ACAT | 414 . . . 417 | ACAT | 348 . . . 349 | AC | 432 . . . 433 | AC | 546 . . . 549 | ACAC |
| | 457 | | | | | | | | | | |
| NLMz | 18GH | 414 . . . 417 | ACAT | 409 . . . 420 | ATTCAACA | 350 . . . 363 | ACACAGA | 436 . . . 439 | ACAG | 550 . . . 553 | ACAG |
| | 460 | | | | TACA (383) | | GAATGGT | | | | |
| | | | | | | | (393) | | | | |
| | | | | 416 . . . 429 | | 351 . . . 354 | CACA | | | | |
| | | | | | ATACAGAG | 353 . . . 354 | CA | | | | |
| | | | | | AATGGT | | | | | | |
| | | | | | (389) | | | | | | |
| NLMz | 18GH | 414 . . . 417 | ACAT | 412 . . . 418 | CAACATA | 348 . . . 349 | AC | 430 . . . 436 | CAACACA | 546 . . . 553 | ACACACAG |
| | 465 | 416 . . . 416 | A | | | | | 435 . . . 436 | CA | 551 . . . 552 | CA |
| NLMz | 18GH | 414 . . . 417 | ACAT | 414 . . . 417 | ACAT | 348 . . . 351 | ACAC | 432 . . . 435 | ACAC | 550 . . . 553 | ACAG |
| | 830 | | | | | | | | | | |
| NLMz | 18GH | 413 . . . 428 | AACATACAGA | 413 . . . 428 | AACATACA | 348 . . . 351 | ACAC | | | 546 . . . 547 | AC |
| | 831 | | GAATGG(381) | | GAGAATGG | | | | | | |
| | | | | | (381) | | | | | | |
| | | | | 417 . . . 420 | TACA | | | | | | |
| NLMz | 18GH | 414 . . . 417 | ACAT | 414 . . . 417 | ACAT | 348 . . . 349 | AC | 432 . . . 433 | AC | 546 . . . 549 | ACAC |
| | 836 | | | | | | | | | | |
| NLMz | 18GH | 415 . . . 421 | CATACAG | 413 . . . 420 | AACATACA | 347 . . . 357 | AACACAC | | | 546 . . . 547 | AC |
| | 841 | 419 . . . 420 | CA | 414 . . . 420 | ACATACA | | AGAG | | | | |
| | | | | | | | (391) | | | | |
| NLMz | 18GH | 411 . . . 420 | TCAACATACA | 411 . . . 420 | TCAACATA | 351 . . . 354 | CACA | 432 . . . 433 | AC | 546 . . . 549 | ACAC |
| | 974 | | (379) | | CA (379) | | | | | | |
| | | 417 . . . 420 | TACA | | | 353 . . . 354 | CA | 436 . . . 439 | ACAG | | |
| NLMz | 18GH | 412 . . . 418 | CAACATA | 414 . . . 417 | ACAT | 346 . . . 352 | CAACACA | 429 . . . 439 | TCAACACA | 546 . . . 552 | ACACACA |
| | 981 | | | | | | | | CAG (396) | | |
| | | | | | | 349 . . . 352 | CACA | 431 . . . 441 | AACACACA | 546 . . . 553 | ACACACAG |
| | | | | | | | | | GAG (391) | | |
| NLMz | 18GH | 414 . . . 417 | ACAT | 412 . . . 418 | CAACATA | 348 . . . 349 | AC | 430 . . . 436 | CAACACA | 546 . . . 553 | ACACACAG |
| | 994 | 416 . . . 416 | A | | | | | 435 . . . 436 | CA | 551 . . . 552 | CA |
| NLMz | 18GH | — | — | 414 . . . 420 | ACATACA | 348 . . . 351 | ACAC | — | — | 546 . . . 547 | AC |
| | 128 | | | 417 . . . 421 | TACAG | 350 . . . 351 | AC | | | | |
| NLMz | 18GH | 414 . . . 437 | ACATACAGAG | 412 . . . 418 | CAACATA | 349 . . . 355 | CACACAG | 430 . . . 436 | CAACACA | 546 . . . 549 | ACAC |
| | 130 | | AATGGTGGAT | | | | | | | | |
| | | | TTCC (382) | | | | | | | | |
| | | 417 . . . 420 | TACA | 415 . . . 418 | CATA | | | | | | |
| NLMz | 18GH | 417 . . . 418 | TA | 416 . . . 419 | ATAC | 348 . . . 353 | ACACAC | — | — | 546 . . . 547 | AC |
| | 131 | | | 417 . . . 419 | TAC | 350 . . . 353 | ACAC | | | | |
| | | | | 418 . . . 419 | AC | | | | | | |
| NLMz | 18GH | 413 . . . 419 | AACATAC | 414 . . . 420 | ACATACA | 348 . . . 351 | ACAC | — | — | 546 . . . 547 | AC |
| | 133 | 414 . . . 419 | ACATAC | 417 . . . 420 | TACA | 350 . . . 351 | AC | | | | |
| | | | | 417 . . . 421 | TACAG | | | | | | |
| NLMz | 18GH | — | — | — | — | 348 . . . 349 | AC | — | — | — | — |
| | 136 | | | | | | | | | | |
| NLMz | 18GH | 412 . . . 418 | CAACATA | 414 . . . 419 | ACATAC | 347 . . . 354 | AACACAC | 432 . . . 433 | AC | 546 . . . 549 | ACAC |
| | 216 | 415 . . . 418 | CATA | 416 . . . 419 | ATAC | | A | | | 548 . . . 549 | AC |
| NLMz | 18GH | 418 . . . 419 | AC | 414 . . . 417 | ACAT | 348 . . . 351 | ACAC | | | 546 . . . 549 | ACAC |
| | 227 | | | | | | | | | | |
| NLMz | 18GH | 414 . . . 417 | ACAT | 414 . . . 420 | ACATACA | 352 . . . 355 | ACAG | 429 . . . 435 | TCAACAC | 546 . . . 551 | ACACAC |
| | 5 | | | 415 . . . 421 | CATACAG | | | 432 . . . 435 | ACAC | 548 . . . 551 | ACAC |
| NLMz | 18GH | 414 . . . 417 | ACAT | 416 . . . 429 | ATACAGAG | 350 . . . 363 | ACACAGA | 436 . . . 439 | ACAG | 550 . . . 553 | ACAG |
| | 6 | | | | AATGGT | | GAATGGT | | | | |
| | | | | | (389) | | (393) | | | | |
| | | | | 417 . . . 422 | TACAGA | 351 . . . 356 | CACAGA | | | | |
| | | | | | | 353 . . . 356 | CAGA | | | | |
| NLMz | 18GH | 416 . . . 423 | ATACAGAG | 414 . . . 419 | ACATAC | 348 . . . 351 | ACAC | 433 . . . 437 | CACAC | 546 . . . 549 | ACAC |
| | 65 | 418 . . . 420 | ACA | 417 . . . 419 | TAC | 350 . . . 351 | AC | 436 . . . 437 | AC | | |
| NLMz | 18GH | 414 . . . 417 | ACAT | 414 . . . 420 | ACATACA | 352 . . . 355 | ACAG | 429 . . . 435 | TCAACAC | 546 . . . 551 | ACACAC |
| | 66 | | | 415 . . . 421 | CATACAG | | | 432 . . . 435 | ACAC | 548 . . . 551 | ACAC |
| NLMz | 18GH | | | 411 . . . 420 | TCAACATA | | | 432 . . . 433 | AC | 546 . . . 549 | ACAC |
| | 69 | | | | CA (379) | | | 436 . . . 439 | ACAG | | |
| NLMz | 18GH | 416 . . . 423 | ATACAGAG | 414 . . . 419 | ACATAC | 348 . . . 351 | ACAC | 433 . . . 437 | CACAC | 546 . . . 549 | ACAC |
| | 72 | 418 . . . 420 | ACA | 417 . . . 419 | TAC | 350 . . . 351 | AC | 436 . . . 437 | AC | | |
| NLMz | 18GH | — | — | 414 . . . 420 | ACATACA | 348 . . . 351 | ACAC | — | — | 546 . . . 547 | AC |
| | 73 | | | 417 . . . 421 | TACAG | 350 . . . 351 | AC | | | | |
| NLMz | 18GH | — | — | 412 . . . 418 | CAACATA | — | — | — | — | — | — |
| | 74 | | | | | | | | | | |
| NLMz | 18GH | 414 . . . 419 | ACATAC | 414 . . . 417 | ACAT | 348 . . . 349 | AC | 431 . . . 431 | A | 546 . . . 549 | ACAC |
| | 78 | | | | | | | 434 . . . 438 | ACACA | 548 . . . 549 | AC |
| | | | | | | | | 435 . . . 438 | CACA | | |
| NLMz | 18GH | — | — | 414 . . . 420 | ACATACA | 348 . . . 351 | ACAC | — | — | 546 . . . 547 | AC |
| | 79 | | | 417 . . . 421 | TACAG | 350 . . . 351 | AC | | | | |
| NLMz | 18GH | 417 . . . 420 | TACA | 416 . . . 421 | ATACAG | 348 . . . 354 | ACACACA | 435 . . . 447 | CACAGAGA | 549 . . . 552 | CACA |
| | 8 | | | | | | | | ATGGT | | |
| | | | | 417 . . . 420 | TACA | 350 . . . 354 | ACACA | | (401) | 549 . . . 553 | CACAG |
| NLMz | 18GH | 417 . . . 418 | TA | 416 . . . 419 | ATAC | 348 . . . 353 | ACACAC | — | — | 546 . . . 547 | AC |
| | 9 | | | 418 . . . 419 | AC | | | | | | |
| NLMz | 18GH | 414 . . . 417 | ACAT | 414 . . . 417 | ACAT | 348 . . . 351 | ACAC | 431 . . . 441 | AACACACA | 546 . . . 549 | ACAC |
| | 71 | | | | | | | | GAG (391) | | |
| NLMz | 17GH | 414 . . . 417 | ACAT | 414 . . . 420 | ACATACA | 352 . . . 355 | ACAG | 429 . . . 435 | TCAACAC | 546 . . . 551 | ACACAC |
| | 1905 | | | 415 . . . 421 | CATACAG | | | 432 . . . 435 | ACAC | 548 . . . 551 | ACAC |

TABLE 4D
Mutant pmt alleles in oriental tobacco produced by genome editing using GET2.

| | | PMT1b | | PMT1a | | PMT2 | | PMT3 | | PMT4 | |
|---|---|---|---|---|---|---|---|---|---|---|---|
| | | | Deleted | | Deleted | | Deleted | | Deleted | | Deleted |
| VARIETY | LINE | Position | sequence | Position | sequence | Position | sequence | Position | sequence | Position | sequence |
| Katerini | 18GH125 | 412 . . . 418 | CAACATA | 416 . . . 419 | ATAC | 348 . . . 355 | ACACACAG | 432 . . . 435 | ACAC | 546 . . . 547 | AC |
| | | 414 . . . 418 | ACATA | 418 . . . 419 | AC | | | 434 . . . 435 | AC | | |
| Basma | 18GH203 | 414 . . . 417 | ACAT | 412 . . . 418 | CAACATA | 348 . . . 357 | ACACACAG | 432 . . . 435 | ACAC | 546 . . . 549 | ACAC |
| | | | | | | | AG (392) | | | | |
| | | | | 415 . . . 418 | CATA | 353 . . . 354 | CA | 434 . . . 435 | AC | | |
| Katerini | 18GH208 | 416 . . . 419 | ATAC | 414 . . . 417 | ACAT | 352 . . . 355 | ACAG | 432 . . . 441 | ACACACAG | 546 . . . 553 | ACACAC |
| | | | | | | | | | AG (392) | | |
| | | 418 . . . 419 | AC | | | | | 435 . . . 438 | CACA | | AG |
| Basma | 18GH341 | 414 . . . 417 | ACAT | 412 . . . 418 | CAACATA | 348 . . . 357 | ACACACAG | 432 . . . 435 | ACAC | 546 . . . 549 | ACAC |
| | | | | | | | AG (392) | | | | |
| | | | | 415 . . . 418 | CATA | 353 . . . 354 | CA | 434 . . . 435 | AC | | |
| Katerini | 18GH403 | 414 . . . 417 | ACAT | 414 . . . 417 | ACAT | 348 . . . 351 | ACAC | 432 . . . 435 | ACAC | | |
| Katerini | 18GH414 | 412 . . . 418 | CAACATA | 414 . . . 417 | ACAT | 348 . . . 349 | AC | 430 . . . 436 | CAACACA | 546 . . . 547 | AC |
| | | 415 . . . 418 | CATA | | | | | | | | |
| Katerini | 18GH434 | 413 . . . 420 | AACATAC | 414 . . . 417 | ACAT | 351 . . . 357 | CACAGAG | 433 . . . 439 | CACACAG | 546 . . . 547 | AC |
| | | | A | | | | | | | | |
| | | 417 . . . 420 | TACA | | | | | 437 . . . 438 | CA | | |
| Katerini | 18GH436 | 412 . . . 418 | CAACATA | 414 . . . 421 | ACATACA | 346 . . . 352 | CAACACA | 432 . . . 433 | AC | 546 . . . 547 | AC |
| | | | | | G | | | 436 . . . 443 | ACAGAGAA | | |
| | | 415 . . . 416 | CA | | | | | 445 . . . 449 | GGTGG | | |
| | | | | | | | | 451 . . . 463 | TTTCCATAC | | |
| | | | | | | | | | ACTG (402) | | |
| Katerini | 18GH437 | 414 . . . 417 | ACAT | 414 . . . 417 | ACAT | 348 . . . 349 | AC | 433 . . . 439 | CACACAG | 544 . . . 553 | CAACAC |
| | | | | | | | | | | | ACAG |
| | | | | | | | | | | | (390) |
| | | | | 416 . . . 417 | AT | | | 435 . . . 438 | CACA | 551 . . . 552 | CA |
| Katerini | 18GH449 | 414 . . . 417 | ACAT | 413 . . . 419 | AACATAC | 348 . . . 349 | AC | 432 . . . 435 | ACAC | 546 . . . 549 | ACAC |
| | | 415 . . . 416 | CA | 418 . . . 419 | AC | | | | | | |
| Katerini | 18GH706 | 416 . . . 420 | ATACA | 414 . . . 417 | ACAT | 348 . . . 351 | ACAC | 432 . . . 439 | ACACACAG | 545 . . . 555 | AACACA |
| | | 419 . . . 420 | CA | | | | | 435 . . . 438 | CACA | | CAGAG |
| | | | | | | | | | | | (391) |
| Katerini | 18GH709 | 412 . . . 418 | CAACATA | 414 . . . 417 | ACAT | 348 . . . 349 | AC | 432 . . . 435 | ACAC | 546 . . . 547 | AC |
| | | 417 . . . 418 | TA | 416 . . . 417 | AT | | | | | | |
| Katerini | 18GH710 | 414 . . . 417 | ACAT | 414 . . . 417 | ACAT | 351 . . . 354 | CACA | 432 . . . 435 | ACAC | 546 . . . 547 | AC |
| | | | | | | 352 . . . 355 | ACAG | 434 . . . 435 | AC | | |
| Katerini | 18GH716 | 416 . . . 419 | ATAC | 417 . . . 420 | TACA | 348 . . . 351 | ACAC | 432 . . . 439 | ACACACAG | 546 . . . 549 | ACAC |
| | | 418 . . . 419 | AC | 417 . . . 421 | TACAG | | | 435 . . . 438 | CACA | 548 . . . 549 | AC |
| Katerini | 18GH729 | 414 . . . 417 | ACAT | 414 . . . 417 | ACAT | 350 . . . 357 | ACACAGAG | 432 . . . 435 | ACAC | 546 . . . 547 | AC |
| | | | | | | 353 . . . 354 | CA | | | | |
| Katerini | 18GH731 | 414 . . . 417 | ACAT | 414 . . . 417 | ACAT | 348 . . . 351 | ACAC | 433 . . . 433 | C | 544 . . . 553 | CAACAC |
| | | | | | | | | 435 . . . 439 | CACAG | | ACAG |
| | | | | | | | | | | | (390) |
| Katerini | 18GH752 | 416 . . . 419 | ATAC | 414 . . . 417 | ACAT | 348 . . . 349 | AC | 432 . . . 435 | ACAC | 546 . . . 549 | ACAC |
| | | 418 . . . 419 | AC | | | | | | | | |
| Katerini | 18GH756 | 416 . . . 419 | ATAC | 414 . . . 417 | ACAT | 353 . . . 354 | CA | 433 . . . 439 | CACACAG | 545 . . . 555 | AACACA |
| | | | | | | | | | | | CAGAG |
| | | | | | | | | | | | (391) |
| | | 416 . . . 419 | ATAC | 414 . . . 417 | ACAT | 353 . . . 354 | CA | 433 . . . 439 | CACACAG | 545 . . . 555 | AACACA |
| | | | | | | | | | | | CAGAG |
| | | | | | | | | | | | (391) |
| | | | | | | | | 437 . . . 438 | CA | 549 . . . 552 | CACA |
| Katerini | 18GH768 | 416 . . . 419 | ATAC | 414 . . . 417 | ACAT | 348 . . . 351 | ACAC | 432 . . . 433 | AC | 544 . . . 550 | CAACAC |
| | | | | | | | | | | | A |
| | | | | | | | | | | 547 . . . 550 | CACA |
| Katerini | 18GH771 | 416 . . . 419 | ATAC | 414 . . . 417 | ACAT | 346 . . . 352 | CAACACA | 432 . . . 435 | ACAC | 546 . . . 547 | AC |
| | | | | | | 349 . . . 352 | CACA | 434 . . . 435 | AC | | |
| Katerini | 18GH776 | 409 . . . 415 | ATTCAAC | 411 . . . 417 | TCAACAT | 348 . . . 351 | ACAC | 441 . . . 441 | G | 546 . . . 549 | ACAC |
| | | 418 . . . 419 | AC | 414 . . . 417 | ACAT | | | | | 548 . . . 549 | AC |
| Katerini | 18GH800 | 412 . . . 418 | CAACATA | 414 . . . 420 | ACATACA | 348 . . . 351 | ACAC | 432 . . . 435 | ACAC | 541 . . . 551 | ATTCAAC |
| | | | | | | | | | | | ACAC |
| | | | | | | | | | | | (403) |
| | | 415 . . . 418 | CATA | 416 . . . 420 | ATACA | | | | | 548 . . . 551 | ACAC |
| Katerini | 18GH818 | 414 . . . 417 | ACAT | 409 . . . 415 | ATTCAAC | 348 . . . 351 | ACAC | 432 . . . 435 | ACAC | — | — |
| | | | | | | | | 434 . . . 435 | AC | | |

TABLE 4E
Mutant pmt alleles in NLM (Ph Ph) tobacco produced by genome editing using GET1.

| | PMT1b | | PMT1a | | PMT2 | | PMT3 | | PMT4 | |
|---|---|---|---|---|---|---|---|---|---|---|
| | | Position | | Position | | Position | | Position | | Position |
| LINE | Modification | from ATG | | from ATG | | from ATG | | from ATG | | from ATG |
| CS15 | A-deleted | 131 | A- | 132 | A-deleted | 98 | A-deleted | 98 | A-del | 131 |
| | T-deleted | 262 | inserted | | T-deleted | 196 | T-deleted | 282 | | |

TABLE 5A
A list of exemplary mutant alleles obtained in the PMT1b gene.
Mutant allele sequences listed here and Tables 5B to 5E
represent about 40-nucleotide-long genomic sequences from each
edited PMT gene with the edited site in the middle of the genomic
sequence (e.g., 20 nucleotides on each side of the deleted or
inserted sequence site). These mutant alleles corresponds to
those listed in Tables 4A to 4E.
PMT1b

| | | Mutant | | Reference | |
|---|---|---|---|---|---|
| | Deleted | Allele | | Allele | |
| | sequence | Sequence | | Sequence | Reference |
| | (SEQ ID | (SEQ | Mutant Allele | (SEQ ID | Allele |
| Position | No.) | ID No.) | Sequence | No.) | Sequence |
| 131 . . . 131 | A | 23 | TGGCATTTCCAAACACCA | 201 | TGGCATTTCCAAACACCAAAaCGGG |
| | | | AACGGGCACCAGAATGG | | CACCAGAATGGCACTT |
| | | | CACTT | | |
| 262 . . . 262 | T | 24 | CCAACTCTATTAAGCCTG | 202 | CCAACTCTATTAAGCCTGGTtGGTTT |
| | | | GTGGTTTTCAGAGTTTAG | | TCAGAGTTTAGCGCA |
| | | | CGCA | | |
| 409 . . . 415 | ATTCAAC | 25 | TTCTGACTTTGGATGGAG | 203 | TTCTGACTTTGGATGGAGCAattcaacA |
| | | | CAATACAGAGAATGGTG | | TACAGAGAATGGTGGATTT |
| | | | GATTT | | |
| 411 . . . 420 | TCAACATACA | 26 | CTGACTTTGGATGGAGCA | 204 | CTGACTTTGGATGGAGCAATtcaacatac |
| | (379) | | ATGAGAATGGTGGATTTC | | aGAGAATGGTGGATTTCCATA |
| | | | CATA | | |
| 412 . . . 418 | CAACATA | 27 | TGACTTTGGATGGAGCA | 205 | TGACTTTGGATGGAGCAATTcaacataC |
| | | | ATTCAGAGAATGGTGGA | | AGAGAATGGTGGATTTCCA |
| | | | TTTCCA | | |
| 412 . . . 421 | CAACATACAG | 28 | TGACTTTGGATGGAGCA | 206 | TGACTTTGGATGGAGCAATTcaacataC |
| | (380) | | ATTAGAATGGTGGATTTC | | agAGAATGGTGGATTTCCATAC |
| | | | CATAC | | |
| 413 . . . 419 | AACATAC | 29 | GACTTTGGATGGAGCAA | 207 | GACTTTGGATGGAGCAATTCaacatacA |
| | | | TTCAGAGAATGGTGGATT | | GAGAATGGTGGATTTCCAT |
| | | | TCCAT | | |
| 413 . . . 420 | AACATACA | 30 | GACTTTGGATGGAGCAA | 208 | GACTTTGGATGGAGCAATTCaacataca |
| | | | TTCGAGAATGGTGGATTT | | GAGAATGGTGGATTTCCATA |
| | | | CCATA | | |
| 413 . . . 421 | AACATACAG | 31 | GACTTTGGATGGAGCAA | 209 | GACTTTGGATGGAGCAATTCaacataca |
| | | | TTCAGAATGGTGGATTTC | | gAGAATGGTGGATTTCCATAC |
| | | | CATAC | | |
| 413 . . . 428 | AACATACAGAGA | 32 | GACTTTGGATGGAGCAA | 210 | GACTTTGGATGGAGCAATTCaacataca |
| | ATGG (381) | | TTCTGGATTTCCATACAC | | gagaatggTGGATTTCCATACACTGAAA |
| | | | TGAAA | | |
| 414 . . . 417 | ACAT | 33 | ACTTTGGATGGAGCAATT | 211 | ACTTTGGATGGAGCAATTCAacatAC |
| | | | CAtACAGAGAATGGTGGA | | AGAGAATGGTGGATTTCC |
| | | | TTTCC | | |
| 414 . . . 418 | ACATA | 34 | ACTTTGGATGGAGCAATT | 212 | ACTTTGGATGGAGCAATTCAacataCA |
| | | | CACAGAGAATGGTGGAT | | GAGAATGGTGGATTTCCA |
| | | | TTCCA | | |
| 414 . . . 419 | ACATAC | 35 | ACTTTGGATGGAGCAATT | 213 | ACTTTGGATGGAGCAATTCAacatacA |
| | | | CAAGAGAATGGTGGATT | | GAGAATGGTGGATTTCCAT |
| | | | TCCAT | | |
| 414 . . . 420 | ACATACA | 36 | ACTTTGGATGGAGCAATT | 214 | ACTTTGGATGGAGCAATTCAacatacaG |
| | | | CAGAGAATGGTGGATTT | | AGAATGGTGGATTTCCATA |
| | | | CCATA | | |
| 414 . . . 437 | ACATACAGAGAA | 37 | ACTTTGGATGGAGCAATT | 215 | ACTTTGGATGGAGCAATTCAacatacag |
| | TGGTGGATTTCC | | CAATACACTGAAATGATT | | agaatggtggatttccATACACTGAA |
| | (382) | | GTTC | | ATGATTGTTC |
| 415 . . . 416 | CA | 38 | CTTTGGATGGAGCAATTC | 216 | CTTTGGATGGAGCAATTCAAcaTACA |
| | | | AATACAGAGAATGGTGG | | GAGAATGGTGGATTTC |
| | | | ATTTC | | |
| 415 . . . 418 | CATA | 39 | CTTTGGATGGAGCAATTC | 217 | CTTTGGATGGAGCAATTCAAcataCA |
| | | | AACAGAGAATGGTGGAT | | GAGAATGGTGGATTTCCA |
| | | | TTCCA | | |
| 415 . . . 420 | CATACA | 40 | CTTTGGATGGAGCAATTC | 218 | CTTTGGATGGAGCAATTCAAcatacaG |
| | | | AAGAGAATGGTGGATTT | | AGAATGGTGGATTTCCATA |
| | | | CCATA | | |
| 415 . . . 421 | CATACAG | 41 | CTTTGGATGGAGCAATTC | 219 | CTTTGGATGGAGCAATTCAAcatacag |
| | | | AAAGAATGGTGGATTTC | | AGAATGGTGGATTTCCATAC |
| | | | CATAC | | |
| 416 . . . 416 | A | 42 | TTTGGATGGAGCAATTCA | 220 | TTTGGATGGAGCAATTCAACaTACA |
| | | | ACTACAGAGAATGGTGG | | GAGAATGGTGGATTTC |
| | | | ATTTC | | |
| 416 . . . 418 | ATA | 43 | TTTGGATGGAGCAATTCA | 221 | TTTGGATGGAGCAATTCAACataCAG |
| | | | ACCAGAGAATGGTGGAT | | AGAATGGTGGATTTCCA |
| | | | TTCCA | | |
| 416 . . . 419 | ATAC | 44 | TTTGGATGGAGCAATTCA | 222 | TTTGGATGGAGCAATTCAACatacAG |
| | | | ACAGAGAATGGTGGATT | | AGAATGGTGGATTTCCAT |
| | | | TCCAT | | |
| 416 . . . 420 | ATACA | 45 | TTTGGATGGAGCAATTCA | 223 | TTTGGATGGAGCAATTCAACatacaGA |
| | | | ACGAGAATGGTGGATTT | | GAATGGTGGATTTCCATA |
| | | | CCATA | | |
| 416 . . . 421 | ATACAG | 46 | TTTGGATGGAGCAATTCA | 224 | TTTGGATGGAGCAATTCAACatacagA |
| | | | ACAGAATGGTGGATTTCC | | GAATGGTGGATTTCCATAC |
| | | | ATAC | | |
| 416 . . . 423 | ATACAGAG | 47 | TTTGGATGGAGCAATTCA | 225 | TTTGGATGGAGCAATTCAACatacagag |
| | | | ACAATGGTGGATTTCCAT | | AATGGTGGATTTCCATACAC |
| | | | ACAC | | |
| 417 . . . 418 | TA | 48 | TTGGATGGAGCAATTCA | 226 | TTGGATGGAGCAATTCAACAtaCAGA |
| | | | ACACAGAGAATGGTGGA | | GAATGGTGGATTTCCA |
| | | | TTTCCA | | |
| 417 . . . 420 | TACA | 49 | TTGGATGGAGCAATTCA | 227 | TTGGATGGAGCAATTCAACAtacaGA |
| | | | ACAGAGAATGGTGGATT | | GAATGGTGGATTTCCATA |
| | | | TCCATA | | |
| 418 . . . 419 | AC | 50 | TGGATGGAGCAATTCAA | 228 | TGGATGGAGCAATTCAACATacAGA |
| | | | CATAGAGAATGGTGGAT | | GAATGGTGGATTTCCAT |
| | | | TTCCAT | | |
| 418 . . . 420 | ACA | 51 | TGGATGGAGCAATTCAA | 229 | TGGATGGAGCAATTCAACATacaGAG |
| | | | CATGAGAATGGTGGATTT | | AATGGTGGATTTCCATA |
| | | | CCATA | | |
| 418 . . . 421 | ACAG | 52 | TGGATGGAGCAATTCAA | 230 | TGGATGGAGCAATTCAACATacagAG |
| | | | CATAGAATGGTGGATTTC | | AATGGTGGATTTCCATAC |
| | | | CATAC | | |
| 418 . . . 423 | ACAGAG | 53 | TGGATGGAGCAATTCAA | 231 | TGGATGGAGCAATTCAACATacagagA |
| | | | CATAATGGTGGATTTCCA | | ATGGTGGATTTCCATACAC |
| | | | TACAC | | |
| 419 . . . 420 | CA | 54 | GGATGGAGCAATTCAAC | 232 | GGATGGAGCAATTCAACATAcaGAG |
| | | | ATAGAGAATGGTGGATT | | AATGGTGGATTTCCATA |
| | | | TCCATA | | |
| 427 . . . 427 | G | 55 | CAATTCAACATACAGAG | 233 | CAATTCAACATACAGAGAATgGTGG |
| | | | AATGTGGATTTCCATACA | | ATTTCCATACACTGAA |
| | | | CTGAA | | |

TABLE 5B
A list of exemplary mutant alleles obtained in the PMT1a gene.
PMT1a

| | | Mutant | | Reference | |
|---|---|---|---|---|---|
| | Deleted | Allele | | Allele | |
| | sequence | Sequence | | Sequence | |
| | (SEQ ID | (SEQ ID | | (SEQ ID | |
| Position | No.) | No.) | Mutant Allele Sequence | No.) | Reference Allele Sequence |
| 132 . . . 132 | A inserted | 56 | GCACTTCCAAACACCAAAACa | 234 | GCACTTCCAAACACCAAAACGGGCA |
| | | | GGGCACCAGAATGGCACTTT | | CCAGAATGGCACTTT |
| 409 . . . 415 | ATTCAAC | 57 | TTCTGACTTTGGATGGAGCAA | 235 | TTCTGACTTTGGATGGAGCAattcaacAT |
| | | | TACAGAGAATGGTGGATTT | | ACAGAGAATGGTGGATTT |
| 409 . . . 420 | ATTCAACA | 58 | TTCTGACTTTGGATGGAGCAG | 236 | TTCTGACTTTGGATGGAGCAattcaacatac |
| | TACA (383) | | AGAATGGTGGATTTCCATA | | aGAGAATGGTGGATTTCCATA |
| 411 . . . 417 | TCAACAT | 59 | CTGACTTTGGATGGAGCAATA | 237 | CTGACTTTGGATGGAGCAATtcaacatAC |
| | | | CAGAGAATGGTGGATTTCC | | AGAGAATGGTGGATTTCC |
| 411 . . . 420 | TCAACATACA | 60 | CTGACTTTGGATGGAGCAATG | 238 | CTGACTTTGGATGGAGCAATtcaacataca |
| | (384) | | AGAATGGTGGATTTCCATA | | GAGAATGGTGGATTTCCATA |
| 412 . . . 418 | CAACATA | 61 | TGACTTTGGATGGAGCAATTC | 239 | TGACTTTGGATGGAGCAATTcaacataCA |
| | | | AGAGAATGGTGGATTTCCA | | GAGAATGGTGGATTTCCA |
| 412 . . . 421 | CAACATACAG | 62 | TGACTTTGGATGGAGCAATTA | 240 | TGACTTTGGATGGAGCAATTcaacatacag |
| | (385) | | GAATGGTGGATTTCCATAC | | AGAATGGTGGATTTCCATAC |
| 413 . . . 419 | AACATAC | 63 | GACTTTGGATGGAGCAATTCA | 241 | GACTTTGGATGGAGCAATTCaacatacA |
| | | | GAGAATGGTGGATTTCCAT | | GAGAATGGTGGATTTCCAT |
| 413 . . . 420 | AACATACA | 64 | GACTTTGGATGGAGCAATTCG | 242 | GACTTTGGATGGAGCAATTCaacatacaG |
| | | | AGAATGGTGGATTTCCATA | | AGAATGGTGGATTTCCATA |
| 413 . . . 421 | AACATACAG | 65 | GACTTTGGATGGAGCAATTCA | 243 | GACTTTGGATGGAGCAATTCaacatacag |
| | | | GAATGGTGGATTTCCATAC | | AGAATGGTGGATTTCCATAC |
| 413 . . . 422 | AACATACAGA | 66 | GACTTTGGATGGAGCAATTCG | 244 | GACTTTGGATGGAGCAATTCaacatacag |
| | (386) | | AATGGTGGATTTCCATACA | | aGAATGGTGGATTTCCATACA |
| 413 . . . 428 | AACATA | 67 | GACTTTGGATGGAGCAATTCT | 245 | GACTTTGGATGGAGCAATTCaacatacag |
| | CAGAG | | GGATTTCCATACACTGAAA | | agaatggTGGATTTCCATACACTGAAA |
| | AATGG | | | | |
| | (387) | | | | |
| 414 . . . 415 | AC | 68 | ACTTTGGATGGAGCAATTCAA | 246 | ACTTTGGATGGAGCAATTCAacATAC |
| | | | TACAGAGAATGGTGGATTT | | AGAGAATGGTGGATTT |
| 414 . . . 417 | ACAT | 69 | ACTTTGGATGGAGCAATTCAA | 247 | ACTTTGGATGGAGCAATTCAacatACA |
| | | | CAGAGAATGGTGGATTTCC | | GAGAATGGTGGATTTCC |
| 414 . . . 419 | ACATAC | 70 | ACTTTGGATGGAGCAATTCAA | 248 | ACTTTGGATGGAGCAATTCAacatacAG |
| | | | GAGAATGGTGGATTTCCAT | | AGAATGGTGGATTTCCAT |
| 414 . . . 420 | ACATACA | 71 | ACTTTGGATGGAGCAATTCAG | 249 | ACTTTGGATGGAGCAATTCAacatacaG |
| | | | AGAATGGTGGATTTCCATA | | AGAATGGTGGATTTCCATA |
| 414 . . . 421 | ACATACAG | 72 | ACTTTGGATGGAGCAATTCAA | 250 | ACTTTGGATGGAGCAATTCAacatacagA |
| | | | GAATGGTGGATTTCCATAC | | GAATGGTGGATTTCCATAC |
| 414 . . . 423 | ACATA | 73 | ACTTTGGATGGAGCAATTCAA | 251 | ACTTTGGATGGAGCAATTCAacatacaga |
| | CAGAG | | ATGGTGGATTTCCATACAC | | gAATGGTGGATTTCCATACAC |
| | (388) | | | | |
| 415 . . . 415 | C | 74 | CTTTGGATGGAGCAATTCAAA | 252 | CTTTGGATGGAGCAATTCAAcATACA |
| | | | TACAGAGAATGGTGGATTT | | GAGAATGGTGGATTT |
| 415 . . . 416 | CA | 75 | CTTTGGATGGAGCAATTCAAT | 253 | CTTTGGATGGAGCAATTCAAcaTACA |
| | | | ACAGAGAATGGTGGATTTC | | GAGAATGGTGGATTTC |
| 415 . . . 418 | CATA | 76 | CTTTGGATGGAGCAATTCAAC | 254 | CTTTGGATGGAGCAATTCAAcataCAG |
| | | | AGAGAATGGTGGATTTCCA | | AGAATGGTGGATTTCCA |
| 415 . . . 419 | CATAC | 77 | CTTTGGATGGAGCAATTCAAA | 255 | CTTTGGATGGAGCAATTCAAcatacAGA |
| | | | GAGAATGGTGGATTTCCAT | | GAATGGTGGATTTCCAT |
| 415 . . . 421 | CATACAG | 78 | CTTTGGATGGAGCAATTCAAA | 256 | CTTTGGATGGAGCAATTCAAcatacagA |
| | | | GAATGGTGGATTTCCATAC | | GAATGGTGGATTTCCATAC |
| 416 . . . 417 | AT | 79 | TTTGGATGGAGCAATTCAACtA | 257 | TTTGGATGGAGCAATTCAACatACAGA |
| | | | CAGAGAATGGTGGATTTCC | | GAATGGTGGATTTCC |
| 416 . . . 419 | ATAC | 80 | TTTGGATGGAGCAATTCAACA | 258 | TTTGGATGGAGCAATTCAACatacAGA |
| | | | GAGAATGGTGGATTTCCAT | | GAATGGTGGATTTCCAT |
| 416 . . . 420 | ATACA | 81 | TTTGGATGGAGCAATTCAACG | 259 | TTTGGATGGAGCAATTCAACatacaGAG |
| | | | AGAATGGTGGATTTCCATA | | AATGGTGGATTTCCATA |
| 416 . . . 421 | ATACAG | 82 | TTTGGATGGAGCAATTCAACA | 260 | TTTGGATGGAGCAATTCAACatacagAG |
| | | | GAATGGTGGATTTCCATAC | | AATGGTGGATTTCCATAC |
| 416 . . . 422 | ATACAGA | 83 | TTTGGATGGAGCAATTCAACG | 261 | TTTGGATGGAGCAATTCAACatacagaG |
| | | | AATGGTGGATTTCCATACA | | AATGGTGGATTTCCATACA |
| 416 . . . 429 | ATACAG | 84 | TTTGGATGGAGCAATTCAACG | 262 | TTTGGATGGAGCAATTCAACatacagaga |
| | AGAAT | | GATTTCCATACACTGAAAT | | atggtGGATTTCCATACACTGAAAT |
| | GGT | | | | |
| | (389) | | | | |
| 417 . . . 417 | T | 85 | TTGGATGGAGCAATTCAACAA | 263 | TTGGATGGAGCAATTCAACAtACAGA |
| | | | CAGAGAATGGTGGATTTCC | | GAATGGTGGATTTCC |
| 417 . . . 418 | TA | 86 | TTGGATGGAGCAATTCAACAC | 264 | TTGGATGGAGCAATTCAACAtaCAGA |
| | | | AGAGAATGGTGGATTTCCA | | GAATGGTGGATTTCCA |
| 417 . . . 419 | TAC | 87 | TTGGATGGAGCAATTCAACAA | 265 | TTGGATGGAGCAATTCAACAtacAGAG |
| | | | GAGAATGGTGGATTTCCAT | | AATGGTGGATTTCCAT |
| 417 . . . 420 | TACA | 88 | TTGGATGGAGCAATTCAACAG | 266 | TTGGATGGAGCAATTCAACAtacaGAG |
| | | | AGAATGGTGGATTTCCATA | | AATGGTGGATTTCCATA |
| 417 . . . 421 | TACAG | 89 | TTGGATGGAGCAATTCAACAA | 267 | TTGGATGGAGCAATTCAACAtacagAG |
| | | | GAATGGTGGATTTCCATAC | | AATGGTGGATTTCCATAC |
| 417 . . . 422 | TACAGA | 90 | TTGGATGGAGCAATTCAACAG | 268 | TTGGATGGAGCAATTCAACAtacagaGA |
| | | | AATGGTGGATTTCCATACA | | ATGGTGGATTTCCATACA |
| 417 . . . 423 | TACAGAG | 91 | TTGGATGGAGCAATTCAACAA | 269 | TTGGATGGAGCAATTCAACAtacagagA |
| | | | ATGGTGGATTTCCATACAC | | ATGGTGGATTTCCATACAC |
| 418 . . . 419 | AC | 92 | TGGATGGAGCAATTCAACATA | 270 | TGGATGGAGCAATTCAACATacAGAG |
| | | | GAGAATGGTGGATTTCCAT | | AATGGTGGATTTCCAT |
| 418 . . . 421 | ACAG | 93 | TGGATGGAGCAATTCAACATA | 271 | TGGATGGAGCAATTCAACATacagAGA |
| | | | GAATGGTGGATTTCCATAC | | ATGGTGGATTTCCATAC |
| 418 . . . 424 | ACAGAGA | 94 | TGGATGGAGCAATTCAACATA | 272 | TGGATGGAGCAATTCAACATacagagaA |
| | | | TGGTGGATTTCCATACACT | | TGGTGGATTTCCATACACT |
| 419 . . . 420 | CA | 95 | GGATGGAGCAATTCAACATAG | 273 | GGATGGAGCAATTCAACATAcaGAGA |
| | | | AGAATGGTGGATTTCCATA | | ATGGTGGATTTCCATA |
| 419 . . . 421 | CAG | 96 | GGATGGAGCAATTCAACATAA | 274 | GGATGGAGCAATTCAACATAcagAGA |
| | | | GAATGGTGGATTTCCATAC | | ATGGTGGATTTCCATAC |
| 424 . . . 425 | AA | 97 | GAGCAATTCAACATACAGAGT | 275 | GAGCAATTCAACATACAGAGaaTGGT |
| | | | GGTGGATTTCCATACACTG | | GGATTTCCATACACTG |

TABLE 5C
A list of exemplary mutant alleles obtained in the PMT2 gene.
PMT2

| | | Mutant | | Reference | |
|---|---|---|---|---|---|
| | Deleted | Allele | | Allele | |
| | sequence | Sequence | | Sequence | |
| | (SEQ | (SEQ ID | | (SEQ ID | |
| Position | ID No.) | No.) | Mutant Allele Sequence | No.) | Reference Allele Sequence |
| 98 . . . 98 | A | 98 | TGGCACTTCCAAACACCA | 276 | TGGCACTTCCAAACACCAAAaCG |
| | | | AACGGCCACAAGAATGGG | | GCCACAAGAATGGGACTT |
| | | | ACTT | | |
| 196 . . . 196 | T | 99 | CCAATTGTATTAAGCCTGG | 277 | CCAATTGTATTAAGCCTGGTtGG |
| | | | TGGTTTTCAGAGTTTAGCG | | TTTTCAGAGTTTAGCGCA |
| | | | CA | | |
| 346 . . . 350 | CAACA | 100 | TGACTTTGGATGGAGCAAT | 278 | TGACTTTGGATGGAGCAATTcaac |
| | | | TCACAGAGAATGGTGGAT | | aCACAGAGAATGGTGGATTTC |
| | | | TTC | | |
| 346 . . . 352 | CAACACA | 101 | TGACTTTGGATGGAGCAAT | 279 | TGACTTTGGATGGAGCAATTcaac |
| | | | TCAGAGAATGGTGGATTTC | | acaCAGAGAATGGTGGATTTCCA |
| | | | CA | | |
| 346 . . . 355 | CAACACACA | 102 | TGACTTTGGATGGAGCAAT | 280 | TGACTTTGGATGGAGCAATTcaac |
| | G (390) | | TAGAATGGTGGATTTCCAT | | acacagAGAATGGTGGATTTCCATA |
| | | | AC | | C |
| 347 . . . 354 | AACACACA | 103 | GACTTTGGATGGAGCAATT | 281 | GACTTTGGATGGAGCAATTCaaca |
| | | | CGAGAATGGTGGATTTCC | | cacaGAGAATGGTGGATTTCCATA |
| | | | ATA | | |
| 347 . . . 357 | AACACACAG | 104 | GACTTTGGATGGAGCAATT | 282 | GACTTTGGATGGAGCAATTCaaca |
| | AG (391) | | CAATGGTGGATTTCCATAC | | cacagagAATGGTGGATTTCCATAC |
| | | | AC | | AC |
| 348 . . . 349 | AC | 105 | ACTTTGGATGGAGCAATTC | 283 | ACTTTGGATGGAGCAATTCAacA |
| | | | AACACAGAGAATGGTGGA | | CACAGAGAATGGTGGATTT |
| | | | TTT | | |
| 348 . . . 351 | ACAC | 106 | ACTTTGGATGGAGCAATTC | 284 | ACTTTGGATGGAGCAATTCAacac |
| | | | AACAGAGAATGGTGGATT | | ACAGAGAATGGTGGATTTCC |
| | | | TCC | | |
| 348 . . . 352 | ACACA | 107 | ACTTTGGATGGAGCAATTC | 285 | ACTTTGGATGGAGCAATTCAacac |
| | | | ACAGAGAATGGTGGATTT | | aCAGAGAATGGTGGATTTCCA |
| | | | CCA | | |
| 348 . . . 353 | ACACAC | 108 | ACTTTGGATGGAGCAATTC | 286 | ACTTTGGATGGAGCAATTCAacac |
| | | | AAGAGAATGGTGGATTTC | | acAGAGAATGGTGGATTTCCAT |
| | | | CAT | | |
| 348 . . . 354 | ACACACA | 109 | ACTTTGGATGGAGCAATTC | 287 | ACTTTGGATGGAGCAATTCAacac |
| | | | AGAGAATGGTGGATTTCC | | acaGAGAATGGTGGATTTCCATA |
| | | | ATA | | |
| 348 . . . 355 | ACACACAG | 110 | ACTTTGGATGGAGCAATTC | 288 | ACTTTGGATGGAGCAATTCAacac |
| | | | AAGAATGGTGGATTTCCAT | | acagAGAATGGTGGATTTCCATAC |
| | | | AC | | |
| 348 . . . 357 | ACACACAGA | 111 | ACTTTGGATGGAGCAATTC | 289 | ACTTTGGATGGAGCAATTCAacac |
| | G (392) | | AAATGGTGGATTTCCATAC | | acagagAATGGTGGATTTCCATACA |
| | | | AC | | C |
| 349 . . . 349 | C | 112 | CTTTGGATGGAGCAATTCA | 290 | CTTTGGATGGAGCAATTCAAcAC |
| | | | AACACAGAGAATGGTGGA | | ACAGAGAATGGTGGATTT |
| | | | TTT | | |
| 349 . . . 350 | CA | 113 | CTTTGGATGGAGCAATTCA | 291 | CTTTGGATGGAGCAATTCAAcaC |
| | | | ACACAGAGAATGGTGGAT | | ACAGAGAATGGTGGATTTC |
| | | | TTC | | |
| 349 . . . 352 | CACA | 114 | CTTTGGATGGAGCAATTCA | 292 | CTTTGGATGGAGCAATTCAAcaca |
| | | | ACAGAGAATGGTGGATTT | | CAGAGAATGGTGGATTTCCA |
| | | | CCA | | |
| 349 . . . 355 | CACACAG | 115 | CTTTGGATGGAGCAATTCA | 293 | CTTTGGATGGAGCAATTCAAcaca |
| | | | AAGAATGGTGGATTTCCAT | | cagAGAATGGTGGATTTCCATAC |
| | | | AC | | |
| 350 . . . 351 | AC | 116 | TTTGGATGGAGCAATTCAA | 294 | TTTGGATGGAGCAATTCAACacA |
| | | | CACAGAGAATGGTGGATT | | CAGAGAATGGTGGATTTCC |
| | | | TCC | | |
| 350 . . . 353 | ACAC | 117 | TTTGGATGGAGCAATTCAA | 295 | TTTGGATGGAGCAATTCAACacac |
| | | | CAGAGAATGGTGGATTTC | | AGAGAATGGTGGATTTCCAT |
| | | | CAT | | |
| 350 . . . 354 | ACACA | 118 | TTTGGATGGAGCAATTCAA | 296 | TTTGGATGGAGCAATTCAACacac |
| | | | CGAGAATGGTGGATTTCC | | aGAGAATGGTGGATTTCCATA |
| | | | ATA | | |
| 350 . . . 355 | ACACAG | 119 | TTTGGATGGAGCAATTCAA | 297 | TTTGGATGGAGCAATTCAACacac |
| | | | CAGAATGGTGGATTTCCAT | | agAGAATGGTGGATTTCCATAC |
| | | | AC | | |
| 350 . . . 357 | ACACAGAG | 120 | TTTGGATGGAGCAATTCAA | 298 | TTTGGATGGAGCAATTCAACacac |
| | | | CAATGGTGGATTTCCATAC | | agagAATGGTGGATTTCCATACAC |
| | | | AC | | |
| 350 . . . 363 | ACACAGAGA | 121 | TTTGGATGGAGCAATTCAA | 299 | TTTGGATGGAGCAATTCAACacac |
| | ATGGT | | CGGATTTCCATACACTGAA | | agagaatggtGGATTTCCATACACTG |
| | (393) | | AT | | AAAT |
| 351 . . . 352 | CA | 122 | TTGGATGGAGCAATTCAA | 300 | TTGGATGGAGCAATTCAACAcaC |
| | | | CACAGAGAATGGTGGATT | | AGAGAATGGTGGATTTCCA |
| | | | TCCA | | |
| 351 . . . 354 | CACA | 123 | TTGGATGGAGCAATTCAA | 301 | TTGGATGGAGCAATTCAACAcaca |
| | | | CAGAGAATGGTGGATTTC | | GAGAATGGTGGATTTCCATA |
| | | | CATA | | |
| 351 . . . 356 | CACAGA | 124 | TTGGATGGAGCAATTCAA | 302 | TTGGATGGAGCAATTCAACAcaca |
| | | | CAGAATGGTGGATTTCCAT | | gaGAATGGTGGATTTCCATACA |
| | | | ACA | | |
| 351 . . . 357 | CACAGAG | 125 | TTGGATGGAGCAATTCAA | 303 | TTGGATGGAGCAATTCAACAcaca |
| | | | CAAATGGTGGATTTCCATA | | gagAATGGTGGATTTCCATACAC |
| | | | CAC | | |
| 351 . . . 362 | CACAGAGAA | 126 | TTGGATGGAGCAATTCAA | 304 | TTGGATGGAGCAATTCAACAcaca |
| | TGG (394) | | CATGGATTTCCATACACTG | | gagaatggTGGATTTCCATACACT |
| | | | AAA | | GAAA |
| 352 . . . 354 | ACA | 127 | TGGATGGAGCAATTCAAC | 305 | TGGATGGAGCAATTCAACACaca |
| | | | ACGAGAATGGTGGATTTC | | GAGAATGGTGGATTTCCATA |
| | | | CATA | | |
| 352 . . . 355 | ACAG | 128 | TGGATGGAGCAATTCAAC | 306 | TGGATGGAGCAATTCAACACacag |
| | | | ACAGAATGGTGGATTTCC | | AGAATGGTGGATTTCCATAC |
| | | | ATAC | | |
| 353 . . . 354 | CA | 129 | GGATGGAGCAATTCAACA | 307 | GGATGGAGCAATTCAACACAcaG |
| | | | CAGAGAATGGTGGATTTC | | AGAATGGTGGATTTCCATA |
| | | | CATA | | |
| 353 . . . 356 | CAGA | 130 | GGATGGAGCAATTCAACA | 308 | GGATGGAGCAATTCAACACAcaga |
| | | | CAGAATGGTGGATTTCCAT | | GAATGGTGGATTTCCATACA |
| | | | ACA | | |
| 353 . . . 361 | CAGAGAATG | 131 | GGATGGAGCAATTCAACA | 309 | GGATGGAGCAATTCAACACAcaga |
| | | | CAGTGGATTTCCATACACT | | gaatgGTGGATTTCCATACACTGAA |
| | | | GAA | | |
| 354 . . . 359 | AGAGAA | 132 | GATGGAGCAATTCAACAC | 310 | GATGGAGCAATTCAACACACagag |
| | | | ACTGGTGGATTTCCATACA | | aaTGGTGGATTTCCATACACTG |
| | | | CTG | | |

TABLE 5D
A list of exemplary mutant alleles obtained in the PMT3 gene.
PMT3

| | | Mutant | | Reference | |
|---|---|---|---|---|---|
| | Deleted | Allele | | Allele | |
| | sequence | Sequence | | Sequence | |
| | (SEQ | (SEQ ID | | (SEQ ID | |
| Position | ID No.) | No.) | Mutant Allele Sequence | No.) | Reference Allele Sequence |
| 98 . . . 98 | A | 133 | TGGCACTTCCAAACACCAAACGGC | 311 | TGGCACTTCCAAACACCAAAaCGGCCA |
| | | | CACCAGAATGGCACTT | | CCAGAATGGCACTT |
| 280 . . . 280 | T | 134 | CCAACTCTATTAAGCCTGGTGGTTT | 312 | CCAACTCTATTAAGCCTGGTtGGTTTTC |
| | | | TCAGAGTTTAGCGCA | | AGAGTTTAGCGCA |
| 413 . . . 414 | CT | 135 | AACATATGGGAAGGTTCTGATTGG | 313 | AACATATGGGAAGGTTCTGActTTGGAT |
| | | | ATGGAGCAATTCAACA | | GGAGCAATTCAACA |
| 418 . . . 419 | GA | 136 | ATGGGAAGGTTCTGACTTTGTGGA | 314 | ATGGGAAGGTTCTGACTTTGgaTGGAGC |
| | | | GCAATTCAACACACAG | | AATTCAACACACAG |
| 426 . . . 427 | AA | 137 | GTTCTGACTTTGGATGGAGCTTCA | 315 | GTTCTGACTTTGGATGGAGCaaTTCAAC |
| | | | ACACACAGAGAATGGT | | ACACAGAGAATGGT |
| 429 . . . 435 | TCAACAC | 138 | CTGACTTTGGATGGAGCAATACAG | 316 | CTGACTTTGGATGGAGCAATtcaacacACA |
| | | | AGAATGGTGGATTTCC | | GAGAATGGTGGATTTCC |
| 429 . . . 438 | TCAACACA | 139 | CTGACTTTGGATGGAGCAATGAGA | 317 | CTGACTTTGGATGGAGCAATtcaacacacaG |
| | CA (395) | | ATGGTGGATTTCCATA | | AGAATGGTGGATTTCCATA |
| 429 . . . 439 | TCAACACA | 140 | CTGACTTTGGATGGAGCAATAGAA | 318 | CTGACTTTGGATGGAGCAATtcaacacacag |
| | CAG (396) | | TGGTGGATTTCCATAC | | AGAATGGTGGATTTCCATAC |
| 430 . . . 436 | CAACACA | 141 | TGACTTTGGATGGAGCAATTCAGA | 319 | TGACTTTGGATGGAGCAATTcaacacaCAG |
| | | | GAATGGTGGATTTCCA | | AGAATGGTGGATTTCCA |
| 431 . . . 431 | A | 142 | GACTTTGGATGGAGCAATTCACAC | 320 | GACTTTGGATGGAGCAATTCaACACACA |
| | | | ACAGAGAATGGTGGAT | | GAGAATGGTGGAT |
| 431 . . . 432 | AA | 143 | GACTTTGGATGGAGCAATTCCACA | 321 | GACTTTGGATGGAGCAATTCaaCACACA |
| | | | CAGAGAATGGTGGATT | | GAGAATGGTGGATT |
| 431 . . . 438 | AACACACA | 144 | GACTTTGGATGGAGCAATTCGAGA | 322 | GACTTTGGATGGAGCAATTCaacacacaGA |
| | | | ATGGTGGATTTCCATA | | GAATGGTGGATTTCCATA |
| 431 . . . 441 | AACACACA | 145 | GACTTTGGATGGAGCAATTCAATG | 323 | GACTTTGGATGGAGCAATTCaacacacagag |
| | GAG (397) | | GTGGATTTCCATACAC | | AATGGTGGATTTCCATACAC |
| 432 . . . 433 | AC | 146 | ACTTTGGATGGAGCAATTCAACAC | 324 | ACTTTGGATGGAGCAATTCAacACACAG |
| | | | AGAGAATGGTGGATTT | | AGAATGGTGGATTT |
| 432 . . . 435 | ACAC | 147 | ACTTTGGATGGAGCAATTCAACAG | 325 | ACTTTGGATGGAGCAATTCAacacACAGA |
| | | | AGAATGGTGGATTTCC | | GAATGGTGGATTTCC |
| 432 . . . 437 | ACACAC | 148 | ACTTTGGATGGAGCAATTCAAGAG | 326 | ACTTTGGATGGAGCAATTCAacacacAGA |
| | | | AATGGTGGATTTCCAT | | GAATGGTGGATTTCCAT |
| 432 . . . 439 | ACACACAG | 149 | ACTTTGGATGGAGCAATTCAAGAA | 327 | ACTTTGGATGGAGCAATTCAacacacagAG |
| | | | TGGTGGATTTCCATAC | | AATGGTGGATTTCCATAC |
| 432 . . . 441 | ACACACAG | 150 | ACTTTGGATGGAGCAATTCAAATG | 328 | ACTTTGGATGGAGCAATTCAacacacagagA |
| | AG (398) | | GTGGATTTCCATACAC | | ATGGTGGATTTCCATACAC |
| 432 . . . 446 | ACACACAG | 151 | ACTTTGGATGGAGCAATTCATGGA | 329 | ACTTTGGATGGAGCAATTCAacacacagaga |
| | AGAATGG | | TTTCCATACACTGAAA | | atggTGGATTTCCATACACTGAAA |
| | (399) | | | | |
| 432 . . . 448 | ACACACAG | 152 | ACTTTGGATGGAGCAATTCAGATT | 330 | ACTTTGGATGGAGCAATTCAacacacagaga |
| | AGAATGGT | | TCCATACACTGAAATG | | atggtgGATTTCCATACACTGAAATG |
| | G (400) | | | | |
| 433 . . . 433 | C | 153 | CTTTGGATGGAGCAATTCAAACAC | 331 | CTTTGGATGGAGCAATTCAAcACACAG |
| | | | AGAGAATGGTGGATTT | | AGAATGGTGGATTT |
| 433 . . . 436 | CACA | 154 | CTTTGGATGGAGCAATTCAACAGA | 332 | CTTTGGATGGAGCAATTCAAcacaCAGAG |
| | | | GAATGGTGGATTTCCA | | AATGGTGGATTTCCA |
| 433 . . . 437 | CACAC | 155 | CTTTGGATGGAGCAATTCAAAGAG | 333 | CTTTGGATGGAGCAATTCAAcacacAGAG |
| | | | AATGGTGGATTTCCAT | | AATGGTGGATTTCCAT |
| 433 . . . 439 | CACACAG | 156 | CTTTGGATGGAGCAATTCAAAGAA | 334 | CTTTGGATGGAGCAATTCAAcacacagAGA |
| | | | TGGTGGATTTCCATAC | | ATGGTGGATTTCCATAC |
| 434 . . . 435 | AC | 157 | TTTGGATGGAGCAATTCAACACAG | 335 | TTTGGATGGAGCAATTCAACacACAGAG |
| | | | AGAATGGTGGATTTCC | | AATGGTGGATTTCC |
| 434 . . . 437 | ACAC | 158 | TTTGGATGGAGCAATTCAACAGAG | 336 | TTTGGATGGAGCAATTCAACacacAGAG |
| | | | AATGGTGGATTTCCAT | | AATGGTGGATTTCCAT |
| 434 . . . 438 | ACACA | 159 | TTTGGATGGAGCAATTCAACGAGA | 337 | TTTGGATGGAGCAATTCAACacacaGAGA |
| | | | ATGGTGGATTTCCATA | | ATGGTGGATTTCCATA |
| 435 . . . 436 | CA | 160 | TTGGATGGAGCAATTCAACACAGA | 338 | TTGGATGGAGCAATTCAACAcaCAGAGA |
| | | | GAATGGTGGATTTCCA | | ATGGTGGATTTCCA |
| 435 . . . 438 | CACA | 161 | TTGGATGGAGCAATTCAACAGAGA | 339 | TTGGATGGAGCAATTCAACAcacaGAGA |
| | | | ATGGTGGATTTCCATA | | ATGGTGGATTTCCATA |
| 435 . . . 439 | CACAG | 162 | TTGGATGGAGCAATTCAACAAGAA | 340 | TTGGATGGAGCAATTCAACAcacagAGAA |
| | | | TGGTGGATTTCCATAC | | TGGTGGATTTCCATAC |
| 435 . . . 447 | CACAGA | 163 | TTGGATGGAGCAATTCAACAGGAT | 341 | TTGGATGGAGCAATTCAACAcacagagaatg |
| | GAAT | | TTCCATACACTGAAAT | | gtGGATTTCCATACACTGAAAT |
| | GGT (401) | | | | |
| 436 . . . 437 | AC | 164 | TGGATGGAGCAATTCAACACAGAG | 342 | TGGATGGAGCAATTCAACACacAGAGA |
| | | | AATGGTGGATTTCCAT | | ATGGTGGATTTCCAT |
| 436 . . . 439 | ACAG | 165 | TGGATGGAGCAATTCAACACAGAA | 343 | TGGATGGAGCAATTCAACACacagAGAA |
| | | | TGGTGGATTTCCATAC | | TGGTGGATTTCCATAC |
| 436 . . . 443 | ACAGAGAA | 166 | TGGATGGAGCAATTCAACACTGGT | 344 | TGGATGGAGCAATTCAACACacagagaaTG |
| | | | GGATTTCCATACACTG | | GTGGATTTCCATACACTG |
| 437 . . . 438 | CA | 167 | GGATGGAGCAATTCAACACAGAG | 345 | GGATGGAGCAATTCAACACAcaGAGAA |
| | | | AATGGTGGATTTCCATA | | TGGTGGATTTCCATA |
| 440 . . . 442 | AGA | 168 | TGGAGCAATTCAACACACAGATGG | 346 | TGGAGCAATTCAACACACAGagaATGGT |
| | | | TGGATTTCCATACACT | | GGATTTCCATACACT |
| 440 . . . 443 | AGAA | 169 | TGGAGCAATTCAACACACAGTGGT | 347 | TGGAGCAATTCAACACACAGagaaTGGT |
| | | | GGATTTCCATACACTG | | GGATTTCCATACACTG |
| 441 . . . 441 | G | 170 | GGAGCAATTCAACACACAGAAATG | 348 | GGAGCAATTCAACACACAGAgAATGGT |
| | | | GTGGATTTCCATACAC | | GGATTTCCATACAC |
| 445 . . . 449 | GGTGG | 171 | CAATTCAACACACAGAGAATATTT | 349 | CAATTCAACACACAGAGAATggtggATTT |
| | | | CCATACACTGAAATGA | | CCATACACTGAAATGA |
| 451 . . . 463 | TTTCC | 172 | AACACACAGAGAATGGTGGAAAA | 350 | AACACACAGAGAATGGTGGAtttccatacact |
| | ATACAC | | TGATTGTTCATCTTCCA | | gAAATGATTGTTCATCTTCCA |
| | TG (402) | | | | |

TABLE 5E
A list of exemplary mutant alleles obtained in the PMT4 gene.
PMT4

| | | Mutant | | Reference | |
|---|---|---|---|---|---|
| | Deleted | Allele | | Allele | |
| | sequence | Sequence | | Sequence | |
| | (SEQ | (SEQ ID | | (SEQ ID | |
| Position | ID No.) | No.) | Mutant Allele Sequence | No.) | Reference Allele Sequence |
| 131 . . . 131 | A | 173 | CGGCACTTCCAAACACCAAACG | 351 | CGGCACTTCCAAACACCAAAaCGGCCAC |
| | | | GCCACCATAATGGCACTT | | CATAATGGCACTT |
| 541 . . . 551 | ATTCAACA | 174 | TTTTGACTTTGGATGGAGCAAG | 352 | TTTTGACTTTGGATGGAGCAattcaacac |
| | CAC (403) | | GAGAATGTGGATTTCCAT | | acAGAGAATGGTGGATTTCCAT |
| 543 . . . 554 | TCAACACA | 175 | TTGACTTTGGATGGAGCAATGA | 353 | TTGACTTTGGATGGAGCAATtcaacacac |
| | CAGA (404) | | ATGGTGGATTTCCATACA | | agaGAATGGTGGATTTCCATACA |
| 544 . . . 550 | CAACACA | 176 | TGACTTTGGATGGAGCAATTCA | 354 | TGACTTTGGATGGAGCAATTcaacacaCAG |
| | | | GAGAATGGTGGATTTCCA | | AGAATGGTGGATTTCCA |
| 544 . . . 553 | CAACACAC | 177 | TGACTTTGGATGGAGCAATTAGA | 355 | TGACTTTGGATGGAGCAATTcaacacacagA |
| | AG (405) | | ATGGTGGATTTCCATAC | | GAATGGTGGATTTCCATAC |
| 545 . . . 555 | AACACACA | 178 | GACTTTGGATGGAGCAATTCAAT | 356 | GACTTTGGATGGAGCAATTCaacacacagag |
| | GAG (406) | | GGTGGATTTCCATACAC | | AATGGTGGATTTCCATACAC |
| 545 . . . 557 | AACAC | 179 | GACTTTGGATGGAGCAATTCTGG | 357 | GACTTTGGATGGAGCAATTCaacacac |
| | ACAGA | | TGGATTTCCATACACTG | | agagaaTGGTGGATTTCCATACACTG |
| | GAA (407) | | | | |
| 546 . . . 547 | AC | 180 | ACTTTGGATGGAGCAATTCAACA | 358 | ACTTTGGATGGAGCAATTCAacACACAG |
| | | | CAGAGAATGGTGGATTT | | AGAATGGTGGATTT |
| 546 . . . 549 | ACAC | 181 | ACTTTGGATGGAGCAATTCAACA | 359 | ACTTTGGATGGAGCAATTCAacacACAGA |
| | | | GAGAATGGTGGATTTCC | | GAATGGTGGATTTCC |
| 546 . . . 551 | ACACAC | 182 | ACTTTGGATGGAGCAATTCAAGA | 360 | ACTTTGGATGGAGCAATTCAacacacAGAG |
| | | | GAATGGTGGATTTCCAT | | AATGGTGGATTTCCAT |
| 546 . . . 552 | ACACACA | 183 | ACTTTGGATGGAGCAATTCAGAG | 361 | ACTTTGGATGGAGCAATTCAacacacaGAG |
| | | | AATGGTGGATTTCCATA | | AATGGTGGATTTCCATA |
| 546 . . . 553 | ACACACAG | 184 | ACTTTGGATGGAGCAATTCAAGA | 362 | ACTTTGGATGGAGCAATTCAacacacagAG |
| | | | ATGGTGGATTTCCATAC | | AATGGTGGATTTCCATAC |
| 547 . . . 550 | CACA | 185 | CTTTGGATGGAGCAATTCAACAG | 363 | CTTTGGATGGAGCAATTCAAcacaCAGAG |
| | | | AGAATGGTGGATTTCCA | | AATGGTGGATTTCCA |
| 547 . . . 551 | CACAC | 186 | CTTTGGATGGAGCAATTCAAAGA | 364 | CTTTGGATGGAGCAATTCAAcacacAGAG |
| | | | GAATGGTGGATTTCCAT | | AATGGTGGATTTCCAT |
| 548 . . . 549 | AC | 187 | TTTGGATGGAGCAATTCAACACA | 365 | TTTGGATGGAGCAATTCAACacACAGAG |
| | | | AGAGATGGTGGATTTCC | | AATGGTGGATTTCC |
| 548 . . . 551 | ACAC | 188 | TTTGGATGGAGCAATTCAACAGA | 366 | TTTGGATGGAGCAATTCAACacacAGAGA |
| | | | GAATGGTGGATTTCCAT | | ATGGTGGATTTCCAT |
| 548 . . . 552 | ACACA | 189 | TTTGGATGGAGCAATTCAACGAG | 367 | TTTGGATGGAGCAATTCAACacacaGAGA |
| | | | AATGGTGGATTTCCATA | | ATGGTGGATTTCCATA |
| 549 . . . 552 | CACA | 190 | TTGGATGGAGCAATTCAACAGAG | 368 | TTGGATGGAGCAATTCAACAcacaGAGAA |
| | | | AATGGTGGATTTCCATA | | TGGTGGATTTCCATA |
| 549 . . . 553 | CACAG | 191 | TTGGATGGAGCAATTCAACAAGA | 369 | TTGGATGGAGCAATTCAACAcacagAGAA |
| | | | ATGGTGGATTTCCATAC | | TGGTGGATTTCCATAC |
| 550 . . . 551 | AC | 192 | TGGATGGAGCAATTCAACACAGA | 370 | TGGATGGAGCAATTCAACACacAGAGAA |
| | | | GAATGGTGGATTTCCAT | | TGGTGGATTTCCAT |
| 550 . . . 552 | ACA | 193 | TGGATGGAGCAATTCAACACGAG | 371 | TGGATGGAGCAATTCAACACacaGAGAA |
| | | | AATGGTGGATTTCCATA | | TGGTGGATTTCCATA |
| 550 . . . 553 | ACAG | 194 | TGGATGGAGCAATTCAACACAGA | 372 | TGGATGGAGCAATTCAACACacagAGAAT |
| | | | ATGGTGGATTTCCATAC | | GGTGGATTTCCATAC |
| 550 . . . 556 | ACAGAGA | 195 | TGGATGGAGCAATTCAACACAT | 373 | TGGATGGAGCAATTCAACACacagagaATG |
| | | | GGTGGATTTCCATACACT | | GTGGATTTCCATACACT |
| 551 . . . 552 | CA | 196 | GGATGGAGCAATTCAACACAGA | 374 | GGATGGAGCAATTCAACACAcaGAGAAT |
| | | | GAATGGTGGATTTCCATA | | GGTGGATTTCCATA |
| 554 . . . 554 | A | 197 | TGGAGCAATTCAACACACAGGA | 375 | TGGAGCAATTCAACACACAGaGAATGGT |
| | | | ATGGTGGATTTCCATACA | | GGATTTCCATACA |
| 558 . . . 563 | TGGTGG | 198 | GCAATTCAACACACAGAGAAAT | 376 | GCAATTCAACACACAGAGAAtggtggATTT |
| | | | TTCCATACACTGAAATGA | | CCATACACTGAAATGA |
| 565 . . . 566 | TT | 199 | AACACACAGAGAATGGTGGATCC | 377 | AACACACAGAGAATGGTGGAttTCCATAC |
| | | | ATACACTGAAATGATTG | | ACTGAAATGATTG |
| 569 . . . 572 | CATA | 200 | CACAGAGAATGGTGGATTTCCAC | 378 | CACAGAGAATGGTGGATTTCcataCACTG |
| | | | TGAAATGATTGTTCATC | | AAATGATTGTTCATC |

### Example 3: Alkaloid Analysis of PMT Edited Lines

Genome edited tobacco plants along with controls are grown in 10″ pots in green house with 75 PPM fertilizer. At flowering stage, plants are topped and 2 weeks post topping lamina samples were collected from 3, 4, 5 leaves from top and alkaloid levels are measured (Tables 6A to 6C and 7) using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pH in Smokeless Tobacco Products, as published in the Federal Register Vol. 64, No. 55 Mar. 23, 1999 (and as amended in Vol. 74, No. 4, Jan. 7, 2009).

TABLE 6A
Alkaloid levels in PMT edited lines in K326 (shown here and Tables
6B, 6C, and 7 as weight percentage per gram leaf lamina (dry weight))

| | | % Alkaloids | | | | |
|---|---|---|---|---|---|---|
| Variety | Plant ID | Nicotine | Nornicotine | Anabasine | Anatabine | Total Alkaloids |
| K326 | 17GH1811 | 1.17 | 0.023 | 0.0043 | 0.03 | 1.23 |
| Control | 17GH1822 | 1.63 | 0.032 | 0.0058 | 0.04 | 1.71 |
| | 17GH1806 | 1.69 | 0.035 | 0.0058 | 0.04 | 1.76 |
| | 17GH1899 | 1.7893 | 0.0395 | 0.0089 | 0.0817 | 1.9194 |
| | 17GH1812 | 1.91 | 0.038 | 0.0072 | 0.05 | 2 |
| | 17GH1900 | 2.088 | 0.049 | 0.0103 | 0.0916 | 2.239 |
| | 17GH1821 | 2.16 | 0.043 | 0.0072 | 0.05 | 2.26 |
| | 17GH1896 | 2.6006 | 0.0573 | 0.0098 | 0.0682 | 2.7359 |
| K326 | 17GH1810 | 0.0013 | 0.019 | 0.0109 | 0.27 | 0.3 |
| Edited | 17GH1808 | 0.0044 | 0.014 | 0.0084 | 0.22 | 0.24 |
| | 17GH1901 | 0.006 | 0.0448 | 0.0238 | 0.6212 | 0.6958 |
| | 17GH1893 | 0.0072 | 0.0446 | 0.0265 | 0.6568 | 0.7351 |
| | 17GH1804 | 0.0078 | 0.028 | 0.0145 | 0.39 | 0.44 |
| | 17GH1902 | 0.008 | 0.037 | 0.0223 | 0.5572 | 0.6245 |
| | 18GH4 | 0.0102 | 0.0174 | 0.0102 | 0.231 | 0.2688 |
| | 17GH1892 | 0.0209 | 0.0131 | 0.0064 | 0.0877 | 0.1281 |

TABLE 6B
Alkaloid levels in PMT edited lines in TN90

| | | % Alkaloids | | | | |
|---|---|---|---|---|---|---|
| Variety | Plant ID | Nicotine | Nornicotine | Anabasine | Anatabine | Total Alkaloids |
| TN90 | 17GH1838 | 1.88 | 0.042 | 0.0063 | 0.06 | 1.98 |
| Control | 17GH1923 | 2.0868 | 0.0619 | 0.0066 | 0.0582 | 2.2136 |
| | 17GH1924 | 2.2099 | 0.0639 | 0.0071 | 0.0585 | 2.3394 |
| | 17GH1718 | 2.29 | 0.056 | 0.0078 | 0.07 | 2.42 |
| | 17GH1839 | 2.6 | 0.059 | 0.0083 | 0.08 | 2.74 |
| | 17GH1909 | 2.7639 | 0.0863 | 0.0107 | 0.082 | 2.9429 |
| | 17GH1910 | 2.9346 | 0.0872 | 0.0114 | 0.0951 | 3.1283 |
| TN90 | 17GH1699 | 0.0011 | 0.032 | 0.0182 | 0.53 | 0.58 |
| Edited | 17GH1708 | 0.0014 | 0.033 | 0.0181 | 0.51 | 0.56 |
| | 17GH1847 | 0.0016 | 0.031 | 0.019 | 0.55 | 0.6 |
| | 17GH1848 | 0.0018 | 0.023 | 0.0135 | 0.38 | 0.42 |
| | 17GH1724 | 0.0022 | 0.034 | 0.0186 | 0.53 | 0.59 |
| | 17GH1846 | 0.0022 | 0.019 | 0.0126 | 0.37 | 0.41 |
| | 17GH1722 | 0.0023 | 0.037 | 0.0196 | 0.56 | 0.62 |
| | 17GH1725 | 0.003 | 0.04 | 0.0219 | 0.63 | 0.69 |
| | 17GH1717 | 0.0035 | 0.033 | 0.023 | 0.64 | 0.7 |
| | 17GH1719 | 0.0042 | 0.042 | 0.0231 | 0.68 | 0.75 |
| | 17GH1845 | 0.0047 | 0.021 | 0.0129 | 0.41 | 0.45 |
| | 17GH1943 | 0.007 | 0.0133 | 0.0112 | 0.315 | 0.3464 |
| | 18GH47 | 0.0072 | 0.07 | 0.0424 | 0.926 | 1.0455 |
| | 17GH1944 | 0.0074 | 0.0171 | 0.0131 | 0.3654 | 0.403 |
| | 17GH1932 | 0.0074 | 0.0257 | 0.0151 | 0.4276 | 0.4758 |
| | 17GH1936 | 0.0074 | 0.0837 | 0.0447 | 1.3036 | 1.4394 |
| | 17GH1918 | 0.0075 | 0.0176 | 0.0142 | 0.4187 | 0.458 |
| | 17GH1912 | 0.0078 | 0.0223 | 0.0167 | 0.4766 | 0.5234 |
| | 18GH31 | 0.0079 | 0.0809 | 0.0411 | 0.9604 | 1.0902 |
| | 18GH28 | 0.008 | 0.0649 | 0.0291 | 0.7728 | 0.8748 |
| | 17GH1928 | 0.0081 | 0.0662 | 0.0369 | 0.9911 | 1.1024 |
| | 17GH1933 | 0.0083 | 0.0368 | 0.0214 | 0.5853 | 0.6517 |
| | 17GH1911 | 0.0088 | 0.0134 | 0.0102 | 0.2486 | 0.281 |

TABLE 6C
Alkaloid levels in PMT edited lines in Narrow Leaf Madole (NLM)

| | | % Alkaloids | | | | |
|---|---|---|---|---|---|---|
| | | | | | | Total |
| Variety | Plant ID | Nicotine | Nornicotine | Anabasine | Anatabine | Alkaloids |
| NLM Control | 18GH126 | 2.0844 | 0.0133 | 0.0084 | 0.0674 | 2.1734 |
| | 18GH7 | 3.3504 | 0.0173 | 0.016 | 0.1299 | 3.5136 |
| NLM | 18GH10 | 0.001 | 0.115 | 0.0392 | 0.99 | 1.14 |
| Edited | 18GH9 | 0.0012 | 0.066 | 0.0297 | 0.82 | 0.92 |
| | 18GH6 | 0.0019 | 0.135 | 0.0431 | 1.28 | 1.46 |
| | 18GH8 | 0.0022 | 0.166 | 0.0481 | 1.24 | 1.46 |
| | 17GH1905 | 0.0032 | 0.145 | 0.0417 | 1.3 | 1.49 |
| | 18GH5 | 0.0038 | 0.072 | 0.0262 | 0.82 | 0.92 |
| | 18GH130 | 0.0041 | 0.0816 | 0.0306 | 0.7593 | 0.8756 |
| | 18GH132 | 0.0044 | 0.0528 | 0.0228 | 0.5534 | 0.6335 |
| | 18GH79 | 0.0045 | 0.0604 | 0.0197 | 0.5336 | 0.6182 |
| | 18GH69 | 0.0069 | 0.062 | 0.0264 | 0.6542 | 0.7495 |
| | 18GH71 | 0.007 | 0.0723 | 0.027 | 0.6664 | 0.7726 |
| | 18GH131 | 0.0077 | 0.0383 | 0.0145 | 0.3684 | 0.4289 |
| | 18GH66 | 0.0081 | 0.059 | 0.0247 | 0.6034 | 0.6951 |
| | 18GH227 | 0.0086 | 0.0923 | 0.0268 | 0.7449 | 0.8726 |
| | 18GH78 | 0.0086 | 0.0546 | 0.0239 | 0.575 | 0.662 |
| | 18GH72 | 0.0087 | 0.0864 | 0.039 | 0.8708 | 1.0048 |
| | 18GH216 | 0.0089 | 0.1574 | 0.047 | 1.0626 | 1.2758 |
| | 18GH65 | 0.0094 | 0.0563 | 0.0259 | 0.6101 | 0.7018 |

TABLE 7
Relative changes in individual and total alkaloid levels in quintuple pmt knock-out
mutants in various varieties. Average percent levels of individual and total alkaloids are
calculated based on percent level data from individual lines as shown in Tables 6A to 6C.
Relative changes reflect the individual or total alkaloid level in a quintuple pmt mutant
relative to its control.

| | | | | | Total |
|---|---|---|---|---|---|
| | Nicotine | Nornicotine | Anabasine | Anatabine | Alkaloids |
| K326 Control | 1.880 | 0.040 | 0.007 | 0.056 | 1.982 |
| K326 quintuple pmt | 0.008 | 0.027 | 0.015 | 0.379 | 0.429 |
| mutant | | | | | |
| Relative change | 0.44% | 68.78% | 207.42% | 671.96% | 21.65% |
| TN90 Control | 2.395 | 0.065 | 0.008 | 0.072 | 2.538 |
| TN90 quintuple pmt | 0.005 | 0.037 | 0.022 | 0.590 | 0.655 |
| mutant | | | | | |
| Relative change | 0.22% | 57.15% | 259.69% | 820.44% | 25.80% |
| NLM Control | 2.717 | 0.015 | 0.012 | 0.099 | 2.844 |
| NLM quintuple pmt | 0.006 | 0.087 | 0.031 | 0.803 | 0.927 |
| mutant | | | | | |
| Relative change | 0.20% | 570.95% | 253.32% | 813.88% | 32.59% |

Briefly, approximately 0.5 g of tobacco is extracted using liquid/liquid extraction into an organic solvent containing an internal standard and analyzed by gas chromatography (GC) with flame ionization detection (FID). Results can be reported as weight percent (Wt %) on either an as is or dry weight basis. Reporting data on a dry weight basis requires an oven volatiles (OV) determination. Unless specified otherwise, total or individual alkaloid levels or nicotine levels shown herein are on a dry weight basis (e.g., percent total alkaloid or percent nicotine).

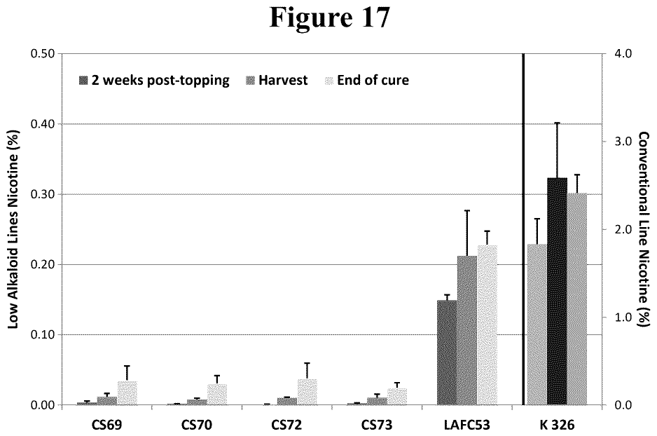

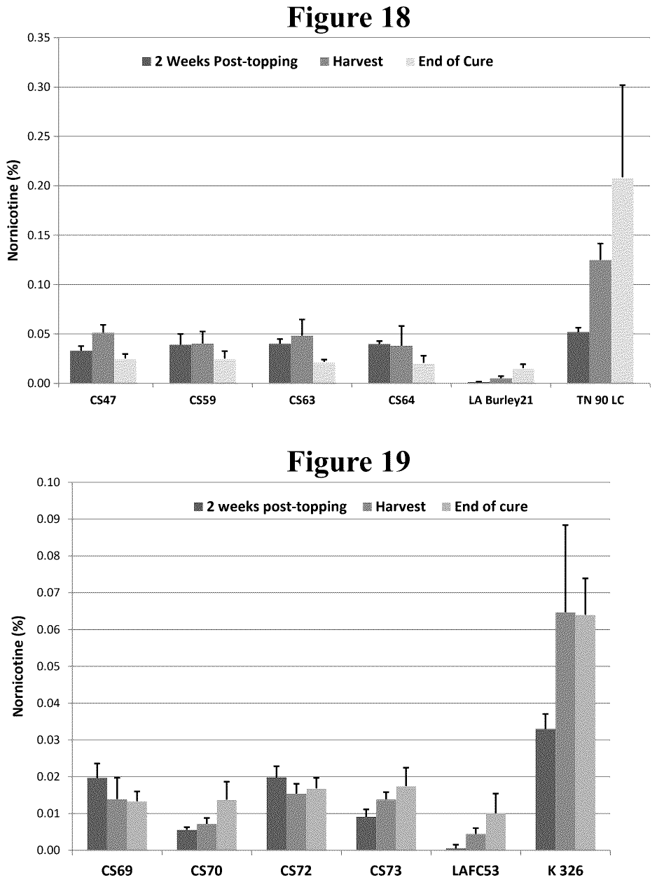

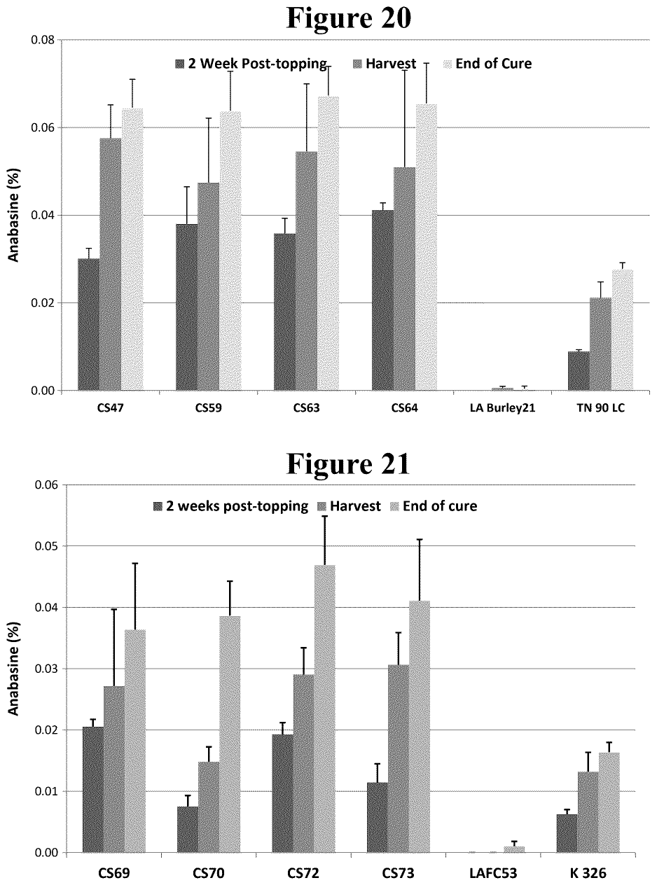

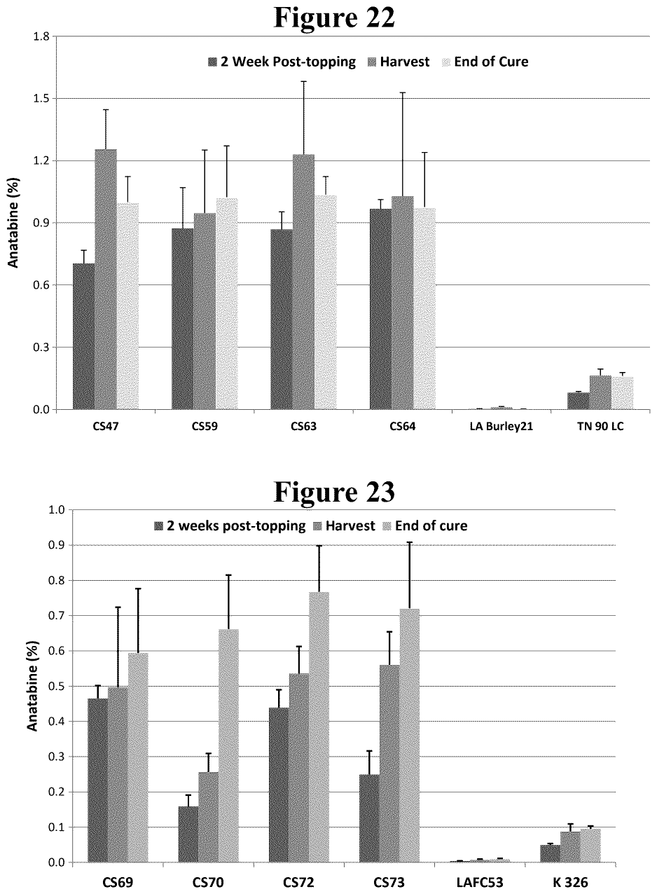

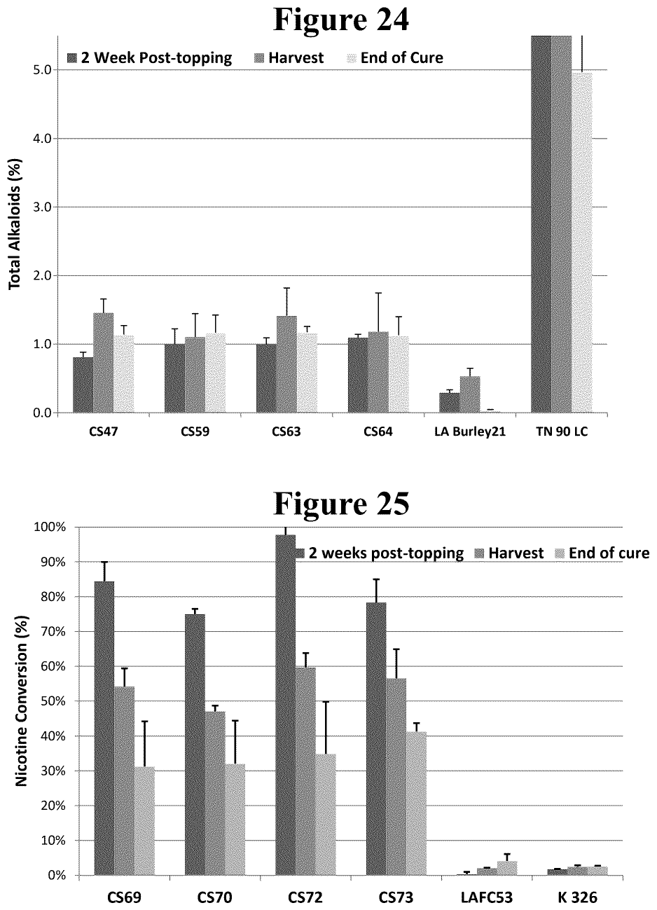

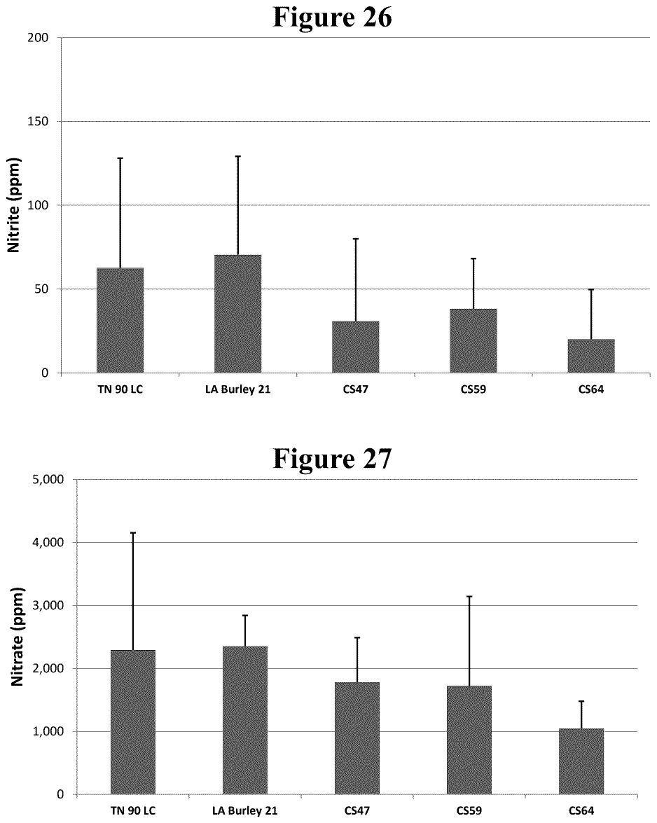

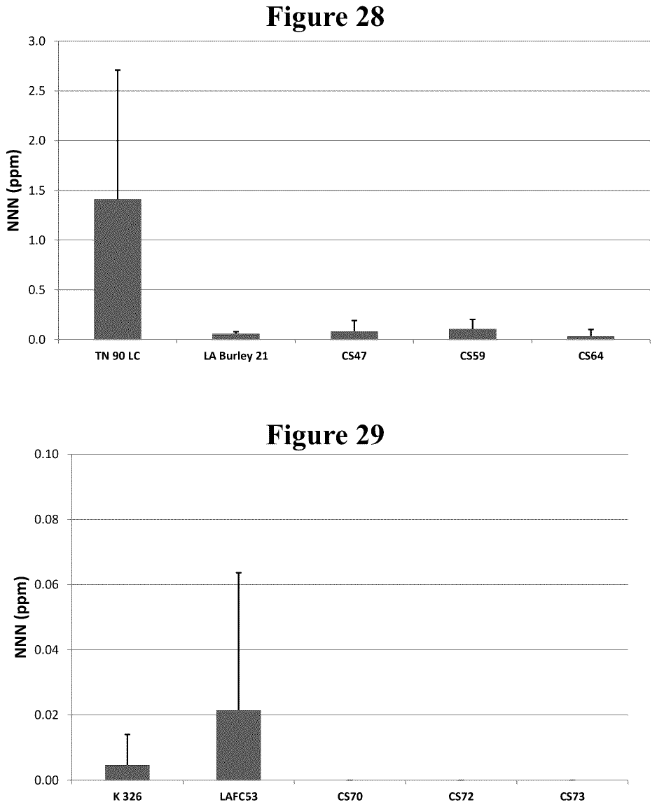

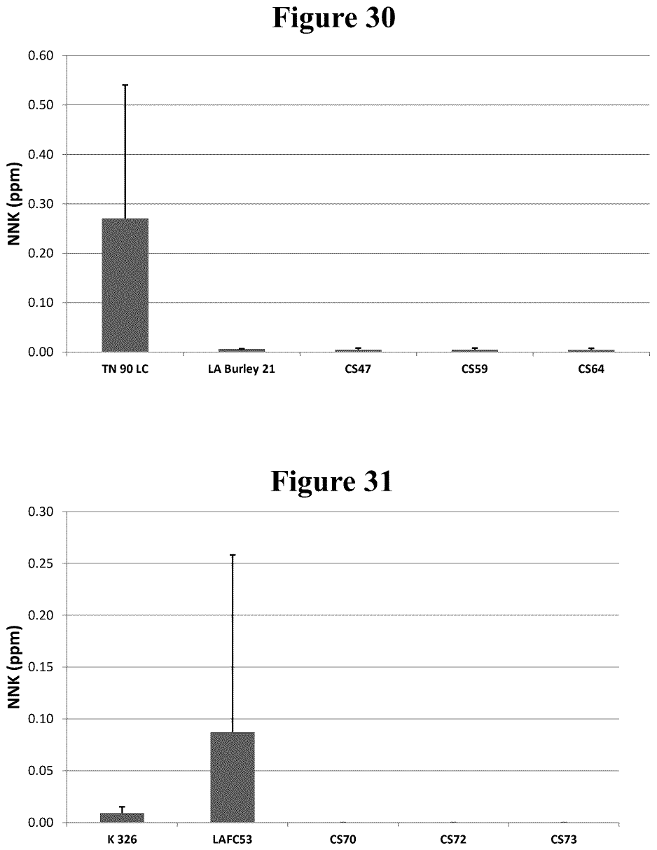

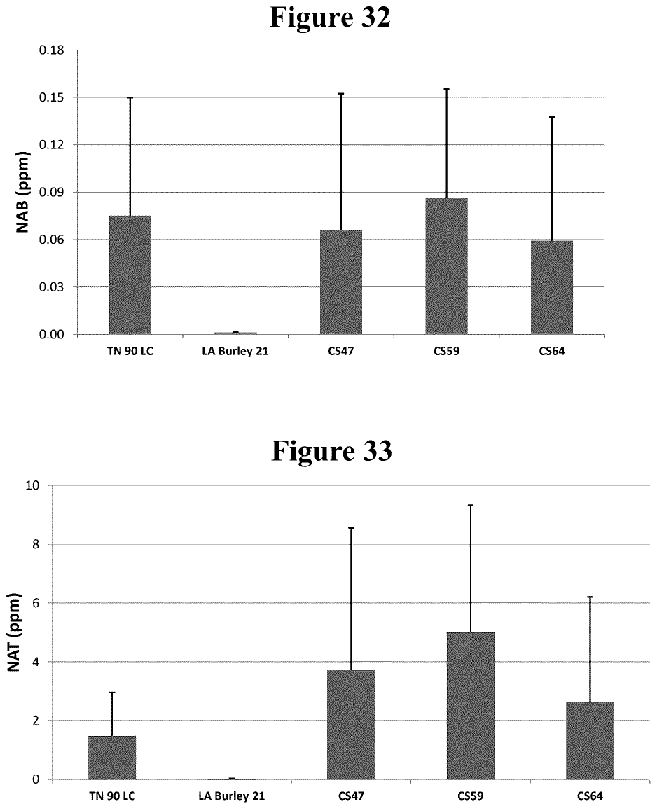

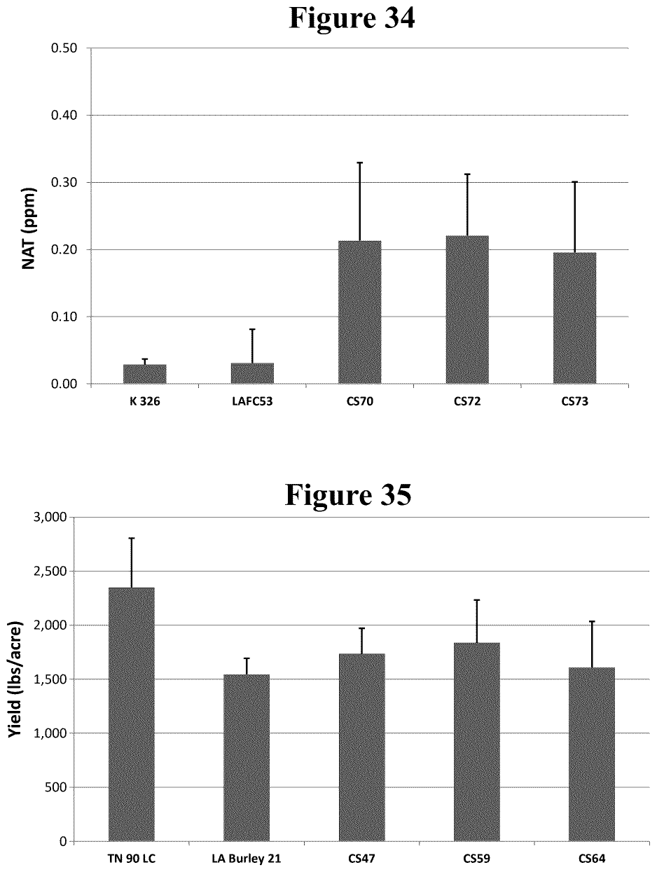

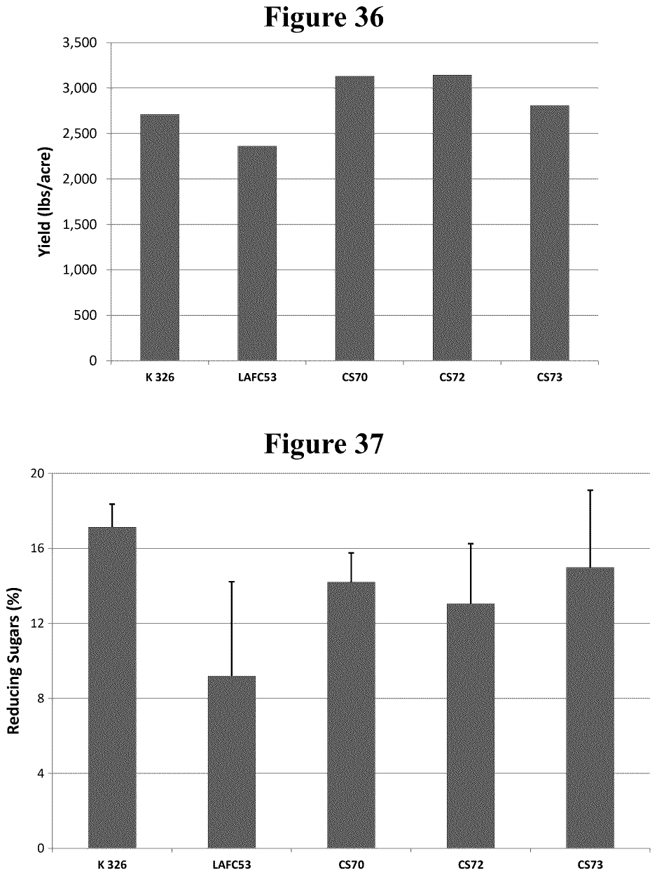

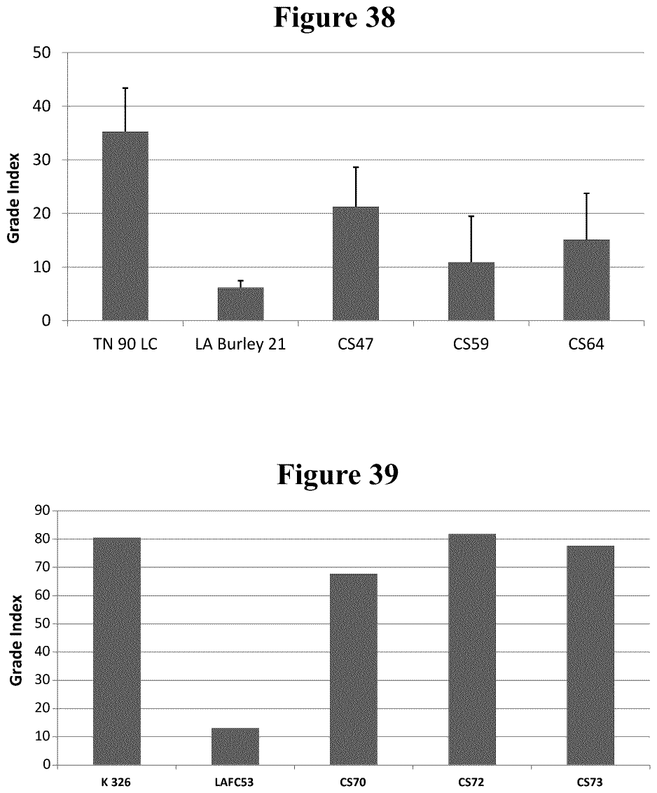

Plants are also planted in the field, harvested, and tested for alkaloids and TSNA levels in cured tobacco; leaf yield and leaf grade are also assessed for PMT edited plants. See FIGS. 16-39. In FIGS. 16-39, four biological replicates are averaged for each line, and error bars represent one standard deviation. Percentages shown are per gram of dried lamina tissue. Further, different mutant combinations of individual PMT genes are generated and tested (e.g., single, double, triple, or quadruple mutants).

### Example 4: Comparing a Quintuple Pmt Knock-Out Mutant with Other Low-Alkaloid Tobacco Plants

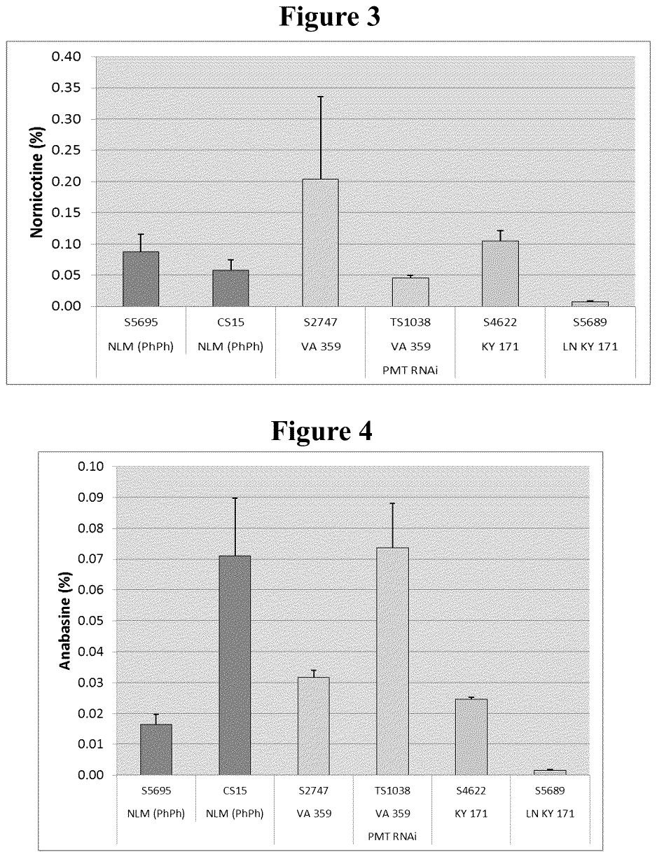

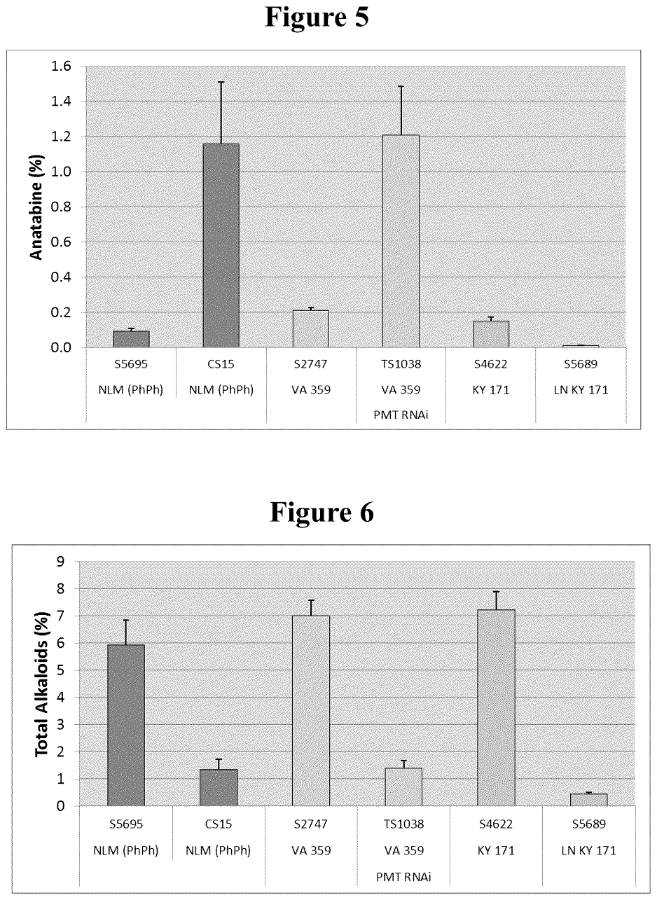

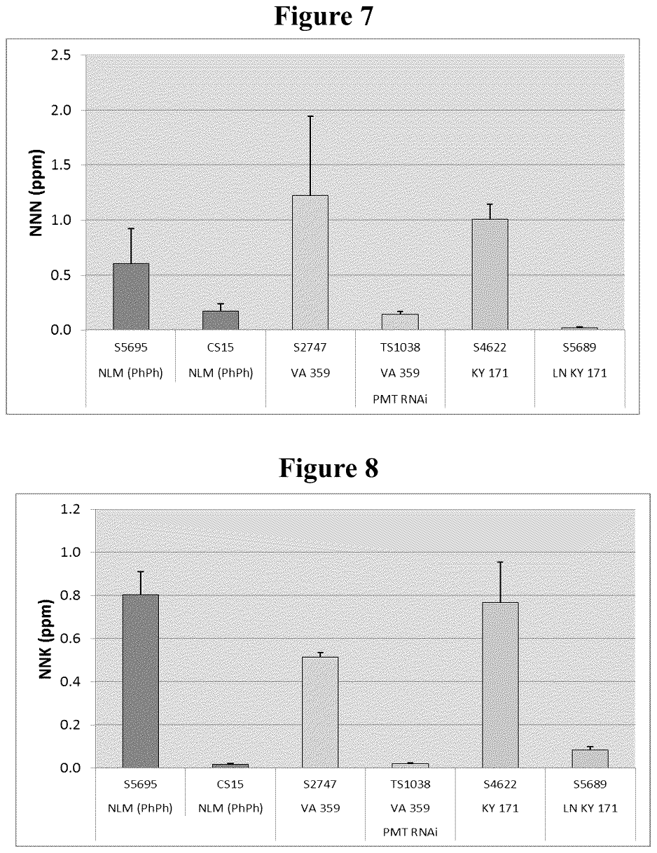

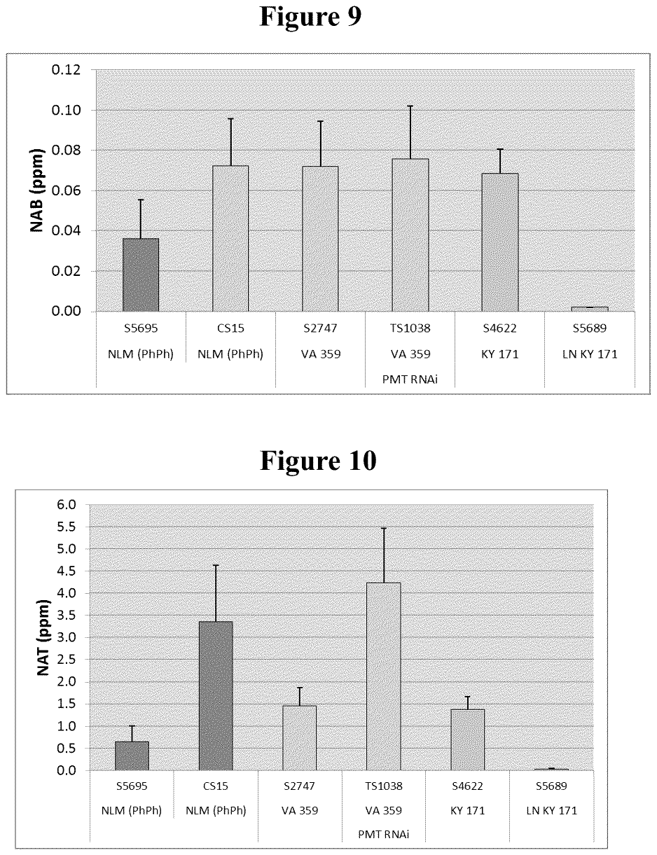

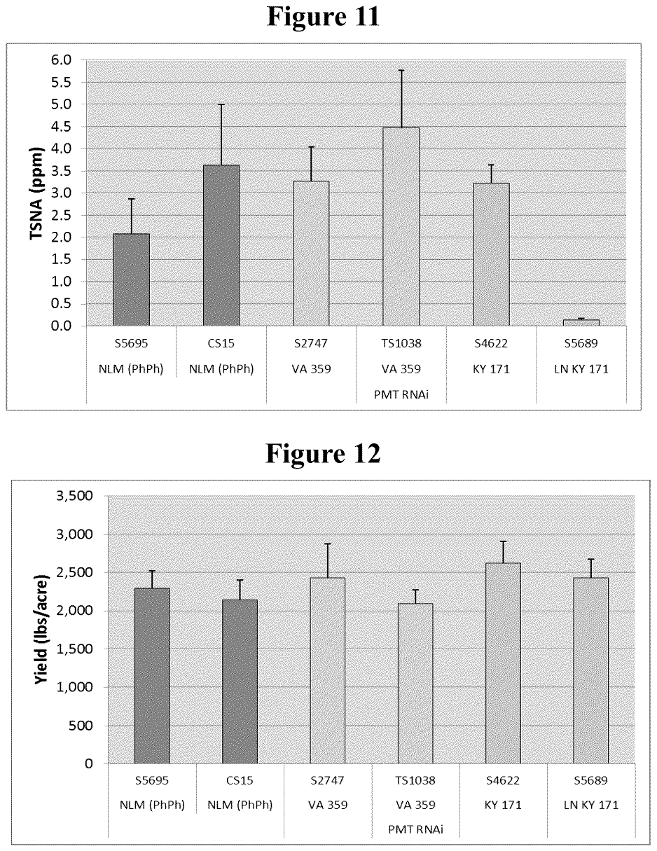

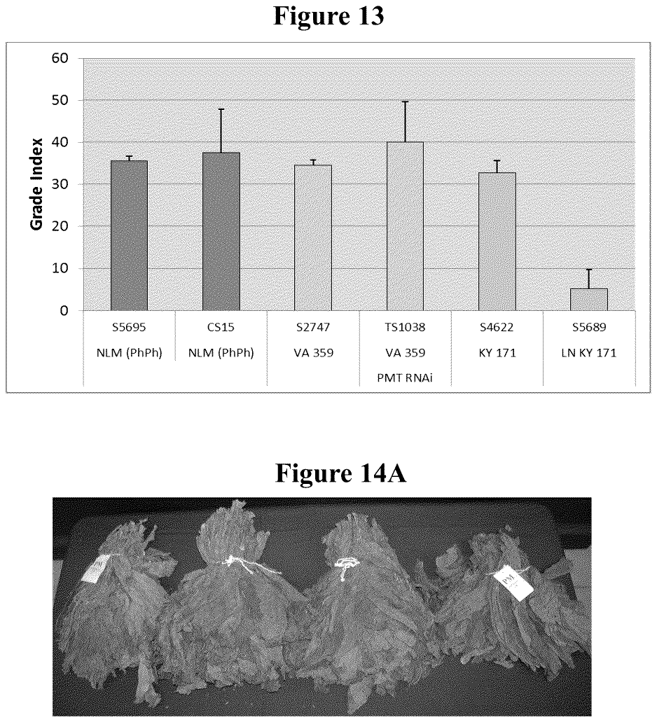

A quintuple pmt knock-out mutant line CS15 (see Table 4E for genotype, in the NLM (Ph Ph) background) is grown side by side with a PMT RNAi transgenic line (in the VA359 background, as described in US 2015/0322451) and a low-nicotine KY171 (“LN KY171”) variety (the KY 171 background harboring nic1 and nic2 double mutations). Leaves are harvested and cured via a dark fire curing method. Each line is analyzed for various individual and total alkaloid level, individual and total TSNA level, leaf yield, and leaf quality (FIGS. 2 to 13). The data shows that suppressing PMT gene activity by editing all five PMT genes reduces nicotine level without compromising leaf yield or quality.

### Example 5: Obtaining Tobacco Lines with Edited Mutant Alleles in One or More PMT Genes

Tobacco lines with mutations in individual PMT genes or selected combinations of PMT genes are obtained from the tobacco lines listed in Table 3. Crossing a quintuple, quadruple, triple, or double mutant (having mutations in five, four, three, or two PMT genes, respectively) to a non-mutated control line and selecting segregating progeny plants can be performed to obtain specific PMT mutation combinations. Tables 8A to 8E represents possible mutant combinations being obtained. Each mutated gene can be either homozygous or heterozygous for the mutation. Each of the mutant alleles listed in Tables 4A to 4E and Table 10 can be used to generate single, double, triple, quintuple, or quadruple mutants. Exemplary individual pmt mutant alleles are listed in Tables 12A to 12E.

### Example 6: Further Reduction of Total Alkaloids by Combining Pmt Mutations with Mutations in Other Genes

To further reduce total alkaloids and/or selected individual alkaloids, pmt mutants are combined with mutations in additional genes related to alkaloid biosynthesis in tobacco, such as quinolate phosphoribosyl transferase (QPT) or quinolinate synthase (QS). Briefly, gene editing is used to mutant selected QPT and/or QS genes in a desired pmt mutant background (e.g., a quadruple or quintuple pmt mutant). In the resulting combined qpt/pmt or qs/pmt mutants, alkaloids and TSNA levels are tested in cured tobacco. Both leaf yield and leaf grade are also assessed.

TABLE 8A
A list of mutants obtained with various genotypic combinations for
five PMT genes: single gene mutations

| | PMT1a | PMT1b | PMT2 | PMT3 | PMT4 |
|---|---|---|---|---|---|
| 1 | Mutant | WT | WT | WT | WT |
| 2 | WT | Mutant | WT | WT | WT |
| 3 | WT | WT | Mutant | WT | WT |
| 4 | WT | WT | WT | Mutant | WT |
| 5 | WT | WT | WT | WT | Mutant |

TABLE 8B
A list of mutants obtained with various genotypic combinations for
five PMT genes: double gene mutations

| | PMT1a | PMT1b | PMT2 | PMT3 | PMT4 |
|---|---|---|---|---|---|
| 1 | Mutant | Mutant | WT | WT | WT |
| 2 | Mutant | WT | Mutant | WT | WT |
| 3 | Mutant | WT | WT | Mutant | WT |
| 4 | Mutant | WT | WT | WT | Mutant |
| 5 | WT | Mutant | Mutant | WT | WT |
| 6 | WT | Mutant | WT | Mutant | WT |
| 7 | WT | Mutant | WT | WT | Mutant |
| 8 | WT | WT | Mutant | Mutant | WT |
| 9 | WT | WT | Mutant | WT | Mutant |
| 10 | WT | WT | WT | Mutant | Mutant |

TABLE 8C
A list of mutants obtained with various genotypic combinations for
five PMT genes: triple gene combinations

| | PMT1a | PMT1b | PMT2 | PMT3 | PMT4 |
|---|---|---|---|---|---|
| 1 | Mutant | Mutant | Mutant | WT | WT |
| 2 | Mutant | Mutant | WT | Mutant | WT |
| 3 | Mutant | Mutant | WT | WT | Mutant |
| 4 | Mutant | WT | Mutant | Mutant | WT |
| 5 | Mutant | WT | Mutant | WT | Mutant |
| 6 | Mutant | WT | WT | Mutant | Mutant |
| 7 | WT | Mutant | Mutant | Mutant | WT |
| 8 | WT | Mutant | Mutant | WT | Mutant |
| 9 | WT | WT | Mutant | Mutant | Mutant |
| 10 | WT | Mutant | Mutant | WT | Mutant |

TABLE 8D
A list of mutants obtained with various genotypic combinations for
five PMT genes: quadruple gene combinations

| | PMT1a | PMT1b | PMT2 | PMT3 | PMT4 |
|---|---|---|---|---|---|
| 1 | Mutant | Mutant | Mutant | Mutant | WT |
| 2 | WT | Mutant | Mutant | Mutant | Mutant |
| 3 | Mutant | WT | Mutant | Mutant | Mutant |
| 4 | Mutant | Mutant | WT | Mutant | Mutant |
| 5 | Mutant | Mutant | Mutant | WT | Mutant |

TABLE 8E
A list of mutants obtained with various genotypic combinations for
five PMT genes: quintuple gene combinations

| | PMT1a | PMT1b | PMT2 | PMT3 | PMT4 |
|---|---|---|---|---|---|
| 1 | Mutant | Mutant | Mutant | Mutant | Mutant |

### Example 7: PMT Genome Editing and Tobacco Line Development

Additional PMT knockout mutants are produced by editing all five PMT genes (PMT1a, PMT1b, PMT2, PMT3, and PMT4) in different tobacco lines. Tobacco protoplasts are transfected using polyethylene glycol (PEG) with plasmids encoding a a genome editing technology (GET2) protein and specific guide RNAs (gRNAs) targeting PMT genes at desired positions. Table 9 lists gRNA sequences used for PMT editing. Some gRNAs (e.g., Nos. 6 and 7) are pooled together for targeting multiple PMT genes in a single transfection.

TABLE 9
Guide RNAs for GET2 used in Example 7. “Y”
indicates that a gRNA is capable of targeting
that PMT gene, while “—” represents that a
gRNA does not target that PMT gene.

| gRNA Sequence | PMT1a | PMT1b | PMT2 | PMT3 | PMT4 |
|---|---|---|---|---|---|
| GATGGAGCAATTCAACATACAGA | Y | Y | — | — | — |
| (SEQ ID NO: 730) | | | | | |
| GATGGAGCAATTCAACACACAGA | — | — | Y | Y | Y |
| (SEQ ID NO: 731) | | | | | |

Transfected protoplasts are then immobilized in 1% agarose bead and subjected to tissue culture. When calli grow up to ~1 mm in diameter, they are spread on TOM2 plates. Calli are screened for insertions or deletions (indels) at the target positions using fragment analysis. Candidates, showing size shifts compared to wildtype control, are selected for further culture and the consequent shoots are tested by fragment analysis again to confirm the presence of indels. Rooted shoots are potted and sequenced for the target positions to determine the exact sequences deleted. Young leaf from each plant is harvested and PCR amplified for PMT fragments using phirekit. PMT Libraries for each line is indexed and 384 lines are pooled and sequenced using Miseq.

SNP analysis is carried out to determine both the exact edited pmt mutant allele sequences and the zygosity state at each PMT gene locus. Table 10 provides indels sequence information in each edited line of various tobacco varieties (e.g., Basma, K326, Katerini, TN90, Izmir).

TABLE 10
Mutant pint alleles in various lines produced by genome editing using GET2. The position of each
edited site (e.g., indels) is relative to the nucleotide number on the corresponding cDNA sequence
of each PMT gene (e.g., SEQ ID NO: 7 for PMT1a; SEQ ID NO: 6 for PMT1b;
SEQ ID NO: 8 for PMT2; SEQ ID NO: 9 for PMT3; SEQ ID NO: 10 for PMT4). SEQ ID Numbers are
assigned and shown for sequences of more than 10 nucleotides.

| | | PMT1a | | PMT1b | | PMT2 | | PMT3 | | PMT4 | |
|---|---|---|---|---|---|---|---|---|---|---|---|
| | | Deleted | | Deleted | | Deleted | | Deleted | | Deleted | |
| Geno- | | Se- | | Se- | | Se- | | Se- | | Se- | Posi- |
| type | Line | quence | Position | quence | Position | quence | Position | quence | Position | quence | tion |
| BASMA | CS107 | CAACAT | 412 . . . | ACAT | 414 . . . | AC | 348 . . . | ACAC | 432 . . . | ACAC | 546 |
| | | A | 418 | | 417 | | 349 | | 435 | | . . . |
| | | | | | | | | | | | 549 |
| BASMA | CS106 | ACAT | 414 . . . | ACAT | 414 . . . | AC | 348 . . . | ACAC | 432 . . . | ACAC | 546 |
| | | | 417 | | 417 | | 349 | | 435 | | . . . |
| | | | | | | | | | | | 549 |
| K326 | CS115 | TCAAC | 411 . . . | ACAT | 414 . . . | AC | 348 . . . | CAC | 433 . . . | ACACA | 548 |
| | | ATACA | 420 | | 417 | | 349 | AC | 437 | | . . . |
| | | (SEQ | | | | | | | | | 552 |
| | | ID NO: | | | | | | | | | |
| | | 379) | | | | | | | | | |
| K326 | 18GH | ACAT | 414 . . . | ACAT | 414 . . . | ACACAC | 348 . . . | AC | 432 . . . | ACACA | 548 |
| | 2162 | | 417 | | 417 | AG | 355 | | 433 | | . . . |
| | | | | | | | | | | | 552 |
| K326 | CS111 | ACAT | 414 . . . | ACAT | 414 . . . | AC | 348 . . . | ACAC | 432 . . . | CACAC | 547 |
| | | | 417 | | 417 | | 349 | | 435 | | . . . |
| | | | | | | | | | | | 551 |
| K326 | CS112 | CATACA | 415 . . . | AC | 418 . . . | AC | 348 . . . | ACAC | 432 . . . | CACAC | 547 |
| | | G | 421 | | 419 | | 349 | | 435 | | . . . |
| | | | | | | | | | | | 551 |
| K326 | 17GH | CATACA | 415 . . . | AC | 418 . . . | AC | 348 . . . | ACAC | 432 . . . | ACAC | 546 |
| | 1678- | G | 421 | | 419 | | 349 | | 435 | | . . . |
| | 60 | | | | | | | | | | 549 |
| K326 | CS131 | ACAT | 414 . . . | ACAT | 414 . . . | ACAC | 348 . . . | AACAC | 431 . . . | ACAC | 546 |
| | | | 417 | | 417 | | 351 | ACAG | 439 | | . . . |
| | | | | | | | | | | | 549 |
| KATERINI | CS164 | AT | 416 . . . | CAACAT | 412 . . . | AC | 348 . . . | ACAC | 432 . . . | AC | 546 |
| | | | 417 | A | 418 | | 349 | | 435 | | . . . |
| | | | | | | | | | | | 547 |
| KATERINI | CS163 | ACAT | 414 . . . | AT | 416 . . . | AC | 348 . . . | ACAC | 432 . . . | AC | 546 |
| | | | 417 | | 417 | | 349 | | 435 | | . . . |
| | | | | | | | | | | | 547 |
| KATERINI | CS146 | GAGCAA | 404 . . . | ACAT | 414 . . . | AC | 348 . . . | CAAC | 430 . . . | AC | 546 |
| | | TTCAACA | 422 | | 417 | | 349 | ACA | 436 | | . . . |
| | | TACAGA | | | | | | | | | 547 |
| | | (SEQ ID | | | | | | | | | |
| | | NO: 408) | | | | | | | | | |
| KATERINI | CS147 | ACAT | 414 . . . | CAACAT | 412 . . . | AC | 348 . . . | CAAC | 430 . . . | AC | 546 |
| | | | 417 | A | 418 | | 349 | ACA | 436 | | . . . |
| | | | | | | | | | | | 547 |
| KATERINI | CS150 | AT | 416 . . . | ACAT | 414 . . . | AC | 348 . . . | CACA | 433 . . . | AC | 546 |
| | | | 417 | | 417 | | 349 | CAG | 439 | | . . . |
| | | | | | | | | | | | 547 |
| KATERINI | CS151 | AT | 416 . . . | ACAT | 414 . . . | AC | 348 . . . | ACAC | 432 . . . | CAACAC | 544 |
| | | | 417 | | 417 | | 349 | | 435 | ACAG | . . . |
| | | | | | | | | | | (SEQ ID | 553 |
| | | | | | | | | | | NO: 390) | |
| KATERINI | CS148 | ACAT | 414 . . . | ACAT | 414 . . . | AC | 348 . . . | CACA | 433 . . . | AC | 546 |
| | | | 417 | | 417 | | 349 | CAG | 439 | | . . . |
| | | | | | | | | | | | 547 |
| KATERINI | CS149 | ACAT | 414 . . . | ACAT | 414 . . . | AC | 348 . . . | ACAC | 432 . . . | CAACAC | 544 |
| | | | 417 | | 417 | | 349 | | 435 | ACAG | . . . |
| | | | | | | | | | | (SEQ ID | 553 |
| | | | | | | | | | | NO: 390) | |
| KATERINI | CS152 | AC | 418 . . . | ACAT | 414 . . . | AC | 348 . . . | ACAC | 432 . . . | ACAC | 546 |
| | | | 419 | | 417 | | 349 | | 435 | | . . . |
| | | | | | | | | | | | 549 |
| KATERINI | CS153 | CAACAT | 412 . . . | AC | 414 . . . | AC | 348 . . . | ACAC | 432 . . . | ACAC | 546 |
| | | A | 418 | | 415 | | 349 | | 435 | | . . . |
| | | | | | | | | | | | 549 |
| KATERINI | CS102 | ACAT | 414 . . . | AACAT | 413 . . . | ACACAC | 348 . . . | AC | 432 . . . | AC | 546 |
| | | | 417 | | 417 | AG | 355 | | 433 | | . . . |
| | | | | | | | | | | | 547 |
| KATERINI | CS103 | AC | 418 . . . | CAACAT | 412 . . . | ACACAC | 348 . . . | AC | 432 . . . | AC | 546 |
| | | | 419 | A | 418 | AG | 355 | | 433 | | . . . |
| | | | | | | | | | | | 547 |
| TN90 | CS143 | TACA | 417 . . . | ACAT | 414 . . . | AC | 348 . . . | AC | 432 . . . | ACAC | 546 |
| | | GAG | 423 | | 417 | | 349 | | 433 | | . . . |
| | | | | | | | | | | | 549 |
| TN90 | 18GH | AC | 418 . . . | ACAT | 414 . . . | ACAC | 348 . . . | ACAC | 432 . . . | AC | 546 |
| | 2169 | | 419 | | 417 | | 351 | | 435 | | . . . |
| | | | | | | | | | | | 547 |
| TN90 | CS120 | ACAT | 414 . . . | ACAG | 418 . . . | ACAC | 348 . . . | AC | 432 . . . | AC | 546 |
| | | | 417 | | 421 | | 351 | | 433 | | . . . |
| | | | | | | | | | | | 547 |
| TN90 | 17GH | ACAT | 414 . . . | ACAT | 414 . . . | AC | 348 . . . | ACAG | 436 . . . | AC | 546 |
| | 1698- | | 417 | | 417 | | 349 | | 439 | | . . . |
| | 22 | | | | | | | | | | 547 |
| TN90 | 17GH | ACAT | 414 . . . | AACAT | 413 . . . | AC | 348 . . . | AC | 432 . . . | ACAC | 546 |
| | 1700- | | 417 | | 417 | | 349 | | 433 | | . . . |
| | 13 | | | | | | | | | | 549 |
| TN90 | 17GH | ACAT | 414 . . . | ACAT | 414 . . . | AC | 348 . . . | AC | 432 . . . | CACAG | 549 |
| | 1702- | | 417 | | 417 | | 349 | | 433 | | . . . |
| | 17 | | | | | | | | | | 553 |
| TN90 | 18GH | ACAT | 414 . . . | ACAT | 414 . . . | ACAC | 348 . . . | CAAC | 430 . . . | AC | 546 |
| | 2171 | | 417 | | 417 | | 351 | ACA | 436 | | . . . |
| | | | | | | | | | | | 547 |
| TN90 | CS165 | ACAT | 414 . . . | ACAT | 414 . . . | ACAC | 348 . . . | ACAC | 432 . . . | ACAC | 546 |
| | | | 417 | | 417 | | 351 | | 435 | | . . . |
| | | | | | | | | | | | 549 |
| TN90 | CS118 | ACAT | 414 . . . | ACAT | 414 . . . | AC | 348 . . . | AC | 432 . . . | ACAC | 546 |
| | | | 417 | | 417 | | 349 | | 433 | | . . . |
| | | | | | | | | | | | 549 |
| TN90 | CS133 | GGAGCA | 403 . . . | CAACAT | 412 . . . | ACAC | 348 . . . | AC | 432 . . . | AC | 546 |
| | | ATTCAAC | 415 | ACAG | 421 | | 351 | | 433 | | . . . |
| | | (SEQ ID | | (SEQ | | | | | | | 547 |
| | | NO: 409) | | ID NO: | | | | | | | |
| | | | | 380) | | | | | | | |
| TN90 | 17GH | CA | 415 . . . | ACAT | 414 . . . | ACAC | 348 . . . | AC | 432 . . . | AC | 546 |
| | 1737- | | 416 | | 417 | | 351 | | 433 | | . . . |
| | 24 | | | | | | | | | | 547 |
| IZMIR | 18GH | CAAC | 412 . . . | ATAGAG | 416 . . . | ACAC | 348 . . . | ACAC | 432 . . . | ACACAC | 546 |
| | 2254- | ATA | 418 | AA | 417 | | 351 | | 435 | AG | . . . |
| | 7 | | | | & | | | | | | 553 |
| | | | | | 420 . . . | | | | | | |
| | | | | | 425 | | | | | | |

Table 11 provides the length (in nucleotides) of each PMT indel for each gene in each line as provided in Table 10.

TABLE 11
The length (in nucleotides) of each indel for selected lines provided in Table 10.

| Genotype | Line | Seed Ids | PMT1a | PMT1b | PMT2 | PMT3 | PMT4 |
|---|---|---|---|---|---|---|---|
| BASMA | CS107 | CS107 | 7 | 4 | 2 | 4 | 4 |
| BASMA | CS106 | CS106 | 4 | 4 | 2 | 4 | 4 |
| K326 | CS115 | CS115 | 10 | 4 | 2 | 5 | 5 |
| K326 | 17GH1809-13 | 18GH2162 | 4 | 4 | 8 | 2 | 5 |
| K326 | CS111 | CS111 | 4 | 4 | 2 | 4 | 5 |
| K326 | CS112 | CS112 | 7 | 2 | 2 | 4 | 5 |
| K326 | 17GH1678-60 | 17GH1678-60 | 7 | 2 | 2 | 4 | 4 |
| K326 | CS131 | CS131 | 4 | 4 | 4 | 9 | 4 |
| KATERINI | 18GH709-01 | CS164 | 2 | 7 | 2 | 4 | 2 |
| KATERINI | 18GH709-08 | CS163 | 4 | 2 | 2 | 4 | 2 |
| KATERINI | 18GH414-11 | CS146 | 19 | 4 | 2 | 7 | 2 |
| KATERINI | 18GH414-19 | CS147 | 4 | 7 | 2 | 7 | 2 |
| KATERINI | 18GH437-04 | CS150 | 2 | 4 | 2 | 7 | 2 |
| KATERINI | 18GH437-08 | CS151 | 2 | 4 | 2 | 4 | 10 |
| KATERINI | 18GH437-32 | CS148 | 4 | 4 | 2 | 7 | 2 |
| KATERINI | 18GH437-39 | CS149 | 4 | 4 | 2 | 4 | 10 |
| KATERINI | 18GH449-26 | CS152 | 2 | 4 | 2 | 4 | 4 |
| KATERINI | 18GH449-33 | CS153 | 7 | 2 | 2 | 4 | 4 |
| KATERINI | 18GH125-48 | CS162 | 2 | 7 | 8 | 2 | 2 |
| KATERINI | CS102 | CS102 | 4 | 5 | 8 | 2 | 2 |
| KATERINI | CS103 | CS103 | 2 | 7 | 8 | 2 | 2 |
| TN90 | 17GH1719-30 | CS143 | 7 | 4 | 2 | 2 | 4 |
| TN90 | 17GH1740-36 | 18GH2169 | 2 | 4 | 4 | 4 | 2 |
| TN90 | 17GH1698-22 | 17GH1698-22 | 4 | 4 | 2 | 4 | 2 |
| TN90 | 17GH1700-13 | 17GH1700-13 | 4 | 5 | 2 | 2 | 4 |
| TN90 | 17GH1702-17 | 17GH1702-17 | 4 | 4 | 2 | 2 | 5 |
| TN90 | 17GH1849-01 | 18GH2171 | 4 | 4 | 4 | 7 | 2 |
| TN90 | 17GH1849-48 | CS165 | 4 | 4 | 4 | 4 | 4 |
| TN90 | 17GH1737-24 | 17GH1737-24 | 2 | 4 | 4 | 2 | 2 |
| TN90 | CS118 | CS118 | 4 | 4 | 2 | 2 | 4 |
| TN90 | CS133 | CS133 | 13 | 10 | 4 | 2 | 2 |
| TN90 | CS120 | CS120 | 4 | 4 | 4 | 2 | 2 |
| IZMIR | 18GH1108-07 | 18GH2254-7 | 7 | 8 | 4 | 4 | 8 |

Tables 12A to 12E provide genomic sequences of approximately 90 nucleotides from each pmt mutant allele with the edited site in the middle of the genomic sequence (e.g., 45 nucleotides on each side of the deleted or inserted sequence site).

TABLE 12A
A list of exemplary mutant alleles obtained in the PMT1a gene. Mutant allele sequences listed here represent approximately 90-nucleotide-
long genomic sequences from each edited PMT1a gene with the edited site in the middle of the genomic sequence (e.g., 45 nucleotides on each side of the
deleted sequence site). The mutant allele corresponds to the indel provided for each line in Table 10. The lowercase letters in the reference allele sequence
(SEQ ID NO: 6) denote which nucleotides are deleted in the mutant allele.

| | | | Mutant | | Reference |
|---|---|---|---|---|---|
| | | | Allele | | Allele |
| | | | SEQ ID | | SEQ ID |
| Genotype | Line | Mutant Allele Sequence | NO. | Reference Allele Sequence | NO. |
| BASMA | CS107 | TCAGCAACTTATGGGAAGGTTCTG | 410 | TCAGCAACTTATGGGAAGGTTCTGAC | 442 |
| | | ACTTTGGATGGAGCAATTCAGAG | | TTTGGATGGAGCAATTCAacatacaGAG | |
| | | AATGGTGGATTTCCATACACTGAA | | AATGGTGGATTTCCATACACTGAAAT | |
| | | ATGATTGTTCATCTA | | GATTGTTCATCTA | |
| BASMA | CS106 | TCAGCAACTTATGGGAAGGTTCTG | 411 | TCAGCAACTTATGGGAAGGTTCTGAC | 443 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| K326 | CS115 | TCAGCAACTTATGGGAAGGTTCTG | 412 | TCAGCAACTTATGGGAAGGTTCTGAC | 444 |
| | | ACTTTGGATGGAGCAATGAGAAT | | TTTGGATGGAGCAATtcaacatacaGAGA | |
| | | GGTGGATTTCCATACACTGAAATG | | ATGGTGGATTTCCATACACTGAAATG | |
| | | ATTGTTCATCTA | | ATTGTTCATCTA | |
| K326 | 18GH2162 | TCAGCAACTTATGGGAAGGTTCTG | 413 | TCAGCAACTTATGGGAAGGTTCTGAC | 445 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| K326 | CS111 | TCAGCAACTTATGGGAAGGTTCTG | 414 | TCAGCAACTTATGGGAAGGTTCTGAC | 446 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| K326 | CS112 | TCAGCAACTTATGGGAAGGTTCTG | 415 | TCAGCAACTTATGGGAAGGTTCTGAC | 447 |
| | | ACTTTGGATGGAGCAATTCAAAG | | TTTGGATGGAGCAATTCAAcatacagAG | |
| | | AATGGTGGATTTCCATACACTGAA | | AATGGTGGATTTCCATACACTGAAAT | |
| | | ATGATTGTTCATCTA | | GATTGTTCATCTATCAAAGAATGG | |
| K326 | 17GH1678- | TCAGCAACTTATGGGAAGGTTCTG | 416 | TCAGCAACTTATGGGAAGGTTCTGAC | 448 |
| | 60 | ACTTTGGATGGAGCAATTCAAAG | | TTTGGATGGAGCAATTCAAcatacagAG | |
| | | AATGGTGGATTTCCATACACTGAA | | AATGGTGGATTTCCATACACTGAAAT | |
| | | ATGATTGTTCATCTA | | GATTGTTCATCTATCAAAGAATGG | |
| K326 | CS131 | TCAGCAACTTATGGGAAGGTTCTG | 417 | TCAGCAACTTATGGGAAGGTTCTGAC | 449 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS164 | TCAGCAACTTATGGGAAGGTTCTG | 418 | TCAGCAACTTATGGGAAGGTTCTGAC | 450 |
| | | ACTTTGGATGGAGCAATTCAACA | | TTTGGATGGAGCAATTCAACatACAG | |
| | | CAGAGAATGGTGGATTTCCATAC | | AGAATGGTGGATTTCCATACACTGAA | |
| | | ACTGAAATGATTGTTCATCTA | | ATGATTGTTCATCTA | |
| KATERINI | CS163 | TCAGCAACTTATGGGAAGGTTCTG | 419 | TCAGCAACTTATGGGAAGGTTCTGAC | 451 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS146 | TCAGCAACTTATGGGAAGGTTCTG | 420 | TCAGCAACTTATGGGAAGGTTCTGAC | 452 |
| | | ACTTTGGATGGAATGGTGGATTTC | | TTTGGATGGAgcaattcaacatacaga | |
| | | CATACACTGAAATGATTGTTCATC | | gaATGGTGGATTTCCATACACTGAAAT | |
| | | TA | | GATTGTTCATCTA | |
| KATERINI | CS147 | TCAGCAACTTATGGGAAGGTTCTG | 421 | TCAGCAACTTATGGGAAGGTTCTGAC | 453 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS150 | TCAGCAACTTATGGGAAGGTTCTG | 422 | TCAGCAACTTATGGGAAGGTTCTGAC | 454 |
| | | ACTTTGGATGGAGCAATTCAACA | | TTTGGATGGAGCAATTCAACatACAG | |
| | | CAGAGAATGGTGGATTTCCATAC | | AGAATGGTGGATTTCCATACACTGAA | |
| | | ACTGAAATGATTGTTCATCTA | | ATGATTGTTCATCTA | |
| KATERINI | CS151 | TCAGCAACTTATGGGAAGGTTCTG | 423 | TCAGCAACTTATGGGAAGGTTCTGAC | 455 |
| | | ACTTTGGATGGAGCAATTCAACA | | TTTGGATGGAGCAATTCAACatACAG | |
| | | CAGAGAATGGTGGATTTCCATAC | | AGAATGGTGGATTTCCATACACTGAA | |
| | | ACTGAAATGATTGTTCATCTA | | ATGATTGTTCATCTA | |
| KATERINI | CS148 | TCAGCAACTTATGGGAAGGTTCTG | 424 | TCAGCAACTTATGGGAAGGTTCTGAC | 456 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS149 | TCAGCAACTTATGGGAAGGTTCTG | 425 | TCAGCAACTTATGGGAAGGTTCTGAC | 457 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS152 | TCAGCAACTTATGGGAAGGTTCTG | 426 | TCAGCAACTTATGGGAAGGTTCTGAC | 458 |
| | | ACTTTGGATGGAGCAATTCAACAT | | TTTGGATGGAGCAATTCAACATacAG | |
| | | AGAGAATGGTGGATTTCCATACA | | AGAATGGTGGATTTCCATACACTGAA | |
| | | CTGAAATGATTGTTCATCTA | | ATGATTGTTCATCTA | |
| KATERINI | CS153 | TCAGCAACTTATGGGAAGGTTCTG | 427 | TCAGCAACTTATGGGAAGGTTCTGAC | 459 |
| | | ACTTTGGATGGAGCAATTCAGAG | | TTTGGATGGAGCAATTCAacatacaGAG | |
| | | AATGGTGGATTTCCATACACTGAA | | AATGGTGGATTTCCATACACTGAAAT | |
| | | ATGATTGTTCATCTA | | GATTGTTCATCTA | |
| KATERINI | CS102 | TCAGCAACTTATGGGAAGGTTCTG | 428 | TCAGCAACTTATGGGAAGGTTCTGAC | 460 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| KATERINI | CS103 | TCAGCAACTTATGGGAAGGTTCTG | 429 | TCAGCAACTTATGGGAAGGTTCTGAC | 461 |
| | | ACTTTGGATGGAGCAATTCAACAT | | TTTGGATGGAGCAATTCAACATacAG | |
| | | AGAGAATGGTGGATTTCCATACA | | AGAATGGTGGATTTCCATACACTGAA | |
| | | CTGAAATGATTGTTCATCTA | | ATGATTGTTCATCTA | |
| TN90 | CS143 | TCAGCAACTTATGGGAAGGTTCTG | 430 | TCAGCAACTTATGGGAAGGTTCTGAC | 462 |
| | | ACTTTGGATGGAGCAATTCAACA | | TTTGGATGGAGCAATTCAACAtacagag | |
| | | AATGGTGGATTTCCATACACTGAA | | AATGGTGGATTTCCATACACTGAAAT | |
| | | ATGATTGTTCATCTA | | GATTGTTCATCTA | |
| TN90 | 18GH2169 | TCAGCAACTTATGGGAAGGTTCTG | 431 | TCAGCAACTTATGGGAAGGTTCTGAC | 463 |
| | | ACTTTGGATGGAGCAATTCAACAT | | TTTGGATGGAGCAATTCAACATacAG | |
| | | AGAGAATGGTGGATTTCCATACA | | AGAATGGTGGATTTCCATACACTGAA | |
| | | CTGAAATGATTGTTCATCTA | | ATGATTGTTCATCTA | |
| TN90 | CS120 | TCAGCAACTTATGGGAAGGTTCTG | 432 | TCAGCAACTTATGGGAAGGTTCTGAC | 464 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | 17GH1698- | TCAGCAACTTATGGGAAGGTTCTG | 433 | TCAGCAACTTATGGGAAGGTTCTGAC | 465 |
| | 22 | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | 17GH1700- | TCAGCAACTTATGGGAAGGTTCTG | 434 | TCAGCAACTTATGGGAAGGTTCTGAC | 466 |
| | 13 | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | 17GH1702- | TCAGCAACTTATGGGAAGGTTCTG | 435 | TCAGCAACTTATGGGAAGGTTCTGAC | 467 |
| | 17 | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | 18GH2171 | TCAGCAACTTATGGGAAGGTTCTG | 436 | TCAGCAACTTATGGGAAGGTTCTGAC | 468 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | CS165 | TCAGCAACTTATGGGAAGGTTCTG | 437 | TCAGCAACTTATGGGAAGGTTCTGAC | 469 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | CS118 | TCAGCAACTTATGGGAAGGTTCTG | 438 | TCAGCAACTTATGGGAAGGTTCTGAC | 470 |
| | | ACTTTGGATATACAGAGAATGGT | | TTTGGATggagcaattcaacATACAGA | |
| | | GGATTTCCATACACTGAAATGATT | | GAATGGTGGATTTCCATACACTGAAAT | |
| | | GTTCATCTA | | GATTGTTCATCTA | |
| TN90 | CS113 | TCAGCAACTTATGGGAAGGTTCTG | 439 | TCAGCAACTTATGGGAAGGTTCTGAC | 471 |
| | | ACTTTGGATGGAGCAATTCAACA | | TTTGGATGGAGCAATTCAacatACAGA | |
| | | GAGAATGGTGGATTTCCATACACT | | GAATGGTGGATTTCCATACACTGAAA | |
| | | GAAATGATTGTTCATCTA | | TGATTGTTCATCTA | |
| TN90 | 17GH1737- | TCAGCAACTTATGGGAAGGTTCTG | 440 | TCAGCAACTTATGGGAAGGTTCTGAC | 472 |
| | 24 | ACTTTGGATGGAGCAATTCAATAC | | TTTGGATGGAGCAATTCAAcaTACAG | |
| | | AGAGAATGGTGGATTTCCATACA | | AGAATGGTGGATTTCCATACACTGAA | |
| | | CTGAAATGATTGTTCATCTA | | ATGATTGTTCATCTA | |
| IZMIR | 18GH2254- | TCAGCAACTTATGGGAAGGTTCTG | 441 | TCAGCAACTTATGGGAAGGTTCTGAC | 473 |
| | 7 | ACTTTGGATGGAGCAATTCAGAG | | TTTGGATGGAGCAATTCAacatacaGAG | |
| | | AATGGTGGATTTCCATACACTGAA | | AATGGTGGATTTCCATACACTGAAAT | |
| | | ATGATTGTTCATCTA | | GATTGTTCATCTA | |

TABLE 12B
A list of exemplary mutant alleles obtained in the PMT1b gene. Mutant allele sequences
listed here represent approximately 90-nucleotide-long genomic sequences
from each edited PMT1b gene with the edited site in the middle of the
genomic sequence (e.g., 45 nucleotides on each side of the
deleted sequence site). The mutant allele corresponds to the indel
provided for each line in Table 10. The lowercase letters in the
reference allele sequence (SEQ ID NO: 7) denote
which nucleotides are deleted in the mutant allele.

| | | | Mutant | | Reference |
|---|---|---|---|---|---|
| | | | Allele | | Allele |
| Geno- | | Mutant Allele | SEQ ID | | SEQ ID |
| type | Line | Sequence | NO. | Reference Allele Sequence | NO. |
| BASMA | CS107 | TCAGCAACTTATGGGAAGGTTCT | 474 | TCAGCAACTTATGGGAAGGTTCTG | 506 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| BASMA | CS106 | TCAGCAACTTATGGGAAGGTTCT | 475 | TCAGCAACTTATGGGAAGGTTCTG | 507 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| K326 | CS115 | TCAGCAACTTATGGGAAGGTTCT | 476 | TCAGCAACTTATGGGAAGGTTCTG | 508 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| K326 | 18GH2162 | TCAGCAACTTATGGGAAGGTTCT | 477 | TCAGCAACTTATGGGAAGGTTCTG | 509 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| K326 | CS111 | TCAGCAACTTATGGGAAGGTTCT | 478 | TCAGCAACTTATGGGAAGGTTCTG | 510 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| K326 | CS112 | TCAGCAACTTATGGGAAGGTTCT | 479 | TCAGCAACTTATGGGAAGGTTCTG | 511 |
| | | GACTTTGGATGGAGCAATTCAAC | | ACTTTGGATGGAGCAATTCAACAT | |
| | | ATAGAGAATGGTGGATTTCCATA | | acAGAGAATGGTGGATTTCCATAC | |
| | | CACTGAAATGATTGTTCATCTA | | ACTGAAATGATTGTTCATCTA | |
| K326 | 17GH1678- | TCAGCAACTTATGGGAAGGTTCT | 480 | TCAGCAACTTATGGGAAGGTTCTG | 512 |
| | 60 | GACTTTGGATGGAGCAATTCAAC | | ACTTTGGATGGAGCAATTCAACAT | |
| | | ATAGAGAATGGTGGATTTCCATA | | acAGAGAATGGTGGATTTCCATAC | |
| | | CACTGAAATGATTGTTCATCTA | | ACTGAAATGATTGTTCATCTA | |
| K326 | CS131 | TCAGCAACTTATGGGAAGGTTCT | 481 | TCAGCAACTTATGGGAAGGTTCTG | 513 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS164 | TCAGCAACTTATGGGAAGGTTCT | 482 | TCAGCAACTTATGGGAAGGTTCTG | 514 |
| | | GACTTTGGATGGAGCAATTCAGA | | ACTTTGGATGGAGCAATTCAacatac | |
| | | GAATGGTGGATTTCCATACACTG | | aGAGAATGGTGGATTTCCATACAC | |
| | | AAATGATTGTTCATCTA | | TGAAATGATTGTTCATCTA | |
| KATERINI | CS163 | TCAGCAACTTATGGGAAGGTTCT | 483 | TCAGCAACTTATGGGAAGGTTCTG | 515 |
| | | GACTTTGGATGGAGCAATTCAAC | | ACTTTGGATGGAGCAATTCAACat | |
| | | ACAGAGAATGGTGGATTTCCATA | | ACAGAGAATGGTGGATTTCCATA | |
| | | CACTGAAATGATTGTTCATCTA | | CACTGAAATGATTGTTCATCTA | |
| KATERINI | CS146 | TCAGCAACTTATGGGAAGGTTCT | 484 | TCAGCAACTTATGGGAAGGTTCTG | 516 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS147 | TCAGCAACTTATGGGAAGGTTCT | 485 | TCAGCAACTTATGGGAAGGTTCTG | 517 |
| | | GACTTTGGATGGAGCAATTCAGA | | ACTTTGGATGGAGCAATTCAacatac | |
| | | GAATGGTGGATTTCCATACACTG | | aGAGAATGGTGGATTTCCATACAC | |
| | | AAATGATTGTTCATCTA | | TGAAATGATTGTTCATCTA | |
| KATERINI | CS150 | TCAGCAACTTATGGGAAGGTTCT | 486 | TCAGCAACTTATGGGAAGGTTCTG | 518 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS151 | TCAGCAACTTATGGGAAGGTTCT | 487 | TCAGCAACTTATGGGAAGGTTCTG | 519 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS148 | TCAGCAACTTATGGGAAGGTTCT | 488 | TCAGCAACTTATGGGAAGGTTCTG | 520 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS149 | TCAGCAACTTATGGGAAGGTTCT | 489 | TCAGCAACTTATGGGAAGGTTCTG | 521 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS152 | TCAGCAACTTATGGGAAGGTTCT | 490 | TCAGCAACTTATGGGAAGGTTCTG | 522 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| KATERINI | CS153 | TCAGCAACTTATGGGAAGGTTCT | 491 | TCAGCAACTTATGGGAAGGTTCTG | 523 |
| | | GACTTTGGATGGAGCAATTCAAT | | ACTTTGGATGGAGCAATTCAAcaT | |
| | | ACAGAGAATGGTGGATTTCCATA | | ACAGAGAATGGTGGATTTCCATA | |
| | | CACTGAAATGATTGTTCATCTA | | CACTGAAATGATTGTTCATCTA | |
| KATERINI | CS102 | TCAGCAACTTATGGGAAGGTTCT | 492 | TCAGCAACTTATGGGAAGGTTCTG | 524 |
| | | GACTTTGGATGGAGCAATTCACA | | ACTTTGGATGGAGCAATTCAacataC | |
| | | GAGAATGGTGGATTTCCATACAC | | AGAGAATGGTGGATTTCCATACA | |
| | | TGAAATGATTGTTCATCTA | | CTGAAATGATTGTTCATCTA | |
| KATERINI | CS103 | TCAGCAACTTATGGGAAGGTTCT | 493 | TCAGCAACTTATGGGAAGGTTCTG | 525 |
| | | GACTTTGGATGGAGCAATTCAGA | | ACTTTGGATGGAGCAATTCAacatac | |
| | | GAATGGTGGATTTCCATACACTG | | aGAGAATGGTGGATTTCCATACAC | |
| | | AAATGATTGTTCATCTA | | TGAAATGATTGTTCATCTA | |
| TN90 | CS143 | TCAGCAACTTATGGGAAGGTTCT | 494 | TCAGCAACTTATGGGAAGGTTCTG | 526 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | 18GH2169 | TCAGCAACTTATGGGAAGGTTCT | 495 | TCAGCAACTTATGGGAAGGTTCTG | 527 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | CS120 | TCAGCAACTTATGGGAAGGTTCT | 496 | TCAGCAACTTATGGGAAGGTTCTG | 528 |
| | | GACTTTGGATGGAGCAATTCAAC | | ACTTTGGATGGAGCAATTCAACAT | |
| | | ATAGAATGGTGGATTTCCATACA | | acagAGAATGGTGGATTTCCATACA | |
| | | CTGAAATGATTGTTCATCTA | | CTGAAATGATTGTTCATCTA | |
| TN90 | 17GH1698- | TCAGCAACTTATGGGAAGGTTCT | 497 | TCAGCAACTTATGGGAAGGTTCTG | 529 |
| | 22 | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | 17GH1700- | TCAGCAACTTATGGGAAGGTTCT | 498 | TCAGCAACTTATGGGAAGGTTCTG | 530 |
| | 13 | GACTTTGGATGGAGCAATTCAGA | | ACTTTGGATGGAGCAATTCAacataC | |
| | | GAGAATGGTGGATTTCCATACAC | | AGAGAATGGTGGATTTCCATACA | |
| | | TGAAATGATTGTTCATCTA | | CTGAAATGATTGTTCATCTA | |
| TN90 | 17GH1702- | TCAGCAACTTATGGGAAGGTTCT | 499 | TCAGCAACTTATGGGAAGGTTCTG | 531 |
| | 17 | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | 18GH2171 | TCAGCAACTTATGGGAAGGTTCT | 500 | TCAGCAACTTATGGGAAGGTTCTG | 532 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | CS165 | TCAGCAACTTATGGGAAGGTTCT | 501 | TCAGCAACTTATGGGAAGGTTCTG | 533 |
| | | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| TN90 | CS118 | TCAGCAACTTATGGGAAGGTTCT | 502 | TCAGCAACTTATGGGAAGGTTCTG | 534 |
| | | GACTTTGGATGGAGCAATTAGAA | | ACTTTGGATGGAGCAATTcaacataca | |
| | | TGGTGGATTTCCATACACTGAAA | | gAGAATGGTGGATTTCCATACACT | |
| | | TGATTGTTCATCTA | | GAAATGATTGTTCATCTA | |
| TN90 | CS113 | TCAGCAACTTATGGGAAGGTTCT | 503 | TCAGCAACTTATGGGAAGGTTCTG | 535 |
| | | GACTTTGGATGGAGCAATTCAAC | | ACTTTGGATGGAGCAATTCAACAT | |
| | | ATAGAATGGTGGATTTCCATACA | | acagAGAATGGTGGATTTCCATACA | |
| | | CTGAAATGATTGTTCATCTA | | CTGAAATGATTGTTCATCTA | |
| TN90 | 17GH1737- | TCAGCAACTTATGGGAAGGTTCT | 504 | TCAGCAACTTATGGGAAGGTTCTG | 536 |
| | 24 | GACTTTGGATGGAGCAATTCAAC | | ACTTTGGATGGAGCAATTCAacatA | |
| | | AGAGAATGGTGGATTTCCATACA | | CAGAGAATGGTGGATTTCCATAC | |
| | | CTGAAATGATTGTTCATCTA | | ACTGAAATGATTGTTCATCTA | |
| IZMIR | 18GH2254- | TCAGCAACTTATGGGAAGGTTCT | 505 | TCAGCAACTTATGGGAAGGTTCTG | 537 |
| | 7 | GACTTTGGATGGAGCAATTCAAC | | ACTTTGGATGGAGCAATTCAAcata | |
| | | ACTGGTGGATTTCCATACACTGA | | cagagACTGGTGGATTTCCATACAC | |
| | | AATGATTGTTCATCTA | | TGAAATGATTGTTCATCTA | |

TABLE 12C
A list of exemplary mutant alleles obtained in the PMT2 gene.
Mutant allele sequences listed here represent approximately
90-nucleotide-long genomic sequences from each
edited PMT2 gene with the edited site in the middle of
the genomic sequence (e.g., 45 nucleotides on each side
of the deleted sequence site). The mutant allele
corresponds to the indel provided for each line in
Table 10. The lowercase letters in the reference allele
sequence (SEQ ID NO: 8) denote which nucleotides
are deleted in the mutant allele.

| | | | Mutant | | Reference |
|---|---|---|---|---|---|
| | | | Allele | | Allele |
| | | | SEQ ID | | SEQ ID |
| Genotype | Line | Mutant Allele Sequence | NO. | Reference Allele Sequence | NO. |
| BASMA | CS107 | TCAGCAACTTATGGGAAGGTTCTG | 538 | TCAGCAACTTATGGGAAGGTTCT | 570 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| BASMA | CS106 | TCAGCAACTTATGGGAAGGTTCTG | 539 | TCAGCAACTTATGGGAAGGTTCT | 571 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| K326 | CS115 | TCAGCAACTTATGGGAAGGTTCTG | 540 | TCAGCAACTTATGGGAAGGTTCT | 572 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| K326 | 18GH2162 | TCAGCAACTTATGGGAAGGTTCTG | 541 | TCAGCAACTTATGGGAAGGTTCT | 573 |
| | | ACTTTGGATGGAGCAATTCAAGA | | GACTTTGGATGGAGCAATTCAaca | |
| | | ATGGTGGATTTCCATACACTGAAA | | cacagAGAATGGTGGATTTCCATAC | |
| | | TGATTGTTCATCTT | | ACTGAAATGATTGTTCATCTT | |
| K326 | CS111 | TCAGCAACTTATGGGAAGGTTCTG | 542 | TCAGCAACTTATGGGAAGGTTCT | 574 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| K326 | CS112 | TCAGCAACTTATGGGAAGGTTCTG | 543 | TCAGCAACTTATGGGAAGGTTCT | 575 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| K326 | 17GH1678- | TCAGCAACTTATGGGAAGGTTCTG | 544 | TCAGCAACTTATGGGAAGGTTCT | 576 |
| | 60 | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| K326 | CS131 | TCAGCAACTTATGGGAAGGTTCTG | 545 | TCAGCAACTTATGGGAAGGTTCT | 577 |
| | | ACTTTGGATGGAGCAATTCAACA | | GACTTTGGATGGAGCAATTCAaca | |
| | | GAGAATGGTGGATTTCCATACACT | | CACAGAGAATGGTGGATTTCCAT | |
| | | GAAATGATTGTTCATCTT | | ACACTGAAATGATTGTTCATCTT | |
| KATERINI | CS164 | TCAGCAACTTATGGGAAGGTTCTG | 546 | TCAGCAACTTATGGGAAGGTTCT | 578 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS163 | TCAGCAACTTATGGGAAGGTTCTG | 547 | TCAGCAACTTATGGGAAGGTTCT | 579 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS146 | TCAGCAACTTATGGGAAGGTTCTG | 548 | TCAGCAACTTATGGGAAGGTTCT | 580 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS147 | TCAGCAACTTATGGGAAGGTTCTG | 549 | TCAGCAACTTATGGGAAGGTTCT | 581 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS150 | TCAGCAACTTATGGGAAGGTTCTG | 550 | TCAGCAACTTATGGGAAGGTTCT | 582 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS151 | TCAGCAACTTATGGGAAGGTTCTG | 551 | TCAGCAACTTATGGGAAGGTTCT | 583 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS148 | TCAGCAACTTATGGGAAGGTTCTG | 552 | TCAGCAACTTATGGGAAGGTTCT | 584 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS149 | TCAGCAACTTATGGGAAGGTTCTG | 553 | TCAGCAACTTATGGGAAGGTTCT | 585 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS152 | TCAGCAACTTATGGGAAGGTTCTG | 554 | TCAGCAACTTATGGGAAGGTTCT | 586 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS153 | TCAGCAACTTATGGGAAGGTTCTG | 555 | TCAGCAACTTATGGGAAGGTTCT | 587 |
| | | ACTTTGGATGGAGCAATTCAACA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| KATERINI | CS102 | TCAGCAACTTATGGGAAGGTTCTG | 556 | TCAGCAACTTATGGGAAGGTTCT | 588 |
| | | ACTTTGGATGGAGCAATTCAAGA | | GACTTTGGATGGAGCAATTCAaca | |
| | | ATGGTGGATTTCCATACACTGAAA | | cacagAGAATGGTGGATTTCCATAC | |
| | | TGATTGTTCATCTT | | ACTGAAATGATTGTTCATCTT | |
| KATERINI | CS103 | TCAGCAACTTATGGGAAGGTTCTG | 557 | TCAGCAACTTATGGGAAGGTTCT | 589 |
| | | ACTTTGGATGGAGCAATTCAAGA | | GACTTTGGATGGAGCAATTCAaca | |
| | | ATGGTGGATTTCCATACACTGAAA | | cacagAGAATGGTGGATTTCCATAC | |
| | | TGATTGTTCATCTT | | ACTGAAATGATTGTTCATCTT | |
| TN90 | CS143 | TCAGCAACTTATGGGAAGGTTCTG | 558 | TCAGCAACTTATGGGAAGGTTCT | 590 |
| | | ACTTTGGATGGAGCAATTCAAGA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| TN90 | 18GH2169 | TCAGCAACTTATGGGAAGGTTCTG | 559 | TCAGCAACTTATGGGAAGGTTCT | 591 |
| | | ACTTTGGATGGAGCAATTCAAGA | | GACTTTGGATGGAGCAATTCAaca | |
| | | GAGAATGGTGGATTTCCATACACT | | CACAGAGAATGGTGGATTTCCAT | |
| | | GAAATGATTGTTCATCTT | | ACACTGAAATGATTGTTCATCTT | |
| TN90 | CS120 | TCAGCAACTTATGGGAAGGTTCTG | 560 | TCAGCAACTTATGGGAAGGTTCT | 592 |
| | | ACTTTGGATGGAGCAATTCAAGA | | GACTTTGGATGGAGCAATTCAaca | |
| | | GAGAATGGTGGATTTCCATACACT | | CACAGAGAATGGTGGATTTCCAT | |
| | | GAAATGATTGTTCATCTT | | ACACTGAAATGATTGTTCATCTT | |
| TN90 | 17GH1698- | TCAGCAACTTATGGGAAGGTTCTG | 561 | TCAGCAACTTATGGGAAGGTTCT | 593 |
| | 22 | ACTTTGGATGGAGCAATTCAAGA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| TN90 | 17GH1700- | TCAGCAACTTATGGGAAGGTTCTG | 562 | TCAGCAACTTATGGGAAGGTTCT | 594 |
| | 13 | ACTTTGGATGGAGCAATTCAAGA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| TN90 | 17GH1702- | TCAGCAACTTATGGGAAGGTTCTG | 563 | TCAGCAACTTATGGGAAGGTTCT | 595 |
| | 17 | ACTTTGGATGGAGCAATTCAAGA | | GACTTTGGATGGAGCAATTCAacA | |
| | | CAGAGAATGGTGGATTTCCATAC | | CACAGAGAATGGTGGATTTCCAT | |
| | | ACTGAAATGATTGTTCATCTT | | ACACTGAAATGATTGTTCATCTT | |
| TN90 | 18GH2171 | TCAGCAACTTATGGGAAGGTTCTG | 564 | TCAGCAACTTATGGGAAGGTTCT | 596 |
| | | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |
| TN90 | CS165 | TCAGCAACTTATGGGAAGGTTCTG | 565 | TCAGCAACTTATGGGAAGGTTCT | 597 |
| | | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |
| TN90 | CS118 | TCAGCAACTTATGGGAAGGTTCTG | 566 | TCAGCAACTTATGGGAAGGTTCT | 598 |
| | | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |
| TN90 | CS113 | TCAGCAACTTATGGGAAGGTTCTG | 567 | TCAGCAACTTATGGGAAGGTTCT | 599 |
| | | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |
| TN90 | 17GH1737- | TCAGCAACTTATGGGAAGGTTCTG | 568 | TCAGCAACTTATGGGAAGGTTCT | 600 |
| | 24 | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |
| IZMIR | 18GH2254- | TCAGCAACTTATGGGAAGGTTCTG | 569 | TCAGCAACTTATGGGAAGGTTCT | 601 |
| | 7 | ACTTTGGATGGAGCAATTCAACA | | GACTTTGGATGGAGCAATTCAaca | |
| | | GAGAATGGTGGATTTCCATACACT | | cACAGAGAATGGTGGATTTCCAT | |
| | | GAAATGATTGTTCATCTT | | ACACTGAAATGATTGTTCATCTT | |

TABLE 12D
A list of exemplary mutant alleles obtained in the PMT3 gene. Mutant allele
sequences listed here represent approximately 90-nucleotide-long
genomic sequences from each edited PMT3 gene with the edited site in the
deleted middle of the genomic sequence (e.g., 45 nucleotides on each side of the
sequence site). The mutant allele corresponds to the indel provided for each
line in Table 10. The lowercase letters in the reference allele sequence (SEQ ID
NO: 9) denote which nucleotides are deleted in the mutant allele.

| | | | Mutant | | Reference |
|---|---|---|---|---|---|
| | | | Allele | | Allele |
| | | | SEQ ID | Reference Allele | SEQ ID |
| Genotype | Line | Mutant Allele Sequence | NO. | Sequence | NO. |
| BASMA | CS107 | TCAGCAACATATGGGAAGGTTCT | 602 | TCAGCAACATATGGGAAGGTTCT | 634 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| BASMA | CS106 | TCAGCAACATATGGGAAGGTTCT | 603 | TCAGCAACATATGGGAAGGTTCT | 635 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| K326 | CS115 | TCAGCAACATATGGGAAGGTTCT | 604 | TCAGCAACATATGGGAAGGTTCT | 636 |
| | | GACTTTGGATGGAGCAATTCAAA | | GACTTTGGATGGAGCAATTCAAC | |
| | | GAGAATGGTGGATTTCCATACA | | acacAGAGAATGGTGGATTTCCAT | |
| | | GCTAAATGATTGTTCATCTT | | ACACTGAAATGATTGTTCATCTT | |
| K326 | 18GH2162 | TCAGCAACATATGGGAAGGTTCT | 605 | TCAGCAACATATGGGAAGGTTCT | 637 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| K326 | CS111 | TCAGCAACATATGGGAAGGTTCT | 606 | TCAGCAACATATGGGAAGGTTCT | 638 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| K326 | CS112 | TCAGCAACATATGGGAAGGTTCT | 607 | TCAGCAACATATGGGAAGGTTCT | 639 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| K326 | 17GH1678- | TCAGCAACATATGGGAAGGTTCT | 608 | TCAGCAACATATGGGAAGGTTCT | 640 |
| | 60 | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| K326 | CS131 | TCAGCAACATATGGGAAGGTTCT | 609 | TCAGCAACATATGGGAAGGTTCT | 641 |
| | | GACTTTGGATGGAGCAATTCAGA | | GACTTTGGATGGAGCAATTCaacac | |
| | | ATGGTGGATTTCCATACACTGAAA | | acagAGAATGGTGGATTTCCATAC | |
| | | TGATTGTTCATCTT | | ACTGAAATGATTGTTCATCTT | |
| KATERINI | CS164 | TCAGCAACATATGGGAAGGTTCT | 610 | TCAGCAACATATGGGAAGGTTCT | 642 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS163 | TCAGCAACATATGGGAAGGTTCT | 611 | TCAGCAACATATGGGAAGGTTCT | 643 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS146 | TCAGCAACATATGGGAAGGTTCT | 612 | TCAGCAACATATGGGAAGGTTCT | 644 |
| | | GACTTTGGATGGAGCAATTCAGA | | GACTTTGGATGGAGCAATTcaacac | |
| | | GAATGGTGGATTTCCATACACTGA | | aCAGAGAATGGTGGATTTCCATA | |
| | | AATGATTGTTCATCTT | | CACTGAAATGATTGTTCATCTT | |
| KATERINI | CS147 | TCAGCAACATATGGGAAGGTTCT | 613 | TCAGCAACATATGGGAAGGTTCT | 645 |
| | | GACTTTGGATGGAGCAATTCAGA | | GACTTTGGATGGAGCAATTcaacac | |
| | | GAATGGTGGATTTCCATACACTGA | | aCAGAGAATGGTGGATTTCCATA | |
| | | AATGATTGTTCATCTT | | CACTGAAATGATTGTTCATCTT | |
| KATERINI | CS150 | TCAGCAACATATGGGAAGGTTCT | 614 | TCAGCAACATATGGGAAGGTTCT | 646 |
| | | GACTTTGGATGGAGCAATTCAAA | | GACTTTGGATGGAGCAATTCAAca | |
| | | GAATGGTGGATTTCCATACACTGA | | cacagAGAATGGTGGATTTCCATAC | |
| | | AATGATTGTTCATCTT | | ACTGAAATGATTGTTCATCTT | |
| KATERINI | CS151 | TCAGCAACATATGGGAAGGTTCT | 615 | TCAGCAACATATGGGAAGGTTCT | 647 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS148 | TCAGCAACATATGGGAAGGTTCT | 616 | TCAGCAACATATGGGAAGGTTCT | 648 |
| | | GACTTTGGATGGAGCAATTCAAA | | GACTTTGGATGGAGCAATTCAAca | |
| | | GAATGGTGGATTTCCATACACTGA | | cacagAGAATGGTGGATTTCCATAC | |
| | | AATGATTGTTCATCTT | | ACTGAAATGATTGTTCATCTT | |
| KATERINI | CS149 | TCAGCAACATATGGGAAGGTTCT | 617 | TCAGCAACATATGGGAAGGTTCT | 649 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS152 | TCAGCAACATATGGGAAGGTTCT | 618 | TCAGCAACATATGGGAAGGTTCT | 650 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS153 | TCAGCAACATATGGGAAGGTTCT | 619 | TCAGCAACATATGGGAAGGTTCT | 651 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| KATERINI | CS102 | TCAGCAACATATGGGAAGGTTCT | 620 | TCAGCAACATATGGGAAGGTTCT | 652 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| KATERINI | CS103 | TCAGCAACATATGGGAAGGTTCT | 621 | TCAGCAACATATGGGAAGGTTCT | 653 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | CS143 | TCAGCAACATATGGGAAGGTTCT | 622 | TCAGCAACATATGGGAAGGTTCT | 654 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | 18GH2169 | TCAGCAACATATGGGAAGGTTCT | 623 | TCAGCAACATATGGGAAGGTTCT | 655 |
| | | GACTTTGGATGGAGCAATTCAAC | | GACTTTGGATGGAGCAATTCAaca | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | CS120 | TCAGCAACATATGGGAAGGTTCT | 624 | TCAGCAACATATGGGAAGGTTCT | 656 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | 17GH1698- | TCAGCAACATATGGGAAGGTTCT | 625 | TCAGCAACATATGGGAAGGTTCT | 657 |
| | 22 | GACTTTGGATGGAGCAATTCAAC | | GACTTTGGATGGAGCAATTCAAC | |
| | | AGAGAATGGTGGATTTCCATACA | | ACacagAGAATGGTGGATTTCCAT | |
| | | CTGAAATGATTGTTCATCTT | | ACACTGAAATGATTGTTCATCTT | |
| TN90 | 17GH1700- | TCAGCAACATATGGGAAGGTTCT | 626 | TCAGCAACATATGGGAAGGTTCT | 658 |
| | 13 | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | 17GH1702- | TCAGCAACATATGGGAAGGTTCT | 627 | TCAGCAACATATGGGAAGGTTCT | 659 |
| | 17 | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | 18GH2171 | TCAGCAACATATGGGAAGGTTCT | 628 | TCAGCAACATATGGGAAGGTTCT | 660 |
| | | GACTTTGGATGGAGCAATTCAGA | | GACTTTGGATGGAGCAATTcaacac | |
| | | GAATGGTGGATTTCCATACACTGA | | aCAGAGAATGGTGGATTTCCATA | |
| | | AATGATTGTTCATCTT | | CACTGAAATGATTGTTCATCTT | |
| TN90 | CS165 | TCAGCAACATATGGGAAGGTTCT | 629 | TCAGCAACATATGGGAAGGTTCT | 661 |
| | | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |
| TN90 | CS118 | TCAGCAACATATGGGAAGGTTCT | 630 | TCAGCAACATATGGGAAGGTTCT | 662 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | CS113 | TCAGCAACATATGGGAAGGTTCT | 631 | TCAGCAACATATGGGAAGGTTCT | 663 |
| | | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| TN90 | 17GH1737- | TCAGCAACATATGGGAAGGTTCT | 632 | TCAGCAACATATGGGAAGGTTCT | 664 |
| | 24 | GACTTTGGATGGAGCAATTCAAC | | GACTTTGGATGGAGCAATTCAacA | |
| | | ACAGAGAATGGTGGATTTCCATA | | CACAGAGAATGGTGGATTTCCAT | |
| | | CACTGAAATGATTGTTCATCTT | | ACACTGAAATGATTGTTCATCTT | |
| IZMIR | 18GH2254- | TCAGCAACATATGGGAAGGTTCT | 633 | TCAGCAACATATGGGAAGGTTCT | 665 |
| | 7 | GACTTTGGATGGAGCAATTCAAC | | GACTTTGGATGGAGCAATTCAaca | |
| | | AGAGAATGGTGGATTTCCATACA | | cACAGAGAATGGTGGATTTCCAT | |
| | | CTGAAATGATTGTTCATCTT | | ACACTGAAATGATTGTTCATCTT | |

TABLE 12E
A list of exemplary mutant alleles obtained in the PMT4 gene.
Mutant allele sequences listed here represent approximately
90-nucleotide-long genomic sequences from each edited
PMT4 gene with the edited site in the middle of the genomic
sequence (e.g., 45 nucleotides on each side of the deleted
sequence site). The mutant allele corresponds to the indel
provided for each line in Table 10. The lowercase letters
in the reference allele sequence (SEQ ID NO: 10)
denote which nucleotides are deleted in the mutant allele.

| | | | Mutant | | Reference |
|---|---|---|---|---|---|
| | | | Allele | | Allele |
| | | | SEQ | Reference Allele | SEQ ID |
| Genotype | Line | Mutant Allele Sequence | ID NO. | Sequence | NO. |
| BASMA | CS107 | TCAGCAACATATGGGAAGGTTTTG | 666 | TCAGCAACATATGGGAAGGTTTT | 698 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| BASMA | CS106 | TCAGCAACATATGGGAAGGTTTTG | 667 | TCAGCAACATATGGGAAGGTTTT | 699 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| K326 | CS115 | TCAGCAACATATGGGAAGGTTTTG | 668 | TCAGCAACATATGGGAAGGTTTT | 700 |
| | | ACTTTGGATGGAGCAATTCAACG | | GACTTTGGATGGAGCAATTCAACa | |
| | | AGAATGGTGGATTTCCATACACTG | | cacaGAGAATGGTGGATTTCCATAC | |
| | | AAATGATTGTTCATCTT | | ACTGAAATGATTGTTCATCTT | |
| K326 | 18GH2162 | TCAGCAACATATGGGAAGGTTTTG | 669 | TCAGCAACATATGGGAAGGTTTT | 701 |
| | | ACTTTGGATGGAGCAATTCAACG | | GACTTTGGATGGAGCAATTCAACa | |
| | | AGAATGGTGGATTTCCATACACTG | | cacaGAGAATGGTGGATTTCCATAC | |
| | | AAATGATTGTTCATCTT | | ACTGAAATGATTGTTCATCTT | |
| K326 | CS111 | TCAGCAACATATGGGAAGGTTTTG | 670 | TCAGCAACATATGGGAAGGTTTT | 702 |
| | | ACTTTGGATGGAGCAATTCAAAG | | GACTTTGGATGGAGCAATTCAACa | |
| | | AGAATGGTGGATTTCCATACACTG | | cacAGAGAATGGTGGATTTCCATA | |
| | | AAATGATTGTTCATCTT | | CACTGAAATGATTGTTCATCTT | |
| K326 | CS112 | TCAGCAACATATGGGAAGGTTTTG | 671 | TCAGCAACATATGGGAAGGTTTT | 703 |
| | | ACTTTGGATGGAGCAATTCAAAG | | GACTTTGGATGGAGCAATTCAAca | |
| | | AGAATGGTGGATTTCCATACACTG | | cacAGAGAATGGTGGATTTCCATA | |
| | | AAATGATTGTTCATCTT | | CACTGAAATGATTGTTCATCTT | |
| K326 | 17GH1678- | TCAGCAACATATGGGAAGGTTTTG | 672 | TCAGCAACATATGGGAAGGTTTT | 704 |
| | 60 | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| K326 | CS131 | TCAGCAACATATGGGAAGGTTTTG | 673 | TCAGCAACATATGGGAAGGTTTT | 705 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| KATERINI | CS164 | TCAGCAACATATGGGAAGGTTTTG | 674 | TCAGCAACATATGGGAAGGTTTT | 706 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS163 | TCAGCAACATATGGGAAGGTTTTG | 675 | TCAGCAACATATGGGAAGGTTTT | 707 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS146 | TCAGCAACATATGGGAAGGTTTTG | 676 | TCAGCAACATATGGGAAGGTTTT | 708 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS147 | TCAGCAACATATGGGAAGGTTTTG | 677 | TCAGCAACATATGGGAAGGTTTT | 709 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS150 | TCAGCAACATATGGGAAGGTTTTG | 678 | TCAGCAACATATGGGAAGGTTTT | 710 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS151 | TCAGCAACATATGGGAAGGTTTTG | 679 | TCAGCAACATATGGGAAGGTTTT | 711 |
| | | ACTTTGGATGGAGCAATTAGAAT | | GACTTTGGATGGAGCAATTcaacaca | |
| | | GGTGGATTTCCATACACTGAAATG | | cagAGAATGGTGGATTTCCATACA | |
| | | ATTGTTCATCTT | | CTGAAATGATTGTTCATCTT | |
| KATERINI | CS148 | TCAGCAACATATGGGAAGGTTTTG | 680 | TCAGCAACATATGGGAAGGTTTT | 712 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS149 | TCAGCAACATATGGGAAGGTTTTG | 681 | TCAGCAACATATGGGAAGGTTTT | 713 |
| | | ACTTTGGATGGAGCAATTAGAAT | | GACTTTGGATGGAGCAATTcaacaca | |
| | | GGTGGATTTCCATACACTGAAATG | | cagAGAATGGTGGATTTCCATACA | |
| | | ATTGTTCATCTT | | CTGAAATGATTGTTCATCTT | |
| KATERINI | CS152 | TCAGCAACATATGGGAAGGTTTTG | 682 | TCAGCAACATATGGGAAGGTTTT | 714 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| KATERINI | CS153 | TCAGCAACATATGGGAAGGTTTTG | 683 | TCAGCAACATATGGGAAGGTTTT | 715 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| KATERINI | CS102 | TCAGCAACATATGGGAAGGTTTTG | 684 | TCAGCAACATATGGGAAGGTTTT | 716 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| KATERINI | CS103 | TCAGCAACATATGGGAAGGTTTTG | 685 | TCAGCAACATATGGGAAGGTTTT | 717 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | CS143 | TCAGCAACATATGGGAAGGTTTTG | 686 | TCAGCAACATATGGGAAGGTTTT | 718 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| TN90 | 18GH2169 | TCAGCAACATATGGGAAGGTTTTG | 687 | TCAGCAACATATGGGAAGGTTTT | 719 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | CS120 | TCAGCAACATATGGGAAGGTTTTG | 688 | TCAGCAACATATGGGAAGGTTTT | 720 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | 17GH1698- | TCAGCAACATATGGGAAGGTTTTG | 689 | TCAGCAACATATGGGAAGGTTTT | 721 |
| | 22 | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | 17GH1700- | TCAGCAACATATGGGAAGGTTTTG | 690 | TCAGCAACATATGGGAAGGTTTT | 722 |
| | 13 | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| TN90 | 17GH1702- | TCAGCAACATATGGGAAGGTTTTG | 691 | TCAGCAACATATGGGAAGGTTTT | 723 |
| | 17 | ACTTTGGATGGAGCAATTCAACA | | GACTTTGGATGGAGCAATTCAAC | |
| | | AGAATGGTGGATTTCCATACACTG | | AcacagAGAATGGTGGATTTCCATA | |
| | | AAATGATTGTTCATCTT | | CACTGAAATGATTGTTCATCTT | |
| TN90 | 18GH2171 | TCAGCAACATATGGGAAGGTTTTG | 692 | TCAGCAACATATGGGAAGGTTTT | 724 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | CS165 | TCAGCAACATATGGGAAGGTTTTG | 693 | TCAGCAACATATGGGAAGGTTTT | 725 |
| | | ACTTTGGATGGAGCAATTCAACA | | GACTTTGGATGGAGCAATTCAacac | |
| | | GAGAATGGTGGATTTCCATACACT | | ACAGAGAATGGTGGATTTCCATA | |
| | | GAAATGATTGTTCATCTT | | CACTGAAATGATTGTTCATCTT | |
| TN90 | CS118 | TCAGCAACATATGGGAAGGTTTTG | 694 | TCAGCAACATATGGGAAGGTTTT | 726 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | CS113 | TCAGCAACATATGGGAAGGTTTTG | 695 | TCAGCAACATATGGGAAGGTTTT | 727 |
| | | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| TN90 | 17GH1737- | TCAGCAACATATGGGAAGGTTTTG | 696 | TCAGCAACATATGGGAAGGTTTT | 728 |
| | 24 | ACTTTGGATGGAGCAATTCAACAC | | GACTTTGGATGGAGCAATTCAacA | |
| | | AGAGAATGGTGGATTTCCATACA | | CACAGAGAATGGTGGATTTCCAT | |
| | | CTGAAATGATTGTTCATCTT | | ACACTGAAATGATTGTTCATCTT | |
| IZMIR | 18GH2254- | TCAGCAACATATGGGAAGGTTTTG | 697 | TCAGCAACATATGGGAAGGTTTT | 729 |
| | 7 | ACTTTGGATGGAGCAATTCAAGA | | GACTTTGGATGGAGCAATTCAacac | |
| | | ATGGTGGATTTCCATACACTGAAA | | acagAGAATGGTGGATTTCCATACA | |
| | | TGATTGTTCATCTT | | CTGAAATGATTGTTCATCTT | |

### Example 8. Alkaloid Analysis of PMT Edited Lines

Homozygous genome edited tobacco lines from Example 7, along with control lines, are grown in in a field. At flowering stage, plants are topped and two-weeks post topping, lamina samples are collected from the third, fourth, and fifth leaves from the top of the plant and alkaloid levels are measured (see Tables 13A-13C) using a method in accordance with CORESTA Method No 62, Determination of Nicotine in Tobacco and Tobacco Products by Gas Chromatographic Analysis, February 2005, and those defined in the Centers for Disease Control and Prevention's Protocol for Analysis of Nicotine, Total Moisture and pHI in Smokeless Tobacco Products, as published in the Federal Register Vol. 64, No. 55 Mar. 23, 1999 (and as amended in Vol. 74, No. 4, Jan. 7, 2009).

Approximately 0.5 g of tobacco is extracted using liquid/liquid extraction into an organic solvent containing an internal standard and analyzed by gas chromatography (GC) with flame ionization detection (FID). Results can be reported as weight percent (Wt %) on either on as is or dry weight basis. Reporting data on a dry weight basis requires an oven volatiles (OV) determination. Unless specified otherwise, total or individual alkaloid levels or nicotine levels shown herein are on a dry weight basis (e.g., percent total alkaloid or percent nicotine).

Plants are also planted in the field, harvested, and tested for alkaloids and TSNA levels in cured tobacco. Both leaf yield and leaf grade are also assessed for PMT edited plants.

TABLE 13A
Nicotine analysis of K326 and TN90 PMT edited lines after
two weeks after flowering.

| | | | Nicotine |
|---|---|---|---|
| Variety | Line | Replicate | (mg/g tissue) |
| K 326 | CS111 | 1 | 0.023 |
| | | 2 | 0.024 |
| | CS131 | 1 | 0.022 |
| | | 2 | 0.018 |
| | | 3 | 0.021 |
| | CS115 | 1 | 0.023 |
| | | 2 | 0.015 |
| | Control | 1 | 16.8 |
| | | 2 | 17.2 |
| | | 3 | 16.6 |
| TN 90 LC | CS116 | 1 | 0.029 |
| | | 2 | 0.022 |
| | CS133 | 1 | 0.016 |
| | | 2 | 0.018 |
| | CS135 | 1 | 0.027 |
| | | 2 | 0.031 |
| | CS120 | 1 | 0.022 |
| | | 2 | 0.045 |
| | CS137 | 1 | 0.07 |
| | | 2 | 0.048 |
| | Control | 1 | 29.5 |
| | | 2 | 29.8 |
| | | 3 | 24.2 |

TABLE 13B
Nicotine analysis of K326 and TN90 PMT
edited lines after two-weeks after topping.

| Variety | Group | Replicate | Nicotine |
|---|---|---|---|
| K 326 | CS111 | 1 | 0.024 |
| | | 2 | 0.025 5 |
| | CS131 | 1 | 0.021 |
| | | 2 | 0.02 |
| | | 3 | 0.019 |
| | CS115 | 1 | 0.018 |
| | Control | 1 | 16.771 |
| | | 2 | 17.212 |
| | | 3 | 16.581 |
| | | 4 | 22.734 |
| TN 90 | CS116 | 1 | 0.015 |
| LC | CS133 | 1 | 0.036 |
| | | 2 | 0.018 |
| | CS135 | 1 | 0.018 |
| | | 2 | 0.019 |
| | CS120 | 1 | 0.04 |
| | | 2 | 0.025 |
| | CS137 | 1 | 0.051 |
| | | 2 | 0.057 |
| | Control | 1 | 29.472 |
| | | 2 | 29.776 |
| | | 3 | 24.22 |
| | | 4 | 24.939 |

'

TABLE 13C
Nicotine analysis of Katerini and Basma PMT edited lines after
two-weeks after flowering.

| | | | Nicotine |
|---|---|---|---|
| Variety | Group | Replicate | (mg/g tissue) |
| Katerini | CS102 | 1 | 0.032 |
| | | 2 | 0.028 |
| | CS103 | 1 | 0.017 |
| | Control | 1 | 26.109 |
| | | 2 | 26.466 |
| | | 3 | 27.091 |
| Basma | CS107 | 1 | 0.029 |
| | CS108 | 1 | 0.014 |
| | | 2 | 0.018 |
| | Control | 1 | 21.979 |
| | | 2 | 20.88 |
| | | 3 | 23.499 |

### Example 9. Development of Male Sterile PMT Edited Lines

PMT edited hybrid lines are developed using the lines from Example 7. Hybrid lines are grown in the field and used as progenitors for male sterile lines. See Table 14.

TABLE 14
PMT edited very low nicotine male sterile lines

| | Pollen source | F1 hybrid seed |
|---|---|---|
| Male Sterile Variety | (line) | (line) |
| MS Katerini | CS102 | dCS11 |
| | CS103 | dCS12 |
| MS Basma | CS106 | dCS13 |
| | CS107 | dCS14 |
| MS K326 | CS111 | dCS15 |
| | CS115 | dCS16 |
| MS TN90 | CS118 | dCS17 |
| | CS120 | dCS18 |
| MS Izmir | 18GH2254 | dS2697 |

### Example 10. PMT Edited Lines Resist Mold During Curing

Tobacco leaf harvested from several low alkaloid tobacco lines is subjected to standard air curing practices. The tobacco leaves are examined for mold after the completion of curing.

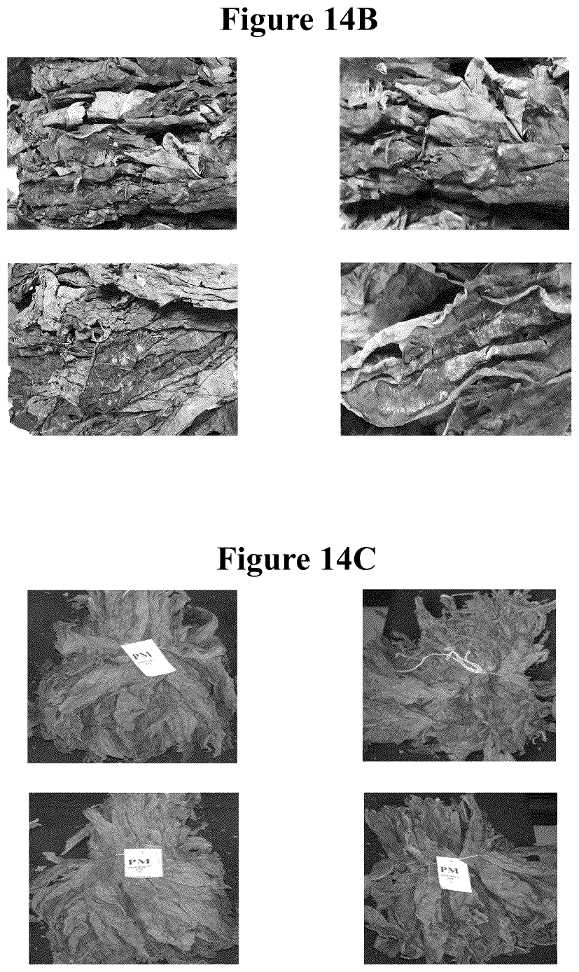

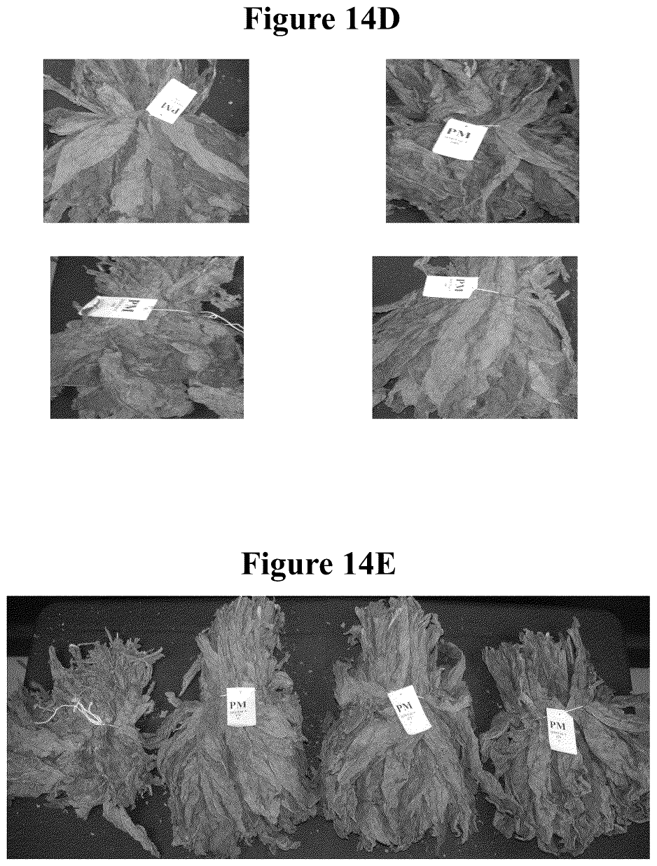

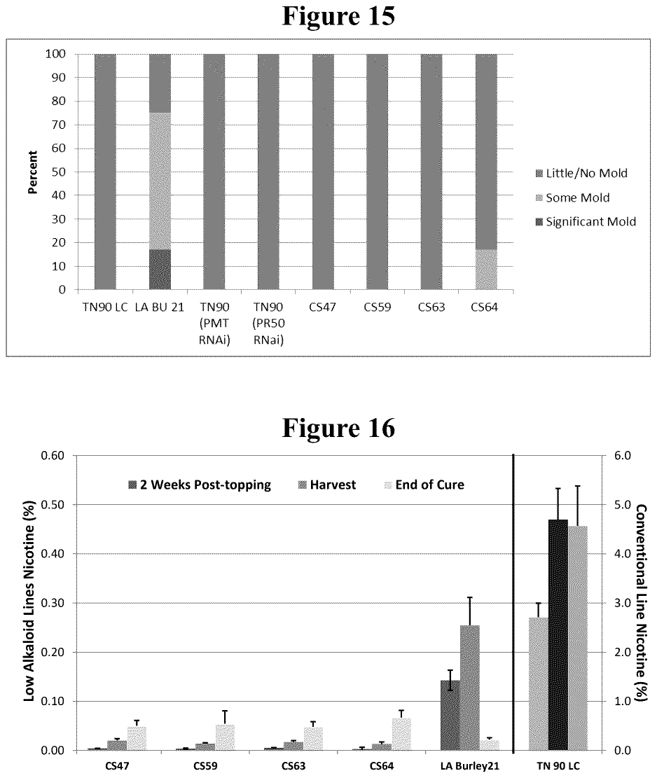

Tobacco from the LA BU 21 exhibits more mold infestation than TN90 LC, a TN90 variety comprising an RNAi construct to downregulate all five PMT genes, a TN90 variety comprising an RNAi construct to downregulate the alkaloid biosynthesis gene PR50, and four PMT edited lines (CS47, CS59, CS63, and CS64) in a TN90 genetic background. See Table 15 and FIGS. 14A to 14E and 15.

TABLE 15
Mold damage exhibited by tobacco lines. “G” refers to little/no mold; “S” refers
to some mold; and “B” refers to significant mold. Percentage of Mold refers to
the percentage of air cured sticks of tobacco exhibited each category of mold damage.

| | | | | | | | | | | | | | Percentage of Mold | | |
|---|---|---|---|---|---|---|---|---|---|---|---|---|---|---|---|
| | Mold Rating | | | | | | | | | | | | Significant | Some | Little/ |
| Variety | Replicate 1 | | | Replicate 2 | | | Replicate 3 | | | Replicate 4 | | | Mold | Mold | No Mold |
| TN90 LC | G | G | G | G | G | G | G | G | G | G | G | G | 0% | 0% | 100% |
| LA BU 21 | G | S | G | B | S | G | S | S | S | B | S | S | 17% | 58% | 25% |
| TN90 (PMT RNAi) | G | G | G | G | G | G | G | G | G | G | G | G | 0% | 0% | 100% |
| TN90 (PR50 RNai) | G | G | G | G | G | G | G | G | G | G | G | G | 0% | 0% | 100% |
| CS47 | G | G | G | G | G | G | G | G | G | G | G | G | 0% | 0% | 100% |
| CS59 | G | G | G | G | G | G | G | G | G | G | G | G | 0% | 0% | 100% |
| CS63 | G | G | G | G | G | G | G | G | G | G | G | G | 0% | 0% | 100% |
| CS64 | G | G | G | G | G | G | G | G | G | S | S | G | 0% | 17% | 83% |

TABLE 16
Flue-cured Tobacco Varieties

| | | |
|---|---|---|
| 400 (TC 225) | K 346 | Reams 134 |
| 401 (TC 226) | K 346 (TC 569) | Reams 158 |
| 401 Cherry Red (TC 227) | K 358 | Reams 713 |
| 401 Cherry Red Free (TC | K 394 (TC 321) | Reams 744 |
| 228) | K 399 | Reams M1 |
| Cash (TC 250) | K 399 (TC 322) | RG 11 (TC 600) |
| Cash (TI278) | K 730 | RG 13 (TC 601) |
| CC 101 | Lonibow (TI 1573) | RG 17 (TC 627) |
| CC 1063 | Lonibow (TI 1613) | RG 22 (TC 584) |
| CC 13 | McNair 10 (TC 330) | RG 8 (TC 585) |
| CC 143 | McNair 135 (TC 337) | RG 81 (TC618) |
| CC 200 | McNair 30 (TC 334) | RGH 51 |
| CC 27 | McNair 373 (TC 338) | RG4H 217 |
| CC 301 | McNair 944 (TC 339) | RGH 12 |
| CC 33 | MK94 (TI 1512) | RGH 4 |
| CC 35 | MS K 326 | RGH 51 |
| CC 37 | MS NC 71 | RGH 61 |
| CC 400 | MS NC 72 | SC 58 (TC 400) |
| CC 500 | NC 100 | SC 72 (TC 403) |
| CC 600 | NC 102 | Sp. G-168 |
| CC 65 | NC 1071 (TC 364) | SPEIGHT 168 |
| CC 67 | NC 1125-2 | Speight 168 (TC 633) |
| CC 700 | NC 12 (TC 346) | Speight 172 (TC 634) |
| CC 800 | NC 1226 | Speight 178 |
| CC 900 | NC 196 | Speight 179 |
| Coker 139 (TC 259) | NC 2326 (TC 365) | Speight 190 |
| Coker 139 yb1, yb2 | NC 27 NF (TC 349) | Speight 196 |
| Coker 140 (TC 260) | NC 291 | SPEIGHT 220 |
| Coker 176 (TC 262) | NC 297 | SPEIGHT 225 |
| Coker 187 (TC 263) | NC 299 | SPEIGHT 227 |
| Coker 187-Hicks (TC 265) | NC 37 NF (TC 350) | SPEIGHT 236 |
| Coker 209 (TC 267) | NC 471 | Speight G-10 (TC 416) |
| Coker 258 (TC 270) | NC 55 | Speight G-102 |
| Coker 298 (TC 272) | NC 567 (TC 362) | Speight G-108 |
| Coker 316 (TC 273) | NC 60 (TC 352) | Speight G-111 |
| Coker 319 (TC 274) | NC 606 | Speight G-117 |
| Coker 347 (TC 275) | NC 6140 | Speight G-126 |
| Coker 371-Gold (TC 276) | NC71 | Speight G-15 (TC418) |
| Coker 411 (TC 277) | NC 72 | Speight G-23 |
| Coker 48 (TC 253) | NC 729 (TC 557) | Speight G-28 (TC 420) |
| Coker 51 (TC 254) | NC 810 (TC 659) | Speight G-33 |
| Coker 86 (TC 256) | NC 82 (TC 356) | Speight G-41 |
| CU 263 (TC619) | NC 8640 | Speight G-5 |
| CU 561 | NC 89 (TC 359) | Speight G-52 |
| DH95-1562-1 | NC 92 | Speight G-5 8 |
| Dixie Bright 101 (TC 290) | NC 925 | Speight G-70 |
| Dixie Bright 102 (TC 291) | NC 95 (TC 360) | Speight G-70 (TC 426) |
| Dixie Bright 244 (TC 292) | NC 98 (TC 361) | Speight G-80 (TC 427) |
| Dixie Bright 27 (TC 288) | NC EX 24 | Speight NF3 (TC 629) |
| Dixie Bright 28 (TC 289) | NC PY 10 (TC 367) | STNCB |
| GF 157 | NC TG 61 | VA 182 |
| GF 318 | Oxford 1 (TC 369) | VA 45 (TC 559) |
| GL 26H | Oxford 1-181 (TC 370) | Vesta 30 (TC 439) |
| GL 338 | Oxford 2 (TC 371) | Vesta 33 (TC 440) |
| GL 350 | Oxford 207 (TC 632) | Vesta 5 (TC 438) |
| GL 368 | Oxford 26 (TC 373) | Vesta 62 (TC 441) |
| GL 395 | Oxford 3 (TC 372) | Virginia (TI 220) |
| GL 600 | Oxford 414 NF | Virginia (TI 273) |
| GL 737 | PD 611 (TC 387) | Virginia (TI 877) |
| GL 939 | PVH 03 | Virginia 115 (TC 444) |
| GL 939 (TC 628) | PVH 09 | Virginia 21 (TC 443) |
| Hicks (TC 310) | PVH 1118 | Virginia Bright (TI 964) |
| Hicks Broadleaf (TC 311) | PVH 1452 | Virginia Bright Leaf (TC 446) |
| K 149 (TC 568) | PVH 1600 | Virginia Gold (TC 447) |
| K 317 | PVH 2110 | White Stem Orinoco (TC 451) |
| K 326 | PVH 2275 | |
| K 326 (TC 319) | R83 (Line 256-1) (TI 1400) | |
| K 340 (TC 320) | | |

TABLE 17
Burley Tobacco Varieties

| | | |
|---|---|---|
| 4407 LC | HB 4108P | KY 54 (TC71) |
| AA-37-1 | HB 4151P | KY 56 (TC 72) |
| Burley 21 (TC 7) | HB 4192P | KY 56 (TC 72) |
| Burley 49 (TC 10) | HB 4194P | KY 57 (TC 73) |
| Burley 64 (TC 11) | HB 4196 | KY 58 (TC 74) |
| Burley Mammoth KY 16 (TC 12) | HB 4488 | KY 8654 (TC 77) |
| Clay 402 | HB 4488P | KY 8959 |
| Clay 403 | HB 04P | KY 9 (TC 54) |
| Clay 502 | HB 4488 LC | KY 907 LC |
| Clays 403 | HIB 21 | KY 908 (TC 630) |
| GR 10 (TC 19) | HPB 21 | NBH 98 (Screened) |
| GR 10 (TC 19) | HY 403 | NC 1206 |
| GR 10A (TC 20) | Hybrid 403 LC | NC 129 |
| GR 13 (TC 21) | Hybrid 404 LC | NC 2000 LC |
| GR 14 (TC 22) | Hybrid 501 LC | NC 2002 LC |
| GR 149 LC | KDH-959 (TC 576) | NC 3 LC |
| GR 153 | KDH-960 (TC 577) | NC 5 LC |
| GR 17 (TC 23) | KT 200 LC | NC 6 LC |
| GR 17B (TC 24) | KT 204 LC | NC 7 LC |
| GR 18 (TC 25) | KT 206 LC | NC BH 129 LC |
| GR 19 (TC 26) | KT 209 LC | NC03-42-2 |
| GR 2 (TC 15) | KT 210 LC | Newton 98 |
| GR 24 (TC 27) | KT 212 LC | R 610 LC |
| GR 36 (TC 28) | KT 215 LC | R 630 LC |
| GR 38 (TC 29) | KY 1 (TC 52) | R7-11 |
| GR 38A (TC30) | KY 10 (TC 55) | R7-12LC |
| GR 40 (TC 31) | KY 12 (TC 56) | RG 17 |
| GR 42 (TC 32) | KY 14 (TC 57) | TKF 1801 LC |
| GR 42C (TC 33) | KY 14 × L8 LC | TKF 2002 LC |
| GR 43 (TC 34) | KY 15 (TC 58) | TKF 4024 LC |
| GR 44 (TC 35) | KY 16 (TC 59) | TKF 4028 LC |
| GR 45 (TC 36) | KY 17 (TC 60) | TKF 6400 LC |
| GR 46 (TC 37) | KY 19 (TC 61) | TKF 7002 LC |
| GR 48 (TC 38) | KY 21 (TC 62) | TKS 2002 LC |
| GR 5 (TC 16) | KY 22 (TC 63) | TN 86 (TC 82) |
| GR 53 (TC 39) | KY 24 (TC 64) | TN 90 LC |
| GR 6 (TC 17) | KY 26 (TC 65) | TN 97 Hybrid LC |
| GR 9 (TC 18) | KY 33 (TC 66) | TN 97 LC |
| GR139 NS | KY 34 (TC 67) | VA 116 |
| GR139 S | KY 35 (TC 68) | VA 119 |
| HB 04P | KY 41A (TC 69) | Virgin A Mutante (TI 1406) |
| HB 04P LC | KY 5 (TC 53) | Virginia 509 (TC 84) |
| HB 3307P LC | KY 52 (TC 70) | |

TABLE 18
Maryland Tobacco Varieties
Maryland 10 (TC 498)
Maryland 14 D2 (TC 499)
Maryland 201 (TC 503)
Maryland 21 (TC 500)
Maryland 341 (TC 504)
Maryland 40
Maryland 402
Maryland 59 (TC 501)
Maryland 601
Maryland 609 (TC 505)
Maryland 64 (TC 502)
Maryland 872 (TC 506)
Maryland Mammoth (TC 507)

TABLE 19
Dark Fire-Cured Tobacco Varieties

| | | |
|---|---|---|
| Black Mammoth (TC 461) | KY 171 (TC 475) | PD 7309 LC |
| Black Mammoth Small Stalk (TC 641) | KY 171 LC | PD 7312 LC |
| Certified Madole (TC 463) | KY 171 NS | PD 7318 LC |
| D-534-A-1 (TC 464) | KY 180 (TC 573) | PD 7319 LC |
| DAC ULT 302 | KY 190 (TC 574) | Petico M PG04 |
| DAC ULT 303 | Little Crittenden | PYKY 160 (TC612) |
| DAC ULT 306 | Little Crittenden (TC 476) | PYKY 171 (TC 613) |
| DAC ULT 308 | Little Crittenden LC (certified) | Shirey |
| DAC ULT 312 | Little Crittenden PhPh | TI 1372 |
| DF 300 (TC 465) | Lizard Tail Turtle Foot | TN D94 |
| DF 485 (TC 466) | Madole (TC 478) | TN D94 (TC 621) |
| DF 516(TC 467) | Madole (TC 479) | TN D950 |
| DF 911 (TC 468) | MS KY 171 | TN D950 (PhPh) |
| DT 508 | MS NL Madole LC | TN D950 |
| DT 518 (Screened) | MS TN D950 LC | TN D950 (TC 622) |
| DT 538 LC | Nance (TC 616) | TR Madole (TC 486) |
| DT 592 | Narrow Leaf Madole LC (certified) | VA 309 |
| Improved Madole (TC 471) | Neal Smith Madole (TC 646) | VA 309 (TC 560) |
| Jernigan's Madole (TC 472) | Newtons VH Madole | VA 309 LC (certified) |
| KT 14 LC | NL Madole | VA 310 (TC 487) |
| KT D17LC | NL Madole (PhPh) | VA 331 (TC 592) |
| KT D4 LC | NL Madole (TC 484) | VA 355 (TC 638) |
| KT D6 LC | NL Madole LC | VA 359 |
| KT D8 LC | NL Madole LC (PhPh) | VA 359 (Screened) |
| KY 153 (TC 216) | NL Madole NS | VA 359 (TC 639) |
| KY 157 (TC 217) | One Sucker (TC 224) | VA 359 LC (certified) |
| KY 160 | OS 400 | VA 403 (TC 580) |
| KY 160 (TC 218) | PD 302H | VA 405 (TC581) |
| KY 163 (TC 219) | PD 312H | VA 409 (TC 562) |
| KY 165 (TC 220) | PD 318H | VA 510 (TC 572) |
| KY 170 (TC 474) | PD 7302 LC | |
| KY 171 (PhPh) | PD 7305 | |

TABLE 20
Oriental Tobacco Varieties.

| | | |
|---|---|---|
| Bafra (TI 1641) | Edirne (TI 1671) | Samsun (TC 536) |
| Bahce (TI 1730) | Ege (TI 1642) | Samsun 959 (TI 1570) |
| Bahia (TI 1416) | Ege-64 (TI 1672) | Samsun Evkaf (TI 1723) |
| Bahia (TI 1455) | Izmir (Akhisar) (TI 1729) | Samsun Holmes NN (TC 540) |
| Baiano (TI 128) | Izmir (Gavurkoy) (TI 1727) | Samsun Maden (TI 1647) |
| Basma | Izmir Ege 64 | Samsun NO 15 (TC 541) |
| Basma (TI 1666) | Izmir-Incekara (TI 1674) | Samsun-BLK SHK Tol (TC 542) |
| Basma Drama | Izmir-Ozbas (TI 1675) | Samsun-Canik (TI 1678) |
| Basma Hybrid (PhPh) | Jaka Dzebel (TI 1326) | Samsun-Maden (TI 1679) |
| Basma Zihna 1 | Kaba-Kulak | Saribaptar 407-Izmir Region |
| Bitlis (TI 1667) | Kagoshima Maruba (TI 158) | Smyrna (TC 543) |
| Bitlis (TI 1725) | Katerini | Smyrna No. 23 (TC 545) |
| Bubalovac (TI 1282) | Katerini S53 | Smyrna No. 9 (TC 544) |
| Bursa (TI 1650) | Krumovgrad 58 | Smyrna-Blk Shk Tol (TC 546) |
| Bursa (TI 1668) | MS Basma | Trabzon (TI 1649) |
| Canik (TI 1644) | MS Katerini S53 | Trabzon (TI 1682) |
| Djebel 174 (TI 1492) | Nevrokop 1146 | Trapezund 161 (TI 1407) |
| Djebel 359 (TI 1493) | Ozbas (TI 1645) | Turkish (TC 548) |
| Djebel 81 | Perustitza (TI 980) | Turkish Angshit (TI 90) |
| Dubec 566 (TI 1409) | Prilep (TI 1291) | Turkish Samsum (TI 92) |
| Dubec 7 (TI 1410) | Prilep (TI 1325) | Turkish Tropizoid (TI 93) |
| Dubek 566 (TI 1567) | Prilep 12-2/1 | Turkish Varotie (TI 89) |
| Duzce (TI 1670) | Prilep 23 | Xanthi (TI 1662) |

TABLE 21
Cigar Tobacco Varieties

| | | |
|---|---|---|
| Bahai (TI 62) | Castillo Negro, Blanco, Pina (TI 449) | Enshu (TI 1586) |
| Beinhart 1000 | Caujaro (TI 893) | Florida 301 |
| Beinhart 1000 (TI 1562) | Chocoa (TI 289) | Florida 301 (TC 195) |
| Beinhart 1000-1 (TI 1561) | Chocoa (TI313) | PA Broadleaf (TC 119) |
| Bergerac C | Connecticut 15 (TC 183) | Pennsylvania Broadleaf |
| Bergerac C (TI 1529) | Connecticut Broadleaf | Pennsylvania Broadleaf (TC 119) |
| Big Cuban (TI 1565) | Connecticut Broadleaf (TC 186) | Petite Havana SR1 |
| Castillo Negro, Blanco, Pina (TI 448) | Connecticut Shade (TC 188) | Petite Havana SR1 (TC 105) |
| Castillo Negro, Blanco, Pina (TI 448A) | Criollo, Colorado (TI 1093) | Enshu (TI 1586) |

TABLE 22
Other Tobacco Varieties

| | |
|---|---|
| Chocoa | (TI 319) |
| Hoja Parada | (TI 1089) |
| HojaParado | (Galpoa) (TI 1068) |
| Perique | (St. James Parrish) |
| Perique | (TC 556) |
| Perique | (TI 1374) |
| Sylvestris | (TI 984) |

TI 179

1.-45. (canceled)

46. A tobacco plant, or part thereof, comprising:
(a) a first knockout mutant allele in a putrescine N-methyltransferase (PMT) 1a (PMT1a) gene, wherein a wildtype allele of the PMT1a gene encodes the polypeptide of SEQ ID NO: 12 and wherein the first knockout mutant allele comprises the nucleic acid sequence of SEQ ID NO: 475; and
(b) a second knockout mutant allele in a PMT1b gene, wherein a wildtype allele of the PMT1b gene encodes the polypeptide of SEQ ID NO: 11 and wherein the second knockout mutant allele comprises the nucleic acid sequence of SEQ ID NO: 410 or SEQ ID NO: 415; and
(c) a third knockout mutant allele in a PMT2 gene, wherein a wildtype allele of the PMT2 gene encodes the polypeptide of SEQ ID NO: 13 and wherein the third knockout mutant allele comprises the nucleic acid sequence of SEQ ID NO: 128; and
(d) a fourth knockout mutant allele in a PMT3 gene, wherein a wildtype allele of the PMT3 gene encodes the polypeptide of SEQ ID NO: 14 and wherein the fourth knockout mutant allele comprises the nucleic acid sequence of SEQ ID NO: 138 or SEQ ID NO: 602; and
(e) a fifth knockout mutant allele in a PMT4 gene, wherein a wildtype allele of the PMT4 gene encodes the polypeptide of SEQ ID NO: 15 and wherein the fifth knockout mutant allele comprises the nucleic acid sequence of SEQ ID NO: 182 or SEQ ID NO: 666,
wherein said tobacco plant produces a dried leaf comprising: (i) an anatabine level greater than the anatabine level of a dried leaf from a control tobacco plant; (ii) an anabasine level greater than the anabasine level of a dried leaf from the control tobacco plant, or (iii) an anatabine level greater than the anatabine level of a dried leaf from the control tobacco plant and an anabasine level greater than the anabasine level of a dried leaf from a control tobacco plant, wherein the control tobacco plant does not comprise the first, second, third, fourth, and fifth knockout mutant alleles.

47. The tobacco plant or part thereof of claim 46, wherein the tobacco plant or part thereof comprises a sixth knockout allele in the PMT1b gene, wherein the sixth knockout mutant allele comprises the nucleic acid sequence of SEQ ID NO: 410; and wherein the second knockout mutant allele comprises the nucleic acid sequence of SEQ ID NO: 415.

48. The tobacco plant or part thereof of claim 46, wherein the tobacco plant or part thereof comprises a sixth knockout allele in the PMT3 gene, wherein the sixth knockout mutant allele comprises the nucleic acid sequence of SEQ ID NO: 138; and wherein the fourth knockout mutant allele comprises the nucleic acid sequence of SEQ ID NO: 602.

49. The tobacco plant or part thereof of claim 46, wherein the tobacco plant or part thereof comprises a sixth knockout allele in the PMT4 gene, wherein the sixth knockout mutant allele comprises the nucleic acid sequence of SEQ ID NO: 182; and wherein the fifth knockout mutant allele comprises the nucleic acid sequence of SEQ ID NO: 666.

50. The tobacco plant or part thereof of claim 46, wherein
(i) the tobacco plant or part thereof comprises a sixth knockout allele in the PMT1b gene, wherein the sixth knockout mutant allele comprises the nucleic acid sequence of SEQ ID NO: 410; and wherein the second knockout mutant allele comprises the nucleic acid sequence of SEQ ID NO: 415;
(ii) the tobacco plant or part thereof comprises a seventh knockout allele in the PMT3 gene, wherein the seventh knockout mutant allele comprises the nucleic acid sequence of SEQ ID NO: 138; and wherein the fourth knockout mutant allele comprises the nucleic acid sequence of SEQ ID NO: 602; and
(iii) wherein the tobacco plant or part thereof comprises an eighth knockout allele in the PMT4 gene, wherein the eighth knockout mutant allele comprises the nucleic acid sequence of SEQ ID NO: 182; and wherein the fifth knockout mutant allele comprises the nucleic acid sequence of SEQ ID NO: 666

51. The tobacco plant, or part thereof, of claim 46, wherein said tobacco plant produces a dried leaf comprising an anatabine level at least 1% greater than the anatabine level of a dried leaf from the control tobacco plant.

52. The tobacco plant, or part thereof, of claim 46, wherein said tobacco plant produces a dried leaf comprising an anabasine level at least 1% greater than the anabasine level of a dried leaf from the control tobacco plant.

53. The tobacco plant, or part thereof, of claim 46, wherein said tobacco plant produces a cured leaf comprising a total tobacco-specific nitrosamine level of between 2 parts per million (ppm) and 0.05 ppm.

54. The tobacco plant, or part thereof, of claim 46, wherein leaves of the tobacco plant, when cured, have a USDA grade index value 50 or more.

55. Cured tobacco material from the tobacco plant of claim 46.

56. The cured tobacco material of claim 55, wherein said cured tobacco material is made by a curing process selected from the group consisting of flue curing, air curing, fire curing, and sun curing.

57. The cured tobacco material of claim 55, wherein said cured tobacco material comprises tobacco leaf, and wherein said tobacco leaf exhibits reduced mold infection as compared to control cured tobacco material from the variety LA Burley 21.

58. A tobacco blend comprising said cured tobacco material of claim 55.

59. The tobacco blend of claim 58, wherein said cured tobacco material constitutes at least 10% of cured tobacco in said tobacco blend by weight.

60. The tobacco blend of claim 58, wherein said cured tobacco material constitutes at least 10% of cured tobacco in said tobacco blend by volume.

61. A tobacco product comprising the cured tobacco material of claim 55.

62. The tobacco product of claim 61, wherein said tobacco product is selected from the group consisting of a cigarette, a cigarillo, a non-ventilated recess filter cigarette, a vented recess filter cigarette, a cigar, snuff, pipe tobacco, cigar tobacco, cigarette tobacco, chewing tobacco, leaf tobacco, shredded tobacco, and cut tobacco.

63. The tobacco product of claim 61, wherein said tobacco product is a smokeless tobacco product.

64. The tobacco product of claim 63, wherein said smokeless tobacco product is selected from the group consisting of loose leaf chewing tobacco, plug chewing tobacco, moist snuff, and nasal snuff.

65. A reconstituted tobacco comprising the cured tobacco material of claim 55.
